(19) United States
(12) Patent Application Publication
(10) Pub. No.: US 2005/0181988 A1
(43) Pub. Date: Aug. 18, 2005

(54) Echinocandin cyclic peptide derivatives

(76) Inventors: Hiroaki Mizuno, Osaka (JP); Takahiro Matsuya, Chuo-ku Osaka (JP); Hiroshi Matsuda, Osaka (JP); David Barrett, Chuo-ku Osaka-shi Osaka (JP); Ayako Toda, Chuo-ku (JP); Keiji Matsuda, Chuo-ku Osaka-shi Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/502,646

(22) PCT Filed: Feb. 4, 2003
(86) PCT No.: PCT/JP03/01107

(30) Foreign Application Priority Data
Feb. 11, 2002 (AU) PS0441

(51) Int. Cl. A61K 38/12; C07K 7/64

(52) U.S. Cl. 514/9; 530/317

(57) ABSTRACT

This invention relates to new lipopeptide compound represented by the following general formula (I) wherein R¹, R², R³, ^{R4} and R⁵ are as defined in the description or a salt thereof which has antimicrobial activities (especially, antifungal activities), inhibitory activity on β-1,3-glucan synthase, to process for preparation thereof, to a pharmaceutical composition comprising the same, and to a method for prophylactic and/or thereapeutic treatment of infectious diseases including Pneumocystis carinii infection (e.g. Pneumocystis carinii pneumonia) in a human being or an animal. In particular, R⁴ is an alkyl moiety which is substituted with an eventually protected and/or further substituted selected from amino, carboxy, guanidino, heterocyclic-carbonyl, alkyl-carbamoyl.

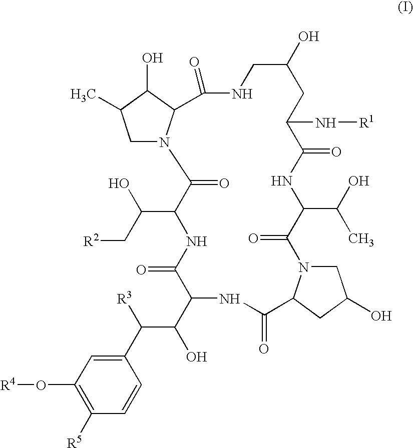

### TECHNICAL FIELD

The present invention relates to new lipopeptide compound and a salt thereof which are useful as a medicament.

### BACKGROUND ART

In U.S. Pat. Nos. 5,376,634, 5,569,646, WO 96/11210 and WO 99/40108, there are disclosed the lipopeptide compound and a pharmaceutically acceptable salt thereof, which have antimicrobial activities (especially antifungal activity).

### DISCLOSURE OF INVENTION

The present invention relates to new lipopeptide compound and a salt thereof.

More particularly, it relates to new lipopeptide compound and a salt thereof, which have antimicrobial activities [especially, antifungal activities, in which the fungi may include Aspergillus, Cryptococcus, Candida, Mucor, Actinomyces, Histoplasma, Dermatophyte, Malassezia, Fusarium and the like.], inhibitory activity on β-1,3-glucan synthase, and further which are expected to be useful for the prophylactic and/or therapeutic treatment of Pneumocystis carinii infection (e.g. Pneumocystis carinii pneumonia) in a human being or an animal, to a process for preparation thereof, to a pharmaceutical composition comprising the same, and to a method for the prophylactic and/or therapeutic treatment of infectious disease including Pneumocystis carinii infection (e.g. Pneumocystis carinii pneumonia) in a human being or an animal.

The object lipopeptide compound of the present invention is new and can be represented by the following general formula (I): 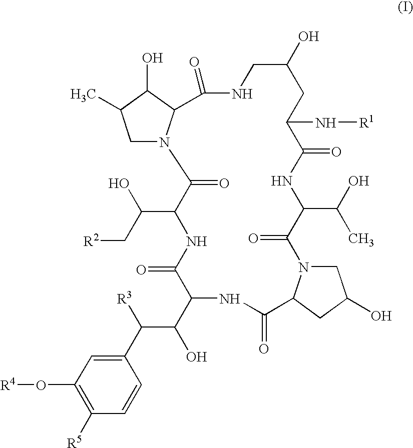
wherein
R¹ is hydrogen or acyl group,
R² is carbamoyl, protected amino(lower)alkyl, amino(lower)alkyl, protected guanidino(lower)alkyl, guanidino(lower)alkyl or lower alkylamino(lower)alkyl substituted with one or more hydroxy,
R³ is hydrogen or hydroxy,
R⁴ is protected amino(lower)alkyl, amino(lower)alkyl, heterocycliccarbonyl(lower)alkyl, lower alkylcarbamoyl(lower)alkyl, protected carboxy(lower)alkyl, carboxy (lower) alkyl, protected guanidino(lower)alkyl, guanidino(lower)alkyl, di-lower alkylamino(lower)alkyl, lower alkylamino(lower)alkyl optionally substituted with one or more suitable substituent(S) selected from the group consisting of hydroxy and lower alkoxy, amino(lower)alkanoylamino(lower)alkyl or hydroxy(lower)alkyl, and
R⁵ is protected hydroxy or hydroxy,
   or a salt thereof.

The new lipopeptide compound (I) or a salt thereof can be prepared by the process as illustrated in the following reaction schemes.

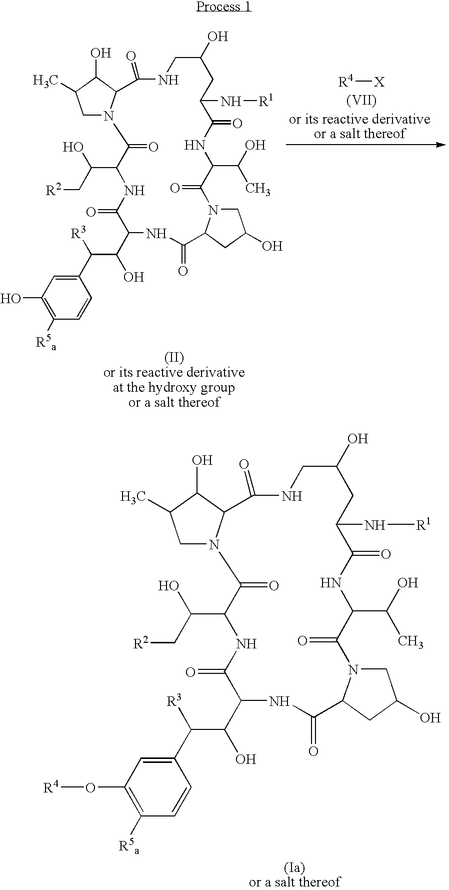

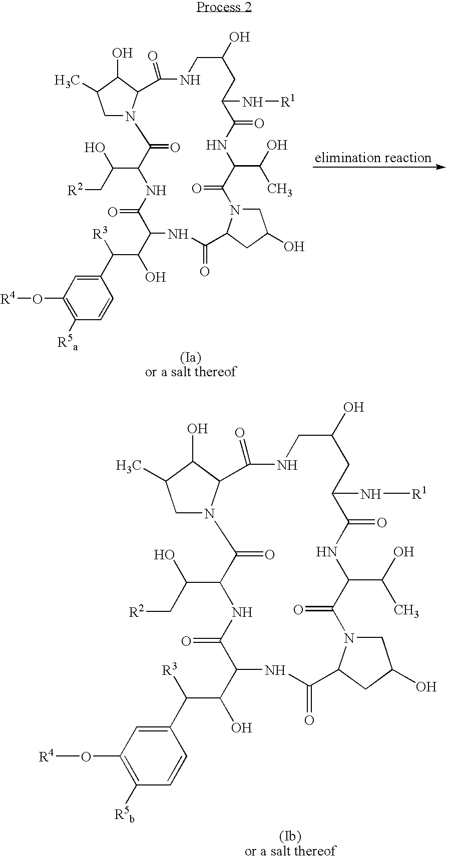

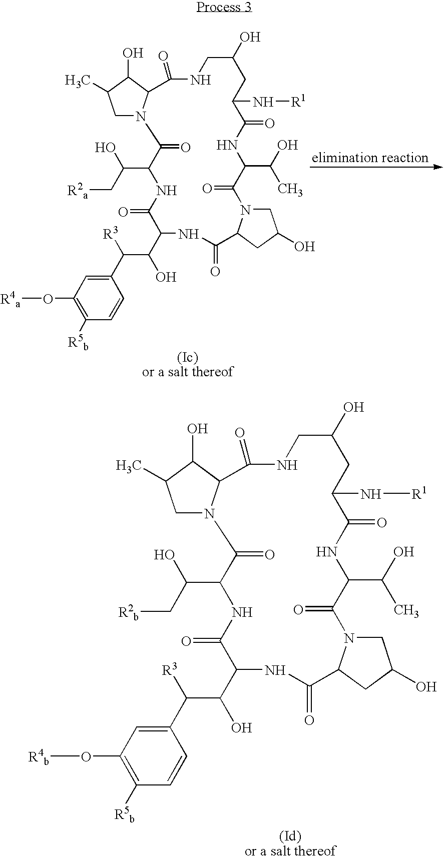

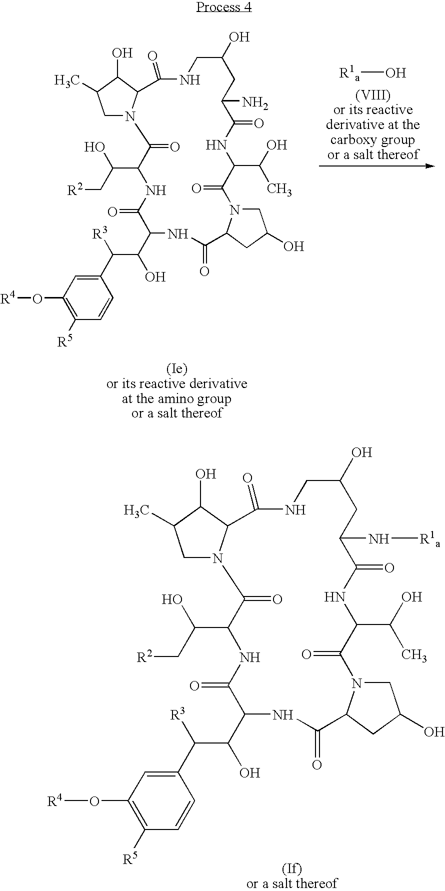

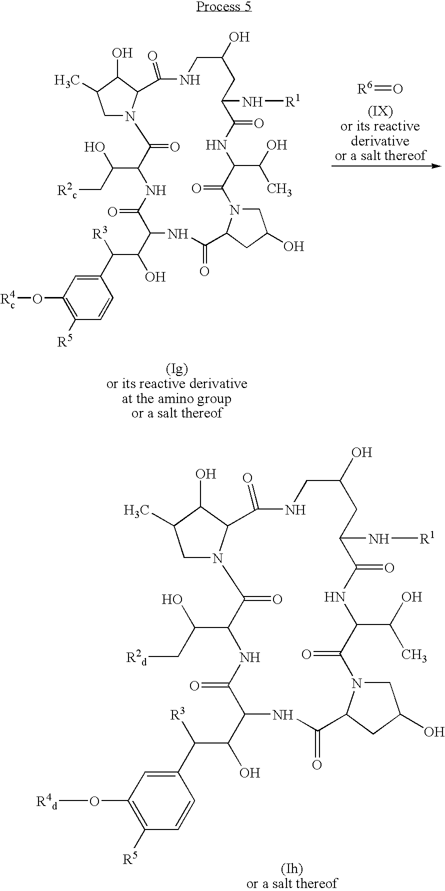

The starting compound (II) or a salt thereof can be prepared by the process as illustrated in the following reaction schemes. 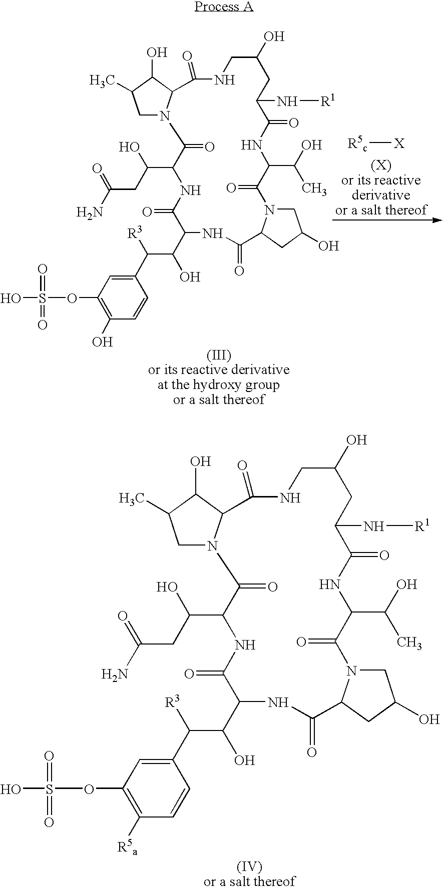 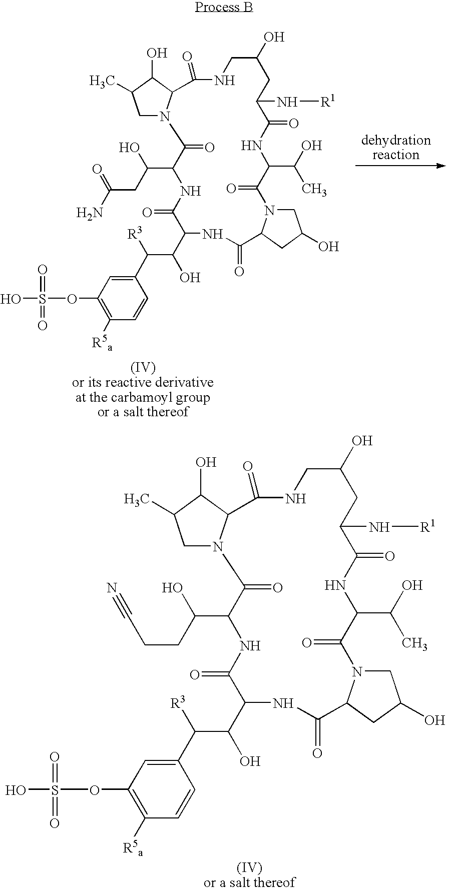 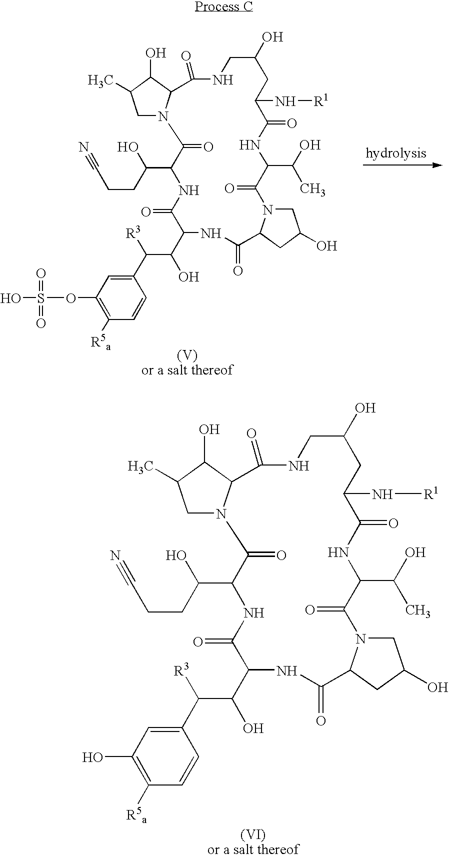 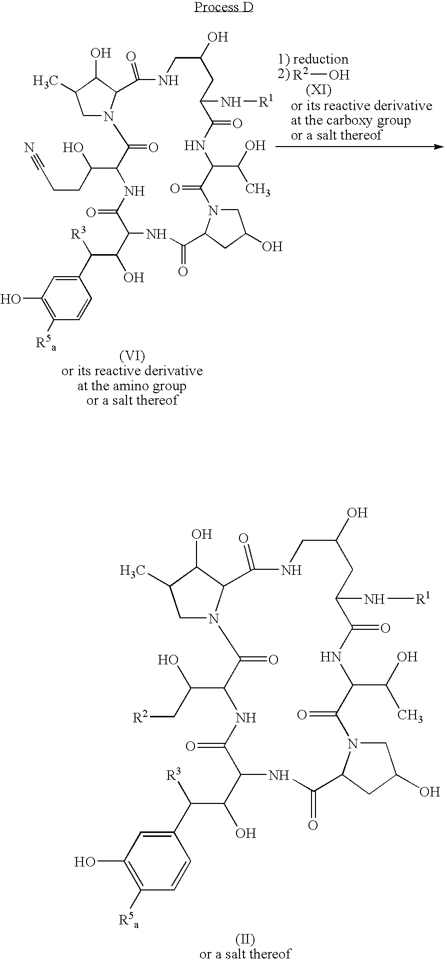
wherein
R¹, R², R³, R⁴ and R⁵ are defined above,
Rₐ¹ is acyl group,
Rₐ² is protected amino(lower)alkyl or protected guanidino(lower)alkyl,
R_{b}² is amino(lower)alkyl or guanidino(lower)alkyl,
R_{c}² is amino(lower)alkyl,
R_{d}² is lower alkylamino(lower)alkyl substituted with one or more hydroxy,
Rₐ⁴ is protected amino(lower)alkyl or protected guanidino(lower)alkyl,
R_{b}⁴ is amino(lower)alkyl or guanidino(lower)alkyl,
R_{c}⁴ is amino(lower)alkyl,
R_{d}⁴ is lower alkylamino(lower)alkyl substituted with one or more hydroxy,
Rₐ⁵ is protected hydroxy,
R_{b}⁵ is hydroxy
R_{c}⁵ is hydroxy protective group, and
X is halogen.

Suitable salt of the new lipopeptide compound (I) is a pharmaceutically acceptable and conventional non-toxic salt, and may include a salt with a base or an acid addition salt such as a salt with an inorganic base, for example, an alkali metal salt (e.g., sodium salt, potassium salt, etc.), an alkaline earth metal salt (e.g., calcium salt, magnesium salt, etc.), an ammonium salt;
a salt with an organic base, for example, an organic amine salt (e.g., triethylamine salt, diisopropylethylamine salt, pyridine salt, picoline salt, ethanolamine salt, triethanolamine salt, dicyclohexylamine salt, N,N′-dibenzylethylenediamine salt, 4-dimethylaminopyridine salt, etc.);
an inorganic acid addition salt (e.g., hydrochloride hydrobromide, sulfate, phosphate, etc.);
an organic carboxylic sulfonic acid addition salt (e.g., formate, acetate, trifluoroacetate, maleate, tartrate, fumarate, methanesulfonate, benzenesulfonate, toluenesulfonate, etc.);
a salt with a basic or acidic amino acid (e.g., arginine, aspartic acid, glutamic acid, etc.).

Suitable examples and illustration of the various definitions in the above and subsequent descriptions of the present specification, which the present invention intends to include within the scope thereof, are explained in detail as follows:

The term “lower” is used to intend a group having 1 to 6 carbon atom(s), unless otherwise provided.

Suitable example of “one or more” may be the number of 1 to 6, in which the preferred one may be the number of 1 to 3, and the most preferred one may be the number of 1 Or 2.

Suitable example of “halogen” may be fluorine, chlorine, bromine, iodine and the like.

Suitable example of “lower alkoxy” may include straight or branched one such as methoxy, ethoxy, propoxy, isopropoxy, butoxy, isobutoxy, tert-butoxy, pentyloxy, tert-pentyloxy, neo-pentyloxy, hexyloxy, isohexyloxy and the like.

Suitable example of “higher alkoxy” may include straight or branched one such as heptyloxy, octyloxy, 3,5-dimethyloctyloxy, 3,7-dimethyloctyloxy, nonyloxy, decyloxy, undecyloxy, dodecyloxy, tridecyloxy, tetradecyloxy, hexadecyloxy, heptadecyloxy, octadecyloxy, nonadecyloxy, icosyloxy, and the like.

Suitable example of “lower alkyl” may include straight or branched one having 1 to 6 carbon atom(s), such as methyl, ethyl, propyl, isopropyl, butyl, isobutyl, sec-butyl, tert-butyl, pentyl, tert-pentyl, neo-pentyl, hexyl, isohexyl and the like.

Suitable example of “higher alkyl” may include straight or branched one such as heptyl, octyl, 3,5-dimethyloctyl, 3,7-dimethyloctyl, nonyl, decyl, undecyl, dodecyl, tridecyl, tetradecyl, hexadecyl, heptadecyl, octadecyl, nonadecyl, icosyl, and the like.

Suitable example of “lower alkenyl” may include straight or branched one having 1 to 6 carbon atom(s), such as methylene, vinyl, propenyl, isopropenyl, butenyl, isobutenyl, sec-butenyl, tert-butenyl, pentenyl, tert-pentenyl, neo-pentenyl, hexenyl, isohexyl, and the like.

Suitable example of “aryl” and “ar” moiety may include phenyl which may have lower alkyl (e.g., phenyl, mesityl, xylyl, tolyl, etc.), naphthyl, anthryl, indanyl, fluorenyl, and the like, and this “aryl” and “ar” moiety may have one or more halogen.

Suitable example of “aroyl” may include benzoyl, toluoyl, naphthoyl, anthrylcarbonyl, and the like.

Suitable example of “heterocyclic group” may include
unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), etc.;
saturated 3 to 8-membered (more preferably 5 or 6-membered) heteromonocyclic group containing 1 to 4 nitrogen atom(s), for example, pyrrolidinyl, imidazolidinyl, piperidyl, piperazinyl, azetidinyl, etc.;
unsaturated condensed heterocyclic group containing 1 to 4 nitrogen atom(s), for example, indolyl, isoindolyl, indolinyl, indolizinyl, benzimidazolyl, quinolyl, isoquinolyl, indazolyl, benzotriazolyl, tetahydroquinolyl, etc.;
unsaturated 3 to 8-membered (more preferably 5 or 6-membered) heteromonocyclic group containing 1 or 2 oxygen atom(s) and 1 to 3 nitrogen atom(s), for example, oxazolyl, isoxazolyl, oxadiazolyl (e.g., 1,2,4-oxadiazolyl, 1,3,4-oxadiazolyl, 1,2,5-oxadiazolyl, etc.), etc.;
saturated 3 to 8-membered (more preferably 5 or 6-membered) heteromonocyclic group containing 1 or 2 oxygen atom(s) and 1 to 3 nitrogen atom(s), for example, morpholinyl, sydnonyl morpholino, etc.;
unsaturated condensed heterocyclic group containing 1 or 2 oxygen atom(s) and 1 to 3 nitrogen atom(s), for example, benzoxazolyl, benzoxadiazolyl, etc.;
unsaturated 3 to 8-membered (more preferably 5 or 6-membered) heteromonocyclic group containing 1 or 2 sulfur atom(s) and 1 to 3 nitrogen atom(s), for example, thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.), dihydrothiazinyl, etc.;
saturated 3 to 8-membered (more preferably 5 or 6-membered) heteromonocyclic group containing 1 or 2 sulfur atom(s) and 1 to 3 nitrogen atom(s), for example, thiazolidinyl, thiomorpholinyl, thiomorpholino, etc.;
unsaturated 3 to 8-membered (more preferable 5 or 6-membered) heteromonocyclic group containing 1 or 2 sulfur atom(s), for example, thienyl, dihydrodithiinyl, dihydrodithionyl, etc.;
unsaturated condensed heterocyclic group containing 1 or 2 sulfur atom(s) and 1 to 3 nitrogen atom(s), for example, benzothiazolyl, benzothiadiazolyl, imidazothiadiazolyl, etc.;
unsaturated 3 to 8-membered (more preferably 5 or 6-membered) heteromonocyclic group containing an oxygen atom, for example, furyl, etc.;
saturated 3 to 8-membered (more preferably 5 or 6membered) heteromonocyclic group containing 1 or 2 oxygen atom(s), for example, tetrahydrofuran, tetrahydropyran, dioxacyclopentane, dioxacyclohexane, etc.;
unsaturated 3 to 8-membered (more preferably 5 or 6-membered) heteromonocyclic group containing an oxygen atom and 1 or 2 sulfur atom(s), for example, dihydrooxathiinyl, etc.;
unsaturated condensed heterocyclic group containing 1 or 2 sulfur atom(s), for example, benzothienyl, benzodithiinyl, etc.;
unsaturated condensed heterocyclic group containing an oxygen atom and 1 or 2 sulfur atom(s), for example, benzoxathiinyl, etc.; and the like, and this “heterocyclic group” may have one or more suitable substituent(s) selected from the group consisting of lower alkyl, oxo, cyclo(lower)alkyl, hydroxy(lower)alkyl, carboxy(lower)alkanoyl which may have amino and heterocycliccarbonyl.

Suitable example of “cyclo(lower)alkyl” may include cyclopropyl, cyclobutyl, cyclopentyl, cyclohexyl, and the like, and this “cyclo(lower)alkyl” may have one or more lower alkyl.

Suitable example of “cyclo(lower)alkyloxy” may include cyclopropyloxy, cyclobutyloxy, cyclopentyloxy, cyclohexyloxy, and the like.

Suitable example of “cyclo(lower)alkenyloxy” may include cyclopropenyloxy, cyclobutenyloxy, cyclopentenyloxy, cyclohexenyloxy, and the like.

Suitable example of “phenyl(lower)alkyl” may include benzyl, phenethyl, phenylpropyl, phenylbutyl, and the like.

Suitable example of “cyclo(lower)alkyl(lower)alkyloxy” may include cyclopropylmethoxy, cyclobutylethoxy, cyclopentylpropoxy, cyclohexylmethoxy, and the like.

Suitable example of “cyclo(higher)alkyl” may include cycloheptyl, cyclooctyl, cyclononyl, cyclodecyl, and the like.

Suitable example of “acyl group” may include aliphatic acyl, aromatic acyl, arylaliphatic acyl and heterocyclic-aliphatic acyl derived from carboxylic acid, carbonic acid, carbamic acid, sulfonic acid, and the like.

Suitable example of said “acyl group” may be illustrated as follows.

carboxy; carbamoyl; mono or di(lower)alkylcarbamoyl (e.g., methylcarbamoyl, dimethylcarbamoyl, ethylcarbamoyl, diethylcarbamoyl, etc.)

Aliphatic acyl such as lower or higher alkanoyl (e.g., formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, etc.);
lower or higher alkoxycarbonyl (e.g., methoxycarbonyl, ethoxycarbonyl, t-butoxycarbonyl, t-pentyloxycarbonyl, heptyloxycarbonyl, etc.); lower alkenyloxycarbonyl (e.g., vinyloxycarbonyl, propenyloxycarbonyl, allyloxycarbonyl, butenyloxycarbonyl, butedienyloxycarbonyl, pentenyloxycarbonyl, hexenyloxycarbonyl, etc.);
lower or higher alkylsulfonyl (e.g., methylsulfonyl, ethylsulfonyl, etc.);
lower or higher alkoxysulfonyl (e.g., methoxysulfonyl, ethoxysulfonyl, etc.); or the like;
Aromatic acyl such as aroyl (e.g., benzoyl, toluoyl, naphthoyl, etc.); ar(lower)alkanoyl [e.g., phenyl(C₁-C₆)alkanoyl (e.g., phenylacetyl, phenylpropanoyl, phenylbutanoyl, phenylisobutanoyl, phenylpentanoyl, phenylhexanoyl, etc.), naphthyl(C₁-C₆)alkanoyl (e.g., naphthylacetyl, naphthylpropanoyl, naphthylbutanoyl, etc.), etc.];
ar(lower)alkenoyl [e.g., phenyl(C₃-C₆)alkenoyl (e.g., phenylpropenoyl, phenylbutenoyl, phenylmethacryloyl, phenylpentanoyl, phenylhexanoyl, etc.), naphthyl(C₃-C₆)alkenoyl (e.g., naphthylpropenoyl, naphthylbutenoyl, etc.), etc.];
ar(lower)alkoxycarbonyl [e.g., phenyl(C₁-C₆)alkoxycarbonyl (e.g., benzyloxycarbonyl, etc.), fluorenyl(C₁-C₆)alkoxy-carbonyl (e.g., fluorenylmethyloxycarbonyl, etc.), etc.]; aryloxycarbonyl (e.g., phenoxycarbonyl, naphthyloxycarbonyl, etc.);
aryloxy(lower)alkanoyl (e.g., phenoxyacetyl, phenoxypropionyl, etc.);
arylcarbamoyl (e.g., phenylcarbamoyl, etc.);
arylthiocarbamoyl (e.g., phenylthiocarbamoyl, etc.);
arylglyoxyloyl (e.g., phenylglyoxyloyl, naphthylglyoxyloyl, etc.);
arylsulfonyl which may have 1 to 4 lower alkyl (e.g., phenylsulfonyl, p-tolylsulfonyl, etc.);
aroyl (e.g., benzoyl) substituted with one or more suitable substituent(s); or the like;
Heterocyclic acyl such as heterocycliccarbonyl;
heterocyclic(lower)alkanoyl (e.g., heterocyclicacetyl, heterocyclicpropanoyl, heterocyclicbutanoyl, heterocyclicpentanoyl, heterocyclichexanoyl, etc.);
heterocyclic(lower)alkenoyl (e.g., heterocyclicpropenoyl, heterocyclicbutenoyl, heterocyclicpentenoyl, heterocyclichexenoyl, etc.);
heterocyclicglyoxyloyl; or the like;
in which suitable “heterocyclic” moiety in the terms “heterocycliccarbonyl”, “heterocyclic(lower)alkanoyl”, “heterocyclic(lower)alkenoyl” and “heterocyclicglyoxyloyl” can be referred to aforementioned “heterocyclic” moiety.

Suitable example of “acyl group” of R¹ can be referred to aforementioned “acyl group”, in which the preferred one may be lower alkoxycarbonyl, higher alkanoyl, ar(lower)alkoxycarbonyl, benzoyl substituted with one or more suitable substituent(s), naphthoyl substituted with one or more suitable substituent(s) and heterocycliccarbonyl substituted with one or more suitable substituent(s).

Suitable example of “suitable substituent(s)” in the term of “benzoyl substituted with one or more suitable substituent(s)” and “naphthoyl substituted with one or more suitable substituent(s)” may be
thiadiazolyl substituted with phenyl substituted with piperidyl substituted with one or more suitable substituent(s) selected from the group consisting of lower alkoxy, chlorophenyl, lower alkoxy(lower)alkoxy(lower)alkyl, cyclo(lower)alkyloxy, cyclo(lower)alkyl, phenyl(lower)alkyloxy, phenyl, lower alkoxy(lower)alkoxy, lower alkoxy(higher)alkoxy, cyclo(lower)alkenyloxy, lower alkoxy(lower)alkyl substituted with phenyl and cyclo(lower)alkyl(lower)alkyloxy,
thiadiazolyl substituted with phenyl substituted with phenyl substituted with one or more suitable substituent(s) selected from the group consisting of lower alkoxy(lower)alkoxy, cyclo(lower)alkyloxy, lower alkoxy(lower)alkoxy(lower)alkyl, lower alkoxy(lower)alkyl and lower alkoxy,
thiadiazolyl substituted with phenyl substituted with higher alkoxy substituted with lower alkoxy,
thiadiazolyl substituted with phenyl substituted with piperazinyl substituted with one or more suitable substituent(s) selected from the group consisting of cyclo(lower)alkyl substituted with lower alkyl, phenyl(lower)alkyl substituted with heterocyclic group, cyclo(lower)alkyl(lower)alkyl, cyclo(lower)alkyl, cyclo(higher)alkyl, lower alkoxy(higher)alkyl, heterocyclic group and cyclo(lower)alkyl substituted with lower alkylidene,
thiadiazolyl substituted with pyridyl substituted with piperazinyl substituted with cyclo(lower)alkyl substituted with lower alkyl,
thiadiazolyl substituted with phenyl substituted with lower alkoxy(lower)alkoxy,
thiadiazolyl substituted with phenyl substituted with lower alkoxy(higher)alkoxy,
imidazothiadiazolyl substituted with phenyl substituted with piperidyl substituted with lower alkoxy(lower)alkoxy,
thiazolyl substituted with phenyl substituted with lower alkoxy,
phenyl substituted with piperazinyl substituted with cyclo(lower)alkyl optionally substituted with one or more suitable substituent(s) selected from the group consisting of cyclo(lower)alkyl, lower alkoxy, lower alkyl and phenyl,
thiadiazolyl substituted with phenyl substituted with heterocyclicoxy substituted with phenyl,
imidazothiadiazolyl substituted with phenyl substituted with lower alkoxy,
phenyl substituted with one or more suitable substituent(s) selected from the group consisting of lower alkoxy(higher)alkoxy, tetrahydroquinolyl optionally substituted with one or more lower alkoxy, piperidyl substituted with cyclo(lower)alkyloxy and phenyl substituted with morpholino,
lower alkoxy(higher)alkoxy,
higher alkoxy,
piperazinyl substituted with phenyl substituted with one or more suitable substituent(s) selected from the group consisting of heterocyclic group substituted with lower alkoxy and phenyl, lower alkoxy(lower)alkoxy, heterocyclic group substituted with tolyl and phenyl substituted with lower alkoxy(lower)alkoxy,
piperazinyl substituted with cyclo(lower)alkyl substituted with one or more suitable substituent(s) selected from the group consisting of cyclo(lower)alkyl and lower alkyl,
phenyl substituted with piperazinyl substituted with phenyl substituted with one or more suitable substituent(s) selected from the group consisting of heterocyclic group substituted with lower alkyl and lower alkoxy,
pyrazolyl substituted with phenyl substituted with lower alkoxy,
in which the preferred example of “suitable substituent(s)” in the term of “benzoyl substituted with one or more suitable substituent(s)” and “naphthoyl substituted with one or more suitable substituent(s)” may be
thiadiazolyl substituted with phenyl substituted with piperidyl substituted with one or two substituent(s) selected from the group consisting of methoxy, chlorophenyl, methoxybutoxymethyl, cyclohexyloxy, cyclohexyl, benzyloxy, phenyl, cyclopentyl, methoxypentyloxy, methoxyheptyloxy, cyclohexenyloxy, methoxybutoxy, methoxymethyl substituted with phenyl, cyclohexylmethoxy and methoxypentyloxymethyl,
thiadiazolyl substituted with phenyl substituted with phenyl substituted with a suitable substituent selected from the group consisting of methoxyethoxy, ethoxyethoxy, methoxybutoxy, cyclohexyloxy, methoxyethoxymethyl, ethoxymethyl, ethoxypropoxy, butoxy, ethoxy and methoxypropoxy,
thiadiazolyl substituted with phenyl substituted with heptyloxy substituted with methoxy,
thiadiazolyl substituted with phenyl substituted with piperazinyl substituted with a suitable substituent selected from the group consisting of cyclohexyl optionally substituted with methyl, benzyl substituted with morpholino, cyclopentylmethyl, cyclopentyl, cycloheptyl, cyclohexylmethyl, methoxyheptyl, pyridyl, pyrimidinyl and cyclohexyl substituted with methylidene,
thiadiazolyl substituted with pyridyl substituted with piperazinyl substituted with a suitable substituent selected from the group consisting of cyclohexyl substituted with methyl and cyclohexyl substituted with ethyl,
thiadiazolyl substituted with phenyl substituted with methoxyhexyloxy,
thiadiazolyl substituted with phenyl substituted with methoxyheptyloxy,
thiadiazolyl substituted with phenyl substituted with methoxyoctyloxy,
imidazothiadiazolyl substituted with phenyl substituted with a suitable substituent selected from the group consisting of piperidyl substituted with methoxypentyloxy and pentyloxy,
thiazolyl substituted with phenyl substituted with pentyloxy,
phenyl substituted with piperazinyl substituted with cyclohexyl optionally substituted with one or two substituent(s) selected from the group consisting of methoxy, cyclohexyl, cyclopentyl, ethyl and phenyl,
thiadiazolyl substituted with phenyl substituted with piperidyloxy substituted with phenyl,
phenyl substituted with a suitable substituent selected from the group consisting of methoxyheptyloxy, methoxyoctyloxy, tetahydroquinolyl optionally substituted with two methoxy, piperidyl substituted with cyclohexyloxy and phenyl substituted with morpholino,
methoxyheptyloxy,
octyloxy,
piperazinyl substituted with phenyl substituted with a suitable substituent selected from the group consisting of piperidyl substituted with methoxy and phenyl, methoxyhexyloxy, piperidyl substituted with tolyl and phenyl substituted with methoxypentyloxy,
piperazinyl substituted with cyclo(lower)alkyl substituted with a suitable substituent selected from the group consisting of cyclohexyl and pentyl,
phenyl substituted with piperazinyl substituted with phenyl substituted with a suitable substituent selected from the group consisting of morpholino substituted with two methyl and hexyloxy,
pyrazolyl substituted with phenyl substituted with hexyloxy.

Suitable example of “suitable substituent(s)” in the term of “heterocycliccarbonyl substituted with one or more suitable substituent(s)” may be higher alkoxy and heterocyclic group substituted with phenyl substituted with phenyl substituted with lower alkoxy(lower)alkoxy, in which the preferred one may be decyloxy and thiadiazolyl substituted with phenyl substituted with phenyl substituted with methoxyethoxy or ethoxyethoxy.

The more suitable example of “acyl group” of R¹ may be tert-butoxycarbonyl, benzyloxycarbonyl,
benzoyl substituted with thiadiazolyl substituted with phenyl substituted with piperidyl substituted with one or two substituent(s) selected from the group consisting of methoxy, chlorophenyl, methoxybutoxymethyl, cyclohexyloxy, cyclohexyl, benzyloxy, phenyl, cyclopentyl, methoxypentyloxy, methoxyheptyloxy, cyclohexenyloxy, methoxybutoxy, methoxymethyl substituted with phenyl, cyclohexylmethoxy and methoxypentyloxymethyl,
benzoyl substituted with thiadiazolyl substituted with phenyl substituted with phenyl substituted with a suitable substituent selected from the group consisting of methoxyethoxy, ethoxyethoxy, methoxybutoxy, cyclohexyloxy, methoxyethoxymethyl, ethoxymethyl, ethoxypropoxy, butoxy, ethoxy and methoxypropoxy,
benzoyl substituted with thiadiazolyl substituted with phenyl substituted with heptyloxy substituted with methoxy,
benzoyl substituted with thiadiazolyl substituted with phenyl substituted with piperazinyl substituted with a suitable substituent selected from the group consisting of cyclohexyl optionally substituted with methyl, benzyl substituted with morpholino, cyclopentylmethyl, cyclopentyl, cycloheptyl, cyclohexylmethyl, methoxyheptyl, pyridyl, pyrimidinyl and cyclohexyl substituted with methylidene,
benzoyl substituted with thiadiazolyl substituted with pyridyl substituted with piperazinyl substituted with a suitable substituent selected from the group consisting of cyclohexyl substituted with methyl and cyclohexyl substituted with ethyl, benzoyl substituted with thiadiazolyl substituted with phenyl substituted with methoxyhexyloxy,
benzoyl substituted with thiadiazolyl substituted with phenyl substituted with methoxyheptyloxy,
benzoyl substituted with thiadiazolyl substituted with phenyl substituted with methoxyoctyloxy,
benzoyl substituted with imidazothiadiazolyl substituted with phenyl substituted with a suitable substituent selected from the group consisting of piperidyl substituted with methoxypentyloxy and pentyloxy,
benzoyl substituted with thiazolyl substituted with phenyl substituted with pentyloxy,
benzoyl substituted with phenyl substituted with piperazinyl substituted with cyclohexyl optionally substituted with one or two substituent(s) selected from the group consisting of methoxy, cyclohexyl, cyclopentyl, ethyl and phenyl,
benzoyl substituted with thiadiazolyl substituted with phenyl substituted with piperidyloxy substituted with phenyl,
benzoyl substituted with phenyl substituted with a suitable substituent selected from the group consisting of methoxyheptyloxy, methoxyoctyloxy, tetahydroquinolyl optionally substituted with two methoxy, piperidyl substituted with cyclohexyloxy and phenyl substituted with morpholino,
naphthoyl substituted with methoxyheptyloxy,
benzoyl substituted with octyloxy,
benzoyl substituted with piperazinyl substituted with phenyl substituted with a suitable substituent selected from the group consisting of piperidyl substituted with methoxy and phenyl, methoxyhexyloxy, piperidyl substituted with tolyl and phenyl substituted with methoxypentyloxy, benzoyl substituted with piperazinyl substituted with cyclo(lower)alkyl substituted with a suitable substituent selected from the group consisting of cyclohexyl and pentyl,
benzoyl substituted with phenyl substituted with piperazinyl substituted with phenyl substituted with a suitable substituent selected from the group consisting of morpholino substituted with two methyl and hexyloxy,
benzoyl substituted with pyrazolyl substituted with phenyl substituted with hexyloxy,
pyridylcarbonyl substituted with thiadiazolyl substituted with phenyl substituted with phenyl substituted with methoxyethoxy, or
pyridylcarbonyl substituted with thiadiazolyl substituted with phenyl substituted with phenyl substituted with ethoxyethoxy.

Suitable example of “protected” moiety in the term of “protected amino(lower)alkyl” can be referred to aforementioned “acyl group”, in which the preferred one may be “amino protective group” mentioned below, and the most preferred one may be acetyl, 2-acetyloxypropionyl, methylsulfonyl, 2,5-diaminopentanoyl, benzyloxycarbonyl, fluorenylmethoxycarbonyl, allyloxycarbonyl and tert-butoxycarbonyl.

Suitable example of “amino protective group” may be included in aforementioned “acyl group”, a conventional protective group such as ar(lower)alkoxycarbonyl and lower alkoxycarbonyl, in which the preferred one may be phenyl-(C₁-C₄)alkoxycarbonyl and fluorenyl(C₁-C₄)alkoxycarbonyl and (C₁-C₄)alkoxycarbonyl, and the most preferred one may be benzyloxycarbonyl, fluorenylmethoxycarbonyl and tert-butoxycarbonyl.

Suitable example of “lower alkyl” in the term of “protected amino(lower)alkyl” and “amino(lower)alkyl” can be referred to aforementioned “lower alkyl”, in which the preferred one may be methyl, ethyl, propyl, isopropyl, butyl, pentyl and hexyl.

Suitable example of “protected amino(lower)alkyl” may be acetylamino(lower)alkyl, 2-acetyloxypropionylamino(lower)-alkyl, methylsulfonylamino(lower)alkyl, 2,5-diaminopentanoylamino(lower)alkyl, benzyloxycarbonylamino(lower)alkyl, fluorenylmethoxycarbonylamino(lower)alkyl, allyloxycarbonyl-amino(lower)alkyl and tert-butoxycarbonylamino(lower)alkyl, in which the preferred one may be phenyl(C₁-C₄)-alkoxycarbonylamino(C₁-C₄)alkyl and fluorenyl(C₁-C₄)-alkoxycarbonylamino(C₁-C₄)alkyl and (C₁-C₄)-alkoxycarbonylamino(C₁-C₄)alkyl, and the most preferred one may be benzyloxycarbonylaminopropyl, benzyloxycarbonylaminoethyl, benzyloxycarbonylaminomethyl, fluorenylmethoxycarbonylaminopropyl, fluorenylmethoxycarbonylaminoethyl, fluorenylmethoxycarbonylaminomethyl, tert-butoxycarbonylaminopropyl, tert-butoxycarbonylaminoethyl, tert-butoxycarbonylaminomethyl.

Suitable example of “amino(lower)alkyl” may be aminomethyl, aminoethyl, aminopropyl, aminoisopropyl, aminobutyl, aminopentyl and aminohexyl.

Suitable example of “heterocyclic” moiety in the term of “heterocycliccarbonyl(lower)alkyl” can be referred to aforementioned “heterocyclic group”, in which the preferred one may be saturated 3 to 8-membered (more preferably 5 or 6-membered) heteromonocyclic group containing 1 to 4 nitrogen atom(s), and the most preferred one may be pyrrolidinyl, imidazolidinyl, piperidyl, piperazinyl and azetidinyl.

Suitable example of “lower alkyl” moiety in the term of “heterocycliccarbonyl(lower)alkyl” can be referred to aforementioned “lower alkyl”, in which the preferred one may be methyl, ethyl, propyl, isopropyl, butyl, pentyl and hexyl.

Suitable example of “heterocycliccarbonyl(lower)alkyl” may be pyrrolidinylcarbonyl(C₁-C₄)alkyl, piperidylcarbonyl-(C₁-C₄)alkyl, and piperazinylcarbonyl(C₁-C₄)alkyl, in which the preferred one may be pyrrolidinylcarbonylmethyl, piperidylcarbonylmethyl, piperazinylcarbonylmethyl, piperidylcarbonylethyl, piperazinylcarbonylethyl, piperidylcarbonylpropyl and piperazinylcarbonylbutyl.

Suitable example of “heterocyclic” moiety in the term of “heterocycliccarbonyl” can be referred to aforementioned “heterocyclic group”, in which the preferred one may be unsaturated condensed heterocyclic group containing 1 to 4 nitrogen atom(s) (e.g., indolyl, isoindolyl, indolinyl, indolizinyl, benzimidazolyl, quinolyl, isoquinolyl, indazolyl, benzotriazolyl, tetahydroquinolyl, etc.) or unsaturated 3 to 8-membered (more preferably 5 or 6-membered) heteromonocyclic group containing 1 to 4 nitrogen atom(s) (e.g., pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), etc.), and most preferred one may be indolyl and pyridyl.

Suitable example of “lower alkyl” in the term of “lower alkylcarbamoyl(lower)alkyl” can be referred to aforementioned “lower alkyl”, in which the preferred one may be methyl, ethyl, propyl, isopropyl, butyl, pentyl and hexyl.

Suitable example of “lower alkylcarbamoyl(lower)alkyl” may be (C₁-C₄)alkylcarbamoyl(C₁-C₄)alkyl, and the most preferred one may be methylcarbamoylmethyl, ethylcarbamoylethyl, ethylcarbamoylpropyl and methylcarbamoylethyl.

Suitable example of “carboxy(lower)alkyl” may include carboxymethyl, 1-carboxyethyl, 2-carboxyethyl, 2-carboxypropyl, 3-carboxybutyl, 2-carboxy-1,1-dimethylethyl, 5-carboxypentyl, 6-carboxyhexyl, and the like, in which the preferred one may be carboxy(C₁-C₄)alkyl and the more preferred one may be carboxymethyl or 2-carboxyethyl.

Suitable “protected carboxy” moiety in “protected carboxy(lower)alkyl” may be an esterified carboxy group, or the like, and concrete examples of the ester moiety in said esterified carboxy group may be the ones such as lower alkyl ester (e.g. methyl ester, ethyl ester, propyl ester, isopropyl ester, butyl ester, isobutyl ester, tert-butyl ester, pentyl ester, hexyl ester, 1-cyclopropylethyl ester, etc.) which may have suitable substituent(s), for example, lower alkanoyloxy(lower)alkyl ester [e.g. acetoxymethyl ester, propionyloxymethyl ester, butyryloxymethyl ester, valeryloxymethyl ester, pivaloyloxymethyl ester, 1-acetoxyethyl ester, 1-propionyloxyethyl ester, pivaloyloxymethyl ester, 2-propionyloxyethyl ester, hexanoyloxymethyl ester, etc.], lower alkanesulfonyl(lower)alkyl ester [e.g. 2-mesylethyl ester, etc.] or mono(or di or tri)halo(lower)alkyl ester [e.g. 2-iodoethyl ester, 2,2,2-trichloroethyl ester, etc.]; lower alkenyl ester [e.g. vinyl ester, allyl ester, etc.]; lower alkynyl ester [e.g. ethynyl ester, propynyl ester, etc.] ar(lower)alkyl ester which may have suitable substituent(s) [e.g. benzyl ester, 4-methoxybenzyl ester, 4-nitrobenzyl ester, phenethyl ester, trityl ester, benzhydryl ester, bis(methoxyphenyl)methyl ester, 3,4-dimethoxybenzyl ester, 4-hydroxy-3,5-di-tert-butylbenzyl ester, etc.]; aryl ester which may have suitable substituent(s) [e.g. phenyl ester, 4-chlorophenyl ester, tolyl ester, 4-tert-butylphenyl ester, xyloy ester, mesityl ester, cumenyl ester, etc.]; or the like, in which the preferred one may be (C₁-C₄)alkyl ester and the most preferred one may be tert-butyl ester.

Suitable “lower alkyl” moiety in “protected carboxy(lower)alkyl” can be referred to aforesaid “lower alkyl”.

Suitable example of said “protected carboxy(lower)-alkyl” may be lower alkoxycarbonyl(lower)alkyl, in which the preferred one may be (C₁-C₄)alkoxycarbonyl(C₁-C₄)alkyl and the more preferred one may be tert-butoxycarbonylmethyl.

Suitable example of “hydroxy protective group” in the term of “protected hydroxy” may include acyl (e.g., lower alkanoyl, etc.) as mentioned above, phenyl(lower)alkyl which may have one or more suitable substituent(s) (e.g., benzyl, 4-methoxybenzyl, trityl, etc.), tri-substituted silyl [e.g., tri(lower)alkylsilyl (e.g., trimethylsilyl, t-butyldimethylsilyl, etc.), etc.], tetrahydropyranyl and the like.

Suitable example of “protected hydroxy” may be benzyloxy, 4-methoxybenzyloxy, trityloxy, trimethylsilyloxy, t-butyldimethylsilyloxy and tetrahydropyranyloxy.

Suitable example of “protected” moiety in the term of “protected guanidino(lower)alkyl” can be referred to aforementioned “acyl group”, in which the preferred one may be “guanidino protective group” mentioned below, and the most preferred one may be acetyl, 2-acetyloxypropionyl, methylsulfonyl, 2,5-diaminopentanoyl, benzyloxycarbonyl, fluorenylmethoxycarbonyl, allyloxycarbonyl and tert-butoxycarbonyl.

Suitable example of “guanidino protective group” may be included in aforementioned “acyl group”, a conventional protective group such as ar(lower)alkoxycarbonyl and lower alkoxycarbonyl, in which the preferred one may be phenyl-(C₁-C₄)alkoxycarbonyl and fluorenyl(C₁-C₄)alkoxycarbonyl and (C₁-C₄)alkoxycarbonyl, and the most preferred one may be benzyloxycarbonyl, fluorenylmethoxycarbonyl and tert-butoxycarbonyl.

Suitable example of “lower alkyl” in the term of “protected guanidino(lower)alkyl” and “guanidino(lower)alkyl” can be referred to aforementioned “lower alkyl”, in which the preferred one may be methyl, ethyl, propyl, isopropyl, butyl, pentyl and hexyl.

Suitable example of “protected guanidino(lower)alkyl” may be acetylguanidino(lower)alkyl, 2-acetyloxypropionylguanidino(lower)alkyl, methylsulfonylguanidino(lower)alkyl, 2,5-diaminopentanoylguanidino(lower)alkyl, benzyloxycarbonylguanidino(lower)alkyl, fluorenylmethoxycarbonylguanidino(lower)alkyl, allyloxycarbonylguanidino(lower)alkyl and tert-butoxycarbonylguanidino(lower)alkyl, in which the preferred one may be phenyl(C₁-C₄)-alkoxycarbonylguanidino(C₁-C₄)alkyl and fluorenyl(C₁-C₄)-alkoxycarbonylguanidino(C₁-C₄)alkyl and (C₁-C₄)—alkoxycarbonylguanidino(C₁-C₄)alkyl, and the most preferred one may be benzyloxycarbonylguanidinopropyl, benzyloxycarbonylguanidinoethyl, benzyloxycarbonylguanidinomethyl, fluorenylmethoxycarbonylguanidinopropyl, fluorenylmethoxycarbonylguanidinoethyl, fluorenylmethoxycarbonylguanidinomethyl, tert-butoxycarbonylguanidinopropyl, tert-butoxycarbonylguanidinoethyl, tert-butoxycarbonylguanidinomethyl.

Suitable example of “guanidino(lower)alkyl” may be guanidinomethyl, guanidinoethyl, guanidinopropyl, guanidinoisopropyl, guanidinobutyl, guanidinopentyl and guanidinohexyl.

Suitable example of “lower alkyl” in the term of “lower alkylamino(lower)alkyl” can be referred to aforementioned “lower alkyl”, in which the preferred one may be methyl, ethyl, propyl, isopropyl, butyl, pentyl and hexyl.

Suitable example of “lower alkylamino(lower)alkyl” may be methylaminomethyl, ethylaminomethyl, ethylaminoethyl, propylaminomethyl, propylaminoethyl, isopropylaminomethyl, isopropylaminoethyl, butylaminomethyl, and hexylaminomethyl.

Suitable example of “lower alkyl” in the term of “di-lower alkylamino(lower)alkyl” can be referred to aforementioned “lower alkyl”, in which the preferred one may be methyl, ethyl, propyl, isopropyl, butyl, pentyl and hexyl.

Suitable example of “di-lower alkylamino(lower)alkyl” may be dimethylaminomethyl, diethylaminomethyl, diethylaminoethyl, dipropylaminomethyl, dipropylaminoethyl, diisopropylaminomethyl, diisopropylaminoethyl, dibutylaminomethyl, and dihexylaminomethyl.

Suitable example of “lower alkyl” in the term of “amino(lower)alkanoylamino(lower)alkyl” can be referred to aforementioned “lower alkyl”, in which the preferred one may be methyl, ethyl, propyl, isopropyl, butyl, pentyl and hexyl.

Suitable example of “lower alkanoyl” in the term of “amino (lower) alkanoylamino (lower) alkyl” can be referred to aforementioned “lower alkanoyl”, in which the preferred one may be formyl, acetyl, propanoyl, butanoyl, 2-methylpropanoyl, pentanoyl, 2,2-dimethylpropanoyl, hexanoyl.

Suitable example of

“amino (lower) alkanoylamino (lower) alkyl” may be aminoacetylaminomethyl, aminoacetylaminoethyl, aminopropanoylaminomethyl, aminopropanaoylaminoethyl, aminopropanoylaminopropayl, aminobutanoylaminoethyl.

Suitable example of “lower alkyl” in the term of “hydroxy(lower)alkyl” can be referred to aforementioned “lower alkyl”, in which the preferred one may be methyl, ethyl, propyl, isopropyl, butyl, pentyl and hexyl.

Suitable example of “hydroxy(lower)alkyl” may be hydroxymethyl, hydroxyethyl, hydroxypropyl, hydroxyisopropyl, hydroxybutyl, hydroxypentyl and hydroxyhexyl.

Particularly, the preferred example of the lipopeptide compound (I) of the present invention are as follows:

the compound (I), wherein

R¹ is acyl group,
R² is amino(lower)alkyl or lower alkylamino(lower)alkyl substituted with two hydroxy,
R³ is hydrogen,
R⁴ is amino(lower)alkyl or lower alkylamino(lower)alkyl substituted with one or two hydroxy, and
R⁵ is hydroxy,
the more preferred example of the lipopeptide compound (I) are
   the compound (I), wherein
R¹ is benzoyl substituted with a substituent selected from the group consisting of
   1) thiadiazolyl substituted with phenyl substituted with piperidyl substituted with one or two substituent(s) selected from the group consisting of lower alkoxy, cyclo(lower)alkyl, lower alkoxy(lower)alkoxy(lower)alkyl, lower alkoxy(lower)alkoxy, lower alkoxy(lower)alkyl substituted with phenyl and cyclo(lower)alkyl(lower)alkyloxy,
   2) thiadiazolyl substituted with phenyl substituted with phenyl substituted with lower alkoxy(lower)alkoxy,
   3) thiadiazolyl substituted with phenyl substituted with piperazinyl substituted with a substituent selected from the group consisting of cyclo(lower)alkyl substituted with lower alkyl, cyclo(lower)alkyl(lower)alkyl, cyclo(lower)alkyl, cyclo(higher)alkyl, lower alkoxy(higher)alkyl and cyclo(lower)alkyl substituted with lower alkylidene,
   4) thiadiazolyl substituted with pyridyl substituted with piperazinyl substituted with cyclo(lower)alkyl substituted with lower alkyl,
   5) imidazothiadiazolyl substituted with phenyl substituted with piperidyl substituted with lower alkoxy(lower)alkoxy,
   6) phenyl substituted with piperazinyl substituted with cyclo(lower)alkyl optionally substituted with one or two substituent(s) selected from the group consisting of cyclo(lower)alkyl, lower alkoxy, lower alkyl and phenyl,
   7) imidazothiadiazolyl substituted with phenyl substituted with lower alkoxy,
   8) phenyl substituted with a substituent selected from the group consisting of piperidyl substituted with cyclo(lower)alkyloxy and phenyl substituted with morpholino,
   8) piperazinyl substituted with phenyl substituted with phenyl substituted with lower alkoxy(lower)alkoxy
   9) piperazinyl substituted with cyclo(lower)alkyl substituted with lower alkyl, and
   10) phenyl substituted with piperazinyl substituted with phenyl substituted with heterocyclic group substituted with lower alkyl and lower alkoxy, or heterocycliccarbonyl substituted with heterocyclic group substituted with lower alkoxy(lower)alkoxy,
R² is amino(lower)alkyl or lower alkylamino(lower)alkyl substituted with two hydroxy,
R³ is hydrogen,
R⁴ is amino(lower)alkyl or lower alkylamino(lower)alkyl substituted with one or two hydroxy, and
R⁵ is hydroxy,
the most preferred example of the lipopeptide compound (I) are
   the compound (I), wherein
R¹ is benzoyl substituted with a substituent selected from the group consisting of
   1) thiadiazolyl substituted with phenyl substituted with piperidyl substituted with a substituent selected from the group consisting of cyclo(lower)alkyl substituted with lower alkyl and cyclo(lower)alkyl, and
   2) thiadiazolyl substituted with pyridyl substituted with piperazinyl substituted with cyclo(lower)alkyl substituted with lower alkyl
R² is amino(lower)alkyl or lower alkylamino(lower)alkyl substituted with two hydroxy,
R³ is hydrogen,
R⁴ is amino(lower)alkyl or lower alkylamino(lower)alkyl substituted with one or two hydroxy, and
R⁵ is hydroxy.

The processes for preparing the lipopeptide compound (I) of the present invention are explained in detail in the following.

Process 1

The compound (Ia) or a salt thereof can be prepared by reacting the compound (II) or its reactive derivative at the hydroxy group or a salt thereof with the compound (VII) or its reactive derivative or a salt thereof.

This reaction is usually carried out in a solvent such as water, alcohol (e.g., methanol, ethanol, etc.), benzene, N,N-dimethylformamide, tetrahydrofuran, toluene, methylene chloride, ethylene dichloride, chloroform, dioxane, diethyl ether or any other solvents which do not adversely affect the reaction, or the mixture thereof.

The reaction temperature is not critical and the reaction is usually carried out under cooling to heating.

The reaction is usually carried out in the presence of an acid including Lewis acid.

Suitable acid may include an organic acid [e.g. formic acid, acetic acid, propionic acid, trichloroacetic acid, trifluoroacetic acid, etc.] and an inorganic acid [e.g. hydrochloric acid, hydrobromic acid, sulfuric acid, hydrogen chloride, hydrogen bromide, zinc halide (e.g., zinc chloride, zinc bromide, etc.), etc.] and the like.

The reaction may be also carried out in the presence of an inorganic or an organic base such as an alkali metal (e.g., sodium potassium, etc.), an alkali metal hydroxide (e.g., sodium hydroxide, potassium hydroxide, etc.), An alkali metal hydrogencarbonate (e.g., sodium hydrogencarbonate, potassium hydrogencarbonate, etc.), alkali metal carbonate (e.g., sodium carbonate, potassium carbonate, etc.), tri(lower)alkylamine (e.g., trimethylamine, triethylamine, diisopropylethylamine, etc.), alkali metal hydride (e.g., sodium hydride, etc.), alkali metal (lower)alkoxide (e.g. sodium methoxide, sodium ethoxide, etc.), pyridine, lutidine, picoline, dimethylaminopyridine, N-(lower)alkylmorpholine, N,N-di(lower)alkylbenzylamine, N,N-di(lower)alkylaniline or the like.

When the base, the acid and/or the starting compound are in liquid, they can be used also as a solvent.

Process 2

The object compound (Ib) or a salt thereof can be prepared by subjecting a compound (Ia) or a salt thereof to elimination reaction of amino protective group.

This reaction is carried out in accordance with a conventional method such as hydrolysis, reduction or the like.

The hydrolysis is preferably carried out in the presence of a base or an acid including Lewis acid. Suitable base may include an inorganic base and an organic base such as an alkali metal [e.g. sodium, potassium, etc.], an alkaline earth metal [e.g. magnesium, calcium, etc.], the hydroxide or carbonate or bicarbonate thereof, trialkylamine [e.g. trimethylamine, triethylamine, etc.], picoline, 1,5-diazabicyclo[4.3.0]non-5-ene, 1,4-diazabicyclo[2.2.2]octane, 1,8-diazabicyclo[5.4.0]undec-7-ene, or the like.

Suitable acid may include an organic acid [e.g. formic acid, acetic acid, propionic acid, trichloroacetic acid, trifluoroacetic acid, etc.] and an inorganic acid [e.g. hydrochloric acid, hydrobromic acid, sulfuric acid, hydrogen chloride, hydrogen bromide, etc.]. The elimination using Lewis acid such as trihaloacetic acid [e.g. trichloroacetic acid, trifluoroacetic acid, etc.] or the like is preferably carried out in the presence of cation trapping agents [e.g. anisole, phenol, etc.].

The reaction is usually carried out in a solvent such as water, an alcohol [e.g. methanol, ethanol, etc.], methylene chloride, tetrahydrofuran, a mixture thereof or any other solvent which does not adversely influence the reaction. A liquid base or acid can be also used as the solvent. The reaction temperature is not critical and the reaction is usually carried out under cooling to warming.

The reduction method applicable for the elimination reaction may include chemical reduction and catalytic reduction.

Suitable reducing agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium, sulfate, palladium on barium carbonate, etc.], nickel catalysts [e.g. reduced nickel, nickel oxide, Raney nickel, etc.], cobalt catalysts [e.g. reduced cobalt, Raney cobalt, etc], iron catalysts [e.g. reduced iron, Raney iron, etc], copper catalysts [e.g. reduced copper, Raney copper, Ullman copper, etc.] and the like.

The reduction is usually carried out in a conventional solvent which does not adversely influence the reaction such as water, methanol, ethanol, propanol, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to warming.

Process 3

The object compound (Id) or a salt thereof can be prepared by subjecting a compound (Ic) or salt thereof to elimination reaction of the amino protective group.

This reaction is carried out in accordance with a conventional method such as hydrolysis, reduction or the like.

The hydrolysis is preferably carried out in the presence of a base or an acid including Lewis acid.

Suitable base may include an inorganic base and an organic base such as an alkali metal [e.g. sodium, potassium, etc.], an alkaline earth metal [e.g. magnesium, calcium, etc.], the hydroxide or carbonate or bicarbonate thereof, trialkylamine [e.g. trimethylamine, triethylamine, N,N-diisopropylethylamine, etc.], pyridine, di(lower)alkylaminopyridine (e.g., 4-dimethylaminopyridine, etc.), N-(lower)alkylmorpholine, N,N-di(lower)alkylbenzylamine, picoline, 1,5-diazabicyclo-[4.3.0]non-5-ene, 1,4-diazabicyclo[2.2.2]octane, 1,8-diazabicyclo[5.4.0]undec-7-ene, or the like.

Suitable acid may include an organic acid [e.g. formic acid, acetic acid, propionic acid, trichloroacetic acid, trifluoroacetic acid, etc.)]and an inorganic acid [e.g. hydrochloric acid, hydrobromic acid, sulfuric acid, hydrogen chloride, hydrogen bromide, etc.]. The elimination using Lewis acid such as trihaloacetic acid [e.g. trichloroacetic acid, trifluoroacetic acid, etc.] or the like is preferably carried out in the presence of cation trapping agents [e.g. anisole, phenol, etc.].

The reaction is usually carried out in a solvent such as water, an alcohol [e.g. methanol, ethanol, etc.], methylene chloride, tetrahydrofuran, a mixture thereof or any other solvent which does not adversely influence the reaction. A liquid base or acid can be also used as the solvent. The reaction temperature is not critical and the reaction is usually carried out under cooling to warming.

The reduction method applicable for the elimination reaction may include chemical reduction and catalytic reduction.

Suitable reducing agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon colloidal palladium, palladium on barium, sulfate, palladium on barium carbonate, etc.], nickel catalysts [e.g. reduced nickel, nickel oxide, Raney nickel, etc.], cobalt catalysts [e.g. reduced cobalt, Raney cobalt, etc.], iron catalysts [e.g. reduced iron, Raney iron, etc.], copper catalysts [e.g. reduced copper, Raney copper, Ullman copper, etc.] and the like.

The reduction is usually carried out in a conventional solvent which does not adversely influence the reaction such as water, methanol, ethanol, propanol, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to warming.

Process 4

The object compound (If) or a salt thereof can be prepared by reacting the compound (Ie) or its reactive derivative at the amino group or a salt thereof with the compound (VIII) of the formula:
Rₐ¹—OH (VIII)
(wherein Rₐ¹ is acyl group)
   or its reactive derivative at the carboxy group or a salt thereof.

Suitable reactive derivative at the carboxy group of the compound (VIII) may include an acid halide, an acid anhydride, an activated amide, an activated ester, and the like. Suitable examples of the reactive derivatives may be an acid chloride; an acid azide; a mixed acid anhydride with an acid such as substituted phosphoric acid [e.g., dialkylphosphoric acid, phenylphosphoric acid, diphenylphosphoric acid, dibenzylphosphoric acid, halogenated phosphoric acid, etc.], dialkylphosphorous acid, sulfurous acid, thiosulfuric acid, sulfuric acid, sulfonic acid [e.g., methanesulfonic acid, etc.], aliphatic carboxylic acid [e.g., acetic acid, propionic acid, butyric acid, isobutyric acid, pivaric acid, pentanoic acid, isopentanoic acid, 2-ethylbutyric acid, trichloroacetic acid, etc.]; or aromatic carboxylic acid [e.g., benzoic acid, etc.]; a symmetrical acid, anhydride; an activated amide with imidazole, 4-substituted imidazole, dimethylpyrazole, triazole, tetrazole or 1-hydroxy-1H-benzotriazole; or an activated ester [e.g., cyanomethyl ester, methoxymethyl ester, dimethyliminomethyl 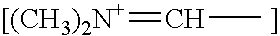
ester, vinyl ester, propargyl ester, p-nitrophenyl ester, 2,4-dinitrophenyl ester, trichlorophenyl ester, pentachloropentyl ester, mesylphenyl ester, phenylazophenyl ester, phenylthioester, p-nitrophenyl thioester, p-cresyl thioester, carboxymethyl thioester, pyranyl ester, pyridyl ester, piperidyl ester, 8-quinolyl thioester, etc.], or an ester with a N-hydroxy compound [e.g. N,N-dimethylhydroxylamine, 1-hydroxy-2-(1H)-pyridone, N-hydroxysuccinimide, N-hydroxyphthalimide, 1-hydroxy-1H-benzotriazole, etc.], and the like. These reactive derivatives can optionally be selected from them according to the kind of the compound (VIII) to be used.

Suitable salts of the compound (VIII) and its reactive derivative can be referred to the ones as exemplified for the lipopeptide compound (I).

The reaction is usually carried out in a conventional solvent such as water, alcohol [e.g., methanol, ethanol, etc.], acetone, dioxane, acetonitrile, chloroform, methylene chloride, ethylene chloride, tetrahydrofuran, ethyl acetate, N,N-dimethylformamide, pyridine or any other organic solvent which does not adversely influence the reaction. These conventional solvents may also be used in a mixture with water.

In this reaction, when the compound (VIII) is used in a free acid form or its salt form, the reaction is preferably carried out in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl)carbodiimide; N,N′-diethylcarbodiimide; N,N′-diisopropylcarbodiimide; N-ethyl-N′-(3-dimethylaminopropyl)carbodiimide; N,N-carbonylbis-(2-methylimidazole); pentamethyleneketene-N-cyclohexylimine; diphenylketene-N-cyclohexylimine, ethoxyacetylene; 1-alkoxy-2-chloroethylene; trialkyl phosphite; ethyl polyphosphate; isopropyl polyphosphate; phosphorus oxychloride (phosphoryl chloride); phosphorus trichloride; thionyl chloride; oxalyl chloride; lower alkyl haloformate [e.g., ethyl chloroformate, isopropyl chloroformate, etc.]; triphenylphosphine; 2-ethyl-7-hydroxybenzisoxazolium salt; 2-ethyl-5-(m-sulfophenyl)isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, trichloromethyl chloroformate, phosphorous oxychloride, methanesulfonyl chloride, etc.; or the like.

The reaction may also be carried out in the presence of an inorganic or organic base such as an alkali metal carbonate, alkali metal bicarbonate, tri(lower)alkylamine (e.g., triethylamine, diisopropylethylamine, etc.), pyridine, di(lower)alkylaminopyridine (e.g., 4-dimethylaminopyridine, etc.), N-(lower)alkylmorpholine, N,N-di(lower)alkylbenzylamine, or the like.

The reaction temperature is not critical, and the reaction is usually carried out under cooling to warming.

Process 5

The object compound (Ih) or a salt thereof can be prepared by reacting the compound (Ig) or its reactive derivative at the amino group or a salt thereof with the compound (IX) of the formula:
R⁶═O (IX)
(wherein R⁶ is lower alkyl substituted with one or more hydroxy.)
or its reactive derivative, or a salt thereof.

Suitable reactive derivative of the compound (IX) may include an acid halide, an acid anhydride, an activated ester, and the like. The suitable example may be an acid chloride; acid azide; a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid, etc.), dialkylphosphorous acid, sulfurous acid, thiosulfuric acid, alkanesulfonic acid (e.g., methanesulfonic acid, ethanesulfonic acid, etc.), sulfuric acid, alkylcarbonic acid, aliphatic carboxylic acid (e.g., pivalic acid, pentanoic acid, isopentanoic acid, 2-ethylbutyric acid, trichloroacetic acid, etc.); aromatic carboxylic acid (e.g., benzoic acid, etc.); a symmetrical acid anydride; an activated amide with imidazole, 4-substitutd imidazole, dimethylpyrazole, triazole or tetrazole; an activated ester (e.g., cyanomethyl, ester methoxymethyl ester, vinyl ester, propargyl ester, p-nitrophenyl ester, 2,4-dinitrophenyl ester, trichlorophenyl ester, pentachlorophenyl ester, esylphenyl ester, phenylazophenyl ester, phenylthioester, p-nitrophenyl thioester, p-cresyl thioester, carboxymethyl thioester, pyranyl ester, pyridyl ester, piperidyl ester, 8-quinolyl thioester, etc.); an ester with a N-hydroxy compound (e.g., N,N-dimethylhydroxylamine, 1-hydroxy-2-(1H)-pyridone, N-hydroxysuccinimide, N-hydroxybenzotriazole, N-hydroxyphthalimide, 1-hydroxy-6-chloro-1H-benzotriazole, etc.); and the like. These reactive derivatives can optionally be selected from them according to the kind of the compound (IX) to be used.

The reaction is usually carried out in a conventional solvent such as water, acetone, dioxane, acetonitrile, chloroform, methylene chloride, ethylene chloride, tetrahydrofuran, ethyl acetate, N,N-dimethylformamide, pyridine or any other organic solvent which do not adversely affect the reaction, or the mixture thereof.

When the compound (IX) is used in free acid form or its salt form in the reaction, the reaction is preferably carried out in the presence of a conventional condensing agent such as N,N′-dicyclohexylcarbodiimide; N-cyclohexyl-N′-morpholinoethylcarbodiimide); N-cyclohexyl-N′-(4-diethylaminocyclohexyl)carbodiimide; N,N′-diisopropylcarboxi-imide; N-ethyl-N′-(3-dimethylaminopropyl)carbodiimide; N,N-carbonyl-bis(2-methylimidazole); pentamethyleneketene-N-cyclohexylimine; diphenylketene-N-cyclohexylimine, ethoxyacetylene; 1-alkoxy-1-chloroethylene; trialkyl phosphite; isopropyl polyphosphate; phosphorous oxychloride (phosphoryl chloride); phosphorous trichloride; thionyl chloride; oxalyl chloride; triphenylphosphite; 2-ethyl-7-hydroxybenzisoxazolium salt; 2-ethyl-5-(m-sulfophenyl)isoxazolium hydroxide intra-molecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, phosphorous oxychloride, etc.; or the like.

The reaction may also be carried out in the presence of an organic or inorganic base such as an alkali metal bicarbonate, tri(lower)alkylamine (e.g., triethylamine, diisopropylethylamine, etc.), pyridine, di(lower)alkylaminopyridine (e.g., 4-dimethylaminopyridine, etc.) N-(lower)alkylmorphorine, N,N-di(lower)alkylbenzylamine, or the like. The reaction temperature is not critical, and the reaction is usually carried out under cooling to heating.

Process A

The object compound (IV) or a salt thereof can be prepared by reacting the compound (III) or its reactive derivative at the hydroxy group or a salt thereof with the compound (X) or its reactive derivative or a salt thereof.

This reaction can be carried out in a similar manner to that of Process 1, and therefore the reagents to be used and the reaction conditions (e.g., solvent, reaction temperature, etc.) can be referred to those of the Process 1.

Process B

The object compound (V) or a salt thereof can be prepared by subjecting a compound (IV) or its reactive derivative at the carbamoyl group or a salt thereof to dehydration reaction.

The reaction can be carried out in the manner disclosed in Preparation 27 or similar manners thereto.

Process C

The compound (VI) or a salt thereof can be prepared by subjecting the compound (V) or its reactive derivative at the sulfonic acid group or a salt thereof to hydrolysis reaction of the sulfonic acid group.

The hydrolysis is preferably carried out in the presence of a base or an acid including Lewis acid.

Suitable base may include an inorganic base and an organic base such as an alkali metal [e.g., sodium, potassium, etc.], an alkaline earth metal [e.g., magnesium, calcium, etc.], the hydroxide or carbonate or hydrogencarbonate thereof, trialkylamine [e.g., trimethylamine, triethylamine, etc.], picoline, 1,5-diazabicyclo[4.3.0]-non-5-ene, or the like.

Suitable acid may include an organic acid [e.g., formic acid, acetic acid, propionic acid, trichloroacetic acid, trifluoroacetic acid, etc.], and an inorganic acid [e.g., hydrochloric acid, hydrobromic acid, sulfuric acid, hydrogen, chloride, hydrogen bromide, etc.].

The elimination using Lewis acid such as trihaloacetic acid [e.g., trichloroacetic acid, trifluoroacetic acid, etc.], or the like is preferably carried out in the presence of cation trapping agents [e.g., anisole, phenol, etc.].

The reaction is usually carried out in a conventional solvent such as water, alcohol [e.g., methanol, ethanol, isopropyl alcohol, etc.], tetrahydrofuran, dioxane, toluene, methylene chloride, ethylene dichloride, chloroform, N,N-dimethylformamide, N,N-dimethylacetamide or any other organic solvent which do not adversely affect the reaction, or the mixture thereof.

The reaction temperature is not critical and the reaction is usually carried out under cooling to warming.

Process D

The compound (II) or a salt thereof can be prepared by reducing the compound (VI) or a salt thereof, and then reacting with the compound (XI) of the formula:
R²—OH (XI)
(wherein R² is acyl group)
   or its reactive derivative, or a salt thereof.

Suitable reactive derivative at the compound (XI) may include an acid halide, an acid anhydride, an activated ester and the like. The suitable examples may be an acid chloride; an acid azide; a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid, etc.), dialkylphosphorous acid, sulfurous acid, thiosulfuric acid, alkanesulfonic acid (e.g., methanesulfonic acid, ethanesulfonic acid, etc.), sulfuric acid, alkylcarbonic acid, aliphatic carboxylic acid (e.g., pivalic acid, pentanoic acid, isopentanoic acid, 2-ethylbutyric acid, trichloroacetic acid, etc.); aromatic carboxylic acid (e.g., benzoic acid, etc.); a symmetrical acid anydride, an activated amide with imidazole, 4-substituted imidazole, dimethylpyrazole, triazole or tetrazole; an activated ester (e.g., cyanomethyl ester, methoxymethyl ester, vinyl ester, propargyl ester, p-nitrophenyl ester, 2,4-dinitrophenyl ester, trichlorophenyl ester, pentachloropentyl ester, mesylphenyl ester, phenylazophenyl ester, phenylthioester, p-nitrophenyl thioester, p-cresyl thioester, carboxymethyl thioester, pyranyl ester, pyridyl ester, piperidyl ester, 8-quinolyl thioester, etc.), an ester with a N-hydroxy compound [e.g. N,N-dimethylhydroxylamine, 1-hydroxy-2-(1H)-pyridone, N-hydroxysuccinimide, N-hydroxybenzotriazole, N-hydroxyphthalimide, 1-hydroxy-6-chloro-1H-benzotriazole, etc.); and the like. These reactive derivatives can optionally be selected from them according to the kind of the compound (X) to be used.

The reaction is usually carried out in a conventional solvent such as water, acetone, dioxane, acetonitrile, chloroform, methylene chloride, ethylene chloride, tetrahydrofuran, ethyl acetate, N,N-dimethylformamide, pyridine or any other organic solvent which do not adversely affect the reaction, or the mixture thereof.

When the compound (X) is used in free acid form or its salt form in the reaction, the reaction is preferably carried out in the presence of a conventional condensing agent such as N,N′-dicyclohexylcarbodiimide; N-cyclohexyl-N′-morpholinoethylcarbodiimide); N-cyclohexyl-N′-(4-diethylaminocyclohexyl)carbodiimide; N,N′-diisopropylcarbodiimide; N-ethyl-N′-(3-dimethylaminopropyl)carbodiimide; N,N-carbonyl-bis(2-methylimidazole); pentamethyleneketene-N-cyclohexylimine; diphenylketene-N-cyclohexylimine, ethoxyacetylene; 1-alkoxy-1-chloroethylene; trialkyl phosphite; isopropyl polyphosphate; phosphorous oxychloride (phosphoryl chloride); phosphorus trichloride; thionyl chloride; oxalyl chloride; triphenylphosphite; 2-ethyl-7-hydroxybenzisoxazolium salt; 2-ethyl-5-(m-sulfophenyl)isoxazolium hydroxide intra-molecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, phosphorous oxychloride, etc.; or the like.

The reaction may also be carried out in the presence of an organic or inorganic base such as an alkali metal bicarbonate, tri(lower)alkylamine (e.g., triethylamine, diisopropylethylamine, etc.), pyridine, di(lower)alkylaminopyridine (e.g., 4-dimethylaminopyridine, etc.), N-(lower)alkylmorpholine, N,N-di(lower)alkylbenzylamine, or the like.

The reaction temperature is not critical, and the reaction is usually carried out under cooling to warming.

The compounds obtained by the above Processes 1 to 5 and Processes A to D can be isolated and purified by a conventional method such as pulverization, recrystallization, column-chromatography, high-performance liquid chromatography (HPLC), reprecipitation, desalting resin column chromatography, or the like.

The compounds obtained by the above Processes 1 to 5 and Processes A to D may be obtained as its solvate (e.g., hydrate, ethanolate, etc.), and its solvate (e.g., hydrate, ethanolate, etc.) is included within the scope of present invention.

It is to be noted that each of the lipopeptide compound (I) may include one or more stereoisomer(s) such as optical isomer(s) and geometrical isomer(s) due to asymmetric carbon atom(s) and double bond(s) and all such isomers and the mixture thereof are included within the scope of the present invention.

The lipopeptide compound (I) or a salt thereof may include solvent compound [e.g., hydrate, ethanolate, etc.].

The lipopeptide compound (I) or a salt thereof may include both its crystal form and non-crystal form.

It should be understood that the lipopeptide compound (I) of the present invention may include the prodrug form.

The patent applications and publications cited herein are incorporated by reference.

In order to show the usefulness of the lipopeptide compound (I) of the present invention, the biological data of the representative compound is explained in the following.

### Biological Property of the Lipopeptide Compound (I) of the Present Invention

Test (Antimicrobial Activity):

In vitro antimicrobial activity of the object compound of Example 23, 46, 58 and 63 disclosed later was determined by MICS in mouse serum as described below.

Test Method:

The MICₛ in mouse serum were determined by the microdilution method using ICR mouse serum buffered with 20 mM HEPES buffer (pH 7.3) as a test medium. Inoculum suspension of 10⁶ cells/ml were prepared by a hemocytometric procedure and diluted to obtain an inoculum size of approximately 1.0×10³ cells/ml. Microplates were incubated at 37° C. for 24 hours in 5% CO₂. The MICs was defined as the lowest concentrations at which no visible growth was observed.

Test Result:

MIC (μg/ml)

| Test organism | |
|---|---|
| Test compound | Candida albicans FP-633 |
| The object compound of | <0.2 |
| Example 23 | |
| The object compound of | <0.2 |
| Example 46 | |
| The object compound of | <0.2 |
| Example 58 | |
| The object compound of | <0.2 |
| Example 63 | |

From the test result, it is realized that the lipopeptide compound (I) of the present invention has an antimicrobial activity (especially, antifungal activity).

In more details, the lipopeptide compound (I) of the present invention has an antifungal activity, particularly against the following fungi.

Acremonium; Absidia (e.g., Absidia corymbifera, etc.); Aspergillus (e.g., Aspergillus clavatus, Aspergillus flavus, Aspergillus fumigatus, Aspergillus nidulans, Aspergillus niger, Aspergillus terreus, Aspergillus versicolor, etc.); Blastomyces (e.g., Blastomyces dermatitidis, etc.); Candida (e.g., Candida albicans, Candida glabrata, Candida guilliermondii, Candida kefyr, Candida krusei, Candida parapsilosis, Candida stellatoidea, Candida tropicalis, Candida utilis, etc.); Cladosporium (e.g., Cladosporium trichloides, etc.); Coccidioides (e.g., Coccidioides immitis, etc.); Cryptococcus (e.g., Cryptococcus neoformans, etc.); Cunninghamella (e.g., Cunninghamella elegans, etc.); Dermatophyte; Exophiala (e.g., Exophiala dermatitidis, Exophiala spinifera, etc.); Epidermophyton (e.g., Epidermophyton floccosum, etc.); Fonsecaea (e.g., Fonsecaea pedrosoi, etc.); Fusarium (e.g., Fusarium solani, etc.); Geotrichum (e.g., Geotrichum candiddum, etc.); Histoplasma (e.g., Histoplasma capsulatum var. capsulatum, etc.). Malassezia (e.g., Malassezia furfur, etc.); Microsporum (e.g., Microsporum canis, Microsporum gypseum, etc.); Mucor; Paracoccidioides (e.g., Paracoccidioides brasiliensis, etc.); Penicillium (e.g., Penicillium marneffei, etc.); Phialophora; Pneumocystis (e.g., Pneumocystis carinii, etc.); Pseudallescheria (e.g., Pseudallescheria boydii, etc.); Rhizopus (e.g., Rhizopus microsporus var. rhizopodiformis, Rhizopus oryzae, etc.); Saccharomyces (e.g., Saccharomyces cerevisiae, etc.); Scopulariopsis; Sporothrix (e.g., Sporothrix schenckii, etc.); Trichophyton (e.g., Trichophyton mentagrophytes, Trichophyton rubrum, etc.); Trichosporon (e.g., Trichosporon asahii, Trichosporon cutaneum, etc.).

The above fungi are well-known to cause various infection diseases in skin, eye, hair, nail, oral mucosa, gastrointestinal tract, bronchus, lung, endocardium, brain, meninges, urinary organ, vaginal protion, oral cavity, ophthalmus, systemic, kidney, bronchus, heart, external auditory canal, bone, nasal cavity, paranasal cavity, spleen, liver, hypodermal tissue, lymph doct, gastrointestine, articulation, muscle, tendon, interstitial plasma cell in lung, blood, and so on.

Therefore, the lipopeptide compound (I) of the present invention are useful for preventing and treating various infectious diseases, such as dermatophytosis (e.g., trichophytosis, etc.), pityriasis versicolor, candidiasis, cryptococcosis, geotrichosis, trichosporosis, aspergillosis, penicilliosis, fusariosis, zygomycosis, sporotrichosis, chromomycosis, coccidioidomycosis, histoplasmosis, blastomycosis, paracoccidioidomycosis, pseudallescheriosis, mycetoma, mycotic keratitis, otomycosis, pneumocystosis, fungemia, and so on.

The combination use of azoles such as fluconazole, voriconazole, itraconazole, ketoconazole, miconazole, ER 30346 and SCH 56592; polyenes such as amphotericin B, nystatin, liposamal and lipid forms thereof such as Abelcet, AmBisome, and Amphocil; purine or pyrimidine nucleotide inhibitors such as flucytosine; or polyxins such as nikkomycines, in particular nikkomycine Z or nikkomycine X; other chitin inhibitors; elongation factor inhibitors such as sordarin and analogs thereof; mannan inhibitors such as predamycin, bactericidal/permeability-inducing (BPI) protein products such as XMP.97 or XMP.127; or complex carbohydrate antifungal agents such as CAN-296; or the combination use of immunosuppressant such as tacrolimus with the lipopeptide compound (I) or a salt thereof is effective against above infectious diseases.

The pharmaceutical composition of the present invention can be used in the form of a pharmaceutical preparation, for example, in solid, semisolid or liquid form, which contains the lipopeptide compound (I) or a pharmaceutically acceptable salt thereof, as an active ingredient in admixture with an organic or inorganic carrier or excipient which is suitable for rectal; pulmonary (nasal or buccal inhalation); ocular; external (topical); oral administration; parenteral (including subcutaneous, intravenous and intramuscular) administrations; insufflation (including aerosols from metered dose inhalator); nebulizer; or dry powder inhalator.

The active ingredient may be compounded, for example, with the usual non-toxic, pharmaceutically acceptable carriers in a solid form such as granules, tablets, dragees, pellets, troches, capsules, or suppositories; creams; ointments; aerosols; powders for insufflation; in a liquid form such as solutions, emulsions, or suspensions for injection; ingestion; eye drops; and any other form suitable for use. And, if necessary, there may be included in the above preparation auxiliary substance such as stabilizing, thickening, wetting, emulsifying and coloring agents; perfumes or buffer; or any other commonly may be used as additives.

The lipopeptide compound (I) or a pharmaceutically acceptable salt thereof is/are included in the pharmaceutical composition in an amount sufficient to produce the desired antimicrobial effect upon the process or condition of diseases.

For applying the composition to human, it is preferable to apply it by intravenous, intramuscular, pulmonary, oral administration, eye drop administration or insufflation. While the dosage of therapeutically effective amount of the lipopeptide compound (I) varies from and also depends upon the age and condition of each individual patient to be treated, in the case of intravenous administration, a daily dose of 0.01-400 mg of the lipopeptide compound (I) per kg weight of human being in the case of intramuscular administration, a daily dose of 0.1-20 mg of the lipopeptide compound (I) per kg weight of human being, in case of oral administration, a daily dose of 0.5-50 mg of the lipopeptide compound (I) per kg weight of human being is generally given for treating or preventing infectious diseases.

Especially in case of the treatment of prevention of Pneumocystis carinii infection, the followings are to be noted.

For administration by inhalation, the compounds of the present invention are conveniently delivered in the form of an aerosol spray presentation from pressurized as powders which may be formulated and the powder compositions may be inhaled with the aid of an insufflation powder inhaler device. The preferred delivery system for inhalation is a metered dose inhalation aerosol, which may be formulated as a suspension or solution of compound in suitable propellants such as fluorocarbons or hydrocarbons.

Because of desirability to directly treat lung and bronchi, aerosol administration is a preferred method of administration. Insufflation is also a desirable method, especially where infection may have spread to ears and other body cavities.

Alternatively, parenteral administration may be employed using drip intravenous administration.

For administration by intravenous administration, the preferred pharmaceutical composition is the lyophilized form containing the lipopeptide compound (I) or its pharmaceutically acceptable salt.

The amount of the lipopeptide compound (I) or its pharmaceutically acceptable salt contained in the composition for a single unit dosage of the present invention is 0.1 to 400 mg, more preferably 1 to 200 mg, still more preferably 5 to 100 mg, specifically 5, 10, 15, 20, 25, 30, 35, 40, 45, 50, 55, 60, 70, 75, 80, 85, 90, 95 and 100 mg.

The present invention further provides the following ones.

An article of manufacture, comprising packaging material and the compound (I) identified in the above contained within said packaging material, wherein said the compound (I) is therapeutically effective for preventing or treating infectious diseases caused by pathogenic microorganism, and wherein said packaging material comprises a label or a written material which indicates that said compound (I) can or should be used for preventing or treating infectious diseases caused by pathogenic microorganism.

A commercial package comprising the pharmaceutical composition containing the compound (I) identified in the above and a written matter associated therewith, wherein the written matter states that the compound (I) can or should be used for preventing or treating infectious diseases caused by pathogenic microorganism.

The following Preparations and Examples are given for the purpose of illustrating the present invention in more detail.

Preparation 1

To a solution of 6-oxoheptanoic acid (10 g) in methanol (MeOH) (200 ml) was added NaBH₄ (2.89 g) at 0° C., and the suspension was stirred for 3.5 hours at room temperature. NaBH₄ (262 mg) was added to the above solution, and the stirring was continued for one more hour. The mixture was quenched with aqueous HCl, and extracted with ethyl acetate (EtOAc). The extract was washed with water and brine, and dried over MgSO₄. Concentration under reduced pressure gave 6-hydroxyheptanoic acid (9.74 g).

NMR (CDCl₃, δ): 1.20 (3H, d, J=6.2 Hz), 1.3-1.8 (6H, m), 2.37 (2H, t, J=7.4 Hz), 2.3-2.5 (1H, m), 3.7-3.9 (1H, m)

ESI MASS (Negative): 145.4 (M−H)⁻

Preparation 2

To a solution of 6-hydroxyheptanoic acid (5 g) in tetrahydrofuran (THF) (50 ml) was added dropwise BH₃ (1N in THF, 75.2 ml) at 0° C., and the mixture was stirred for 2 hours at 0° C. Water (5 ml) was added to the solution, and the whole was extracted with EtOAc. The extract was washed with water and brine, and dried over MgSO₄. Concentration under reduced pressure gave 1,6-heptanediol (4.5 g).

NMR (CDCl₃, δ): 0.94 (1H, t, J=7.3 Hz), 1.19 (3H, d, J=6.2 Hz), 1.2-1.8 (9H, m), 3.65 (2H, t, J=6.5 Hz), 3.7-3.9 (1H, m)

ESI MASS (Negative): 131.2 (M−H)⁻

Preparation 3

To a stirred solution of 1,6-heptanediol (2.5 g) in N,N-dimethylformamide (DMF) (20 ml) and dichloromethane (CHCl₃) (10 ml) was added imidazole (3.22 g) and tert-butyl(dimethyl)silylchloride (2.85 g) at 0° C., and the mixture was stirred overnight at room temperature. Aqueous NaHCO₃ (5 ml) was added, and the whole was extracted with EtOAc. The extract was washed with water and brine, and dried over MgSO₄. Usual workup followed by flash chromatography over silica gel with n-hexane-EtOAc (1:5) gave 7-[tert-butyl(dimethyl)silyloxy]-2-heptanol (1.86 g).

NMR (CDCl₃, δ): 0.0-0.1 (6H, m), 0.89 (9H, s), 1.19 (3H, d, J=6.1 Hz), 1.3-1.6 (9H, m), 3.60 (2H, t, J=3.6 Hz), 3.7-3.9 (1H, m)

ESI MASS (Positive): 269.4 (M+Na)⁺

Preparation 4

To a stirred solution of 7-[tert-butyl(dimethyl)-silyloxy3-2-heptanol (2.2 g) in DMF (22 ml) was added NaH (60% oil suspension, 428 mg) at 0° C., and the suspension was stirred for 3 hours at room temperature. MeI (12.7 g) was added to the above suspension, and the mixture was stirred overnight at room temperature. H₂O (5 ml) was added, and the whole was extracted with EtOAc, and dried over MgSO₄. Usual workup followed by flash chromatography over silica gel with n-hexane-EtOAc (8:1) gave tert-butyl (6-methoxyheptyloxy)dimethylsilane (1.37 g).

NMR (CDCl₃, δ): 0.05 (9H, s), 0.89 (6H, s), 1.12 (3H, d, J=2.1 Hz), 1.2-1.6 (8H, m), 3.2-3.4 (1H, m), 3.31 (3H, s), 3.60 (2H, t, J=3.3 Hz)

(+) APCI MASS (Positive): 283.4 (M+Na)⁺

Preparation 5

To a solution of tert-butyl(6-methoxyheptyloxy)-dimethylsilane (1.3 g) was added tetrabutylammonium fluoride (TBAF) (1N in THF, 15 ml) at 0° C., and the solution was stirred overnight at room temperature. Water (5 ml) was added to the solution, and the whole was extracted with EtOAc. The extract was washed with water and brine, and dried over MgSO₄. Usual workup followed by flash chromatography over silica gel with n-hexane-EtOAc (3:1) gave 6-methoxy-1-heptanol (581 mg).

NMR (CDCl₃, δ): 1.12 (3H, d, J=6.1 Hz), 1.2-1.7 (9H, m), 3.2-3.4 (1H, m), 3.33 (3H, s), 3.6-3.7 (2H, m)

(+) APCI MASS (Positive): 169.3 (M+Na)⁺

Preparation 6

To a stirred solution of 6-methoxy-1-heptanol (570 mg) was added triethylamine (Et₃N) (1.63 ml) and tosylchloride (TsCl) (817 mg), and the mixture was stirred overnight at room temperature. TsCl (743 mg) and Et₃N (1.63 ml) was added to the above solution, and stirring was continued for 24 hours. Aqueous NaHCO₃ (3 ml) was added, and the whole was extracted with EtOAc. The extract was washed with water and brine, and dried over MgSO₄. Usual workup followed by flash chromatography over silica gel with n-hexane-EtOAc (5:1) gave 6-methoxyheptyl 4-methylbenzenesulfonate (487 mg).

NMR (CDCl₃, δ): 1.09 (3H, d, J=6.1 Hz), 1.2-1.8 (8H, m), 2.45 (3H, s), 3.2-3.4 (1H, m), 3.29 (3H, s), 4.02 (2H, t, J=6.5 Hz), 7.34 (2H, d, J=8.0 Hz), 7.7-7.9 (2H, m)

(+) APCI MASS (Positive): 323.3 (M+Na)⁺

Preparation 7

To a stirred solution of ethyl 4-hydroxybenzoate in DMF (10 ml) was added NaH (60% oil suspension, 77 mg), and the suspension was stirred for 1.5 hours with warming to room temperature. 6-Methoxyheptyl 4-methylbenzenesulfonate (480 mg) in DMF (5 ml) was added to the suspension, and the mixture was stirred overnight. H₂O (2 ml) was added, and the whole was extracted with EtOAc. The extract was washed with water and brine and dried over MgSO₄. Concentration nder reduced pressure gave a residual oil, which was chromatographed over silica gel with n-hexane-EtOAc (10:1) gave ethyl 4-(6-methoxyheptyloxy)benzoate (420 mg).

NMR (CDCl₃, δ): 1.13 (3H, d, J-6.1 Hz), 1.3-1.6 (9H, m), 1.7-1.9 (2H, m), 3.2-3.4 (1H, m), 3.32 (3H, s), 4.00 (2H, t, J=4.0 Hz), 4.34 (2H, q, J=7.1 Hz), 6.8-6.9 (2H, m), 7.9-8.1 (2H, m)

(+) APCI MASS (Positive): 317.3 (M+Na)⁺

Preparation 8

To a stirred solution of ethyl 4-(6-methoxyheptyloxy)-benzoate (410 mg) in ethanol (EtOH) (10 ml) was added hydrazine monohydrate (2 ml), and the solution was heated at reflux for 4 hours. Hydrazine monohydrate (2 ml) was added to the solution, which was stirred three more hours to complete the reaction. The solvent was evaporated under reduced pressure and the residue was extracted with EtOAc. The extract was washed with water and brine, and dried over MgSO₄. Concentration under reduced pressure gave 4-(6-methoxyheptyloxy)benzohydrazide (362 mg).

NMR (CDCl₃, δ): 1.13 (3H, d, J=6.1 Hz), 1.3-1.9 (8H, m), 3.2-3.4 (1H, m), 3.32 (3H, s), 3.9-4.2 (4H, m), 6.8-7.0 (2H, m), 7.3-7.4 (1H, m), 7.6-7.8 (2H, m)

(+) APCI MASS (Positive): 303.3 (M+Na)⁺

Preparation 9

To a stirred solution of ethyl 4-(6-methoxyheptyloxy)-benzoate (350 mg) and pyridine (303 μl) was added 4-methoxycarbonylbenzoylchloride (260 mg) at 0° C., and the mixture was stirred for 1 hour, with warming to room temperature. The solvent was evaporated under reduced pressure, and the residue was poured into water (100 ml) with stirring, and the precipitation was collected and dried under reduced pressure to give methyl 4-[2-[4-(6-methoxyheptyloxy)benzoyl]hydrazinocarbonyl]benzoate (540 mg).

NMR (CDCl₃, δ): 1.14 (3H, d, J=6.1 Hz), 1.3-1.6 (6H, m), 1.7-1.9 (2H, m), 3.2-3.4 (1H, m), 3.32 (3H, s), 3.95 (3H, s), 4.0-4.1 (2H, m), 6.93 (2H, d, J=8.8 Hz), 7.82 (2H, d, J=8.8 Hz), 7.92 (2H, d, J=8.6 Hz), 8.11 (2H, d, J=8.5 Hz), 9.2-9.4 (1H, m), 9.5-9.7 (1H, m)

(+) APCI MASS (Negative): 441.40 (M−H)⁻

Preparation 10

To a stirred solution of methyl 4-[2-[4-(6-methoxyheptyloxy)benzoyl]hydrazinocarbonyl]benzoate (530 mg) was added P₂S₅ (319 mg), and the whole was heated at 120° C. for 3 hours. The mixture was poured into water (40 ml) and was made basic (pH=11) with aqueous NaOH. The precipitation was collected and dried under reduced pressure to give methyl 4-[5-[4-(6-methoxyheptyloxy)phenyl]-1,3,4-thiadiazol-2-yl]benzoate (485 mg).

NMR (CDCl₃, δ): 1.14 (3H, d, J=6.1 Hz), 1.3-1.9 (8H, m), 3.2-3.4 (1H, m), 3.33 (3H, s), 3.96 (3H, s), 4.0-4.1 (2H, m), 6.9-7.1 (2H, m), 7.9-8.0 (2H, m), 8.0-8.1 (2H, m), 8.1-8.2 (2H, m)

(+) APCI MASS (Positive): 441.20 (M+H)⁺

Preparation 11

To a solution of methyl 4-[5-[4-(6-methoxyheptyloxy)-phenyl]-1,3,4-thiadiazol-2-yl]benzoate (470 mg) in EtOH (10 ml) and THF (15 ml) was added NaOH (10% in water, 0.85 ml) and the mixture was heated under reflux for 2 hours. The whole was poured into water (40 ml) and made acidic with aqueous HCl. The precipitation was collected by filtration and dried under reduced pressure to give 4-(5-[4-(6-methoxyheptyloxy)phenyl]-1,3,4-thiadiazol-2-yl]benzoic acid (420 mg).

NMR (DMSO-d₆, δ): 1.06 (3H, d, J=6.1 Hz), 1.2-1.6 (6H, m), 1.6-1.9 (2H, m), 3.20 (3H, s), 3.2-3.4 (1H, m), 4.0-4.2 (2H, m), 7.13 (2H, d, J=8.7 Hz), 7.97 (2H, d, J=8.6 Hz), 8.12 (4H, s), 13.2-13.4 (1H, m)

(+) APCI MASS (Negative): 425.20 (M−H)⁻

Preparation 12

To a suspension of 4-[5-[(4-(6-methoxyheptyloxy)phenyl]-1,3,4-thiadiazol-2-yl]benzoic acid (410 mg) in CH₂Cl₂ (40 ml) was added 1-hydroxybenzotriazole (HOBT) (195 mg) and 1-ethyl 3-(3′-dimethylaminopropyl)carbodiimide hydrochloride (WSCD.HCl) (369 mg), and the mixture was stirred overnight at room temperature. The solvent was evaporated under reduced pressure, and the residue was poured into water (100 ml). Precipitation was collected and washed with water and dried under reduced pressure to give 1-[4-[5-[4-(6-methoxyheptyloxy)phenyl]-1,3,4-thiadiazol-2-yl]benzoyloxy]-1H-1,2,3-benzotriazole (437 mg).

IR (KBr): 1774, 1603, 1441, 1261, 1230, 1176, 1092, 987, 831 cm⁻¹

NMR (CDCl₃, δ): 1.14 (3H, d, J=6.1 Hz), 1.3-2.0 (8H, m), 3.2-3.4 (1H, m), 3.30 (3H, s), 4.05 (2H, t, J=6.4 Hz), 6.9-7.1 (2H, m), 7.4-7.7 (3H, m), 7.9-8.0 (2H, m), 8.1-8.2 (1H, m), 8.2-8.3 (2H, m), 8.3-8.5 (2H, m)

(+) APCI MASS (Positive): 566.2 (M+Na)⁺

Preparation 13

To a solution of 40% methylamine in water (5.78 g) and potassium carbonate (22.6 g) in a mixture of tert-butyl methyl ether (100 ml) and water (100 ml) was added dropwise bromoacetyl bromide (15 g) with stirring under ice-cooling, and the mixture was stirred at 0° C. for 2 hours. The reaction mixture was added to a mixture of ethyl acetate and water. The organic layer was washed with sodium hydrogen carbonate solution and sodium chloride solution. The organic layer was taken and dried over magnesium sulfate. The magnesium sulfate was filtered off, and the filtrate was concentrated under reduced pressure to give N-methyl-2-bromoacetamide (2.14 g).

NMR (CDCl₃, δ): 2.88 (3H, d, J=4.9 Hz), 3.89 (2H, s), 6.51 (1H, br s)

MASS (m/z): 152.1, 176.1 (M⁺+H)

Preparation 14

To a suspension of 3-bromopropylamine hydrobromide (5 g) in dichloromethane (50 ml) was added dropwise benzylchloroformate (3.9 g) in dichloromethane (20 ml) under ice-cooling. To the mixture was added dropwise triethylamine (7.96 ml) in dichloromethane (30 ml) and the mixture was stirred at ambient temperature for 1 hour. Water (100 ml) was added and resulting organic layer separated, washed with 1N—HCl, water, saturated sodium hydrogencarbonate and brine, and dried, and the solvent was evaporated under reduced pressure. The residue was chromatographed on a column of silica gel (100 g) eluting with n-hexane/ethyl acetate (2:1) to give benzyl 3-bromopropylcarbamate (5.33 g).

IR (KBr): 1689.3, 1538.9, 1261.2 cm⁻¹

NMR (CDCl₃, δ): 2.07 (2H, d, J=6.5 Hz), 3.31-3.47 (4H, m), 4.90 (1H, bm), 5.10 (2H, s), 7.30-7.45 (5H, m)

ESI MASS (Positive)(m/z): 294.1 (M+Na)⁺

The Starting Compounds used and the Object Compounds obtained in the following Preparation 15 to 27 are given in the table as below, in which the formulas of the starting compounds are in the upper column, and the formulas of the object compounds are in the lower column, respectively.

| Preparation | |
|---|---|
| No. | Formula |
| 15 | 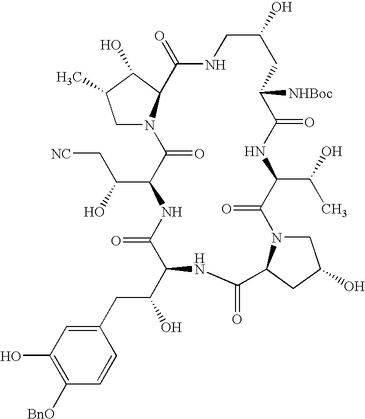 |
| | 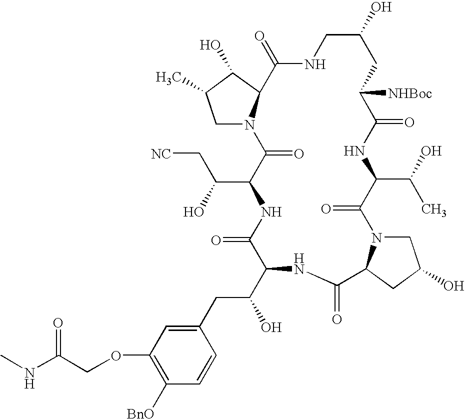 |
| 16 | 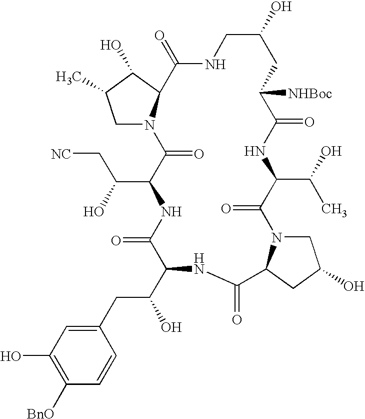 |
| | 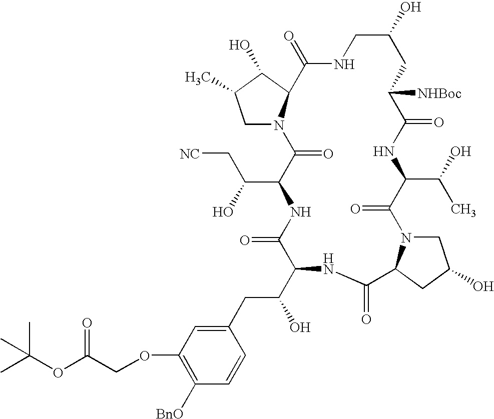 |
| 17 | 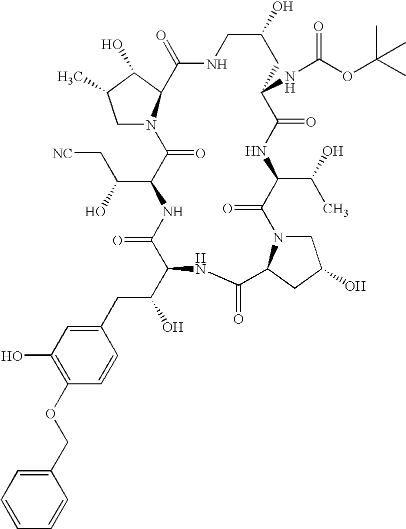 |
| | 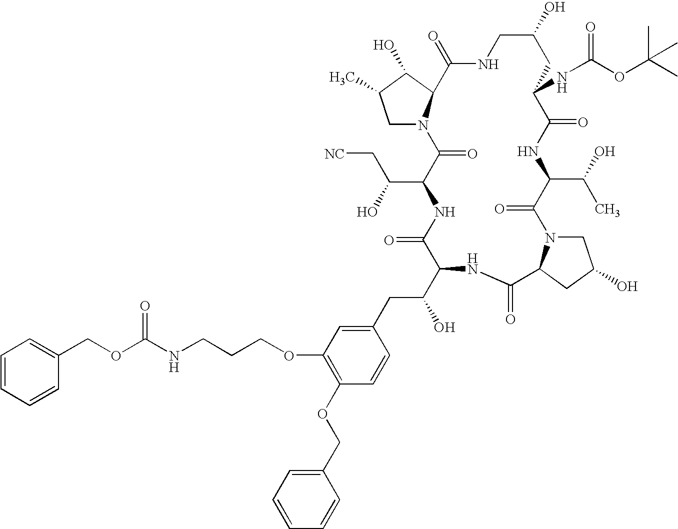 |
| 18 | 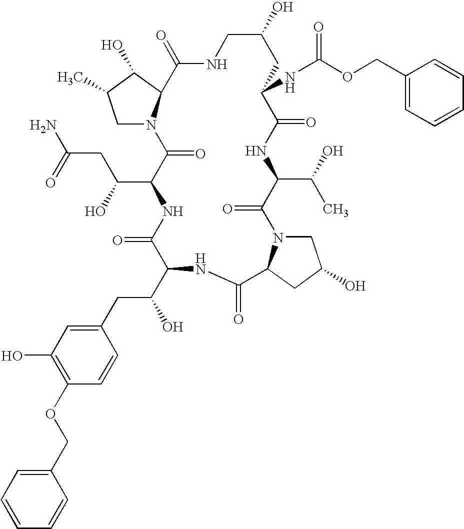 |
| | 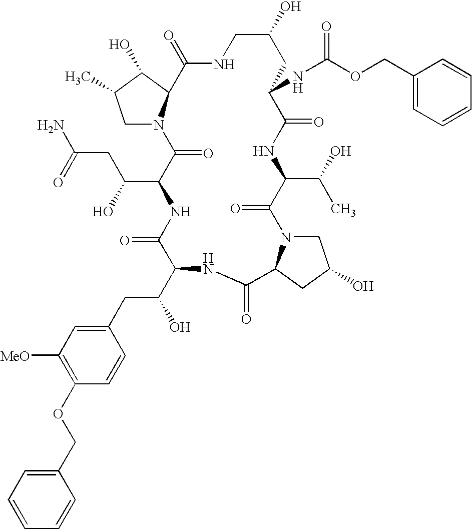 |
| 19 | 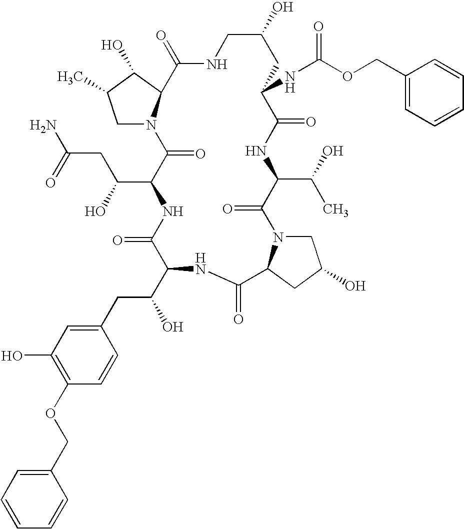 |
| | 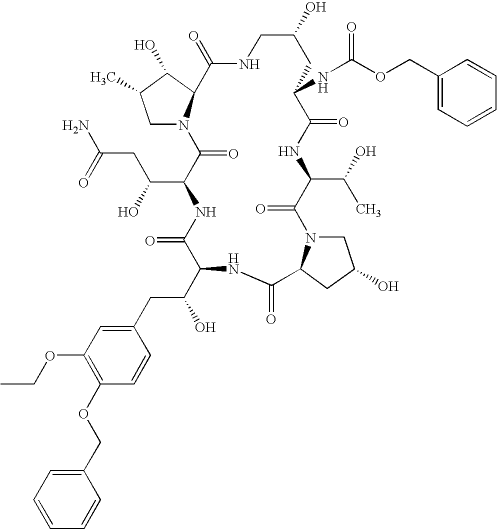 |
| 20 | 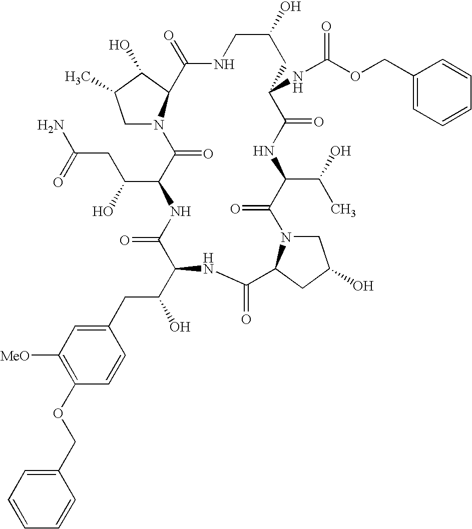 |
| | 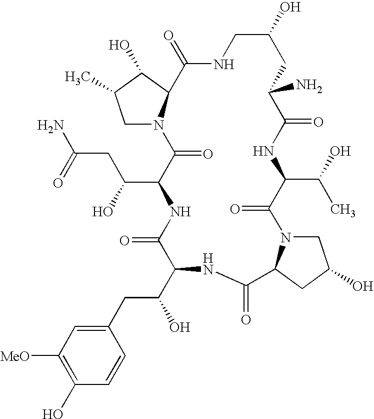 |
| 21 | 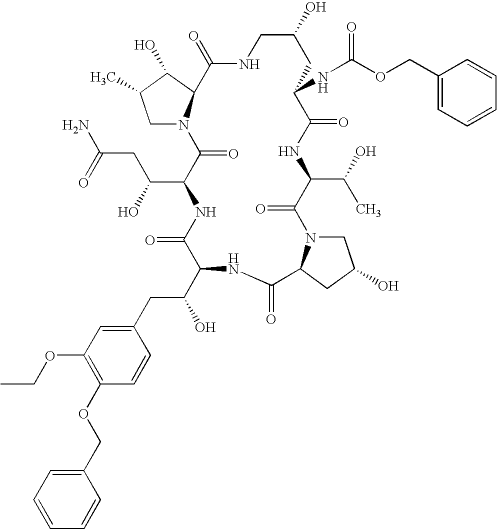 |
| | 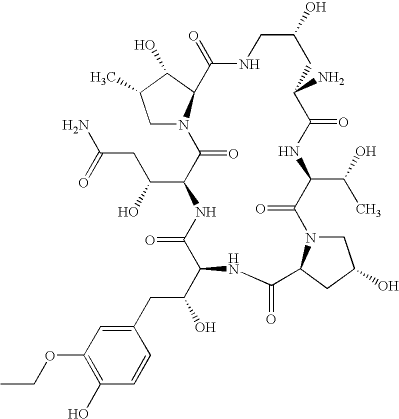 |
| 22 | 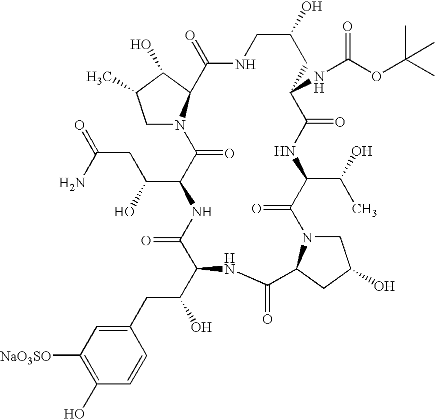 |
| | 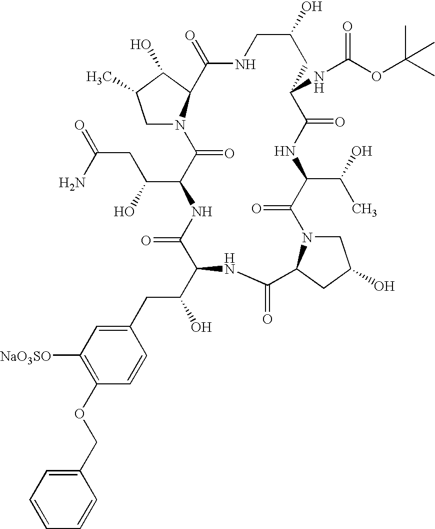 |
| 23 | 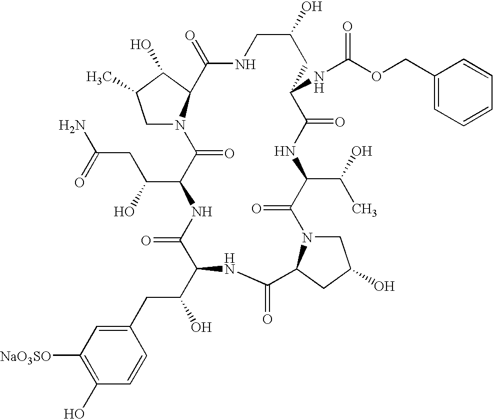 |
| | 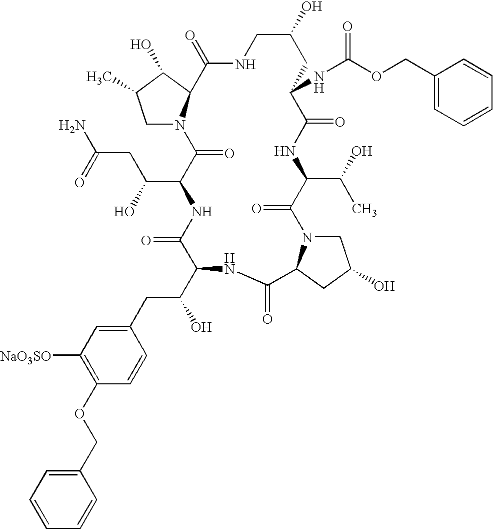 |
| 24 | 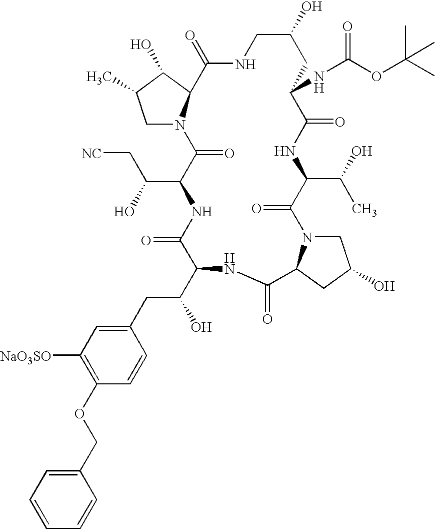 |
| | 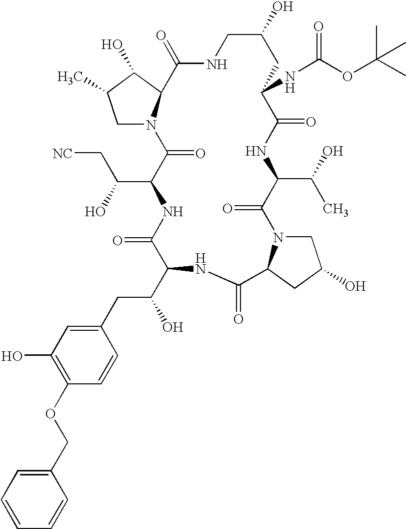 |
| 25 | 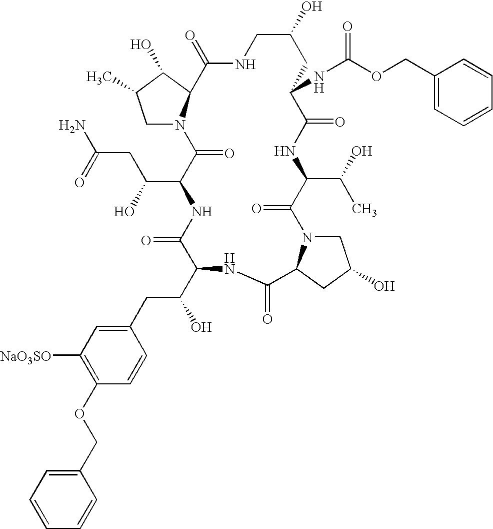 |
| | 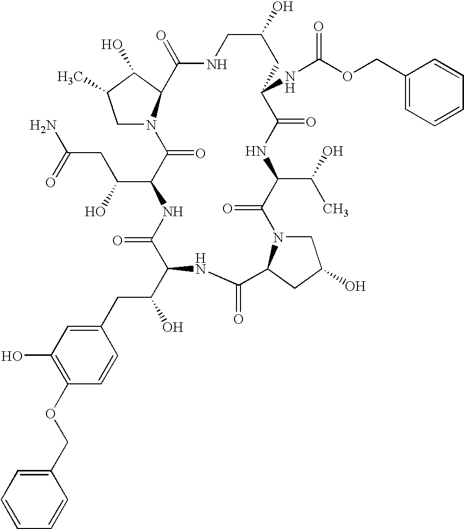 |
| 26 | 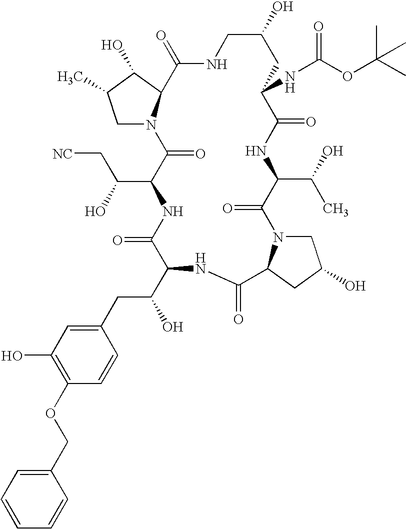 |
| | 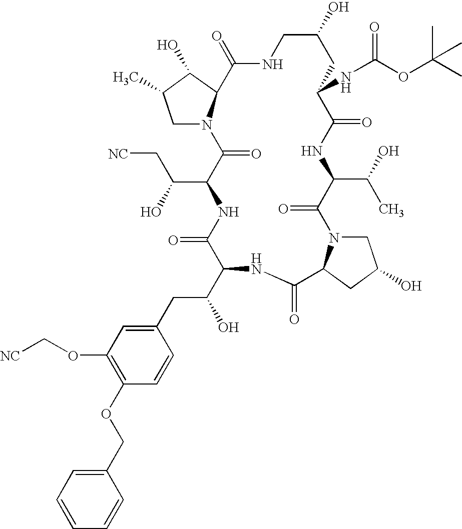 |
| 27 | 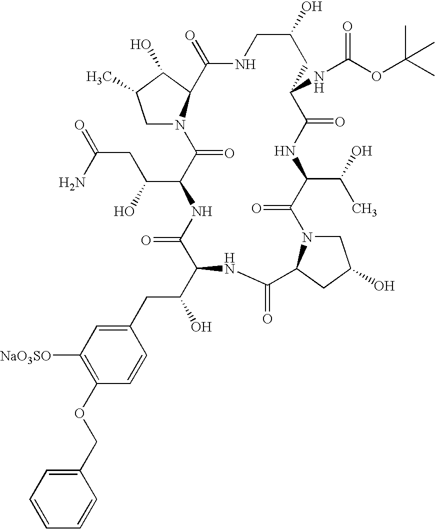 |
| | 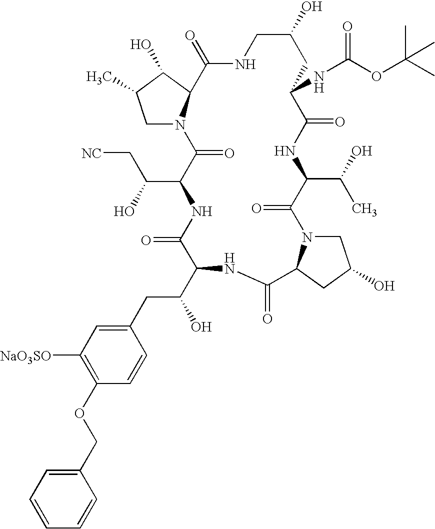 |

Preparation 15

A mixture of the starting compound (15) (1 g), N-methyl-2-bromoacetamide (229 mg) and potassium carbonate (416 mg) in N,N-dimethylformamide (10 ml) was stirred for 2 days at room temperature. Water was added to the mixture. The solution was subjected to column chromatography on ODS eluting with 25% acetonitrile in water. The fractions containing the object compound were collected and evaporated under reduced pressure to remove acetonitrile. The residue was lyophilized to give the object compound (15) (398 mg).

NMR (DMSO-d₆+D₂O, δ): 0.95 (3H, d, J=6.8 Hz), 1.08 (3H, d, J=5.1 Hz), 1.36 (9H, s), 1.5-4.5 (30H, m), 4.75-7.9 (2H, m), 5.0-5.15 (2H, m), 6.7-6.8 (1H, m), 7.00 (2H, d, J=8.7 Hz), 7.3-7.55 (5H, m)

MASS (m/z): 1090.3 (M⁺+Na)

Preparation 16

A mixture of the starting compound (16) (3 g), tert-butyl bromoacetate (0.666 ml) and potassium carbonate (1.25 g) in N,N-dimethylformamide (30 ml) was stirred for 5 hours at 60° C. Water and acetonitrile were added to the mixture. The mixture was purified by column chromatography on ODS to give the object compound (16) (2.48 g).

NMR (DMSO-d₆+D₂O, δ): 0.95 (3H, d, J=6.9 Hz), 1.08 (3H, d, J=5.8 Hz), 1.36 (9H, s), 1.42 (9H, s), 1.5-3.5 (11H, m), 3.7-5.2 (20H, m), 6.6-6.8 (2H, m), 6.96 (1H, d, J=8.2 Hz), 7.25-7.5 (5H, m)

MASS (m/z): 1133.4 (M⁺+Na)

Preparation 17

A solution of the starting compound (17) (5 g) in DMF (50 ml) was treated with benzyl 3-bromopropylcarbamate (1.64 g) and potassium carbonate (970 mg) and stirred at 70° C. for 10 hours. The reaction mixture was dissolved in a mixture of water (500 ml) and acetonitrile (200 ml). The mixture was filtered, and filtrate was subjected to column chromatography on ODS (Daiso-gel, SP-120-40/60-ODS-B (Trademark: prepared by Daiso Co., Ltd.)) (1L) eluting with 30% acetonitrile in water. The fractions containing the object compound were collected and evaporated under reduced pressure to remove acetonitrile. The residue was lyophilized to give the object compound (17) (2.80 g).

IR (KBr): 3359.4, 1668.1, 1650.8, 1633.4, 1513.8 cm⁻¹

NMR (DMSO-d₆+D₂O, δ): 0.95 (3H, d, J=6.7 Hz), 1.06 (3H, d, J=6.0 Hz), 1.35 (9H, s), 1.46-2.50 (11H, m), 2.60-3.38 (8H, m), 3.58-4.41 (14H, m), 4.80-4.87 (2H, m), 5.02 (2H, s), 5.06 (2H, s), 6.64-6.93 (3H, m), 7.30-7.48 (10H, m)

ESI MASS (Negative)(m/z): 1186.5 (M−H)⁻

The following compounds [Preparation 18 to 19] were obtained according to a similar manner to that of Preparation 17.

Preparation 18

IR (KBr): 3352, 1664, 1632, 1531, 1514, 1448, 1325, 1263, 1140, 1086 cm⁻¹

NMR (DMSO-d₆+D₂O, δ): 0.96 (3H, d, J=6.7 Hz), 1.05 (3H, d, J=5.8 Hz), 1.5-1.7 (1H, m), 1.8-2.1 (2H, m), 2.1-2.6 (5H, m), 2.8-3.0 (1H, m), 3.1-3.3 (1H, m), 3.75 (3H, s), 3.4-4.1 (8H, m), 4.1-4.5 (7H, m), 4.7-5.1 (6H, m), 6.6-6.8 (2H, m), 6.93 (1H, d, J=8.3 Hz), 7.2-7.5 (10H, m)

ESI MASS: 1085.4 (M+Na)⁺ (Positive), 1061.4 (M+H)⁺ (Negative)

Analysis Calculated for C₅₁H₆₆N₈O₁₇.4H₂O: C, 53.96; H, 6.57; N, 9.87 Found: C, 53.99; H, 6.57; N, 9.84

Preparation 19

IR (KBr): 3361, 3344, 2935, 1666, 1633, 1512, 1448, 1435, 1261, 1086 cm⁻¹

NMR (DMSO-d₆+D₂O, δ): 0.96 (3H, d, J=6.8 Hz), 1.04 (3H, d, J=5.8 Hz), 1.33 (3H, t, J=6.9 Hz), 1.5-2.6 (8H, m), 2.8-3.0 (1H, m), 3.1-3.3 (1H, m), 3.3-4.5 (17H, m), 4.7-5.1 (6H, m), 6.6-7.0 (3H, m), 7.2-7.5 (10H, m)

ESI MASS (Positive): 1099.4 (M+Na)⁺

Analysis Calculated for C₅₂H₆₈N₈O₁₇.3H₂O: C, 55.21; H, 6.59; N, 9.91 Found: C, 55.45; H, 6.49; N, 9.92

Preparation 20

A mixture of the starting compound (20) (3.04 g) and 10% palladium on carbon (50% wet) (1.5 g) in methanol (60 ml) was stirred at room temperature under hydrogen atmosphere for 5 hours. After removal of insoluble solids, the filtrate was concentrated in vacuo and lyophilized to give the object compound (20) (2.19 g).

IR (KBr): 3350, 1660, 1633, 1518, 1437, 1275, 1084 cm⁻¹

NMR (DMSO-d₆+D₂O, δ): 0.81 (3H, d, J=6.7 Hz), 1.09 (3H, d, J=6.0 Hz), 1.8-2.6 (8H, m), 2.6-2.8 (1H, m), 3.0-3.2 (1H, m), 3.74 (3H, s), 3.3-4.5 (15H, m), 4.7-4.9 (2H, m), 6.5-6.7 (3H, m)

ESI MASS (Positive): 839.4 (M+H)⁺, 861.3 (M+Na)⁺, 1699.5 (2M+Na)⁺

Analysis Calculated for C₃₆H₅₄N₈O₁₅.4.5H₂O: C, 47.00; H, 6.90; N, 12.18 Found: C, 47.02; H, 6.80; N, 12.04

The following compound was obtained according to a similar manner to that of Preparation 20.

Preparation 21

NMR (DMSO-d₆+D₂O, δ): 0.96 (3H, d, J=6.7 Hz), 1.09 (3H, d, J=6.1 Hz), 1.32 (3H, t, J=6.9 Hz), 1.8-2.8 (9H, m), 3.0-4.5 (18H, m), 4.7-4.9 (2H, m), 6.5-6.7 (3H, m)

ESI MASS (Positive): 853.3 (M+H)⁺, 875.3 (M+Na)⁺

Preparation 22

To a solution of a mixture of the starting compound (22) (186 g), benzylbromide (43.1 ml) in DMF (1674 ml) was added lithium hydroxide monohydrate (11.4 g) with stirring and the mixture was stirred at ambient temperature for 5 hours. The reaction mixture was added a mixture of acetonitrile (1674 ml) and water (19 l). The mixture was filtered, and filtrate was subjected to column chromatography on ODS (Daiso-gel, SP-120-40/60-ODS-B (Trademark: prepared by Daiso Co., Ltd.)) (2 l) eluting with 25% acetonitrile in water. The fractions containing the object compound were collected and evaporated under reduced pressure to remove acetonitrile. The residue was lyophilized to give the object compound (22) (172.26 g).

IR (KBr): 3353.6, 1668.1, 1654.1, 1631.5, 1267.0 cm⁻¹

NMR (DMSO-d₆+D₂O, δ): 0.97 (3H, d, J=6.75 Hz), 1.05 (3H, d, J=6.0 Hz), 1.34 (9H, s), 1.45-2.50 (9H, m), 2.87-3.25 (6H, m), 3.56-4.49 (12H, m), 4.80-4.87 (2H, m), 5.06 (2H, s), 6.75-7.03 (3H, m), 7.27-7.50 (5H, m)

ESI MASS (Negative)(m/z): 1093.3 (M⁺−Na) The following compound was obtained according to a similar manner to that of Preparation 22.

Preparation 23

IR (KBr): 3352, 1664, 1628, 1531, 1512, 1446, 1267, 1084, 1047 cm⁻¹

NMR (DMSO-d₆+D₂O, δ): 0.97 (3H, d, J=6.8 Hz), 1.04 (3H, d, J=5.8 Hz), 1.5-1.8 (1H, m), 1.8-2.0 (3H, m), 2.0-2.6 (5H, m), 2.8-3.0 (1H, m), 3.1-3.3 (1H, m), 3.4-4.1 (8H, m), 4.1-4.5 (7H, m), 4.7-5.1 (6H, m), 6.7-6.8 (1H, m), 6.88 (1H, d, J=8.4 Hz), 7.3-7.5 (11H, m)

ESI MASS (Positive): 1173.3 (M+Na)⁺

Analysis Calculated for C₅₀H₆₃N₈O₂₀SNa.4H₂O: C, 49.10; H, 5.85; N, 9.16 Found: C, 48.84; H, 6.06; N, 9.02

Preparation 24

To a solution of the starting compound (24) (26.3 g) in methanol (263 ml) was added 10% HCl (131 ml) with stirring under ice-cooling, and the mixture was stirred at the ambient temperature for 9 hours. The reaction mixture was concentrated in vacuo. To a solution of the resulting residue in DMF (263 ml) was added N,N-diisopropylethylamine (42 ml) and di-tert-butyldicarbonate (6.8 g) with stirring under ice-cooling, and the mixture was stirred at ambient temperature overnight. The reaction mixture was added water (5260 ml). The mixture was filtered, and filtrate was subjected to column chromatography on ODS (Daiso-gel, SP-120-40/60-ODS-B (Trademark: prepared by Daiso Co., Ltd.))(1 l) eluting with 30% acetonitrile in water. The fractions containing the object compound were collected and evaporated under reduced pressure to remove acetonitrile. The residue was lyophilized to give the object compound (24) (20.55 g).

IR (KBr): 3361.3, 2362.4, 1666.2, 1631.5, 1249.6 cm⁻¹

NMR (DMSO-d₆+D₂O, δ): 0.95 (3H, d, J=6.7 Hz), 1.08 (3H, d, J=5.6 Hz), 1.35 (9H, s), 1.46-2.50 (9H, m), 2.60-3.38 (6H, m), 3.58-4.41 (12H, m), 4.80-4.87 (2H, m), 5.06 (2H, s), 6.47-6.87 (3H, m), 7.30-7.48 (5H, m)

ESI MASS (Negative)(m/z): 995.5 (M−H)⁻

The following compound was obtained according to a similar manner to that of Preparation 24.

Preparation 25

IR (KBr): 3352, 3331, 1666, 1632, 1531, 1514, 1446, 1441, 1270, 1248, 1086 cm⁻¹ NMR (DMSO-d₆+D₂O, δ): 0.96 (3H, d, J=6.7 Hz), 1.05 (3H, d, J=5.9 Hz), 1.5-1.8 (1H, m), 1.8-2.0 (2H, m), 2.1-2.5 (5H, m), 2.8-3.0 (1H, m), 3.1-3.3 (1H, m), 3.4-4.1 (8H, m), 4.1-4.5 (7H, m), 4.7-5.1 (6H, m), 6.4-6.6 (1H, m), 6.66 (1H, d, J=1.8 Hz), 6.85 (1H, d, J=8.3 Hz), 7.3-7.5 (10H, m)

ESI MASS (Positive): 1071.3 (M+Na)⁺

Analysis Calculated for C₅₀H₆₄N₈O₁₇.3H₂O′ C, 54.44; H, 6.40; N, 10.16 Found: C, 54.41; H, 6.59; N, 10.12

Preparation 26

A mixture of the starting compound (26) (10 g), romoacetonitrile (1.4 ml) and lithium hydroxide monohydrate (842 mg) in N,N-dimethylformamide (100 ml) was stirred at ambient temperature for 3 hours. The reaction mixture was poured into a mixture of water (1000 ml) and acetonitrile (130 ml). The solution was subjected to column chromatography on ODS (Daiso-gel, SP-120-40/60-ODS-B (Trademark: prepared by Daiso Co., Ltd.)) (500 ml) eluting with 50% acetonitrile in water. The fractions containing the object compound were collected and evaporated under reduced pressure to remove acetonitrile. The residue was lyophilized to give the object compound (26) (9.1 g).

IR (KBr): 3351.7, 2360.4, 1648.8, 1631.5, 1257.4 cm⁻¹

NMR (DMSO-d₆+D₂O; δ): 0.95 (3H, d, J=6.8 Hz), 1.08 (3H, d, J=5.8 Hz), 1.36 (9H, s), 1.67-5.19 (31H, m), 6.83-7.48 (8H, m)

ESI MASS (Positive)(m/z): 1058.3 (M+Na)⁺

Preparation 27

A mixture of the starting compound (27) (60 g), zeorite (60 g) and sodium hydrogen carbonate (4.96 g) in dimethylformamide (600 ml) was stirred at room temperature. To the mixture was added N,N-diisopropylethylamine (10.3 ml) then added methanesulfonylchloride (4.57 ml), and the reaction mixture was stirred for 1 hour at room temperature. Previous treatment was repeated four times. The mixture was filtered, and filtrate was poured into a mixture of sodium hydrogen carbonate (199 g) in ice-water (8400 ml). The mixture was subjected to column chromatography on ODS (Daiso-gel, SP-120-40/6-ODS-B (Trademark: prepared by Daiso Co., Ltd.)) (2 l) eluting with 20% acetonitrile in water. The fractions containing the object compound were collected and evaporated under reduced pressure to remove acetonitrile. The residue was lyophilized to give the object compound (27) (24.1 g).

IR (KBr): 3355.5, 2256.3, 1666.6, 1631.5, 1267.0 cm⁻¹

NMR (DMSO-d₆+D₂O, δ): 0.95 (3H, d, J=6.8 Hz), 1.08 (3H, d, J=5.7 Hz), 1.35 (9H, s), 1.46-2.50 (9H, m), 2.81-3.27 (6H, m), 3.59-4.41 (12H, m), 4.80-4.87 (2H, m), 5.06 (2H, s), 6.71-6.96 (3H, m), 7.33-7.50 (5H, m)

ESI MASS (Negative)(m/z): 1075.3 (M⁺−Na)⁺

Preparation 28

To a solution of 4′-(4-morpholinyl)-1,1′-biphenyl-4-ol (1.02 g) in dichloromethane (31 ml) and pyridine (1.47 ml) was added dropwise trifluoromethanesulfonic anhydride (2.66 ml) at 0° C. The reaction mixture was stirred for 1 hour at 0° C. and poured into 50% acetonitrile in water. The precipitate was collected by filtration and dried under reduced pressure to give 4′-(4-morpholinyl)-1,1′-biphenyl-4-yl trifluoromethanesulfonate (3.219 g).

NMR (CDCl₃, δ): 3.15-3.3 (4H, m), 3.85-3.95 (4H, m), 6.99 (2H, d, J=8.7 Hz), 7.30 (2H, d, J=8.8 Hz), 7.49 (2H, d, J=8.7 Hz), 7.60 (2H, d, J=8.8 Hz)

MASS (m/z): 388.2 (M+H)

Preparation 29

A mixture of 4-(ethoxycarbonyl)phenylboronic acid (1 g), 4′-(4-morpholinyl)-1,1′-biphenyl-4-yl trifluoromethane-sulfonate (2 g), tetrakis(triphenylphosphine)palladium(0) (298 mg) and sodium carbonate (1.15 g) in a mixed solvent of dioxane (20 ml) and water (2.5 ml) was refluxed for 2.5 hours. After cooling to ambient temperature, the reaction mixture was poured into water. The resulting precipitates were filtered, washed with acetonitrile and dried under reduced pressure to give 4″-(4-morpholinyl)-1,1′:4′,1″-terphenyl-4-carboxylate (1.043 g).

NMR (CDCl₃, δ): 1.42 (3H, t, J=7.1 Hz), 3.15-3.3 (4H, m), 3.8-4.0 (4H, m), 4.41 (2H, q, J=7.1 Hz), 7.01 (2H, d, J=8.8 Hz), 7.5-7.85 (8H, m), 8.05-8.2 (2H, m)

MASS (m/z): 410.2 (M+Na)

Preparation 30

To a solution of phenyl (4-piperidyl)methanone hydrochloride (5 g) in tetrahydrofuran (50 ml) and triethylamine (6.79 ml) was added dropwise a solution of di(tert-butyl) dicarbonate (5.32 g) in tetrahydrofuran (50 ml) and stirred for 2 hours at room temperature. The reaction mixture was added to a mixture of water and ethyl acetate. The organic layer was washed with brine and dried over magnesium sulfate. The magnesium sulfate was filtered off, and the filtrate was concentrated under reduced pressure to give tert-butyl 4-benzoyl-1-piperidinecarboxylate (7.018 g).

NMR (CDCl₃, δ): 1.47 (9H, s), 1.55-1.95 (4H, m), 2.8-3.0 (2H, m), 3.3-3.5 (1H, m), 4.05-4.25 (2H, m), 7.4-7.6 (3H, m), 7.9-8.0 (2H, m)

MASS (m/z): 312.3 (M+Na)

Preparation 31

To a solution of tert-butyl 4-benzoyl-1-piperidinecarboxylate (7 g) in methanol (80 ml) was added sodium borohydride (1.1 g) at 0° C. The mixture was stirred for 2 hours at 0° C. The reaction mixture was added to a mixture of water and ethyl acetate. The organic layer was washed with brine and dried over magnesium sulfate. The magnesium sulfate was filtered off, and the filtrate was concentrated under reduced pressure to give tert-butyl 4-[hydroxy(phenyl)methyl]-1-piperidinecarboxylate (6.806 g).

NMR (CDCl₃, δ): 1.05-1.4 (4H, m), 1.44 (9H, s), 1.6-1.8 (1H, m), 1.86 (1H, d, J=3 Hz), 1.9-2.05 (1H, m), 2.45-2.75 (2H, m), 3.9-4.25 (2H, m), 4.39 (1H, dd, J=3, 7.4 Hz), 7.2-7.4 (5H, m)

MASS (m/z): 314.2 (M+Na)

Preparation 32

To a solution of tert-butyl 4-[hydroxy(phenyl)methyl]-1-piperidinecarboxylate (5.8 g) in N,N-dimethylformamide (58 ml) was added sodium hydride (60% dispersion in mineral oil) (1.19 g). The solution was stirred for 1 hour at 60° C. To the reaction mixture was added iodomethane (3.72 ml). The mixture was stirred for 4 hours at room temperature. The reaction mixture was added to a mixture of water and ethyl acetate. The organic layer was washed with brine and dried over magnesium sulfate. The magnesium sulfate was filtered off, and the filtrate was concentrated under reduced pressure. The residue was purified by silica gel chromatography (10:1 dichloromethane-methanol elution) to give tert-butyl 4-[methoxy(phenyl)methyl]-1-piperidinecarboxylate (5.019 g).

NMR (CDCl₃, δ): 1.0-1.35 (3H, m), 1.43 (9H, s), 1.6-1.85 (1H, m), 1.9-2.1 (1H, m), 2.45-2.75 (2H, m), 3.18 (3H, s), 3.79 (1H, d, J=7.7 Hz), 3.85-4.2 (2H, m), 7.15-7.4 (5H, m)

MASS (m/z): 328.3 (M+Na)

Preparation 33

To a solution of tert-butyl 4-[methoxy(phenyl)methyl]-1-piperidinecarboxylate (5 g) and anisole (12.5 ml) in dichloromethane (25 ml) was added dropwise with stirring trifluoroacetic acid (25.2 ml) at 0° C. The mixture was stirred for 2 hours at room temperature. The reaction mixture was added to a mixture of water and ethyl acetate and was adjusted to pH 3 with 1.0 mol/1 hydrochloric acid. The aqueous layer was separated and ethyl acetate was added. The mixture was adjusted to pH 12 with 1.0 mol/1 sodium hydroxide solution. The organic layer was separated, washed with brine and dried over magnesium sulfate. The magnesium sulfate was filtered off, and the filtrate was concentrated under reduced pressure to give 4-[methoxy(phenyl)methyl]-piperidine (2.632 g).

NMR (CDCl₃, δ): 1.0-1.35 (3H, m), 1.6-1.8 (1H, m), 1.9-2.1 (1H, m), 2.35-2.65 (2H, m), 2.9-3.2 (2H, m), 3.18 (3H, s), 3.78 (1H, d, J=7.7 Hz), 7.15-7.4 (5H, m)

MASS (m/z): 206.4 (M+H)

Preparation 34

To a solution of 4-[methoxy(phenyl)methyl]piperidine (1.34 g) and 1-bromo-4-fluorobenzene (1.92 ml) in dimethylsulfoxide (13.4 ml) was added potassium carbonate (3.61 g). The solution was stirred for 7 hours at 150° C. The reaction mixture was added to a mixture of water and ethyl acetate. The organic layer was washed with brine and dried over magnesium sulfate. The magnesium sulfate was filtered off, and the filtrate was concentrated under reduced pressure. The residue was purified by silica gel chromatography (10:1 hexane-ethyl acetate elution) to give ethyl 4-[4-[methoxy(phenyl)methyl]-1-piperidyl]benzoate (1.124 g).

NMR, (CDCl₃, δ): 1.2-1.55 (6H, m), 1.6-1.9 (1H, m), 2.0-2.2 (1H, m), 2.6-2.9 (2H, m), 3.19 (3H, s), 3.7-4.0 (3H, m), 4.31 (2H, q, J=7.1 Hz), 6.82 (2H, d, J=9.0 Hz), 7.2-7.4 (5H, m), 7.89 (2H, d, J=9.0 Hz)

MASS (m/z): 354.2 (M+H)

Preparation 35

To a solution of ethyl 4-[4-[methoxy(phenyl)methyl]-1-piperidyl]benzoate (1.12 g) in ethanol (22 ml) and tetrahydrofuran (9 ml) was added hydrazine monohydrate (13.83 ml) and the mixture was refluxed for 16.5 hours. After cooling, the solvent was removed under reduced pressure. Water was added and the precipitate was collected by filtration, washed with water and dried under reduced pressure to give 4-[4-[methoxy(phenyl)methyl]-1-piperidyl]benzoylhydrazine (1.022 g).

NMR (CDCl₃, δ): 1.2-1.9 (4H, m), 2.05-2.2 (1H, m), 2.6-2.9 (2H, m), 3.19 (3H, s), 3.75-3.95 (5H, m), 6.85 (2H, d, J=9.0 Hz), 7.15 (1H, s), 7.15-7.4 (5H, m), 7.61 (2H, d, J=9.0 Hz)

MASS (m/z): 240.3 (M+H)

Preparation 36

To a solution of 4-[4-[methoxy(phenyl)methyl]-1-piperidyl]benzoylhydrazine (1.02 g) in tetrahydrofuran (30 ml) and pyridine (0.729 ml) was added 4-methoxycarbonylbenzoyl chloride (627 mg) at 0° C. The reaction mixture was stirred for 2 hours at room temperature and poured into water. The precipitate was collected by filtration, washed with water and dried under reduced pressure to give methyl 4-[2-[4-[4-[methoxy(phenyl)methyl]-1-piperidyl]benzoyl]hydrazinocarbonyl]benzoate (1.45 g).

NMR (CDCl₃, δ): 1.2-1.9 (4H, m), 2.05-2.25 (1H, m), 2.6-2.9 (2H, m), 3.20 (3H, s), 3.7-3.95 (3H, m), 3.95 (3H, s), 6.86 (2H, d, J=8.9 Hz), 7.2-7.45 (5H, m), 7.74 (2H, d, J=8.9 Hz), 7.92 (2H, d, J=8.4 Hz), 8.13 (2H, d, J=8.4 Hz), 9.07 (1H, d, J=6.3 Hz), 9.44 (1H, d, J=6.3 Hz)

MASS (m/z): 524.2 (M+Na)

Preparation 37

A suspension of methyl 4-[2-[4-[4-[methoxy(phenyl)-methyl]-1-piperidyl]benzoyl]hydrazinocarbonyl]benzoate (1.35 g) and diphopshorus pentasulfide (897 mg) in dimethoxyethane (39 ml) was stirred for 2 hours at 100° C. To the reaction mixture was added water. The precipitate was collected by filtration, washed with water and acetonitrile and dried under reduced pressure to give methyl 4-[5-[4-[4-[methoxy(phenyl)methyl]-1-piperidyl]phenyl]-1,3,4thiadiazol-2-yl]benzoate (1.035 g).

NMR (CDCl₃, δ): 1.2-1.9 (4H, m), 2.1-2.3 (1H, m), 2.7-3.0 (2H, m), 3.20 (3H, s), 3.7-4.0 (3H, m), 3.96 (3H, s), 7.0-7.15 (2H, m), 7.2-7.45 (5H, m), 7.91 (2H, d), 8.05 (2H, d), 8.15 (2H, d)

MASS (m/z): 500.1 (M+H)

Preparation 38

A mixture of ethyl 4-fluorobenzoate (3 g), 2-(1-piperazinyl)pyrimidine dihydrochloride (4.23 g) and potassium carbonate (3.21 g) in dimethylsulfoxide (15 ml) was stirred for 3 hours at 150° C. The reaction mixture was poured into water (150 ml). The resulting precipitate was collected by filtration, and dried under reduced pressure to give ethyl 4-[4-(2-pyrimidinyl)-1-piperazinyl]benzoate (3.44 g).

IR (KBr): 1697.1, 1589.1, 1373.1, 1240.0 cm⁻¹

NMR (CDCl₃, δ): 1.37 (3H, t, J=7.1 Hz), 3.40-3.45 (4H, m), 3.96-4.02 (4H, m), 4.35 (2H, q, J=7.1 Hz), 6.52-6.56 (1H, m), 6.88-6.94 (2H, m), 7.93-7.98 (2H, m), 8.33-8.36 (2H, m)

ESI MASS (Positive)(m/z): 335.2 (M+Na)⁺

Preparation 39

A solution of ethyl 4-[4-(2-pyrimidinyl)-1-piperazinyl)benzoate (3.3 g) and hydrazine monohydrate (10.2 ml) in ethylalcohol (20 ml) was stirred at reflux for 10 hours. The reaction mixture was poured into water (40 ml). The resulting precipitate was collected by filtration, and dried under reduced pressure to give 4-[4-(2-pyrimidinyl)-1-piperazinyl]benzohydrazide (1.89 g).

IR (KBr): 3284.2, 1619.9, 1583.3, 1513.8, 1492.6, 1361.5 cm⁻¹

NMR (CDCl₃, δ): 3.36-3.45 (4H, m), 3.96-4.07 (6H, m), 6.52-6.56 (1H, m), 6.88-6.97 (3H, m), 7.65-7.72 (2H, m), 8.33-8.36 (2H, m)

ESI MASS (Positive)(m/z): 321.2 (M+Na)⁺

Preparation 40

To a solution of 4-[4-(2-pyrimidinyl)-1-piperazinyl]benzohydrazide (1.65 g) and pyridine (1.34 ml) in N,N-dimethylformamide (28 ml) was added methyl 4-(chlorocarbonyl)benzoate (1.1 g) under ice-cooling, and the mixture was stirred at ambient temperature for 5 hours. The reaction mixture was poured into water (140 ml). The resulting precipitate was collected by filtration, and dried under reduced pressure to give methyl 4-[2-[4-[4-(2-pyrimidinyl)-1-piperazinyl]benzoyl]hydrazinocarbonyl]-benzoate (1.86 g).

IR (KBr): 3268.8, 1722.1, 1641.1, 1587.1, 1234.2 cm⁻¹ NMR (DMSO-d₆, δ): 3.35-3.90 (11H, m), 6.65-6.70 (1H, m), 7.04-7.08 (2H, m), 7.83-7.87 (2H, m), 8.01-8.12 (4H, m), 8.39-8.42 (2H, m), 10.30 (1H, bs), 10.59 (1H, bs)

ESI MASS (Positive) (m/z): 461.3 (M+H)⁺, 483.3 (M+Na)⁺

Preparation 41

A mixture of methyl 4-[2-[4-[4-(2-pyrimidinyl)-1-piperazinyl]benzoyl]hydrazinocarbonyl]benzoate (1.53 g) and phosphorus pentasulfide (0.96 g) in pyridine (23 ml) was stirred at reflux for 16 hours. The reaction mixture was poured into water (138 ml). The mixture was adjusted to pH 9 with 1N-sodiumhydroxide solution. The resulting precipitate was collected by filtration, and washed acetonitrile (50 ml). The precipitate was collected by filtration, and dried under reduced pressure to give methyl 4-[5-[4-[4-(2-pyrimidinyl)-1-piperazinyl]phenyl]-1,3,4-thiadiazol-2-yl]benzoate (1.29 g).

IR (KBr): 1724.0, 1606.4, 1585.2, 1490.7, 1438.6, 1280.5 cm⁻¹

ESI MASS (Positive)(m/z): 459.0 (M+H)⁺

Preparation 42

A mixture of methyl 4-[5-[4-[4-(2-pyrimidinyl)-1-piperazinyl]phenyl]-1,3,4-thiadiazol-2-yl]benzoate (1.2 g) and sodiumhydroxide (0.209 g) in a mixture of water (4 ml), ethylalcohol (18 ml) and tetrahydrofuran (18 ml) was stirred at reflux for 7 hours. The reaction mixture was poured into water (40 ml). The mixture was adjusted to pH 1-2 with 1N-hydrochloric acid. The resulting precipitate was collected by filtration, and washed tetrahydrofuran, and dried under reduced pressure to give 4-[5-[4-[4-(2-pyrimidinyl)-1-piperazinyl]phenyl]-1,3,4-thiadiazol-2-yl]benzoic acid (1.14 g).

IR (KBr): 1695.1, 1606.4, 1585.2, 1417.4, 1234.2 cm⁻¹

ESI MASS (Negative)(m/z): 443.2 (M−H)⁻

Preparation 43

A mixture of 4-[5-[4-[4-(2-pyrimidinyl)-1-piperazinyl]phenyl]-1,3,4-thiadiazol-2-yl]benzoic acid (1.0 g), O-(benzotriazol-1-yl)-N,N,N′,N′-tetramethyluronium hexafluorophosphate (1.02 g) and N,N-diisopropylethylamine (0.8 ml) in N-methylpyrrolididone (10 ml) was stirred at ambient temperature for 4 hours. The reaction mixture was poured into water (80 ml). The resulting precipitate was collected by filtration, and washed tetrahydrofuran, and dried under reduced pressure to give 1-[4-[5-[4-[4-(2-pyrimidinyl)-1-piperazinyl]phenyl]-1,3,4-thiadiazol-2-yl]benzoyloxy]-1H-1,2,3-benzotriazole (1.23 g).

IR (KBr): 1780.0, 1606.4, 1585.2, 1234.2 cm⁻¹

Preparation 44

A mixture of ethyl 4-fluorobenzoate (5.1 g), 1-(2-pyridyl)piperazine (4.95 g) and potassium carbonate (5.03 g) in dimethylsulfoxide (26 ml) was stirred for 4 hours at 150° C. The reaction mixture was poured into water (260 ml). The resulting precipitate was collected by filtration, and dried under reduced pressure to give ethyl 4-[4-(2-pyridyl)-1-piperazinyl]benzoate (5.22 g).

IR (KBr): 1693.2, 1606.4, 1479.1, 1288.2, 1234.2 cm⁻¹

NMR (CDCl₃, δ): 1.37 (3H, t, J=7.1 Hz), 3.40-3.50 (4H, m), 3.70-3.75 (4H, m), 4.34 (2H, q, J=7.1 Hz), 6.64-6.71 (2H, m), 6.88-6.92 (2H, m), 7.47-7.52 (1H, m), 7.93-7.98 (2H, m), 8.20-8.23 (1H, m)

ESI MASS (Positive)(m/z): 312.3 (M+H)⁺, 334.3 (M+Na)⁺

Preparation 45

A solution of ethyl 4-[4-(2-pyridyl)-1-piperazinyl]benzoate (5.1 g) and hydrazine monohydrate (15.9 ml) in ethylalcohol (31 ml) was stirred at reflux for 14 hours. The reaction mixture was poured into water (47 ml). The resulting precipitate was collected by filtration, and dried under reduced pressure to give 4-[4-(2-pyridyl)-1-piperazinyl]benzohydrazide (3.82 g).

IR (KBr): 1654.6, 1606.4, 1481.1, 1436.7, 1234.2 cm⁻¹

NMR (CDCl₃, δ): 3.41-3.46 (4H, m), 3.96-3.74 (4H, m), 4.08 (2H, bs), 6.64-6.71 (2H, m), 6.91-6.95 (2H, m), 7.34 (1H, s), 7.48-7.56 (1H, m), 7.67-7.71 (2H, m), 8.20-8.23 (1H, m)

ESI MASS (Positive) (m/z): 298.3 (M+H)⁺, 320.2 (M+Na)⁺

Preparation 46

To a solution of 4-[4-(2-pyridyl)-1-piperazinyl]-benzohydrazide (3.29 g) and pyridine (2.69 ml) in N,N-dimethylformamide (55 ml) was added methyl 4-(chlorocarbonyl)benzoate (2.2 g) under ice-cooling, and the mixture was stirred at ambient temperature for 6 hours. The reaction mixture was poured into water (275 ml). The resulting precipitate was collected by filtration, and dried under reduced pressure to give methyl 4-[2-[4-[4-(2pyridyl)-1-piperazinyl]benzoyl]hydrazinocarbonyl]benzoate (4.6 g).

IR (KBr): 1722.1, 1683.1, 1598.7, 1436.7, 1234.2 cm⁻¹

NMR (DMSO-d₆, δ): 3.33-3.90 (11H, m), 6.65-6.71 (1H, m), 6.88-6.92 (1H, m), 7.04-7.09 (2H, m), 7.53-7.61 (1H, m), 7.83-7.87 (2H, m), 8.01-8.16 (5H, m), 10.30 (1H, bs), 10.59 (1H, bs)

ESI MASS (Positive)(m/z): 460.2 (M+H)⁺, 482.1 (M+Na)⁺

Preparation 47

A mixture of methyl 4-[2-[4-[4-(2-pyridyl)-1-piperazinyl]benzoyl]hydrazinocarbonyl]benzoate (4.6 g) and phosphorus pentasulfide (2.68 g) in pyridine (69 ml) was stirred at reflux for 17 hours. The reaction mixture was poured into water (138 ml). The mixture was adjusted to pH 9 with 1N-sodiumhydroxide solution. The resulting precipitate was collected by filtration, and washed acetonitrile (100 ml). The precipitate was collected by filtration, and dried under reduced pressure to give methyl 4-[5-[4-[4-(2-pyridyl)-1-piperazinyl]phenyl]-1,3,4-thiadiazol-2-yl]benzoate (3.84 g).

IR (KBr): 1726.0, 1600.6, 1438.6, 1282.4, 1234.2 cm¹

ESI MASS (Positive)(m/z): 458.1 (M+H)⁺

Preparation 48

A mixture of methyl 4-[5-[4-[4-(2-pyridyl)-1-piperazinyl]phenyl]-1,3,4-thiadiazol-2-yl]benzoate (3.7 g) and sodiumhydroxide (0.647 g) in a mixture of water (12.3 ml), ethylalcohol (56 ml) and tetrahydrofuran (56 ml) was stirred at reflux for 7 hours. The reaction mixture was poured into water (100 ml). The mixture was adjusted to pH 1-2 with 1N-hydrochloric acid. The resulting precipitate was collected by filtration, and washed tetrahydrofuran, and dried under reduced pressure to give 4-[5-[4-[4-(2-pyridyl)-1-piperazinyl]phenyl]-1,3,4-thiadiazol-2-yl]benzoic acid (3.69 g).

IR (KBr): 1706.7, 1602.6, 1415.5, 1230.4 cm⁻¹

ESI MASS (Positive)(m/z): 442.1 (M−H)⁻, 443.2 (M)⁻

Preparation 49

A mixture of 4-[5-(4-[4-(2-pyridyl)-1-piperazinyl]phenyl]-1,3,4-thiadiazol-2-yl]benzoic acid (3.5 g), O-(benzotriazol-1-yl)-N,N,N′,N′-tetramethyluronium hexafluorophosphate (3.59 g) and N,N-diisopropylethylamine (2.75 ml) in N-methylpyrrolidone (35 ml) was stirred at ambient temperature for 4 hours. The reaction mixture was poured into water (280 ml). The resulting precipitate was collected by filtration, and washed tetrahydrofuran, and dried under reduced pressure to give 1-[4-[5-[4-[4-(2-pyridyl)-1-piperazinyl]phenyl]-1,3,4-thiadiazol-2-yl]benzoyloxy]-1H-1,2,3-benzotriazole (4.15 g).

IR (KBr): 1780.0, 1664.3, 1602.6, 1438.6, 1234.2 cm⁻¹

Preparation 50

To a solution of tert-butyl 4-cyclopentyl-4-methoxy-1-piperidinecarboxylate (3.53 g) in ethyl acetate (18 ml) was added dropwise 4N-hydrogen chloride in ethyl acetate (31 ml) under ice-cooling. The mixture was stirred for 1 hour at room temperature. The reaction mixture was evaporated under reduced pressure to give 4-cyclopentyl-4-methoxypiperidine hydrochloride (2.82 g).

IR (KBr): 2944.8, 2723.0, 1456.0 cm⁻¹

NMR (CDCl₃, δ): 1.32-2.23 (13H, m), 3.05-3.30 (8H, m), 9.42 (1H, bm)

ESI MASS (Positive) (m/z): 184.4 (M+H)+(free)

Preparation 51

A mixture of ethyl 4-fluorobenzoate (2.14 g), 4-cyclopentyl-4-methoxypiperidine hydrochloride (2.8 g) and potassium carbonate (5.28 g) in dimethylsulfoxide (25 ml) was stirred for 15 hours at 140° C. The reaction mixture was poured into water, and extracted with ethyl acetate. The organic layer was washed with brine and dried, and the solvent was evaporated under reduced pressure. The residue was chromatographed on a column of silica gel eluting with n-hexane/ethyl acetate (5:1) to give ethyl 4-(4-cyclopentyl-4-methoxy-1-piperidyl)benzoate (3.27 g).

IR (KBr): 2954.4, 1699.0, 1604.5, 1076.1 cm⁻¹

NMR (CDCl₃, δ): 1.33-2.24 (16H, m), 3.06-3.66 (7H, m), 4.32 (2H, q, J=7.1 Hz), 6.83-6.90 (2H, m), 7.87-7.94 (2H, m)

ESI MASS (Positive)(m/z): 332.40 (M+H)⁺

Preparation 52

A mixture of ethyl 4-(4-cyclopentyl-4-methoxy-1-piperidyl)benzoate (3.15 g) and hydrazine monohydrate (18.4 ml) in a mixture of ethanol (31.5 ml) was refluxed for 9 hours. The reaction mixture was evaporated under reduced pressure. The residue was washed with water, and dried under pressure to give 4-(4-cyclopentyl-4-methoxy-1-piperidyl)benzohydrazide (2.85 g).

IR (KBr): 3295.7, 2948.6, 1635.3, 1606.4, 1068.4 cm⁻¹

NMR (CDCl₃, δ): 1.33-2.28 (13H, m), 3.03-3.62 (7H, m), 4.05 (2H, bs), 6.86-6.93 (2H, m), 7.24 (1H, bs), 7.60-7.67 (2H, m)

ESI MASS (Positive)(m/z): 318.40 (M+H)⁺

Preparation 53

To a solution of 4-(4-cyclopentyl-4-methoxy-1-piperidyl)benzohydrazide (2.8 g) and pyridine (2.63 ml) in tetrahydrofuran (84 ml) was added methyl 4-(chlorocarbonyl)benzoate (1.84 g) under ice-cooling. The mixture was stirred for 1.5 hours at room temperature. The reaction mixture was poured into water (420 ml). The resulting precipitate was collected by filtration, and dried under pressure to give methyl 4-[2-[4-(4-cyclopentyl-4-methoxy-1-piperidyl)benzoyl]hydrazinocarbonyl]benzoate (4.19 g).

IR (KBr): 3241.8, 2952.5, 1722.1, 1606.4, 1278.6, 1076.1 cm⁻¹

NMR (DMSO-d₆, δ): 1.28-1.79 (13H, m), 2.94-3.05 (2H, m), 3.14 (3H, s), 3.57-3.69 (2H, m), 3.90 (3H, s), 6.97-7.01 (2H, m), 7.77-7.82 (2H, m), 8.00-8.12 (4H, m), 10.22 (1H, s), 10.56 (1H, s)

ESI MASS (Positive)(m/z): 480.47 (M+H)⁺

Preparation 54

A mixture of 4-[2-[4-(4-cyclopentyl-4-methoxy-1-piperidyl)benzoyl]hydrazinocarbonyl]benzoate (4.12 g) and phosphorus pentasulfide (2.29 g) in pyridine (82 ml) was stirred at 120° C. for 2 hours. The reaction mixture was poured into water, and extracted with dichloromethane/methanol (5:1). The extract was washed successively with 1N-hydrochloric acid, saturated sodium hydrogen carbonate and brine and dried. After removal of the solvent, to give methyl 4-[5-[4-(4-cyclopentyl-4-methoxy-1-piperidyl)phenyl]-1,3,4-thiadiazol-2-yl]benzoate (0.8 g).

NMR (CDCl₃, δ): 1.33-2.27 (13H, m), 3.14-3.65 (7H, m), 4.31 (3H, s), 6.91-7.11 (2H, m), 7.72-7.94 (2H, m), 7.98-8.19 (4H, m)

ESI MASS (Positive)(m/z): 478.2 (M+H)⁺

Preparation 55

The mixture of methyl 4-[5-[4-(4-cyclopentyl-4-methoxy-1-piperidyl)phenyl]-1,3,4-thiadiazol-2-yl]benzoate (0.76 g) and 10% sodium hydroxide/water (1.6 ml) in a mixture of ethanol (7.6 ml) and tetrahydrofuran (7.6 ml) was refluxed for 15 hours. After cooling, the reaction mixture was acidified with 1N-hydrochloric acid. The resulting precipitate was collected by filtration, and washed with water, and dried to give 4-[5-[4-(4-cyclopentyl-4-methoxy-1-piperidyl)phenyl]-1,3,4-thiadiazol-2-yl]benzoic acid (0.62 g).

IR (KBr): 2950.6, 1687.4, 1602.6, 1078.0 cm⁻¹ NMR (DMSO-d₆, δ): 1.25-2.21 (13H, m), 3.11-3.63 (7H, m), 6.86-7.00 (2H, m), 7.75-8.23 (6H, m)

Preparation 56

A mixture of 4-[5-[4-(4-cyclopentyl-4-methoxy-1-piperidyl)phenyl]-1,3,4-thiadiazol-2-yl]benzosic acid (0.6 g), 1-hydroxybenzotriazole (0.21 g) and 1-ethyl-3-(3′-dimethylaminopropyl)carbodiimide hydrochloride (0.298 g) in dichloromethane (12 ml) was stirred at room temperature for 9 hours. The reaction mixture was poured into water and extracted with dichloromethane. The extract was washed with brine and dried. After removal of the solvent, to give 1-[4-[5-[4-(4-cyclopentyl-4-methoxy-1-piperidyl)phenyl]-1,3,4-thiadiazol-2-yl]benzoyloxy]-1H-1,2,3-benzotriazole (0.695 g).

IR (KBr): 2948.6, 1781.9, 1602.6, 1230.4, 1081.9 cm⁻¹

NMR (CDCl₃, δ): 1.37-2.26 (13H, m), 2.77-3.74 (7H, m), 6.91-7.00 (2H, m), 7.46-8.42 (10H, m)

ESI MASS (Positive)(m/z): 581.07 (M+H)⁺

Preparation 57

Thionyl chloride (10.9 ml) was added dropwise to methanol (70 ml) at 0 to 10° C. for 40 minutes. To the mixture was added 2,5-pyridinedicarboxylic acid (5 g). To the mixture was stirred at room temperature for 17 hours 30 minutes. The reaction mixture was evaporated under reduced pressure, and the residue was dissolved with water and adjusted to pH 11 with potassium carbonate. The precipitate was collected by filtration and washed with water, and dried to give dimethyl 2,5-pyridinedicarboxylate (4.54 g).

IR (KBr): 1726.0, 1716.3, 1132.0 cm⁻¹

NMR (CDCl₃, δ): 4.00 (3H, s), 4.05 (3H, s), 8.22 (1H, d, J=16.0 Hz), 8.46 (1H, dd, J=4.0 and 6.2 Hz), 9.31 (1H, d, J=3.8 Hz)

ESI MASS (Positive)(m/z): 218.2 (M+Na)⁺

Preparation 58

To a mixture of dimethyl 2,5-pyridinedicarboxylate (19.5 g) in methanol (585 ml) was added potassium hydroxide (7.29 g) and stirred at room temperature for 14 hours 30 minutes. The precipitate was collected by filtration. The solid was dissolved with water (300 ml), and acidified with 1N-hydrochloric acid. The precipitate was collected by filtration, and washed with water, and dried to give 5-(methoxycarbonyl)-2-pyridinecarboxylic acid (12.78 g).

IR (KBr): 3448.1, 1727.9, 1699.0, 1286.3, 1276.6 cm⁻¹

NMR (DMSO-d₆, δ): 3.93 (3H, sj, 8.16-8.18 (1H, m), 8.43-8.47 (1H, m), 9.165-9.172 (1H, m)

ESI MASS (Positive)(m/z): 180.2 (M−H)⁻

Preparation 59

A mixture of 5-(methoxycarbonyl)-2-pyridinecarboxylic acid (1.5 g), 4′-(2-methoxyethoxy)(1,1′-biphenyl)-4-carbohydrazide (2.25 g), 1-hydroxybenzotriazole (1.12 g), 1-ethyl-3-(3′-dimethylaminopropyl)carbodiimide (1.59 g) and diisopropylamine (1.44 ml) in N,N-dimethylformamide (24 ml) was stirred at room temperature for 2.5 hours. The reaction mixture was poured into water (115 ml), and the precipitate was collected by filtration, and washed with water, and dried to give methyl 6-[2-[4′-(2-methoxyethoxy)-1,1′-biphenyl-4-yl]carbonylhydrazino]carbonylnicotinate (2.99 g).

IR (KBr): 1722.1, 1699.0, 1652.7, 1128.2 cm⁻¹

NMR (CDCl₃, δ): 3.48 (3H, s), 3.79 (2H, t, J=4.6 Hz), 4.00 (3H, s), 4.18 (2H, t, J=4.6 Hz), 7.03 (2H, d, J=8.8 Hz), 7.55 (2H, d, J=8.8 Hz), 7.64 (2H, d, J=8.4 Hz), 7.94 (2H, d, J=8.4 Hz), 8.24 (1H, d, J=8.2 Hz), 8.47 (1H, dd, J=2.0 and 8.0 Hz), 9.20 (1H, s)

ESI MASS (Positive)(m/z): 472.2 (M+Na)⁺

Preparation 60

A mixture of methyl 6-[2-[4′-(2-methoxyethoxy)-1,1′-biphenyl-4-yl]carbonylhydrazino]carbonylnicotinate (2.91 g), triethylamine (1.08 ml) and phosphorus pentasulfide (1.73 g) in 1,2-dimethoxyethane (58 ml) was refluxed for 2.5 hours. The reaction mixture was poured into water (170 ml), and the precipitate was collected by filtration, and washed with water. The solid was refluxed with acetonitrile (58 ml) for 15 minutes, and the precipitate was collected by filtration, and dried to give methyl 6-[5-[4′-(2-methoxyethoxy)-1,1′-biphenyl-4-yl]-1,3,4-thiadiazol-2-yl]nitocinate (2.52 g).

IR (KBr): 1724.0, 1286.3, 1130.1, 1114.7 cm⁻¹

NMR (DMSO-d₆, δ): 3.28 (3H, s), 3.60-3.80 (2H, m), 3.95 (3H, s), 4.16-4.17 (2H, m), 7.08-7.10 (2H, m), 7.73-7.75 (2H, m), 7.86-7.88 (2H, m), 8.1-8.16 (2H, m), 8.46-8.56 (2H, m), 9.22 (1H, s)

ESI MASS (Positive)(m/z): 470.2 (M+Na)⁺

Preparation 61

A mixture of methyl 6-[5-[4′-(2-methoxyethoxy)-1,1′-biphenyl-4-yl]-1,3,4-thiadiazol-2-yl]nicotinate (2.4 g) and 10% sodium hydroxide (6.5 g) in a mixture of ethanol (24 ml) and tetrahydrofuran (36 ml) was refluxed for 10 hours. To the reaction mixture was added water (120 ml), and acidified with 1N-hydrochloric acid. The precipitate was collected by filtration and washed with water, and dried to give methyl 6-[5-[4′-(2-methoxyethoxy)-1,1′-biphenyl-4-yl]-1,3,4-thiadiazol-2-yl]nicotinic acid (2.22 g).

IR (KBr): 1685.5, 1596.8, 1249.6, 1132.0 cm⁻¹

NMR (DMSO-d₆, δ): 3.33 (3H, s), 3.69 (2H, t, J=4.4 Hz), 4.17 (2H, t, J=4.4 Hz), 7.09 (2H, d, J=8.4 Hz), 7.7.3 (2H, d, J=8.4 Hz), 7.87 (2H, d, J=8.2 Hz), 8.14 (2H, d, J=8.0 Hz), 8.44 (1H, d, J=7.6 Hz), 8.50-8.52 (1H, m), 9.19 (1H, s), 13.75 (1H, bs)

ESI MASS (Positive)(m/z): 432.1 (M−Na)⁻

Preparation 62

A mixture of methyl 6-[5-[4′-(2-methoxyethoxy)-1,1′-biphenyl-4-yl]-1,3,4-thiadiazol-2-yl]nicotinic acid (2.12 g), 1H-1,2,3-benzotriazol-1-ol (991 mg) and N-[3-(dimethylamino)propyl]-N′-ethylcarbodiimide hydrochloride (1.88 g) in dichloromethane (42 ml) was stirred for 18 hours at room temperature. The reaction mixture was evaporated under reduced pressure. The residue was washed with water, and dried to give 1-[6-[5-[4′-(2-methoxyethoxy)-1,1′-biphenyl-4-yl]-1,3,4-thiadiazol-2-yl]-3-pyridylcarbonyloxy]-1H-1,2,3-benzotriazole (2.51 g).

IR (KBr): 1787.7, 1594.8, 1251.6, 1234.2, 1126.2 cm⁻¹

NMR (DMSO-d₆, δ): 3.32 (3H, s), 3.68-3.70 (2H, m), 4.16-4.18 (2H, m), 7.08-7.10 (2H, m), 7.39-7.43 (1H, m), 7.52-7.56 (1H, m), 7.70-7.75 (3H, m), 7.86-7.88 (2H, m), 7.97-7.99 (1H, m), 8.13-8.15 (2H, m), 8.43-8.45 (1H, m), 8.50-8.52 (1H, m), 9.189-9.194 (1H, m)

Preparation 63

A mixture of 5-(methoxycarbonyl)-2-pyridinecarboxylic acid (1.5 g), 4′-(2-ethoxyethoxy)(1,1′-biphenyl)-4-carbohydrazide (2.36 g), 1-hydroxybenzotriazole (1.12 g), 1-ethyl-3-(3′-dimethylaminopropyl)carbodiimide (1.59 g) and diisopropylamine (1.44 ml) in N,N-dimethylformamide (24 ml) was stirred at room temperature for 2.5 hours. The reaction mixture was poured into water (120 ml), and the precipitate was collected by filtration, and washed with water, and dried to give methyl 6-[2-[4′-(2-ethoxyethoxy)-1,1′-biphenyl-4-yl]carbonylhydrazino]carbonylnicotinate (3.09 g).

IR (KBr): 1724.0, 1596.8, 1286.3, 1116.6 cm⁻¹

NMR (CDCl₃, δ): 1.26 (3H, t, J=7.0 Hz), 3.63 (2H, q, J=7.0 Hz), 3.83 (2H, t, J=4.8 Hz), 4.00 (3H, s), 4.18 (2H, t, J=4.6 Hz), 7.02 (2H, d, J=8.8 Hz), 7.55 (2H, d, J=8.6 Hz), 7.63 (2H, d, J=8.2 Hz), 7.94 (2H, d, J=8.2 Hz), 8.23 (1H, d, J=8.2 Hz), 8.45-8.48 (1H, m), 9.19 (1H, s)

ESI MASS (Positive)(m/z): 486.3 (M+Na)⁺

Preparation 64

A mixture of methyl 6-[2-[4′-(2-ethoxyethoxy)-1,1′-biphenyl-4-yl]carbonylhydrazino]carbonylnicotinate (3.67 g), triethylamine (1.32 ml) and phosphorus pentasulfide (2.11 g) in 1,2-dimethoxyethane (73 ml) was refluxed for 2.5 hours. The reaction mixture was poured into water (220 ml), and the precipitate was collected by filtration, and washed with water. The solid was refluxed with acetinitrile (73 ml) for 15 minutes, and the precipitate was collected by filtration, and dried to give methyl 6-[5-[4′-(2-ethoxyethoxy)-1,1′-biphenyl-4-yl]-1,3,4-thiadiazol-2-yl]nicotinate (3.14 g).

IR (KBr): 1685.5, 1249.6, 1133.9 cm⁻¹

NMR (DMSO-d₆, δ): 1.15 (3H, t, J=7.0 Hz), 3.52 (2H, q, J=7.0 Hz), 3.73 (2H, t, J=4.6 Hz), 3.95 (3H, s), 4.16 (2H, t, J=4.6 Hz), 7.09 (2H, d, J=8.6 Hz), 7.74 (2H, d, J=8.8 Hz), 7.87 (2H, d, J=8.4 Hz), 8.15 (2H, d, J=8.2 Hz), 8.47 (1H, d, J=8.2 Hz), 8.53-8.56 (1H, m), 9.22 (1H, s)

ESI MASS (Positive)(m/z): 484.2 (M+Na)⁺

Preparation 65

A mixture of methyl 6-[5-[4′-(2-ethoxyethoxy)-1,1′-biphenyl-4-yl]-1,3,4-thiadiazol-2-yl]nicotinate (3 g) and 10% sodium hydroxide (7.8 g) in a mixture of ethanol (30 ml) and tetrahydrofuran (45 ml) was refluxed for 10 hours. To the reaction mixture was added water (150 ml), and acidified with 1N-hydrochloric acid. The precipitate was collected by filtration and washed with water, and dried to give methyl 6-[5-[4′-(2-ethoxyethoxy)-1,1′-biphenyl-4-yl]-1,3,4-thiadiazol-2-yl]nicotinic acid (2.80 g).

NMR (DMSO-d₆, δ): 1.15 (3H, t, J=7.0 Hz), 3.52 (2H, q, J=7.0 Hz), 3.73 (2H, t, J=4.6 Hz), 4.15 (2H, t, J=4.6 Hz), 7;08 (2H, d, J=8.8 Hz), 7.72 (2H, d, J=8.8 Hz), 7.85 (2H, d, J=8.4 Hz), 8.07-8.14 (2H, m), 8.42-8.51 (2H, m), 9.18 (1H, s), 13.73 (1H, bs)

ESI MASS (Positive)(m/z): 446.2 (M−H)⁻

Preparation 66

A mixture of methyl 6-[5-[4′-(2-ethoxyethoxy)-1,1′-biphenyl-4-yl]-1,3,4-thiadiazol-2-yl]nicotinic acid (2.71 g), 1H-1,2,3-benzotriazol-1-ol (1.23 g) and N-[3-(dimethylamino)propyl]-N′-ethylcarbodiimide hydrochloride (2.32 g) in dichloromethane (54 ml) was stirred for 18 hours at room temperature. The reaction mixture was evaporated under reduced pressure. The residue was washed with water, and dried to give 1-[6-[5-[4′-(2-ethoxyethoxy)-1,1′-biphenyl-4-yl]-1,3,4-thiadiazol-2-yl]-3-pyridylcarbonyloxy]-1H-1,2,3-benzotriazole (3.29 g).

IR (KBr): 1803.1, 1787.7, 1594.8, 1253.5 cm¹

NMR (DMSO-d₆, δ): 1.15 (3H, t, J=7.0 Hz), 3.52 (2H, q, J=7.0 Hz), 3.72-3.74 (2H, m), 4.15-4.17 (2H, m), 7.09 (2H, d, J=8.8 Hz), 7.38-7.42 (1H, m), 7.51-7.55 (1H, m), 7.70-7.75 (2H, m), 7.86-7.88 (2H, m), 7.96-7.98 (1H, m), 8.13-8.15 (2H, m), 8.42-8.52 (2H, m), 9.18-9.19 (1H, m)

Preparation 67

To a mixture of cesium carbonate (2.53 g), palladium(II) acetate (62.3 mg) and 2,2′-bis(diphenylphosphino)-1,1′-binaphthyl (259 mg) in toluene (20 ml) was successively added methyl 4′(trifluoromethylsulfonyloxy)(1,1′-biphenyl)-4-carboxylate (2.00 g) and 1,2,3,4-tetrahydroisoquinoline (813 mg) in stream of nitrogen. The mixture was stirred at ambient temperature for 30 minutes and at 110° C. for further 17 hours. After cooling to room temperature, water and acetonitrile was added to the reaction mixture. The resulting precipitate was collected by filtration and washed by 50% acetonitrile in water and diisopropylether. Chloroform was added to the resulting precipitate. And the mixture was filtered by membrane filter, and the filtrate was concentrated under reduced pressure to give methyl 4′-[3,4-dihydro-2(1H)-isoquinolinyl]-1,1′-biphenyl-4-carboxylate (1.702 g).

NMR (CDCl₃, δ): 3.02 (2H, t, J=5.8 Hz), 3.64 (2H, t, J=5.8 Hz), 4.49 (2H, s), 7.04 (2H, d, J=8.9 Hz), 7.22 (4H, s), 7.55-7.7 (4H, m), 8.06 (2H, d, J=8.4 Hz)

MASS (m/z): 344.3 (M+H)

The following compounds [Preparation 68 and 69] were obtained according to a similar manner to that of Preparation 67.

Preparation 68

### 4′-(4-Morpholinyl)-1,1′-biphenyl-4-ol

NMR (CDCl₃, δ): 3.3-3.45 (4H, m), 3.8-3.95 (4H, m), 6.88 (2H, d, J=8.6 Hz), 6.97 (2H, d, J=8.8 Hz), 7.41 (2H, d, J=8.6 Hz), 7.47 (2H, d, J=8.8 Hz)

MASS (m/z): 254.2 (M+H)

Preparation 69

### Methyl 4′-(6,7-dimethoxy-3,4-dihydro-2(1H)-isoquinolinyl)-1,1′-biphenyl-4-carboxylate

NMR (CDCl₃, δ): 2.92 (2H, t, J=5.8 Hz), 3.62 (2H, t, J=5.8 Hz), 3.87 (3H, s), 3.89 (3H, s), 3.93 (3H, s), 4.41 (2H, s), 6.66 (1H, s), 6.68 (1H, s), 7.04 (2H, d, J=8.9 Hz), 7.5-7.7 (4H, m), 8.06 (2H, d, J=8.5 Hz)

MASS (m/z): 426.4 (M+Na)

Preparation 70

A mixture of methyl 4′-(3,4-dihydro-2(1H)-isoquinolinyl)-1,1′-blphenyl-4-carboxylate (1.7 g) and 10% sodium hydroxide solution (7.9 ml) in a mixed solvent of methanol (17 ml) and tetrahydrofuran (34 ml) was refluxed for 4 hours. After cooling to ambient temperature, the reaction mixture was poured into cold water and the mixture was adjusted to pH 7.2 with 1.0 mol/1 hydrochloric acid. The resulting precipitates were filtered, washed with water and acetonitrile then dried to give 4′-(3,4-dihydro-2(1H)-isoquinolinyl)-1,1′-biphenyl-4-carboxylic acid (1.53 g).

NMR (CDCl₃, δ): 2.93 (2H, t, J=5.8 Hz), 3.62 (2H, t, J=5.8 Hz), 4.48 (2H, s), 7.10 (2H, d, J=8.9 Hz), 7.6-8.8 (4H, m), 7.96 (2H, d, J=8.4 Hz)

MASS (m/z): 328.3 (M−H)

The following compounds [Preparation 71 to 73] were obtained according to a similar manner to that of Preparation 70.

Preparation 71

### 4′-(6,7-Dimethoxy-3,4-dihydro-2(1H)-isoquinolinyl)-1,1′-biphenyl-4-carboxylic acid

NMR (CDCl₃, δ): 2.83 (2H, d, J=5.7 Hz), 3.59 (2H, d, J=5.7 Hz), 3.73 (3H, s), 3.74 (3H, s), 4.39 (2H, s), 6.75 (1H, s), 6.85 (1H, s), 7.08 (2H, d, J=8.8 Hz), 7.65 (2H, d, J=8.8 Hz), 7.74 (2H, d, J=8.4 Hz), 7.96 (2H, d, J=9.4 Hz)

MASS (m/z): 388.3 (M−H)

Preparation 72

### 4-[5-[4-[4-[Methoxy(phenyl)methyl]-1-piperidyl]phenyl]-1,3,4-thiadiazol-2-yl]benzoic acid

NMR (CDCl₃, δ): 1.1-2.2 (5H, m), 2.55-2.95 (2H, m), 3.10 (3H, s), 3.7-4.05 (3H, m), 7.03 (2H, d, J=8.9 Hz), 7.2-7.45 (5H, m), 7.82 (2H, d, J=8.9 Hz), 8.10 (4H, s)

MASS (m/z): 484.3 (M−H)

Preparation 73

### 4″-(4-Morpholinyl)-1,1′:4′,1″-terphenyl-4-carboxylic acid

MASS (m/z): 358.3 (M−H)

Preparation 74

A mixture of 4′-(3,4-dihydro-2(1H)-isoquinolinyl)-1,1′-biphenyl-4-carboxylic acid (1.52 g), O-(benzotiazol-1-yl)-N,N,N′,N′-tetramethyluronium hexafluorophosphate (3.29 g) and diisopropylethylamine (1.19 ml) in N-methylpiperidone (15 ml) was stirred for 4 hours at room temperature. Water was added to the mixture and the resulting precipitate was collected by filtration, washed with water and acetonitrile and then dried to give 2-[4′-(1H-1,2,3-benzotriazol-1-yloxycarbonyl)-1,1′-biphenyl-4-yl]-1,2,3,4-tetrahydroisoquinoline (1.878 g).

NMR (CDCl₃, δ): 2.9-3.1 (2H, m), 3.6-3.75 (2H, m), 4.53 (2H, s), 7.0-8.4 (16H, m)

MASS (m/z): 447.07 (M+H) The following compounds [Preparation 75 to 771] were obtained according to a similar manner to that of Preparation 74.

Preparation 75

### 2-[4′-(1H-1,2,3-Benzotriazol-1-yloxycarbonyl)-1,1′biphenyl-4-yl]-6,7-dimethoxy-1,2,3,4-tetrahydroisoquinoline

NMR (CDCl₃, δ): 2.94 (2H, t, J=5.7 Hz), 3.66 (2H, t, J=5.7 Hz), 3.88 (3H, s), 3.90 (3H, s), 4.46 (2H, s), 6.68 (1H, s), 6.70 (1H, s), 7.07 (2H, d, J=8.9 Hz), 7.4-7.9 (7H, m), 8.12 (1H, d, J=8.1 Hz), 8.25 (2H, d, J=9.5 Hz)

MASS (m/z): 507.13 (M+H)

Preparation 76

### 1-[4-[5-[4-[Methoxy(phenyl)methyl-1-piperidyl]phenyl]-1,3,4-thiadiazol-2-yl]benzoyloxy]-1H-1,2,3-benzotriazole

NMR (CDCl₃, δ): 1.1-1.9 (4H, m), 2.1-2.25 (1H, m), 2.6-2.9 (2H, m), 3.20 (3H, s), 3.7-4.0 (3H, m), 6.94 (2H, d, J=9.0 Hz), 7.2-7.65 (8H, m), 7.8-8.45 (7H, m)

Preparation 77

### 1-[[4″-(4-Morpholinyl)-1,1′:4′,1″-terphenyl-4-yl]-carbonyloxy]-1H-1,2,3-benzotriazole

NMR (CDCl₃, δ): 3.15-3.35 (4H, m), 3.8-4.0 (4H, m), 7.02 (2H, d, J=8.5 Hz), 7.35-7.95 (11H, m), 8.13 (1H, d, J=8.5 Hz), 8.37 (2H, d, J=8.1 Hz)

MASS (m/z): 477.20 (M+H)

The Starting Compound used and the Object Compounds obtained in the following Preparation 78 to 96 are given in the table as below, in which the formulas of the starting compounds are in the upper column, and the formulas of the object compounds are in the lower column, respectively.

| Preparation | |
|---|---|
| No. | Formula |
| 78 | 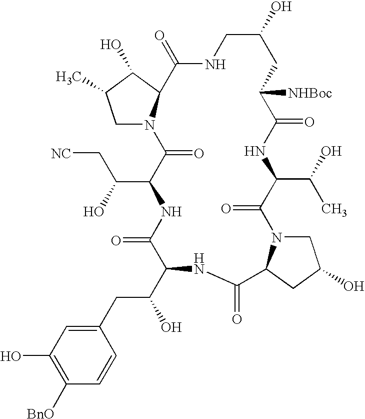 |
| | 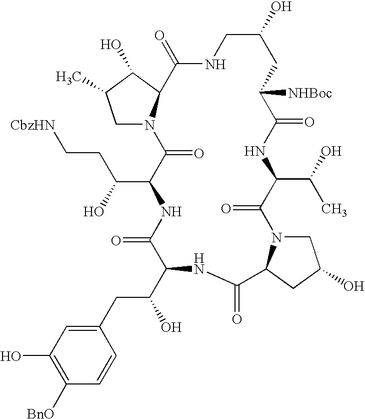 |
| 79 | 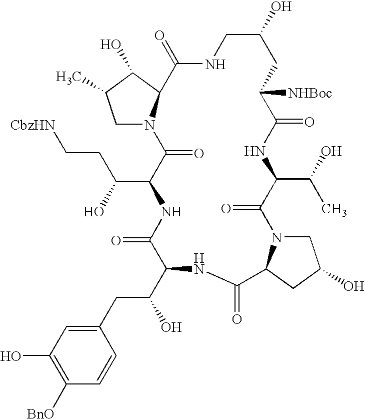 |
| | 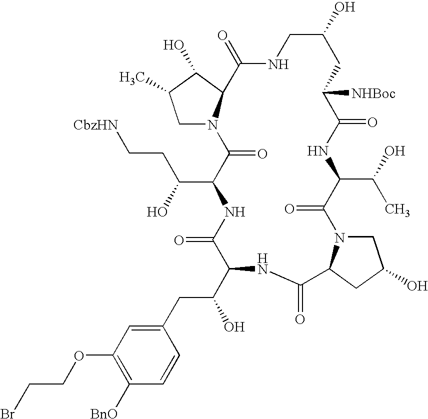 |
| 80 | 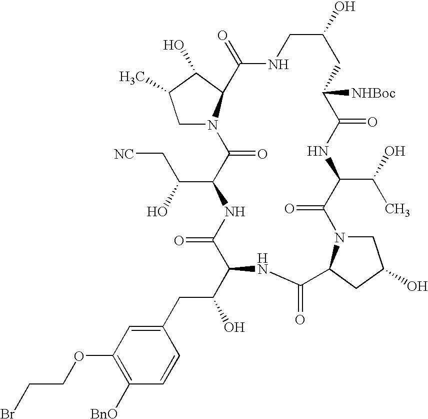 |
| | 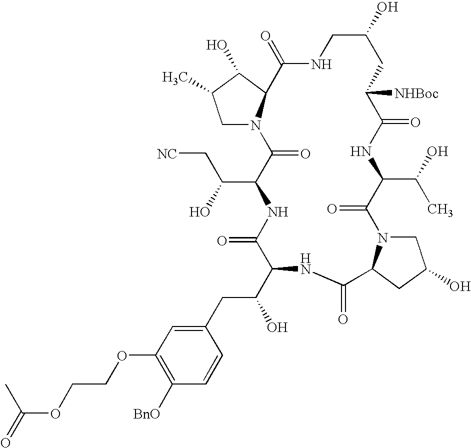 |
| 81 | 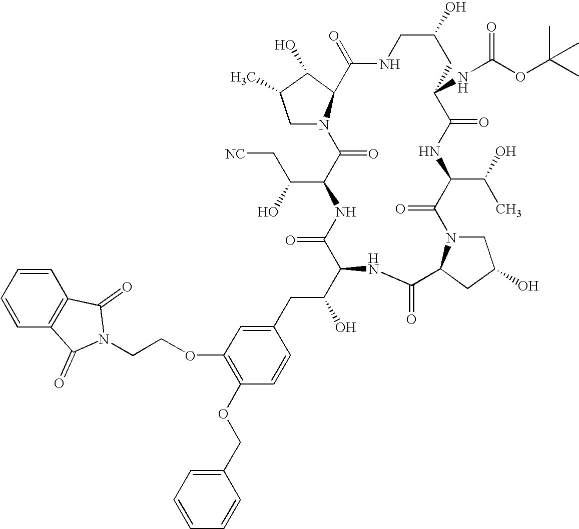 |
| | 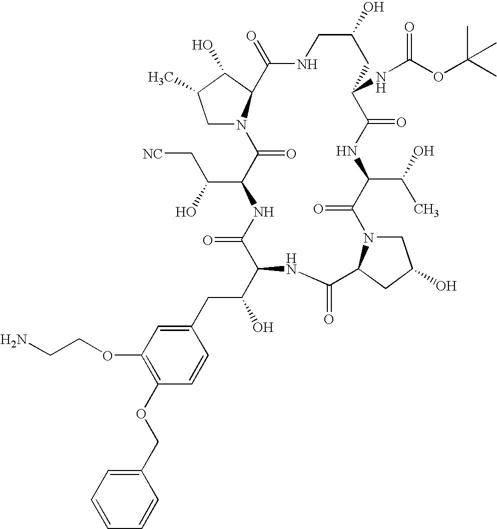 |
| 82 | 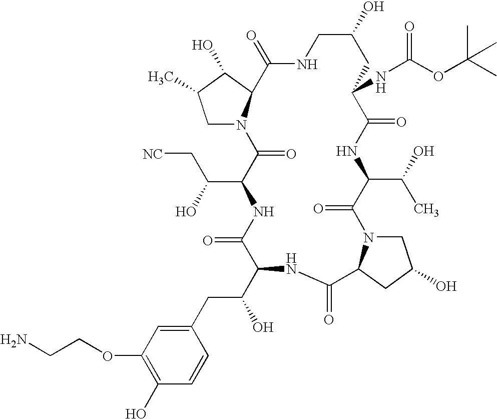 |
| | 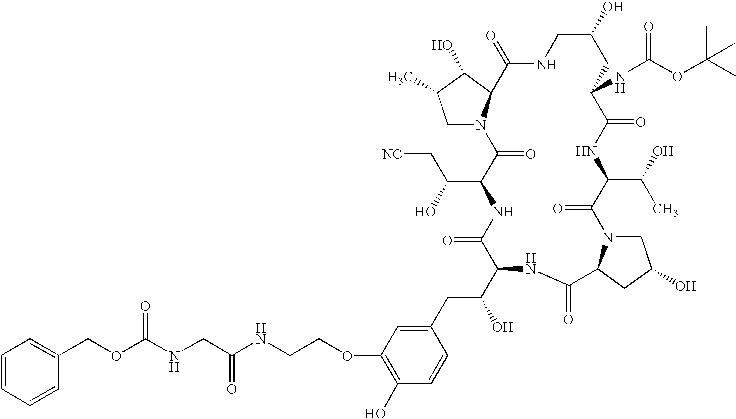 |
| 83 | 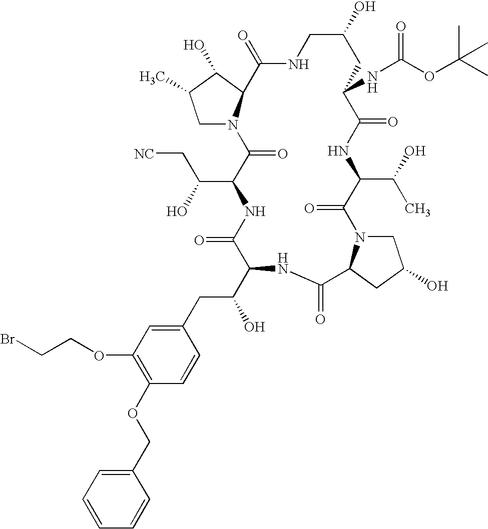 |
| | 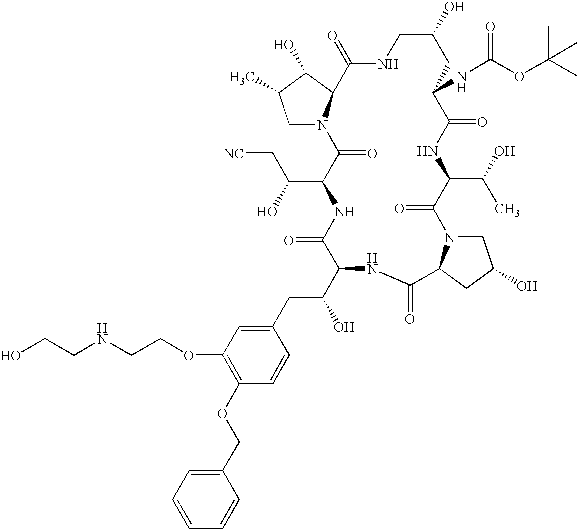 |
| 84 | 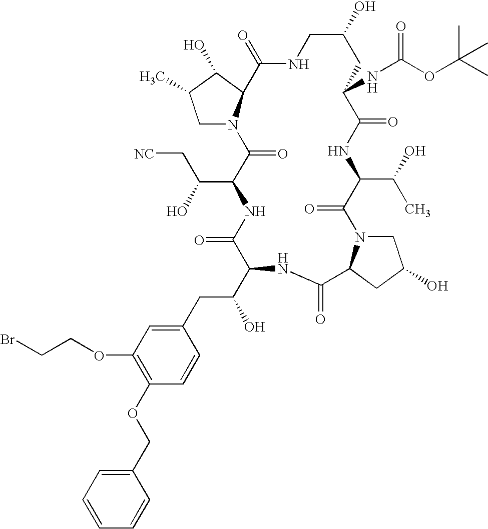 |
| | 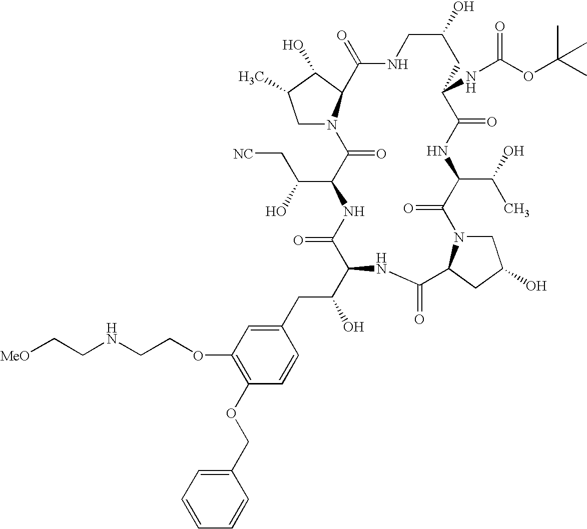 |
| 85 | 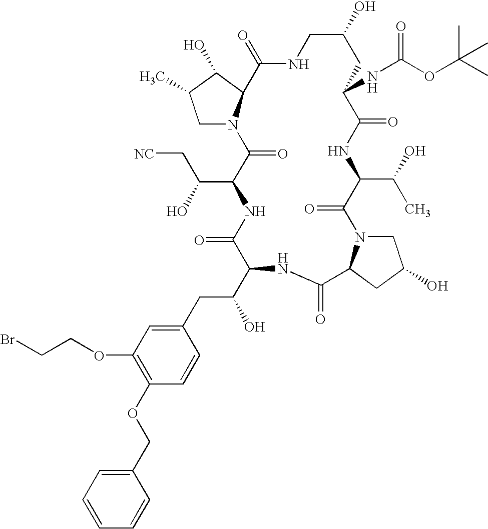 |
| | 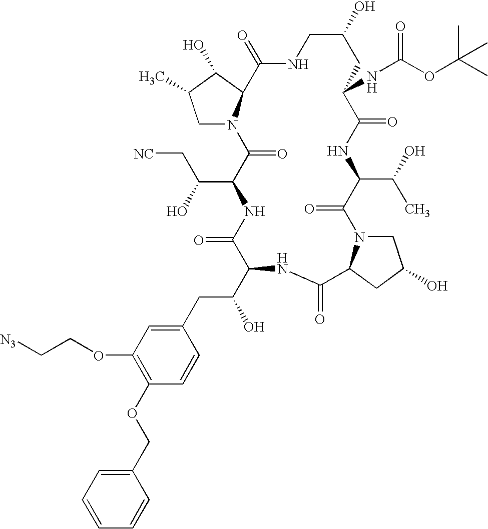 |
| 86 | 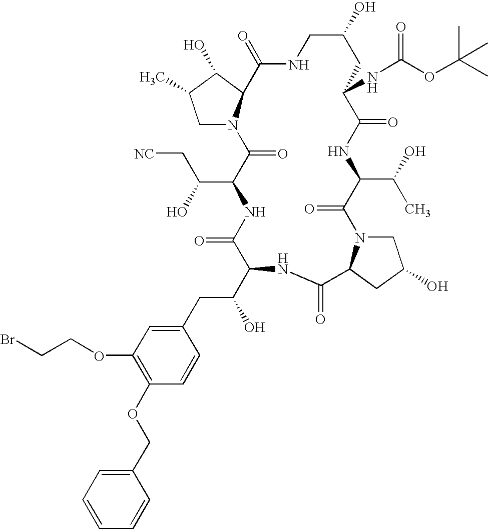 |
| | 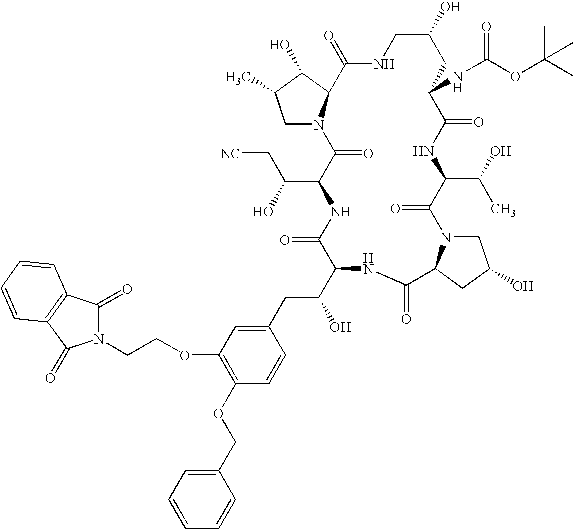 |
| 87 | 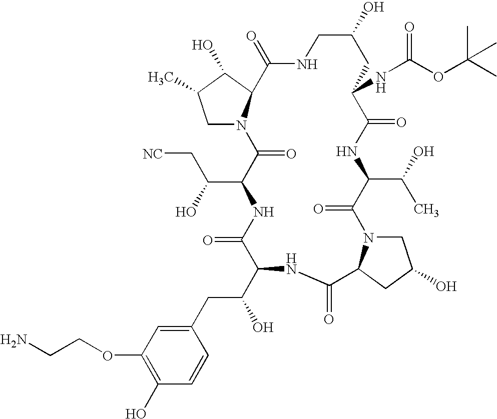 |
| | 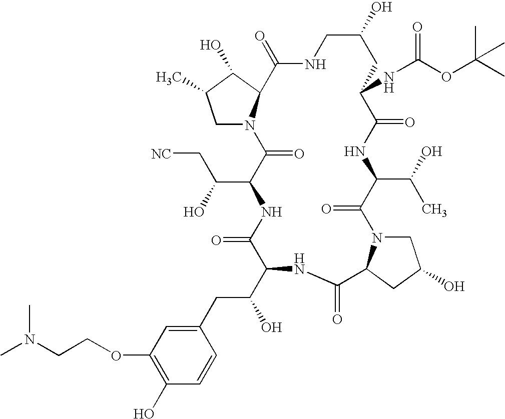 |
| 88 | 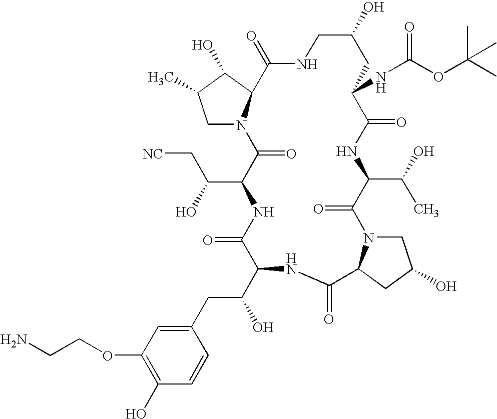 |
| | 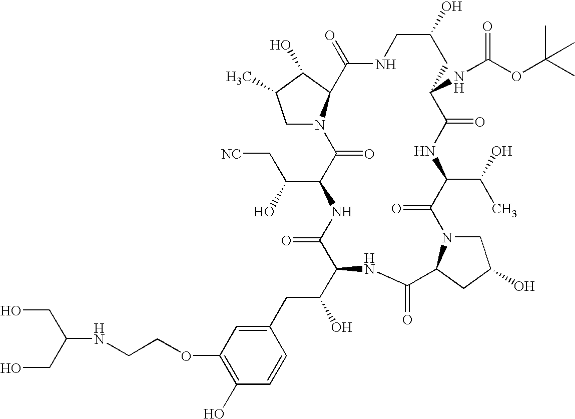 |
| 89 | 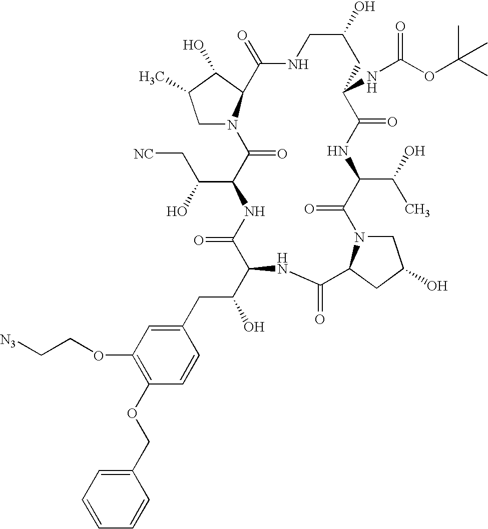 |
| | 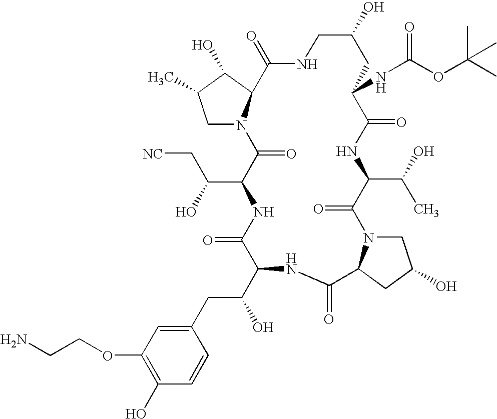 |
| 90 | 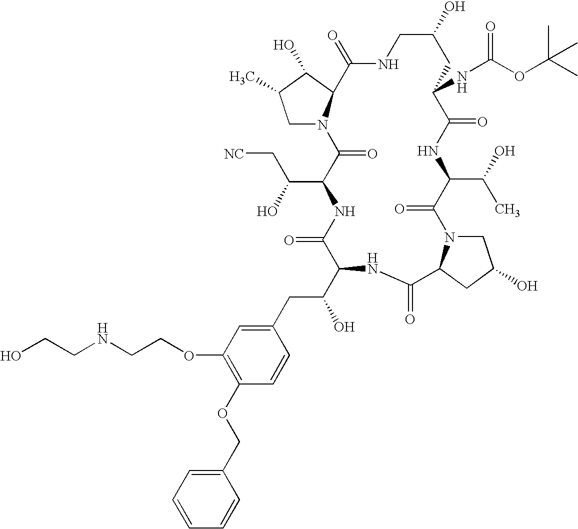 |
| | 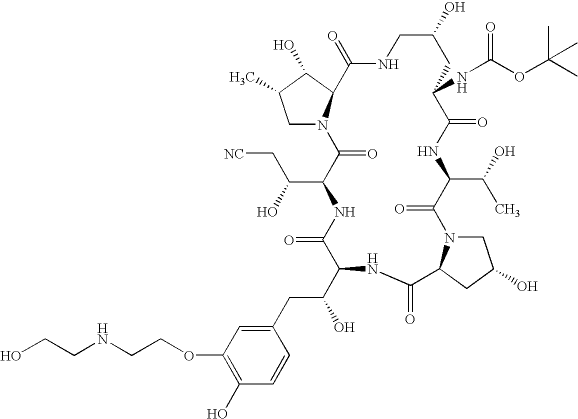 |
| 91 | 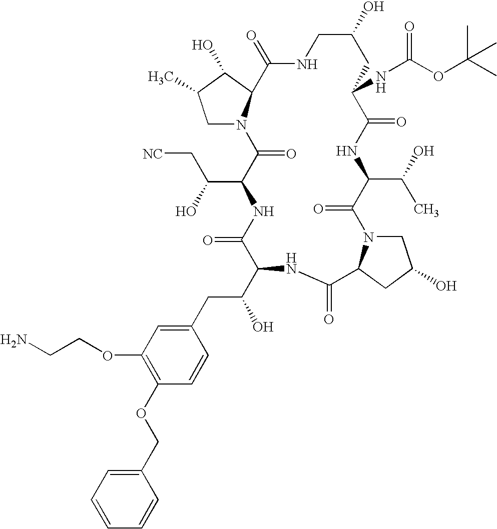 |
| | 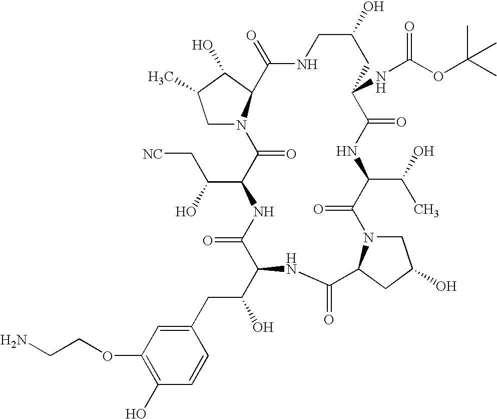 |
| 92 | 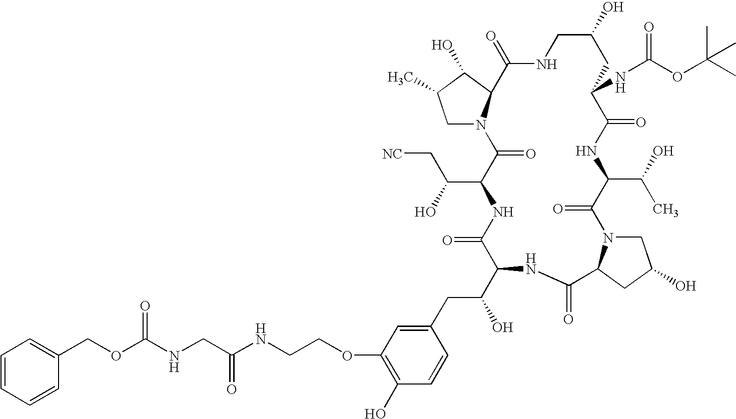 |
| | 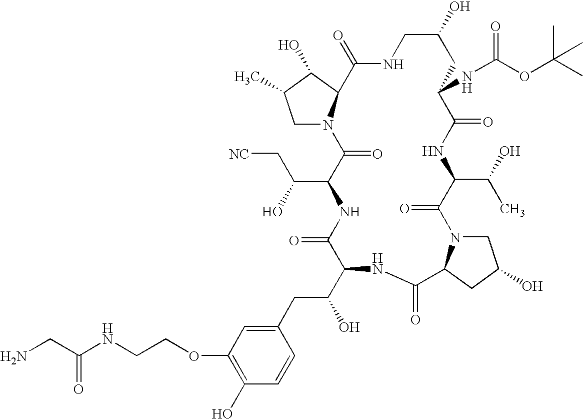 |
| 93 | 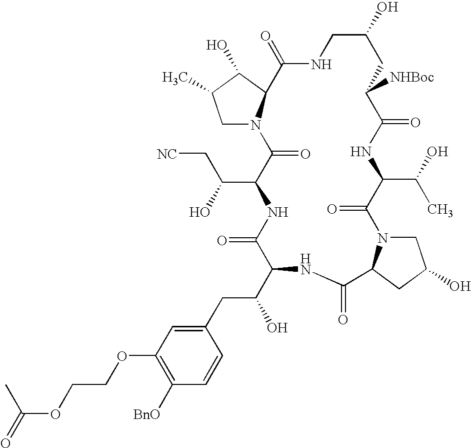 |
| | 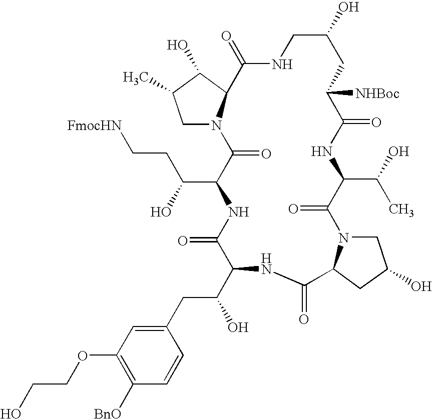 |
| 94 | 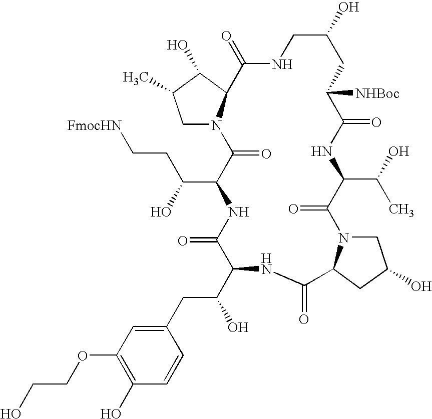 |
| | 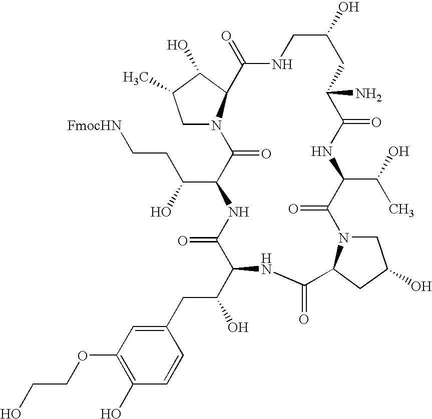 |
| 95 | 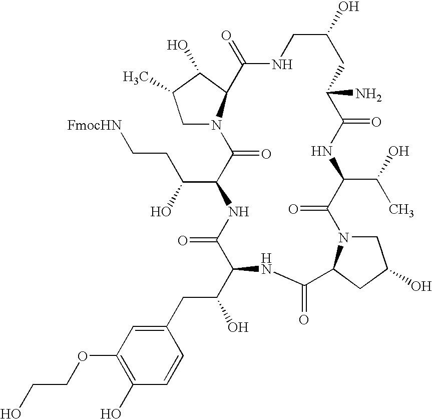 |
| | 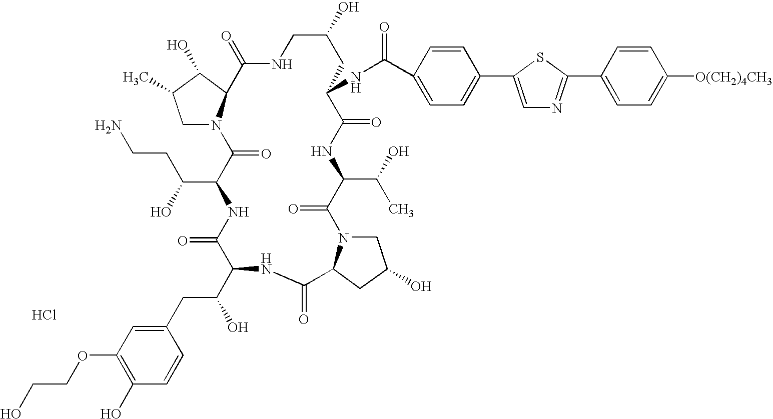 |
| 96 | 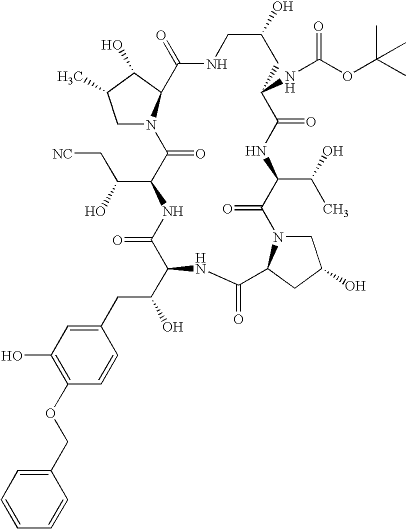 |
| | 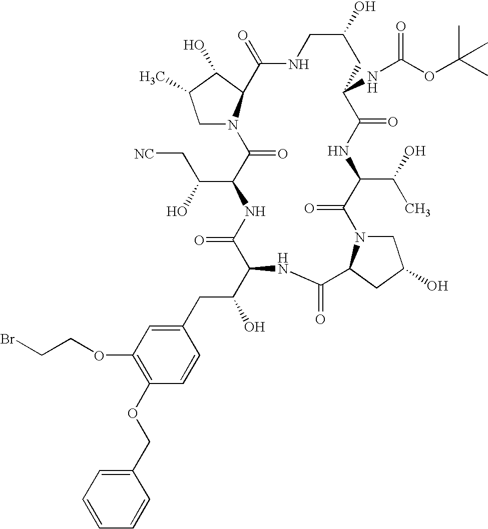 |

Preparation 78

To a solution of starting compound (78) (2 g) and cobalt(II) chloride hexahydrate (1.91 g) in a mixture of methanol (60 ml) and water (12 ml) was added sodium borohydride (1.52 g) with stirring at room temperature and the mixture was stirred at the same temperature for 2 hours. The reaction mixture was filtered through celite. The filtrate was adjusted pH 8 with 1N-hydrochloric acid, evaporated under reduced pressure to remove methanol and was lyophilized. To the mixture of resulting residue and N,N-diisopropylethylamine (1.4 ml) in N,N-dimethylformamide (60 ml) was added benzyl chloridocarbonate (0.43 ml) at room temperature, and the mixture was stirred for 24 hours. Water was added. And the solution was subjected to column chromatography on ODS eluting with 40% acetonitrile in water. The fractions containing the object compound were collected and evaporated under reduced pressure to remove acetonitrile. The residue was lyophilized to give object compound (78) (927 mg).

NMR (DMSO-d₆+D₂O, δ): 0.94 (3H, d, J=6.7 Hz), 1.09 (3H, d, J=5.3 Hz), 1.32 (9H, s), 1.4-4.5 (27H, m), 4.6-4.85 (2H, m), 5.00 (2H, s), 5.06 (2H, s), 6.4-6.7 (2H, m), 6.84 (1H, d, J=8.3 Hz), 7.2-7.5 (10H, m)

MASS (m/z): 1157.5 (M+Na)

Preparation 79

A mixture of starting compound (79) (920 mg), dibromoethane (2.1 ml) and lithium hydroxide (510 mg) in N,N-dimethylformamide (9.2 ml) was stirred for 21 hours at room temperature. Water was added to the mixture. The solution was subjected to column chromatography on ODS eluting with 45% acetonitrile in water. The fractions containing the object compound were collected and evaporated under reduced pressure to remove acetoniterile. The residue was lyophilized to give object compound (79) (792.5 mg).

NMR (DMSO-d₆+D₂O, δ): 0.94 (3H, d, J=6.6 Hz), 1.0-1.05 (3H, m), 1.33 (9H, s), 1.4-4.5 (32H, m), 4.7-4.85 (2H, m), 5.00 (2H, s), 5.09 (2H, s), 6.65-6.85 (2H, m), 5.96 (1H, d, J=8.3 Hz), 7.2-7.5 (10H, m)

MASS (m/z): 1265.4, 1267.4 (M⁺+Na)

Preparation 80

A mixture of starting compound (80) (1 g) and sodium acetate (149 mg) in N,N-dimethylformamide (10 ml) was stirred for 17 hours at 80° C. 10% acetonitrile in water was added to the mixture. The solution was subjected to column chromatography on ODS eluting with 35% acetonitrile in water. The fractions containing the object compound were collected and evaporated under reduced pressure to remove acetonitrile. The residue was lyophilized to give object compound (80) (668 mg).

NMR (DMSO-d₆+D₂O, δ): 0.96 (3H, d, J=6.8 Hz), 1.07 (3H, d, J=5.7 Hz), 1.36 (9H, s), 1.6-2.0 (3H, m), 2.01 (3H, s), 2.2-2.9 (6H, m), 3.2-3.3 (1H, m), 3.6-4.5 (19H, m), 4.8-4.9 (2H, m), 5.07 (2H, s), 6.71 (1H, d, J=8.2 Hz), 6.79 (1H, s), 6.95 (1H, s), 7.3-7.5 (5H, m)

MASS (m/z): 1105.3 (M+Na)

Preparation 81

A mixture of starting compound (81) (9.9 g) and hydrazine monohydrate (2.1 ml) in ethanol (200 ml) was stirred for 4.5 hours at 70° C. The reaction mixture was poured into water (1.8 l). The solution was subjected to column chromatography on ODS (Daiso-gel, SP-120-40/60-ODS-B) (400 ml) eluting with 40% acetonitrile in water. The eluted fractions containing the desired product were collected and evaporated in vacuo. The residue was lyophilized to give object compound (81) (8.22 g).

NMR (DMSO-d₆+D₂O, δ): 0.94 (3H, d, J-6.7 Hz), 1.08 (3H, d, J=5.4 Hz), 1.36 (9H, s), 1.5-2.9 (11H, m), 3.1-4.5 (18H, m), 4.7-4.9 (2H, m), 5.06 (2H, s), 6.6-7.0 (3H, m), 7.2-7.5 (5H, m)

ESI MASS (Positive): 1062.4 (M+Na)⁺

Preparation 82

A mixture of starting compound (82) (2.0 g), 1-[3-(dimethylamino)propyl]-3-ethylcarbodiimide hydrochloride (0.49 g) and 1-hydroxybenzotriazole (0.34 g) in N,N-dimethylformamide (200 ml) was stirred for 25 hours at room temperature. The reaction mixture was poured into a mixture of acetonitrile (100 ml) and water (900 ml). The solution was subjected to column chromatography on ODS (Daiso-gel, SP-120-40/60-ODS-B) (200 ml) eluting with 40% acetonitrile in water. The eluted fractions containing the desired product were collected and evaporated in vacuo. The residue was lyophilized to give object compound (82) (2.13 g).

NMR (DMSO-d₆+D₂O, δ): 0.95 (3H, d, J=6.7 Hz), 1.08 (3H, d, J=5.4 Hz), 1.36 (9H, s), 1.5-2.9 (9H, m), 3.1-4.5 (22H, m), 4.8-4.9 (2H, m), 5.03 (2H, s), 6.5-6.8 (3H, m), 7.2-7.5 (5H, m)

ESI MASS (Positive): 1163.3 (M+Na)⁺

Preparation 83

A mixture of starting compound (83) (3.0 g), 2-aminoethanol (0.82 ml) and triethylamine (1.9 ml) in N,N-dimethylformamide (30 ml) was stirred for 99 hours at room temperature. The reaction mixture was poured into a mixture of acetonitrile (200 ml) and water (800 ml). The solution was subjected to column chromatography on ODS (Daiso-gel, SP-120-40/60-ODS-B) (400 ml) eluting with 40% acetonitrile in water. The eluted fractions containing the desired product were collected and evaporated in vacuo. The residue was lyophilized to give object compound (83) (2.32 g).

NMR (DMSO-d₆+D₂O, δ): 0.95 (3H, d, J=6.8 Hz), 1.08 (3H, d, J=5.5 Hz), 1.36 (9H, s), 1.5-3.0 (23H, m), 3.1-4.5 (20H, m), 4.8-4.9 (2H, m), 5.06 (2H, s), 6.6-7.0 (3H, m), 7.2-7.5 (5H, m)

ESI MASS (Positive): 1106.3 (M+Na)⁺

The following compound was obtained according to a similar manner to that of Preparation 83.

Preparation 84

NMR (DMSO-d₆+D₂O, δ): 0.95 (3H, d, J=6.8 Hz), 1.08 (3H, d, J=5.9 Hz), 1.36 (9H, s), 1.5-2.9 (13H, m), 3.1-4.5 (20H, m), 3.21 (3H, s), 4.8-4.9 (2H, m), 5.06 (2H, s), 6.6-7.0 (3H, m), 7.3-7.5 (5H, m)

ESI MASS (Positive): 1098.4 (M+H)⁺, 1120.3 (M+Na)⁺

Preparation 85

A mixture of starting compound (85) (16.9 g) and sodium azide (2.0 g) in N,N-dimethylformamide (170 ml) was stirred for 3 hours at 60° C. The reaction mixture was poured into a mixture of acetonitrile (1 l) and water (2.8 l). The solution was subjected to column chromatography on ODS (Daiso-gel, SP-120-40/60-ODS-B) (1 l) eluting with 60% acetonitrile in water. The eluted fractions containing the desired product were collected and evaporated in vacuo. The residue was lyophilized to give object compound (85) (15.9 g).

NMR (DMSO-d₆+D₂O, δ): 0.93 (3H, d, J=6.7 Hz), 1.06 (3H, d, J=5.7 Hz), 1.33 (9H, s), 1.5-2.9 (11H, m), 3.1-4.5 (18H, m), 4.7-4.9 (2H, m), 5.05 (2H, s), 6.6-7.0 (3H, m), 7.2-7.5 (5H, m)

ESI MASS (Positive): 1088.4 (M+Na)⁺

The following compound was obtained according to a similar manner to that of Preparation 85.

Preparation 86

NMR (DMSO-d₆+D₂O, δ): 0.95 (3H, d, J=6.8 Hz), 1.08 (3H, d, J=5.7 Hz), 1.36 (9H, s), 1.5-2.9 (9H, m), 3.1-4.5 (20H, m), 4.8-5.0 (4H, m), 6.6-7.0 (3H, m), 7.2-7.4 (5H, m), 7.8-8.0 (4H, m)

ESI MASS (Positive): 1192.3 (M+Na)⁺

Preparation 87

To a mixture of starting compound (87) (0.2 g), formaldehyde (35% solution in water) (58 μl) and acetic acid (36 μl) in N,N-dimethylformamide (3 ml) and methanol (6 ml) was added sodium cyanoborohydride (27 mg). After stirring for 24 hours at room temperature, the reaction mixture was poured into water (100 ml). The solution was subjected to column chromatography on ODS (Daiso-gel, SP-120-40/60-ODS-B) (100 ml) eluting with 15% acetonitrile in water. The eluted fractions containing the desired product were collected and evaporated in vacuo. The residue was lyophilized to give object compound (87) (0.12 g).

ESI MASS (Positive): 1000.5 (M+Na)⁺

The following compound was obtained according to a similar manner to that of Preparation 87.

Preparation 88

NMR (DMSO-d₆+D₂O, δ): 0.95 (3H, d, J=6.7 Hz), 1.08 (3H, d, J=5.7 Hz), 1.36 (9H, s), 1.5-3.0 (12H, m), 3.2-4.5 (22H, m), 4.8-4.9 (2H, m), 6.5-6.8 (3H, m)

ESI MASS (Positive): 1046.3 (M+Na)⁺

The following compounds [Preparation 89 to 92] were obtained according to a similar manner to that of Preparation 20.

Preparation 89

NMR (DMSO-d₆+D₂O, δ): 0.95 (3H, d, J=6.7 Hz), 1.08 (3H, d, J=5.6 Hz), 1.35 (9H, s), 1.5-2.9 (11H, m), 3.1-4.5 (11H, m), 4.7-5.0 (2H, m), 6.5-6.8 (3H, m) ESI MASS (Positive): 972.4 (M+Na)⁺

Preparation 90

NMR (DMSO-d₆+D₂O, δ): 0.95 (3H, d, J=6.7 Hz), 1.08 (3H, d, J=5.7 Hz), 1.35 (9H, s), 1.5-4.5 (33H, m), 4.7-4.9 (2H, m), 6.5-6.8 (3H, m)

ESI MASS (Positive): 1016.3 (M+Na)⁺

Preparation 91

NMR (DMSO-d₆+D₂O, δ): 0.95 (3H, d, J=6.8 Hz), 1.08 (3H, d, J=5.7 Hz), 1.36 (9H, s), 1.5-2.9 (11H, m), 3.1-4.5 (18H, m), 4.8-4.9 (2H, m), 6.5-6.8 (3H, m)

ESI MASS (Positive): 950.3 (M+H)⁺, 972.3 (M+Na)⁺

Preparation 92

NMR (DMSO-d₆+D₂O, δ): 0.95 (3H, d, J=6.8 Hz), 1.08 (3H, d, J=5.6 Hz), 1.35 (9H, s), 1.5-2.9 (9H, m), 3.1-4.5 (22H, m), 4.7-4.9 (2H, m), 6.5-6.8 (3H, m)

ESI MASS (Positive): 1007.3 (M+H)+1029.4 (M+Na)⁺

Preparation 93

To a solution of starting compound (93) (760 mg) and cobalt(II) chloride hexahydrate (668 mg) in a mixture of methanol (22.8 ml) and water (4.6 ml) was added sodium borohydride (531 mg) with stirring at room temperature and the mixture was stirred at the same temperature for 1.5 hours. The reaction mixture was filtered through celite. The filtrate was adjusted pH 8.3 with 1N-hydrochloric acid, evaporated under reduced pressure to remove methanol and was lyophilized. To the mixture of resulting residue and N,N-diisopropylethylamine (0.367 ml) in N,N-dimethylformamide (15.2 ml) was added 9-fluorenylmethyl chloroformate (272 mg) at room temperature, and the mixture was stirred for 2 hours. Water was added to the mixture and the resulting precipitate was collected by filtration. And the precipitate was dissolved in 10% acetonitrile in water. The solution was subjected to column chromatography on ODS eluting with 40% acetonitrile in water. The fractions containing the object compound were collected and evaporated under reduced pressure to remove acetonitrile. The residue was lyophilized to give object compound (93)(290 mg).

NMR (DMSO-d₆+D₂O, δ): 0.89 (3H, d, J=6.7 Hz), 1.05-1.15 (3H, m), 1.34 (9H, s), 1.4-3.3 (13H, m), 3.4-4.5 (21H, m), 4.7-4.85 (2H, m), 6.55-6.75 (3H, m), 7.3-7.5 (4H, m), 7.67 (2H, d, J=7.4 Hz), 7.89 (2H, d, J=7.6 Hz)

MASS (m/z): 1199.4 (M+Na)

Preparation 94

To a solution of starting compound (94) (290 mg) and triethylsirane (0.315 ml) in dichloromethane (6 ml) was added dropwise trifluoroacetic acid (0.719 ml) with stirring under ice-cooling, and the mixture was stirred at room temperature for 4 hours. The reaction mixture was added a mixture of pH 6.86 buffer (40 ml) and sodium hydrogen carbonate solution (10 ml). The solution was subjected to column chromatography on ODS eluting with 40% acetonitrile in water. The fractions containing the object compound were collected and evaporated under reduced pressure to remove acetonitrile. The residue was lyophilized to give object compound (94) (159 mg).

MASS (m/z): 1099.3 (M+Na)

Preparation 95

A mixture of starting compound (95) (80 mg), 1-[4-[2-(4-pentyloxyphenyl)-1,3-thiazol-5-yl]benzoyloxy]-1H-1,2,3-benzotriazol (39.6 mg) and diisopropylethylamine (38.8 μl) in N,N-dimethylformamide (0.8 ml) was stirred at room temperature. The solution was stirred for 15 hours at the same temperature. Then to the reaction mixture was added piperidine (73.5 μl) and the mixture was stirred for 2.5 hours. Ethyl acetate was added to the reaction mixture. The resulting precipitates were collected by filtration and dried in vacuo. The precipitates was subjected to column chromatography on ODS eluting with 40% acetonitrile in water. The fractions containing the object compound were collected and evaporated under reduced pressure to remove acetonitrile. The residue was lyophilized to give object compound (95) (25.5 mg).

NMR (DMSO-d₆+D₂O, δ): 0.8-1.05 (6H, m), 1.09 (3H, d, J=5.3 Hz), 1.25-4.55 (39H, m), 4.7-4.85 (2H, m), 6.5-6.8 (3H, m), 7.08 (2H, d, J=8.9 Hz), 7.81 (2H, d, J=8.5 Hz), 7.92 (2H, d, J=8.9 Hz), 7.97 (2H, d, J=8.5 Hz), 8.37 (1H, s)

MASS (m/z): 1226.4 (M+Na)

Analysis Calculated for C₅₈H₇₈ClN₉O₁₇S.5H₂O: C, 52.34; H, 6.66; N, 9.47 Found: C, 52.15; H, 6.57; N, 9.23

The following compound was obtained according to a similar manner to that of Preparation 26.

Preparation 96

NMR (DMSO-d₆+D₂O, δ): 0.95 (3H, d, J=6.8 Hz), 1.08 (3H, d, J=5.6 Hz), 1.36 (9H, s), 1.5-2.9 (9H, m), 3.1-4.5 (20H, m), 4.8-4.9 (2H, m), 5.09 (2H, s), 6.6-7.1 (3H, m), 7.2-7.5 (5H, m)

ESI MASS (Positive): 1125.4, 1127.3 (M+Na)⁺

The Starting Compounds used and the Object Compounds obtained in the following Examples 1 to 170 are given in the table as below, in which the formulas of the starting compounds are in the upper column, and the formulas of the object compounds are in the lower column, respectively.

| Example | |
|---|---|
| No. | Formula |
| 1 | 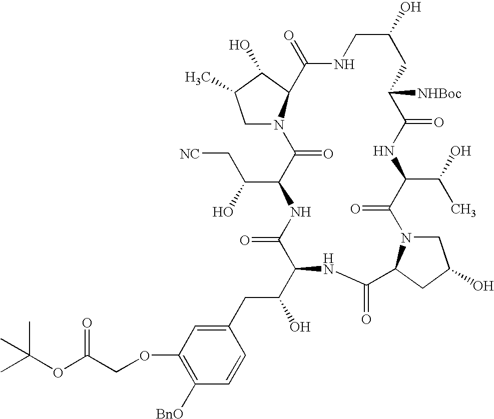 |
| | 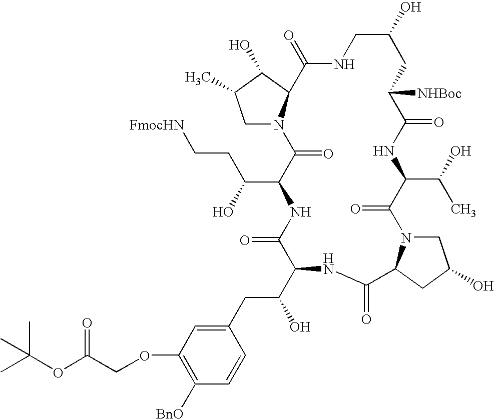 |
| 2 | 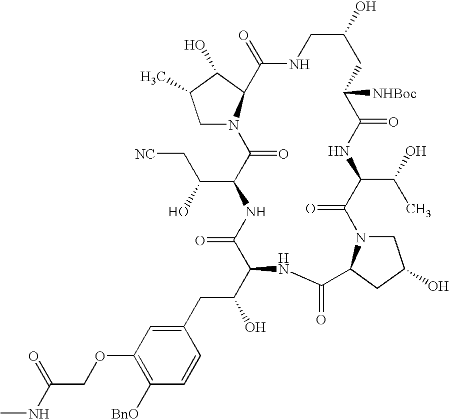 |
| | 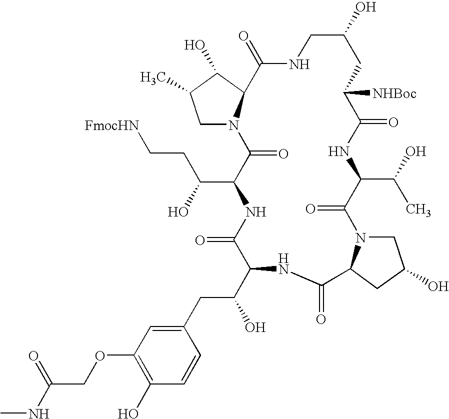 |
| 3 | 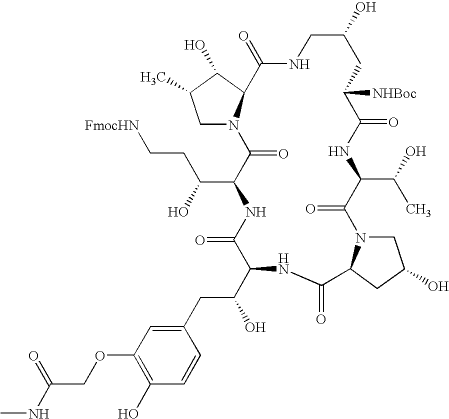 |
| | 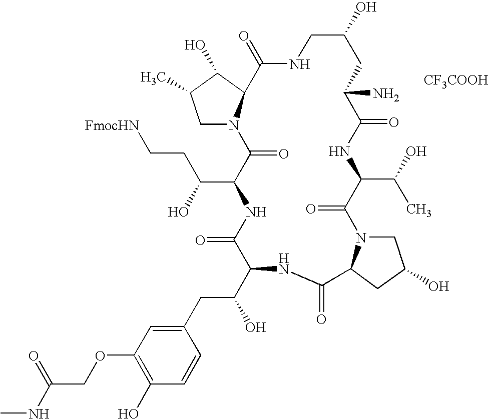 |
| 4 | 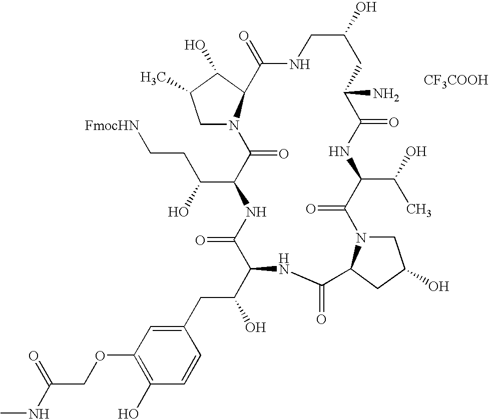 |
| | 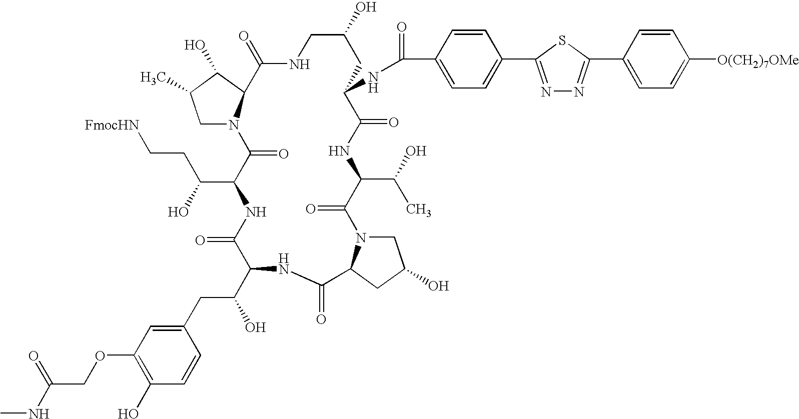 |
| 5 | 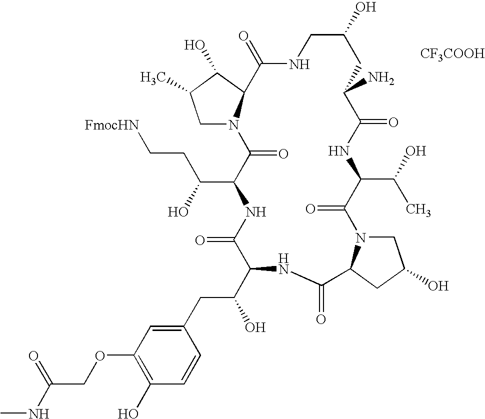 |
| | 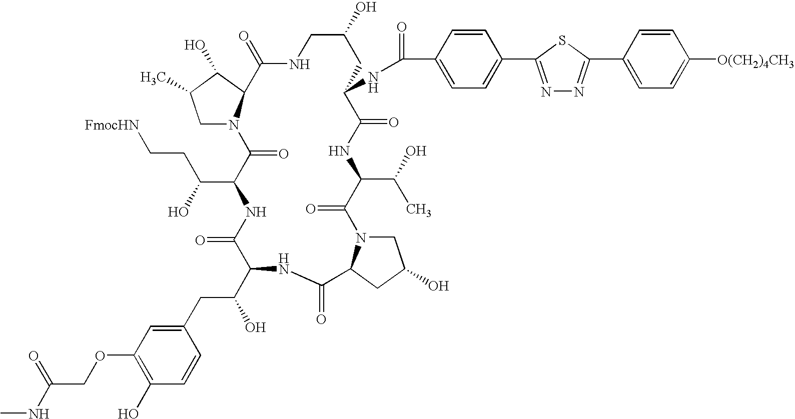 |
| 6 | 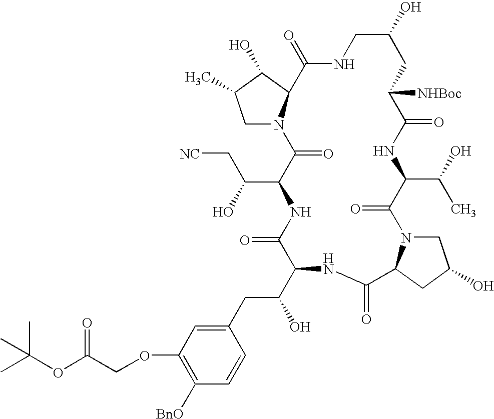 |
| | 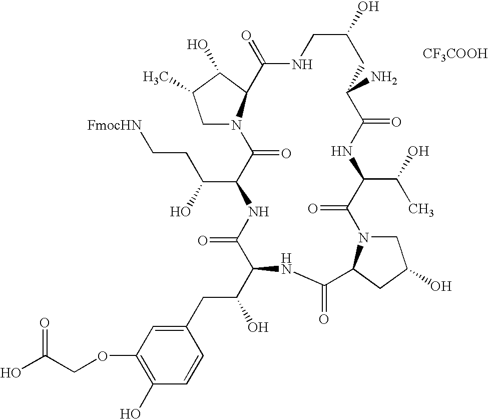 |
| 7 | 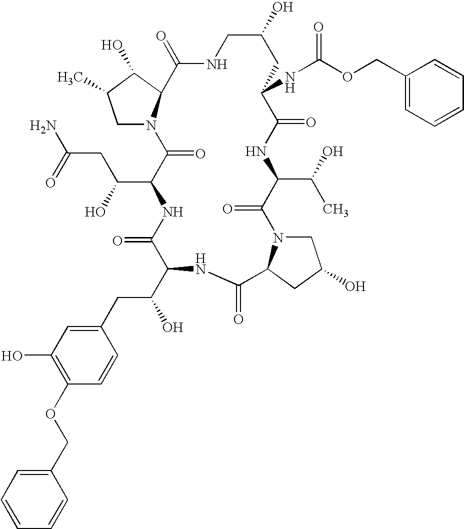 |
| | 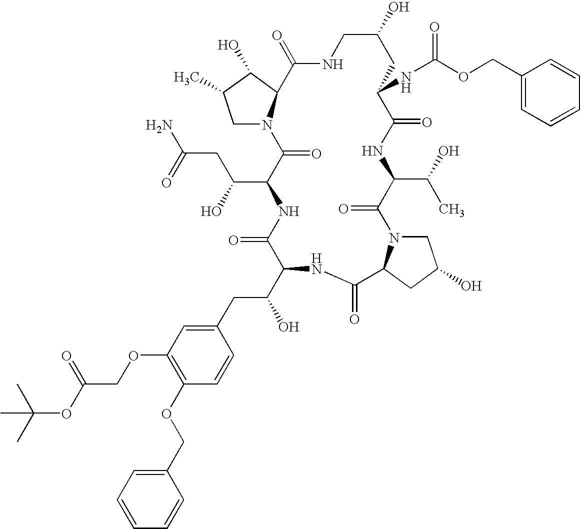 |
| 8 | 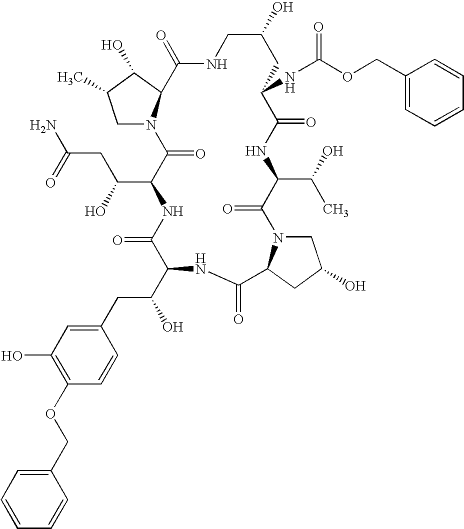 |
| | 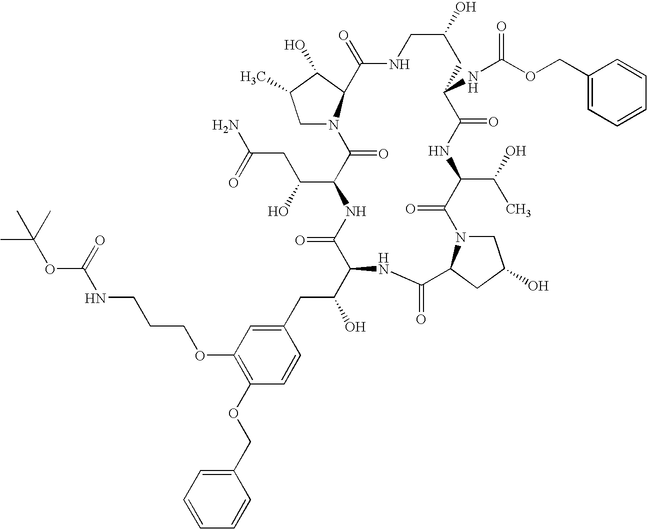 |
| 9 | 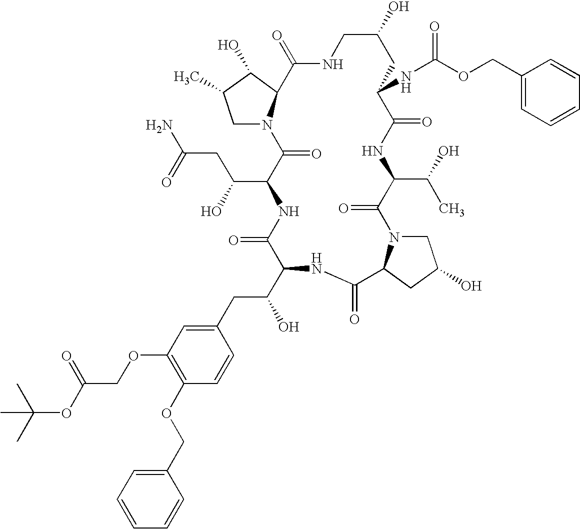 |
| | 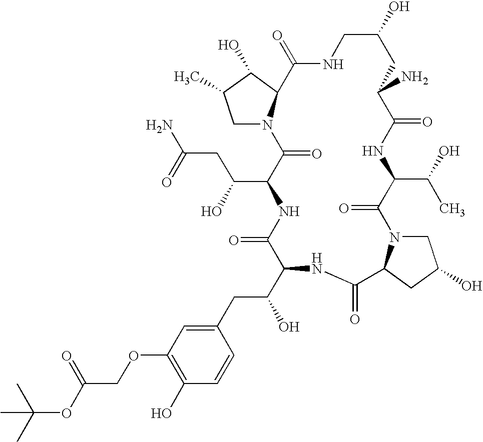 |
| 10 | 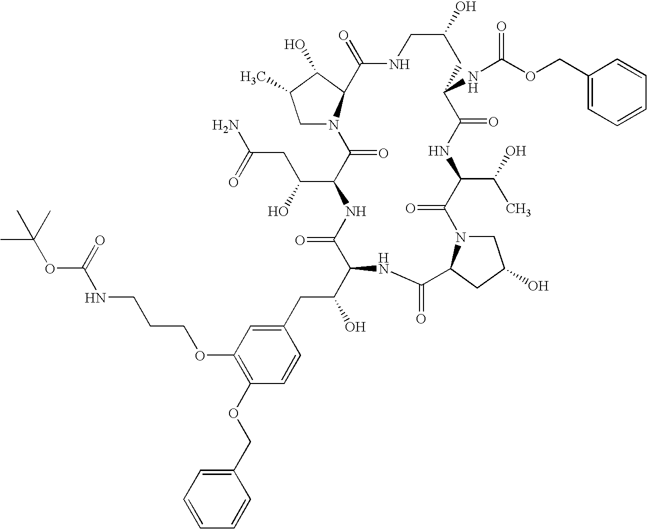 |
| | 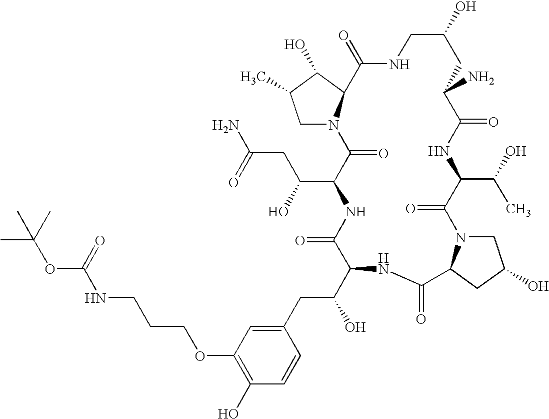 |
| 11 | 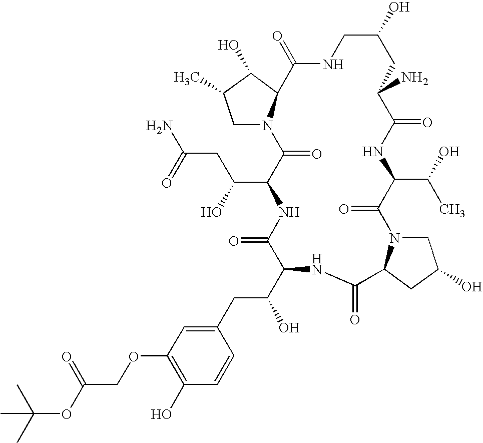 |
| | 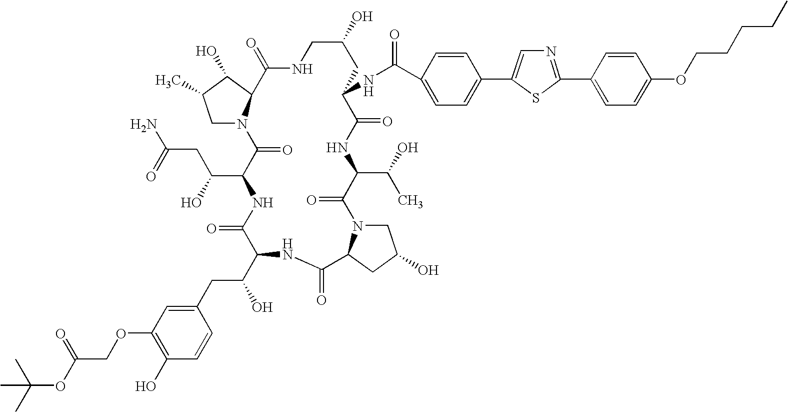 |
| 12 | 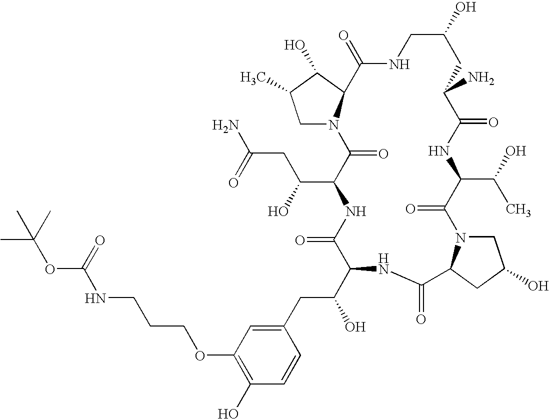 |
| | 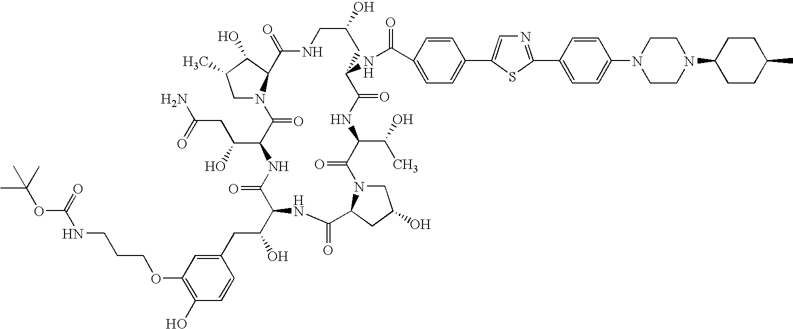 |
| 13 | 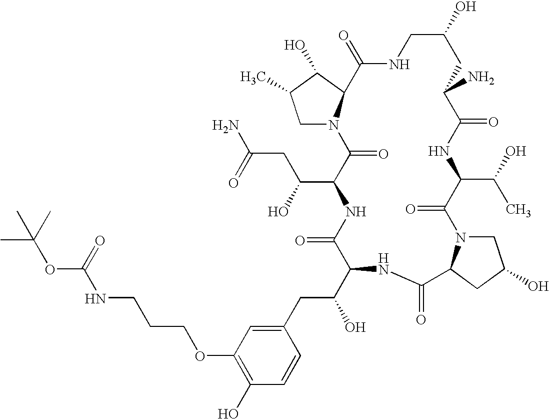 |
| | 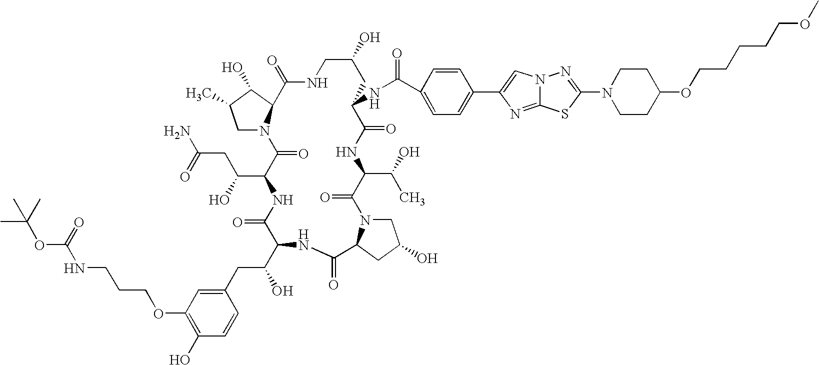 |
| 14 | 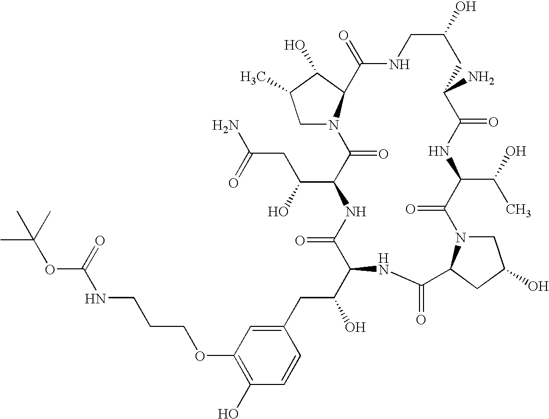 |
| | 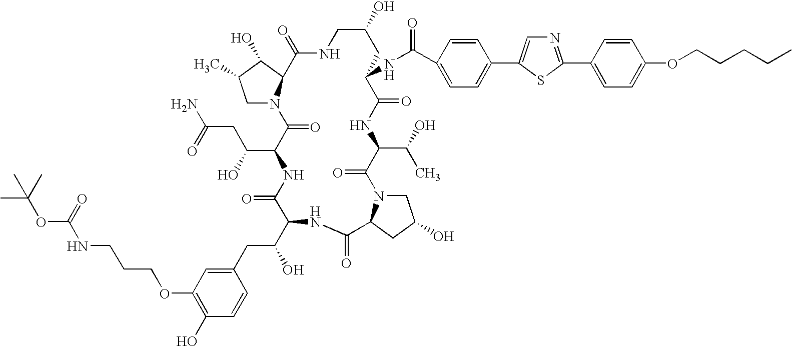 |
| 15 | 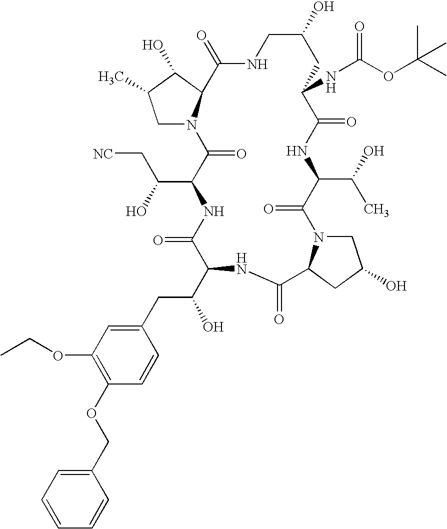 |
| | 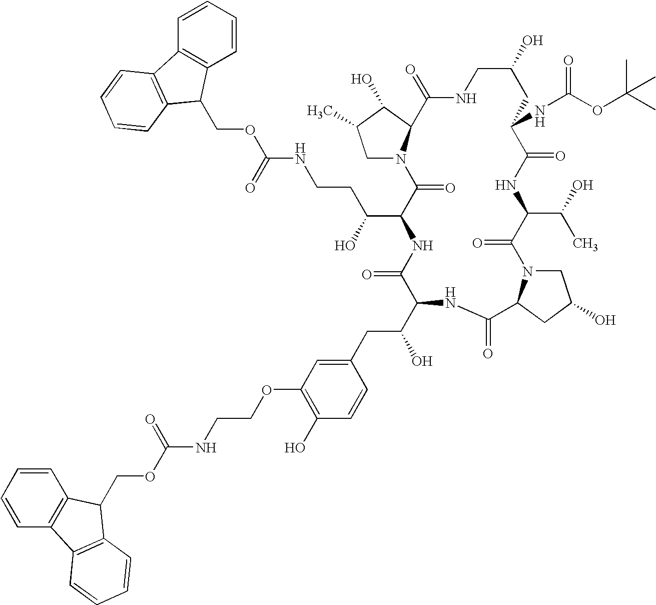 |
| 16 | 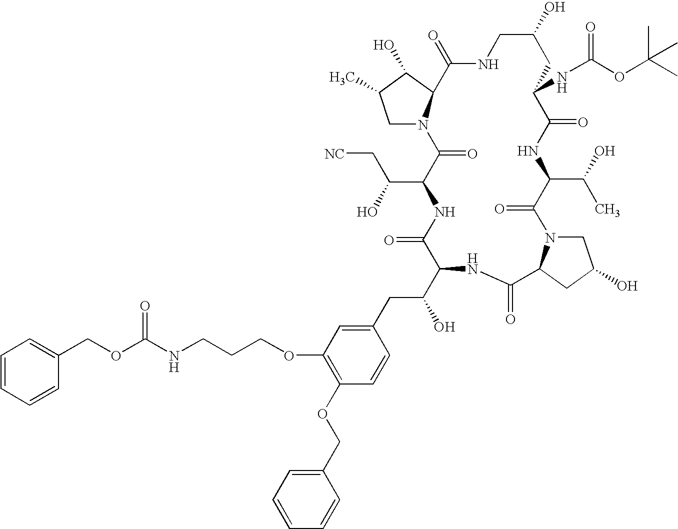 |
| | 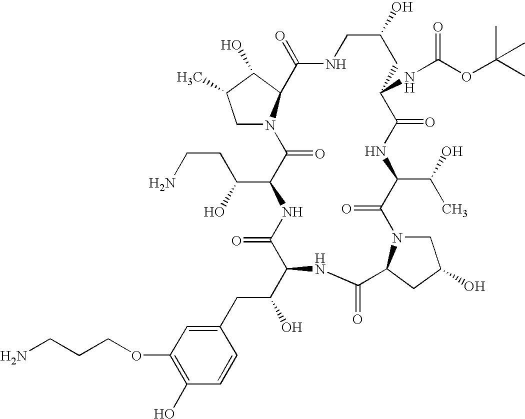 |
| 17 | 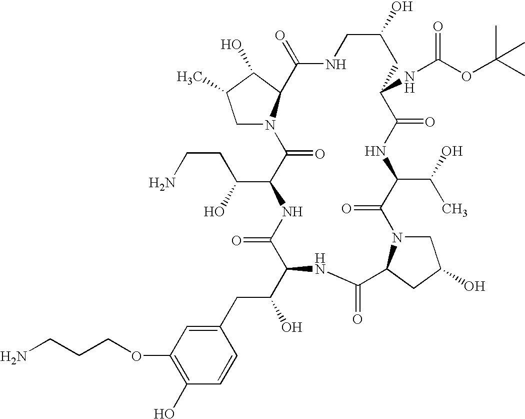 |
| | 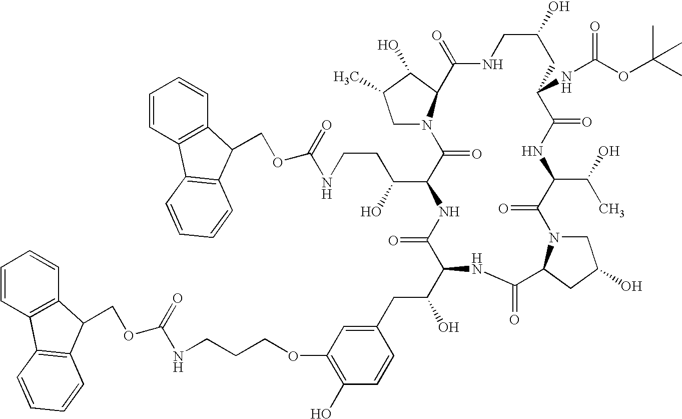 |
| 18 | 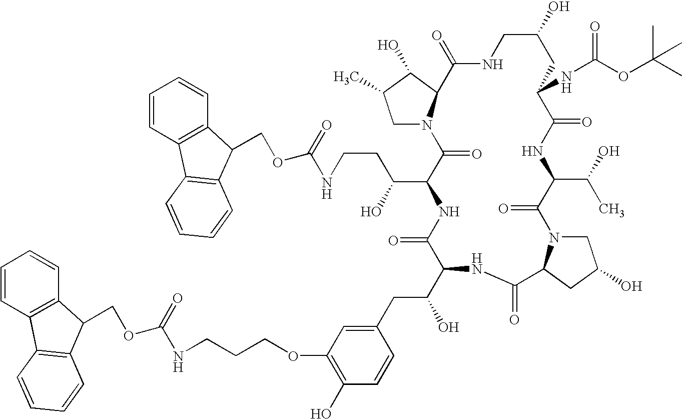 |
| | 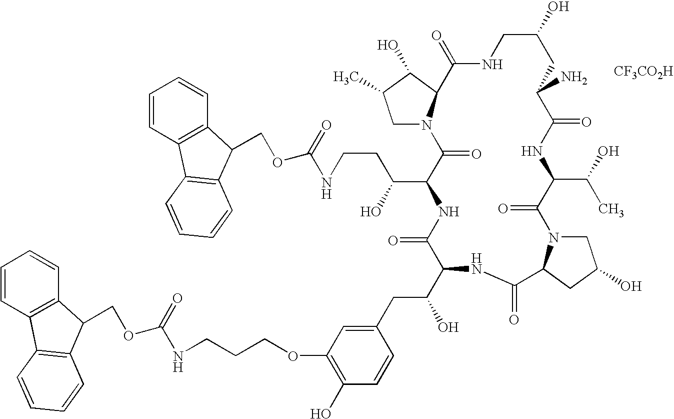 |
| 19 | 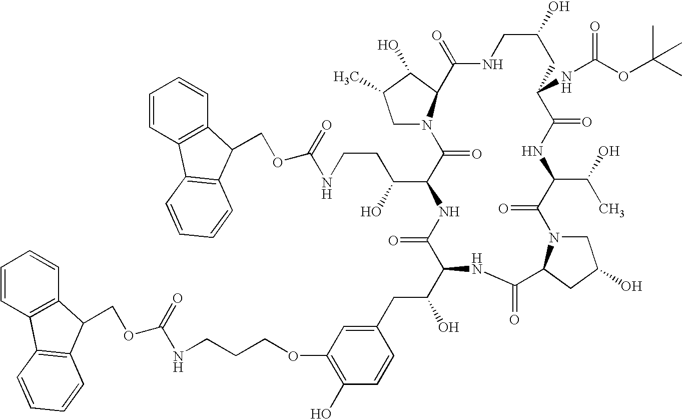 |
| | 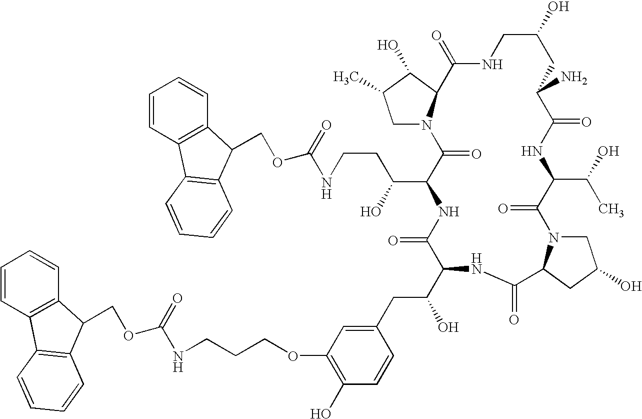 |
| 20 | 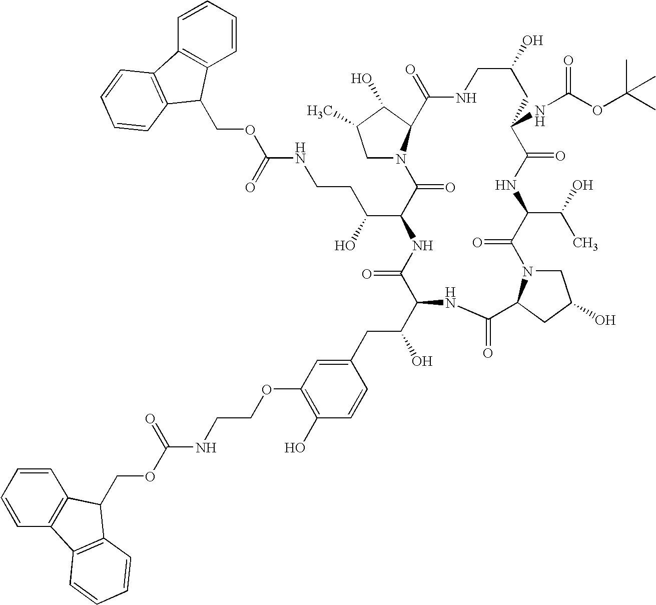 |
| | 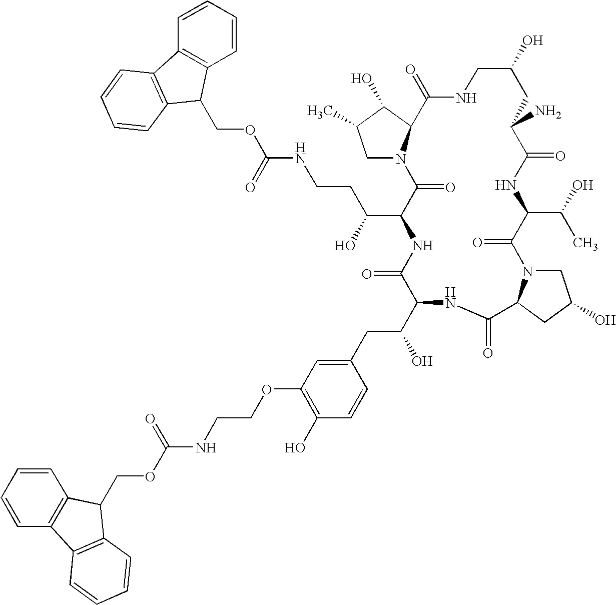 |
| 21 | 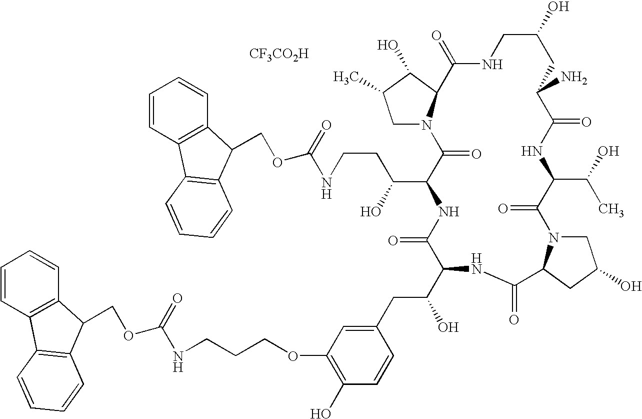 |
| | 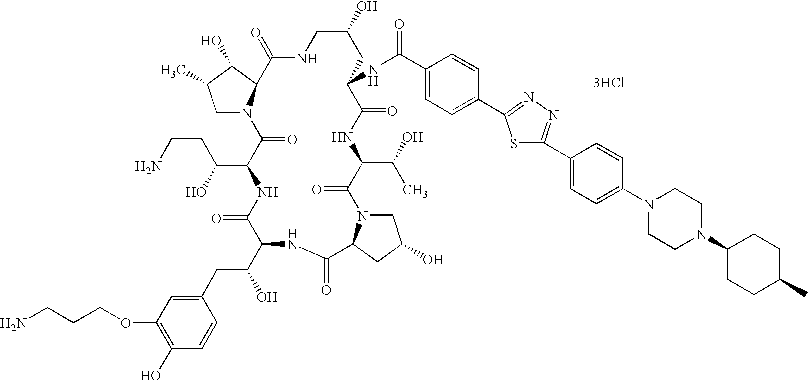 |
| 22 | 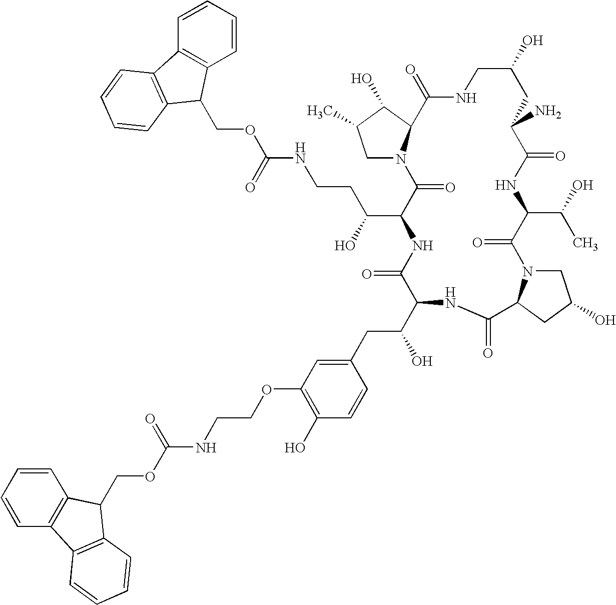 |
| | 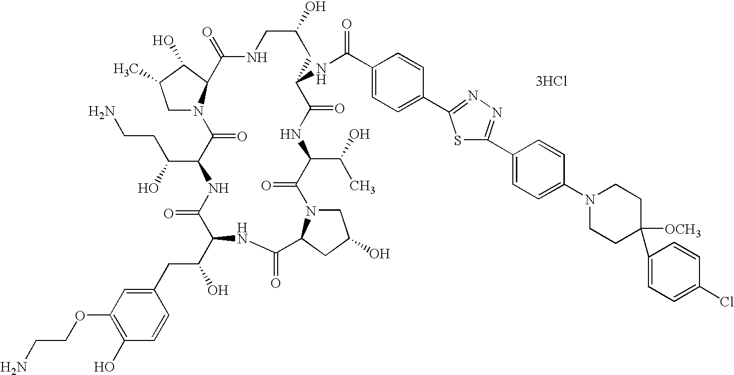 |
| 23 | 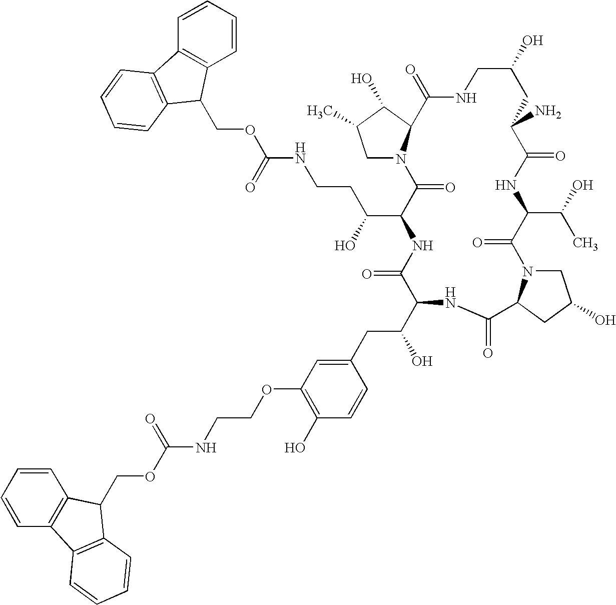 |
| | 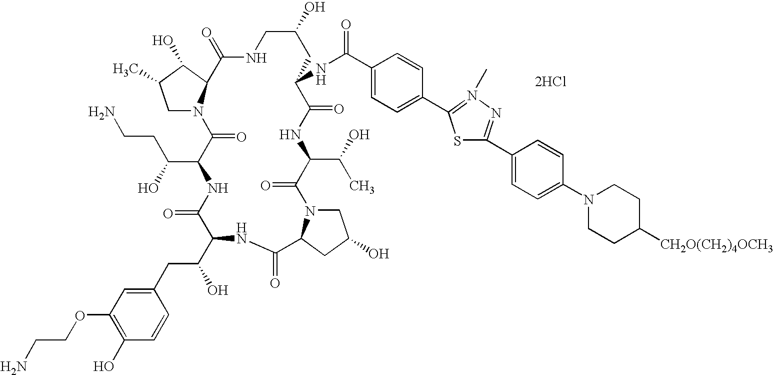 |
| 24 | 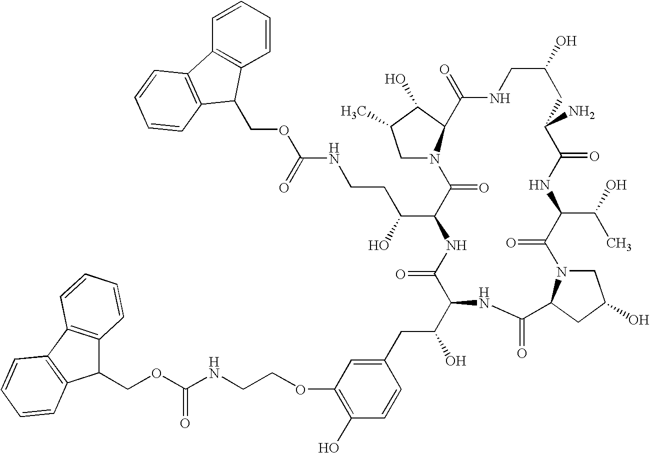 |
| | 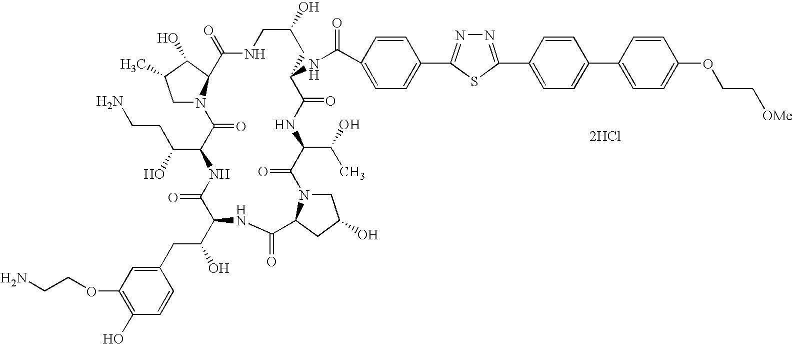 |
| 25 | 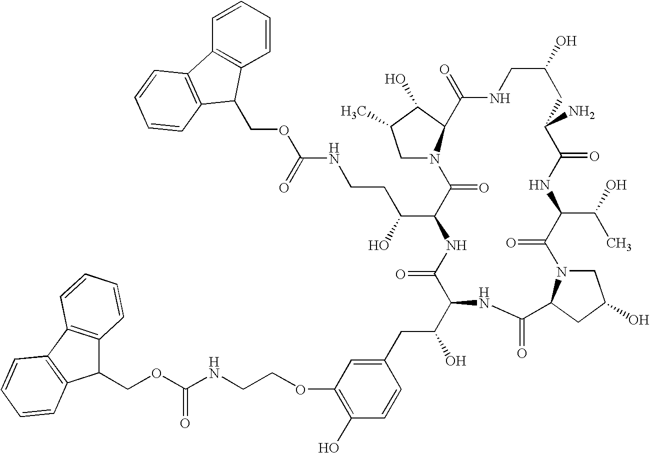 |
| | 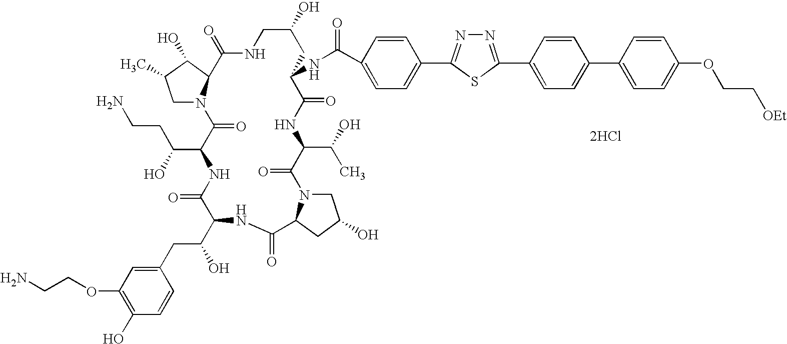 |
| 26 | 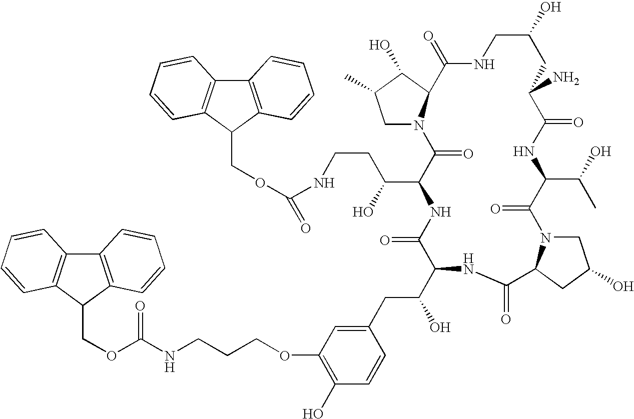 |
| | 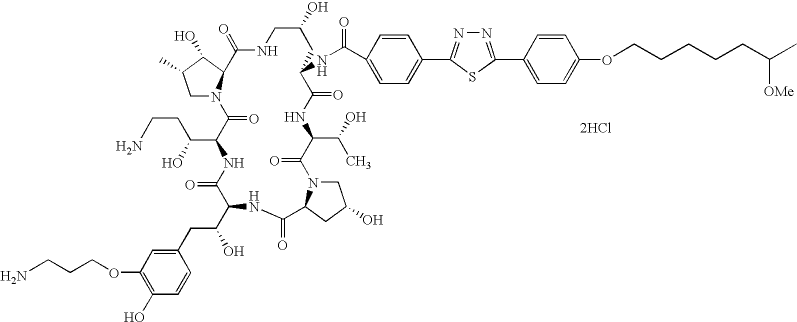 |
| 27 | 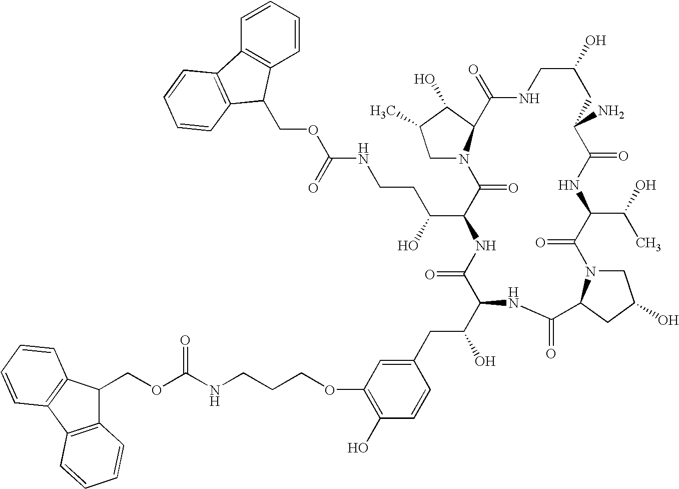 |
| | 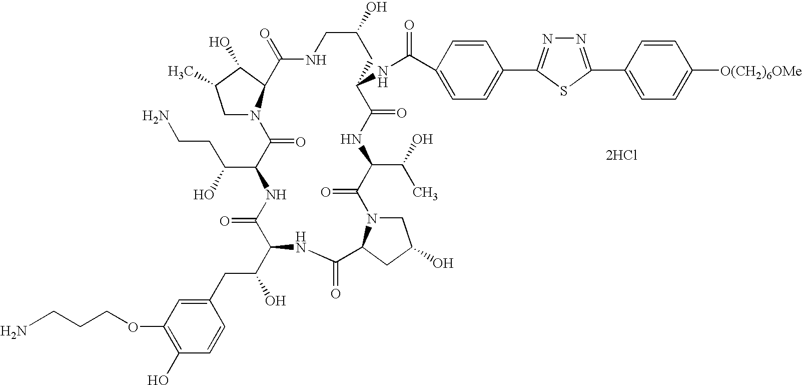 |
| 28 | 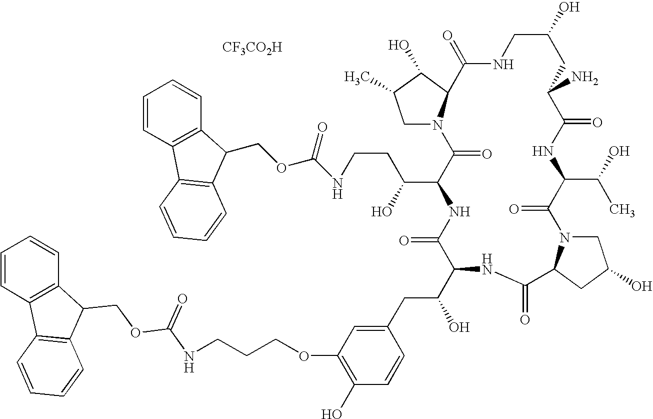 |
| | 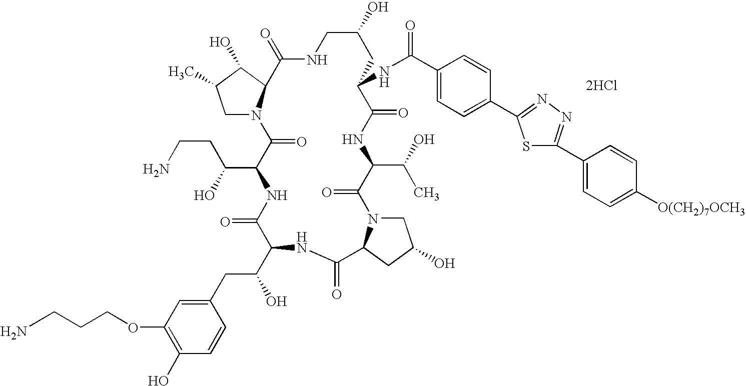 |
| 29 | 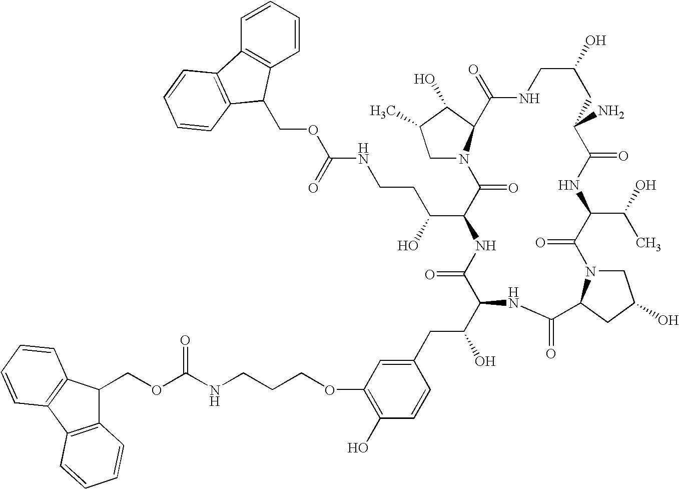 |
| | 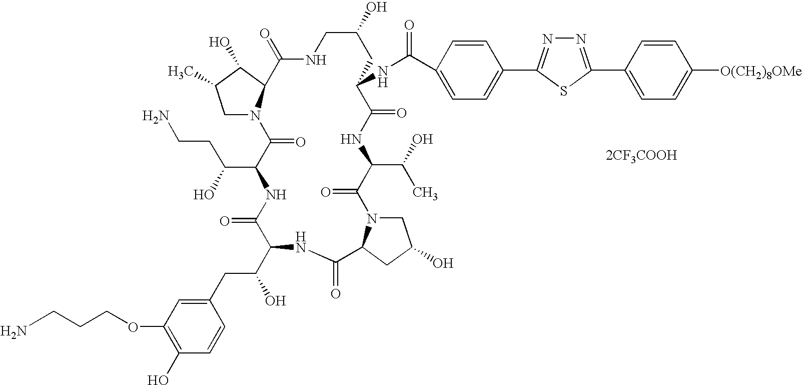 |
| 30 | 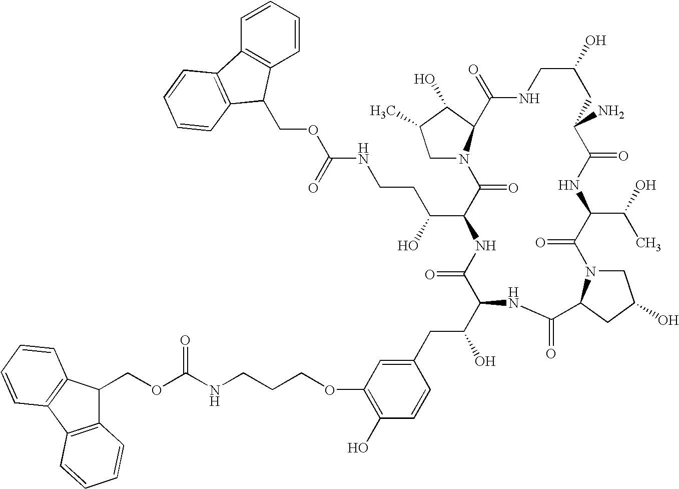 |
| | 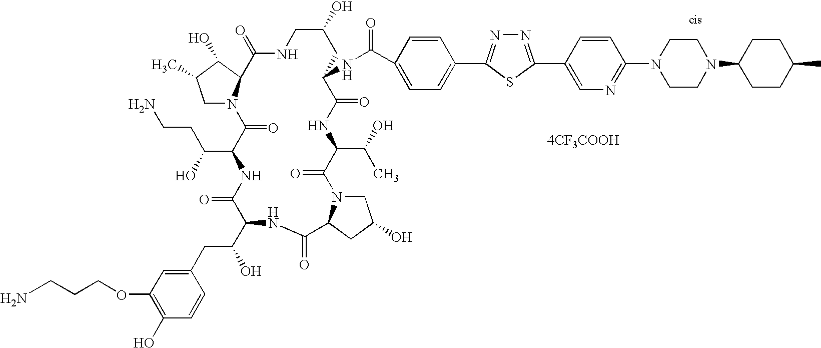 |
| 31 | 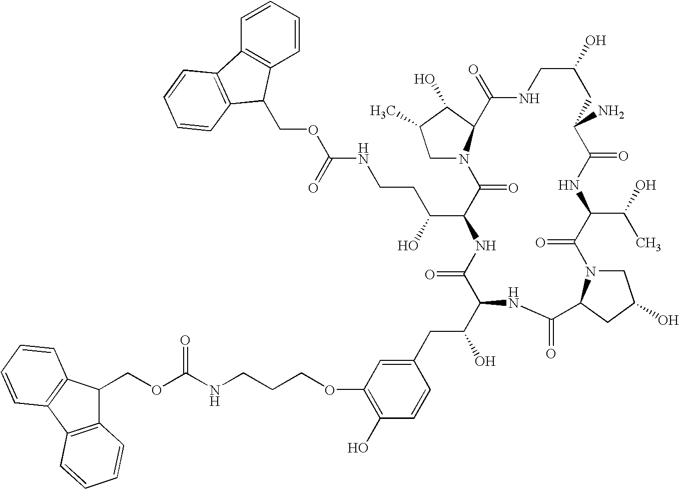 |
| | 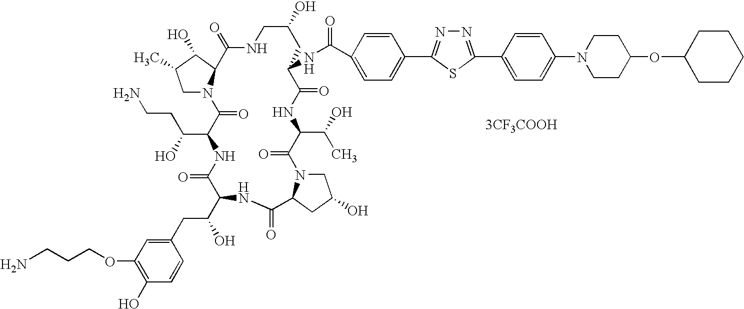 |
| 32 | 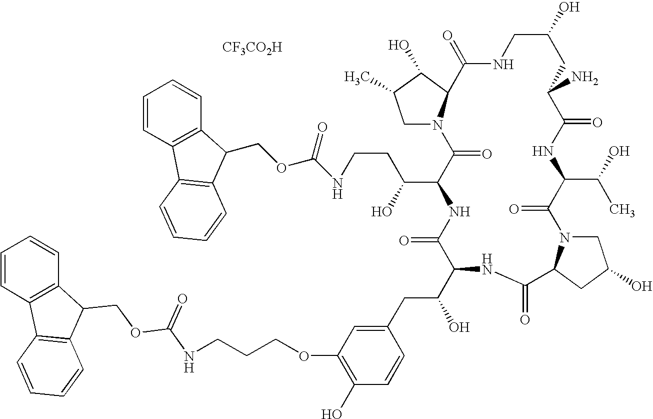 |
| | 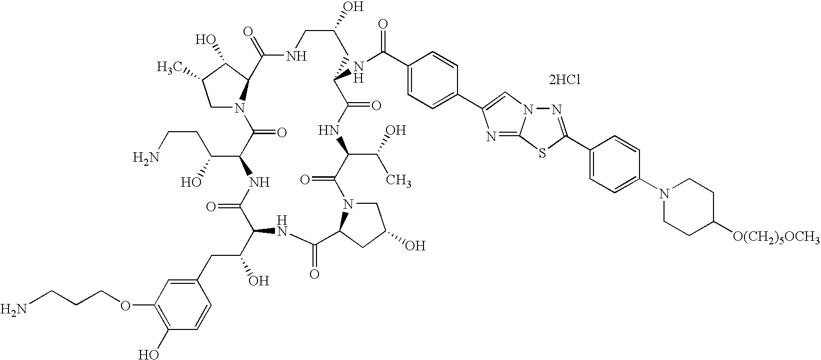 |
| 33 | 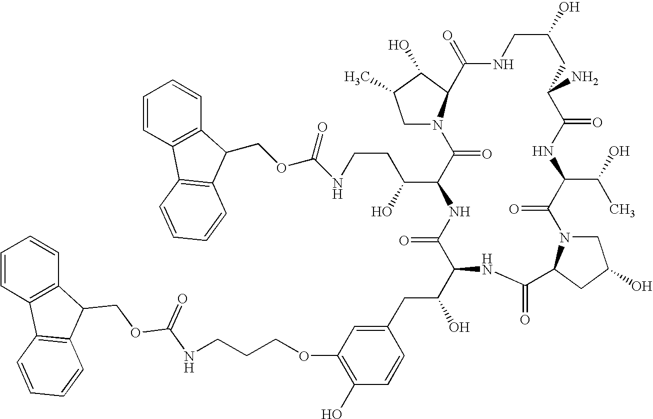 |
| | 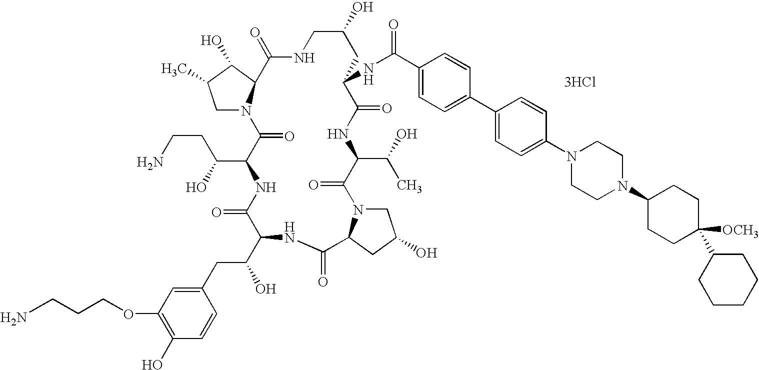 |
| 34 | 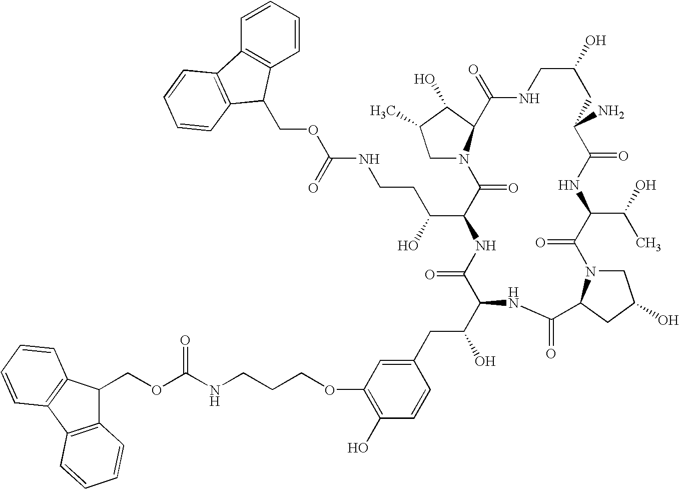 |
| | 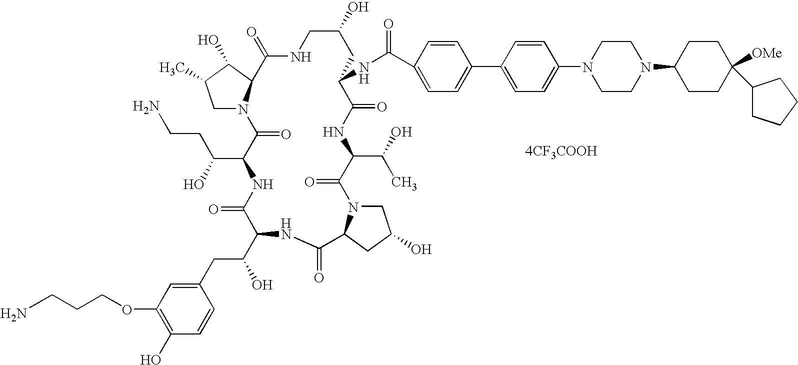 |
| 35 | 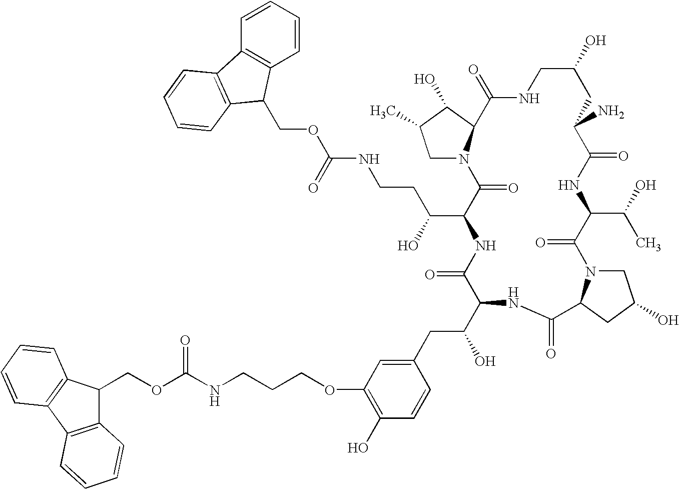 |
| | 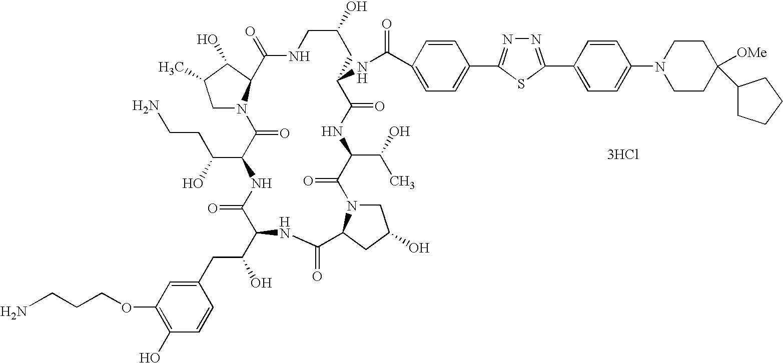 |
| 36 | 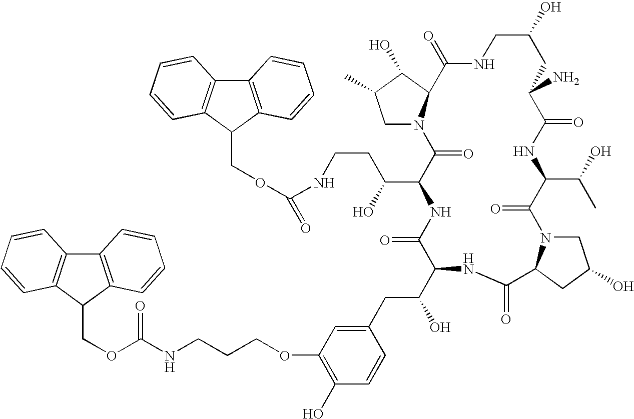 |
| | 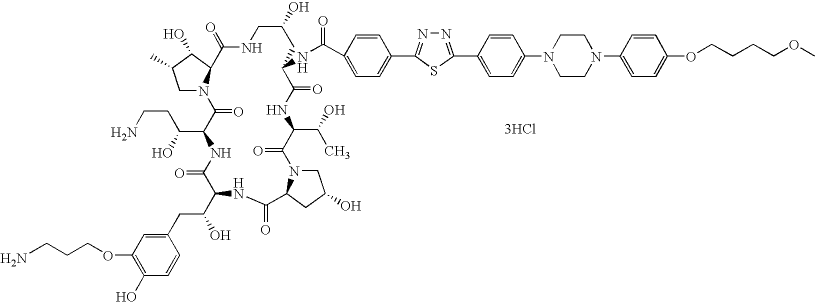 |
| 37 | 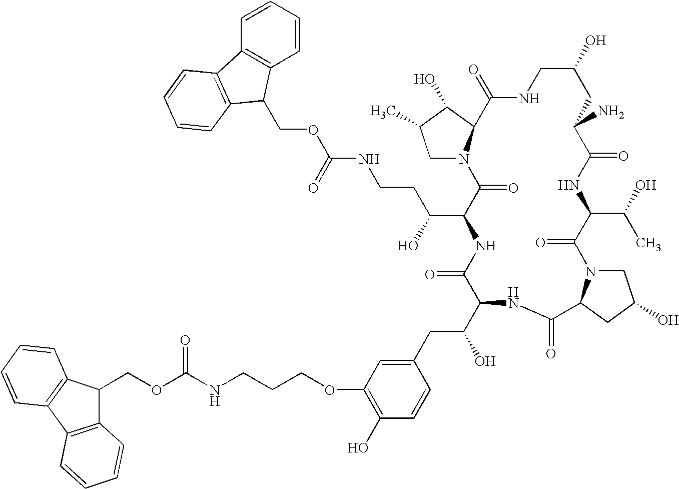 |
| | 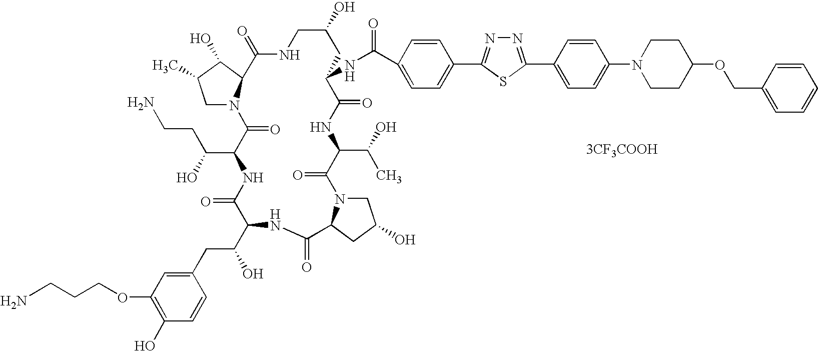 |
| 38 | 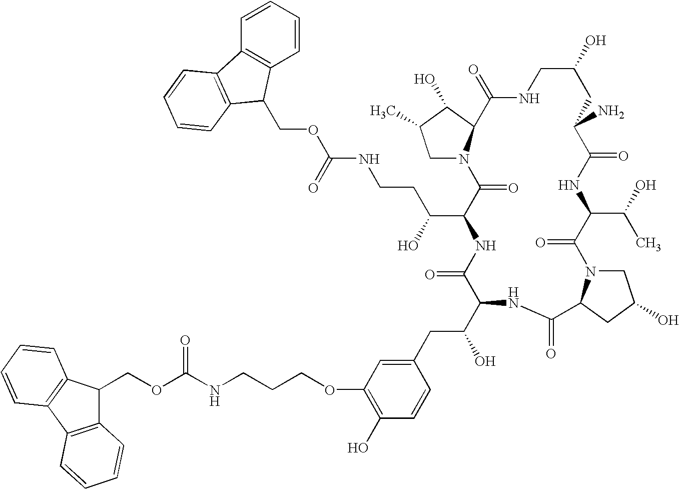 |
| | 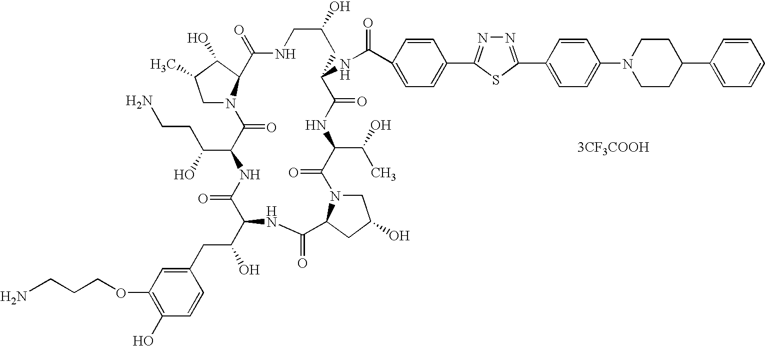 |
| 39 | 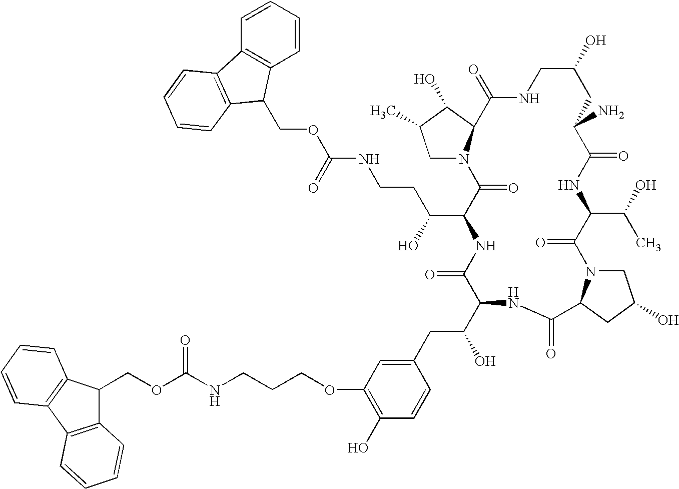 |
| | 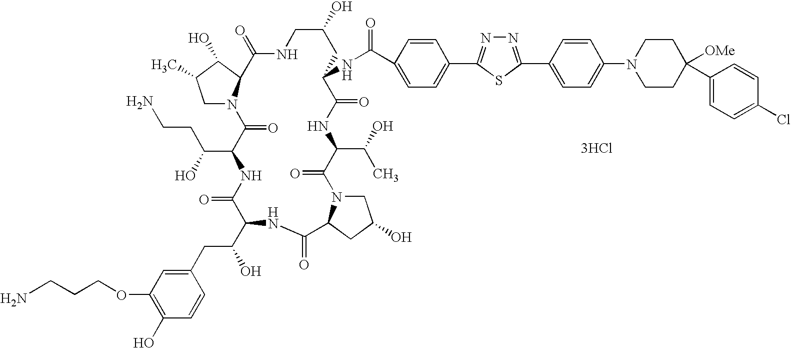 |
| 40 | 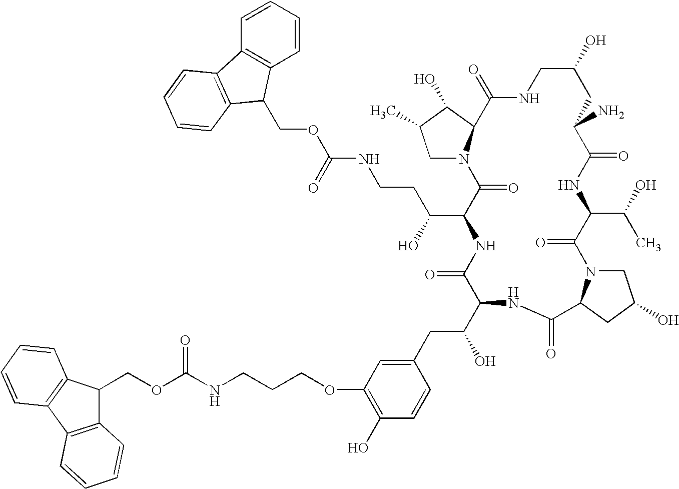 |
| | 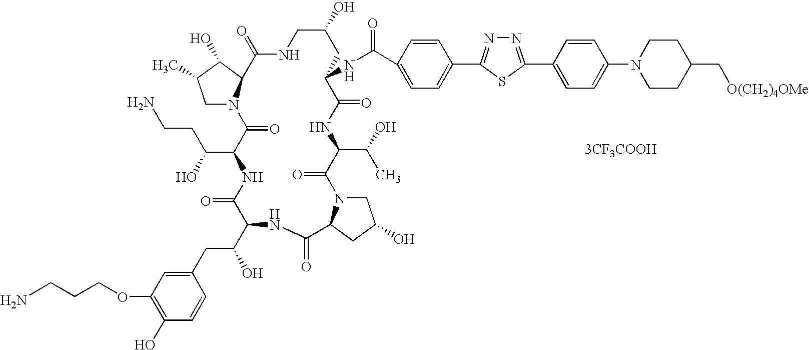 |
| 41 | 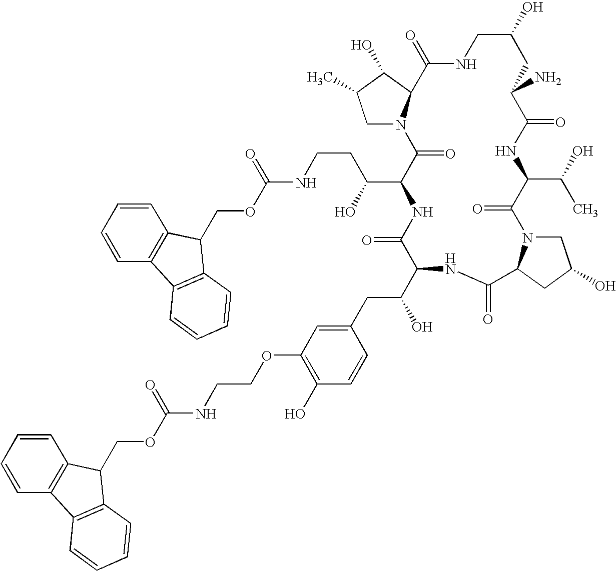 |
| | 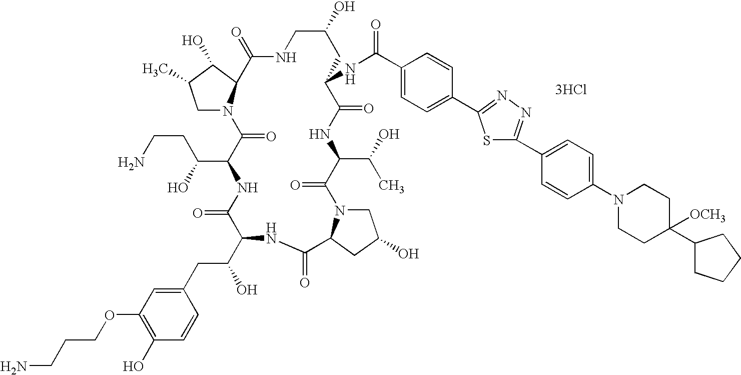 |
| 42 | 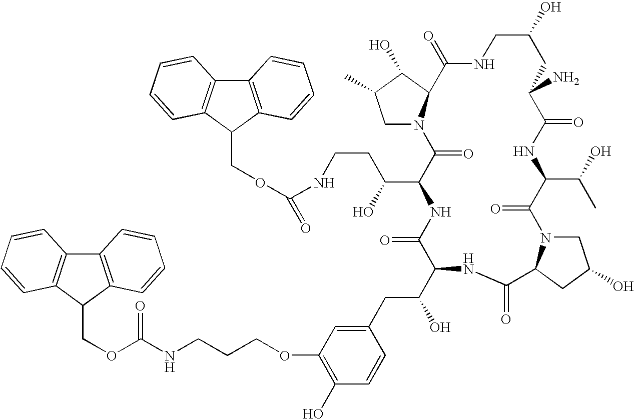 |
| | 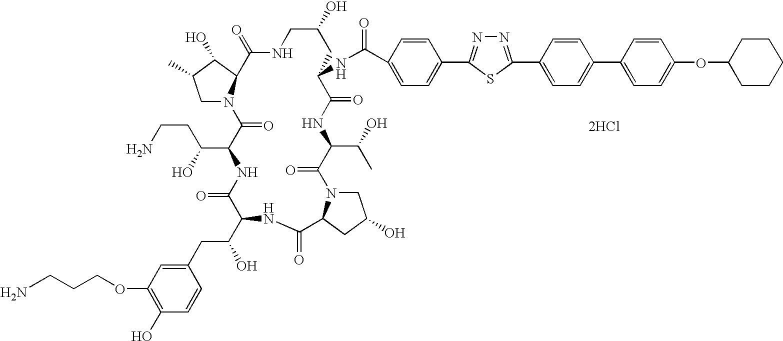 |
| 43 | 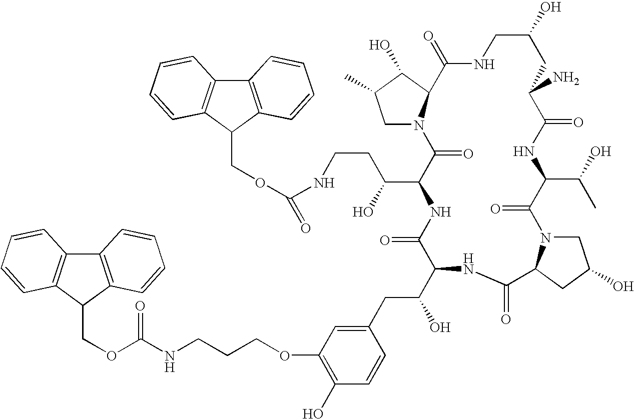 |
| | 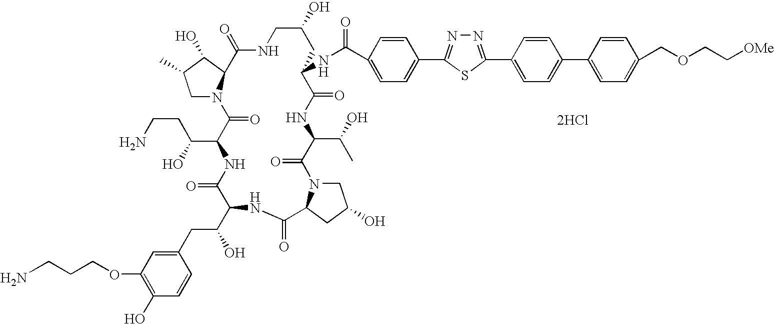 |
| 44 | 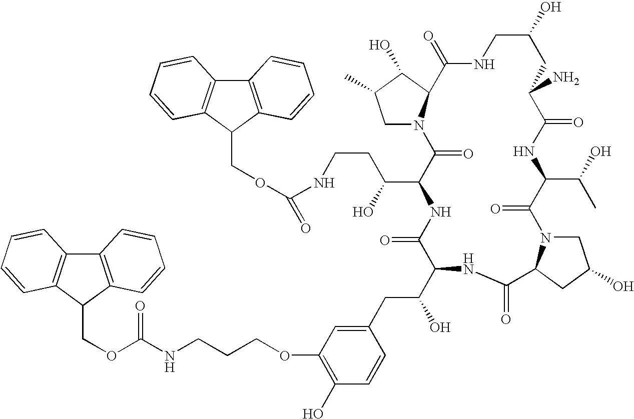 |
| | 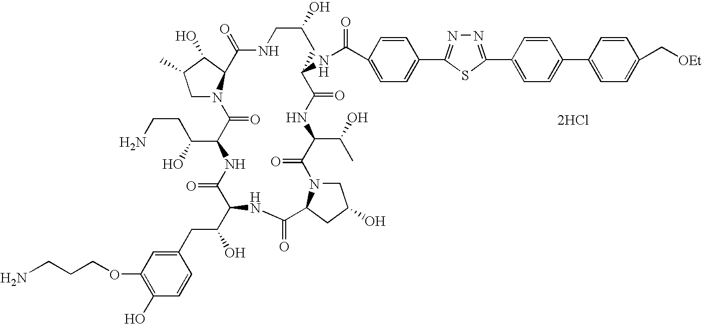 |
| 45 | 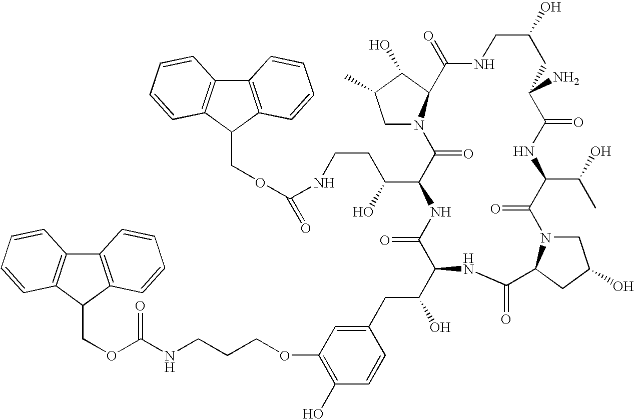 |
| | 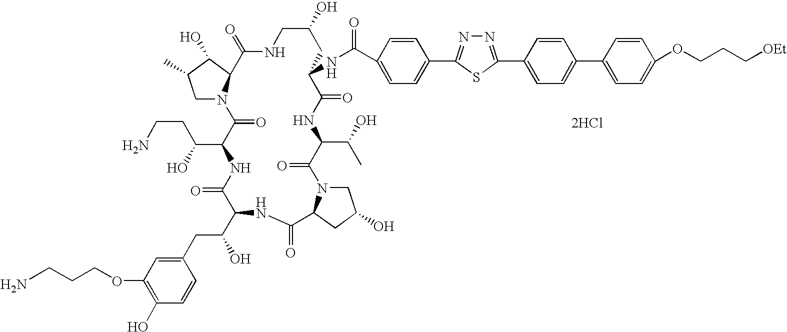 |
| 46 | 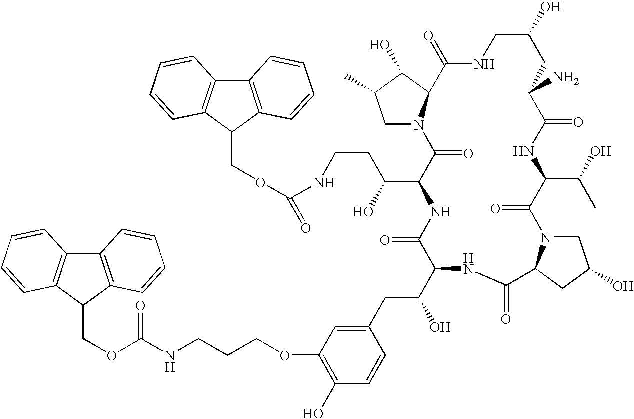 |
| | 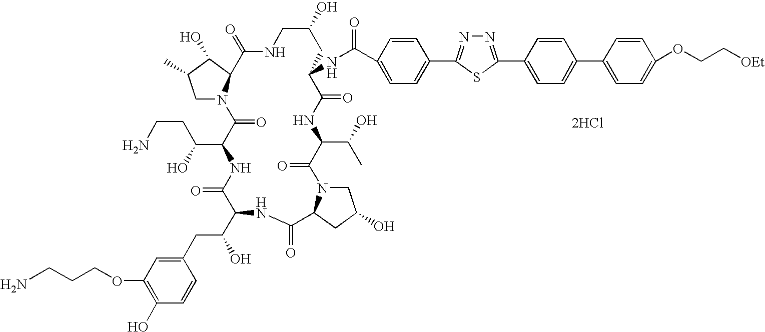 |
| 47 | 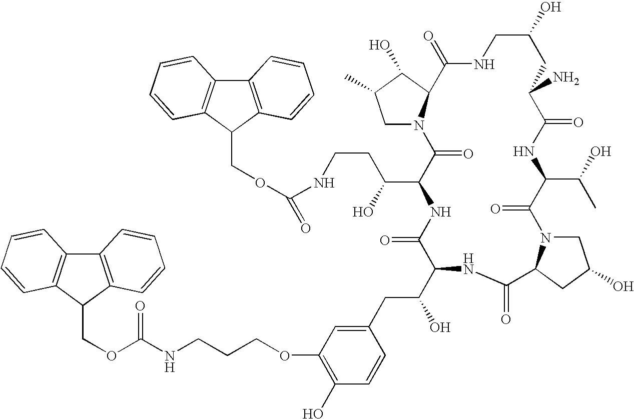 |
| | 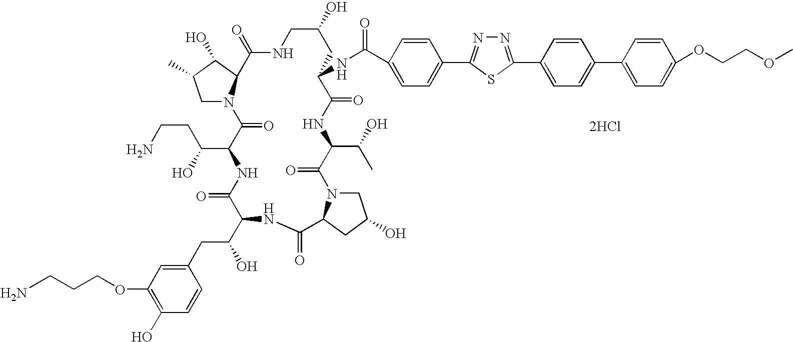 |
| 48 | 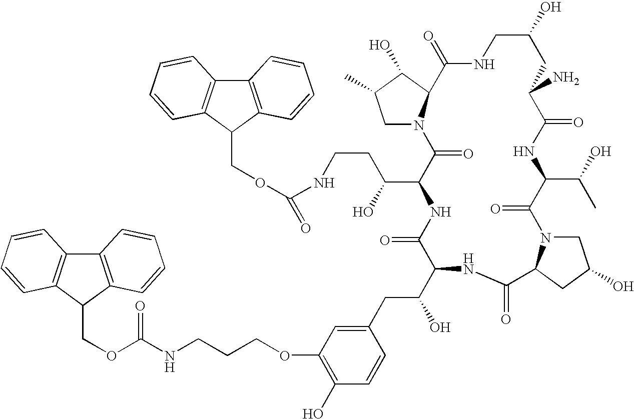 |
| | 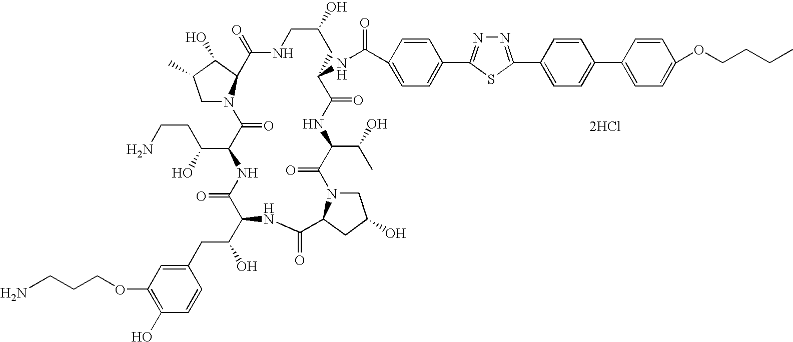 |
| 49 | 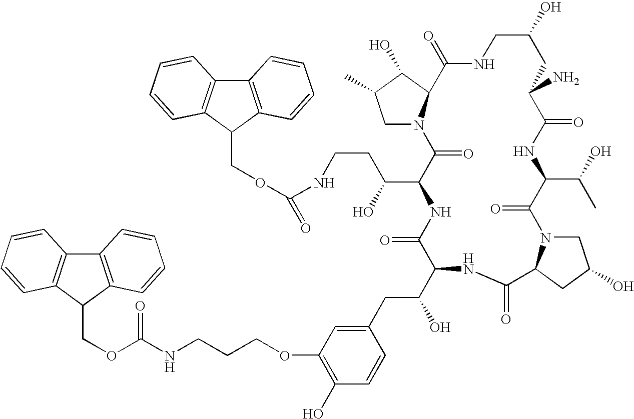 |
| | 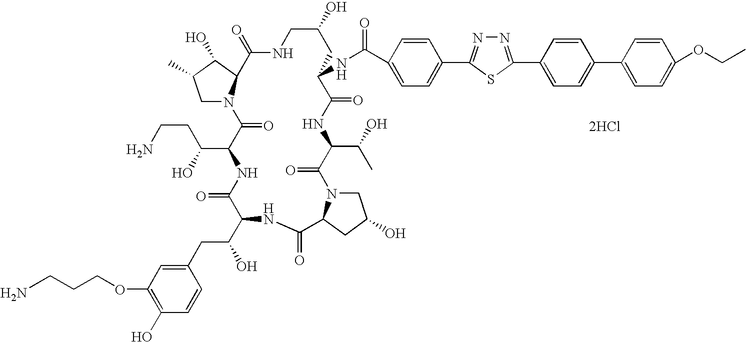 |
| 50 | 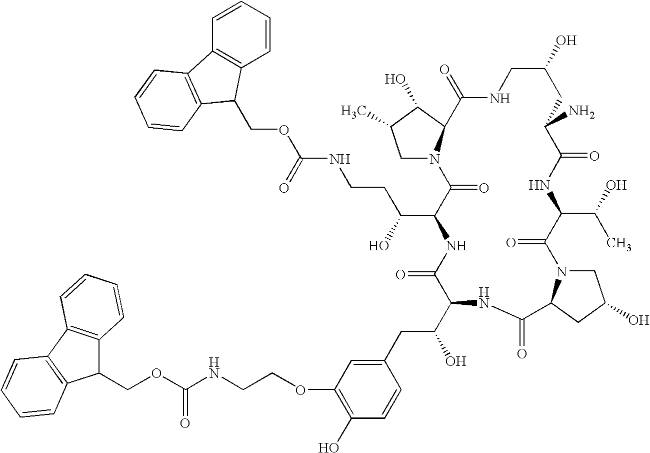 |
| | 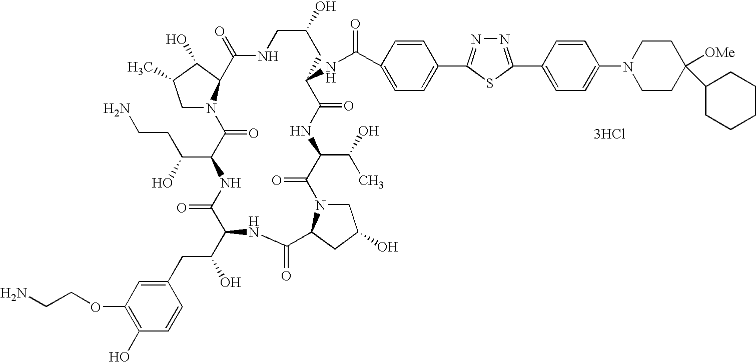 |
| 51 | 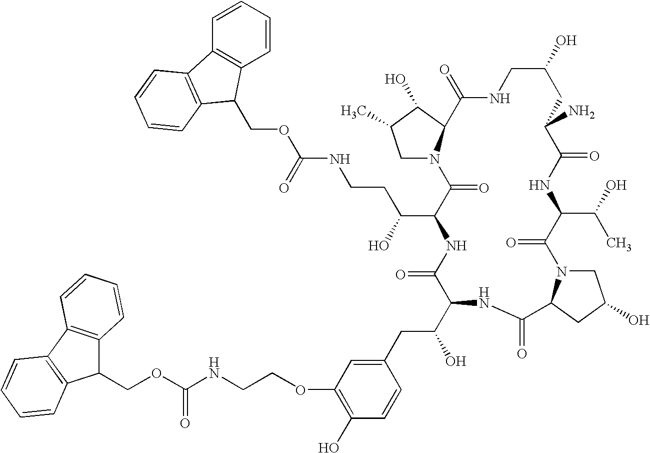 |
| | 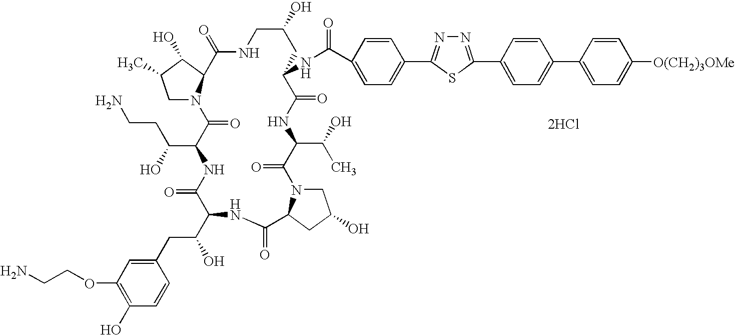 |
| 52 | 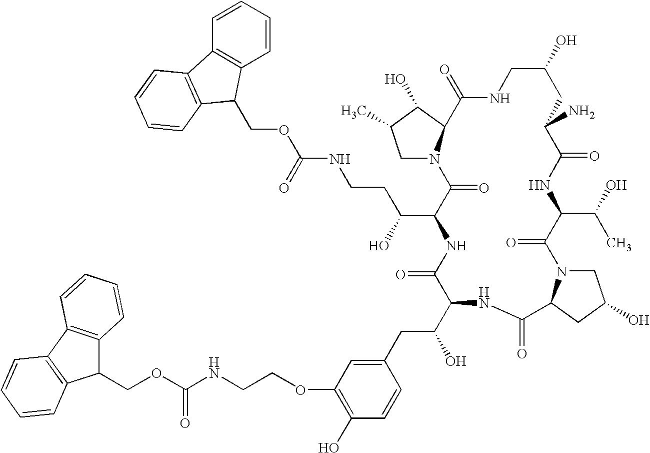 |
| | 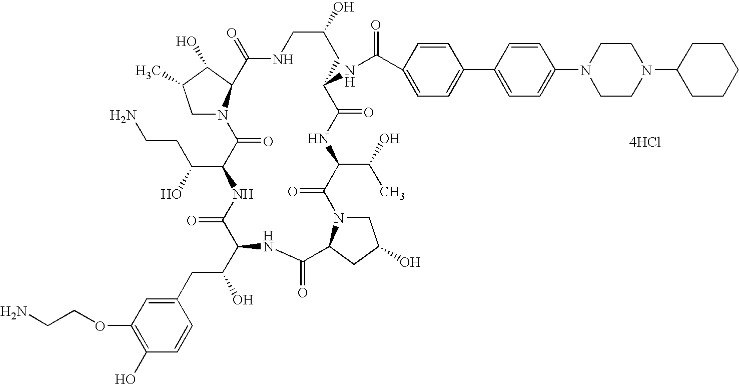 |
| 53 | 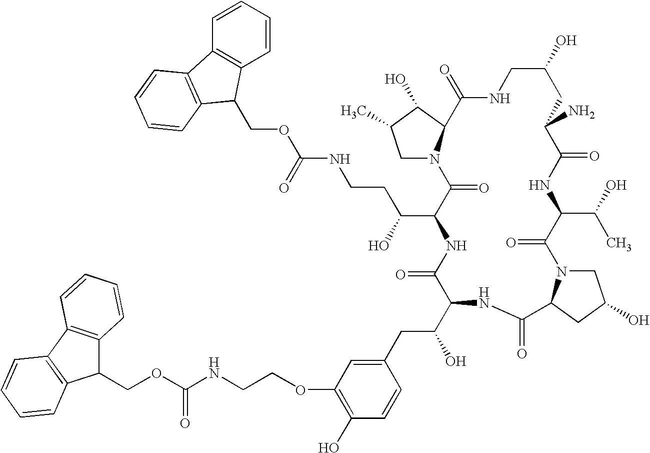 |
| | 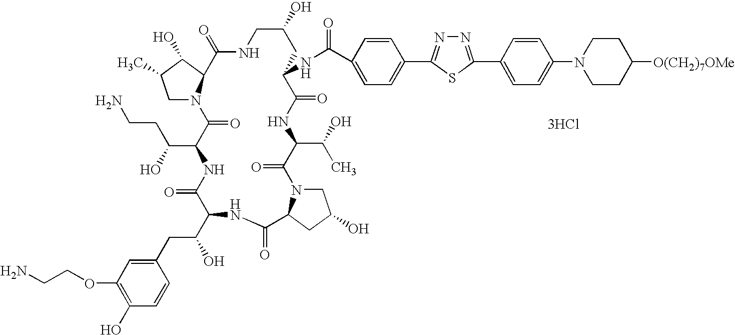 |
| 54 | 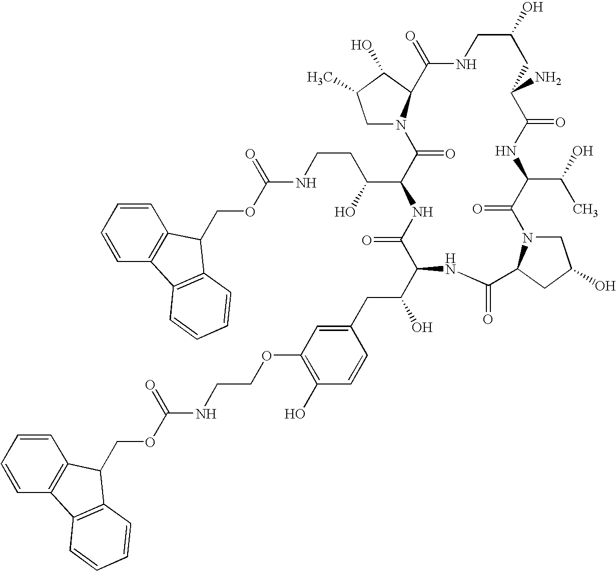 |
| | 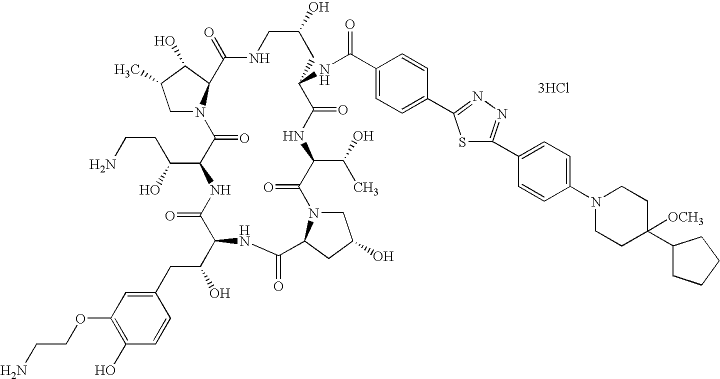 |
| 55 | 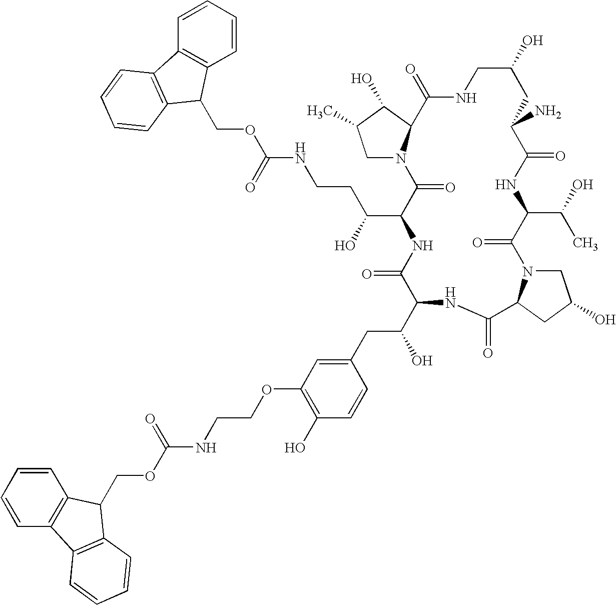 |
| | 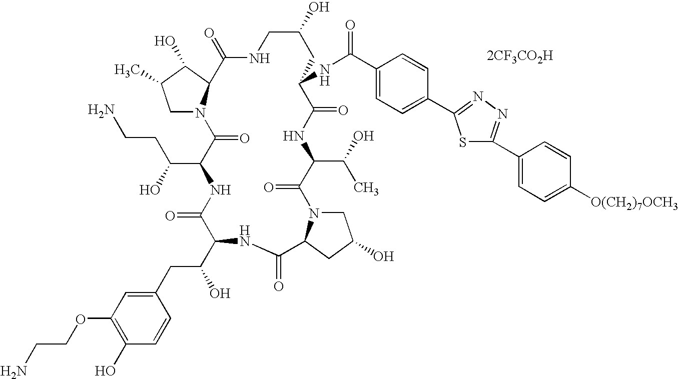 |
| 56 | 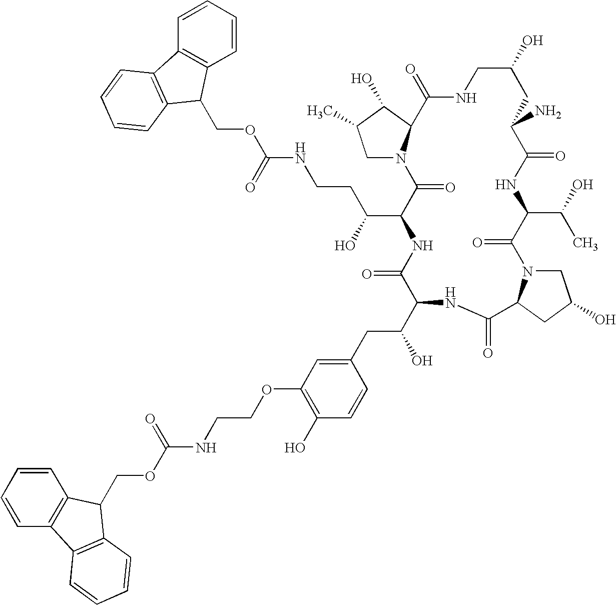 |
| | 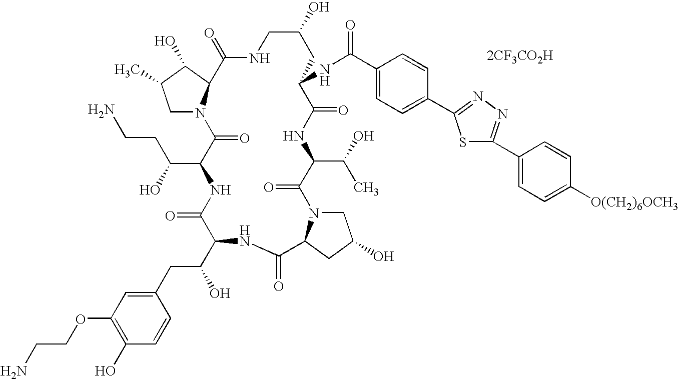 |
| 57 | 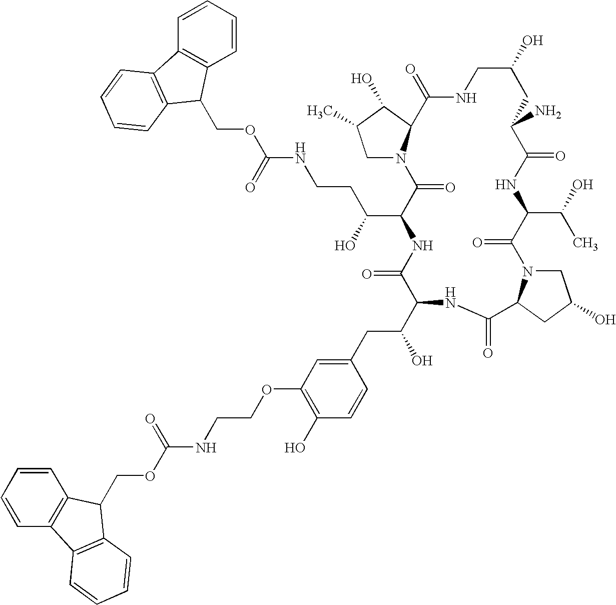 |
| | 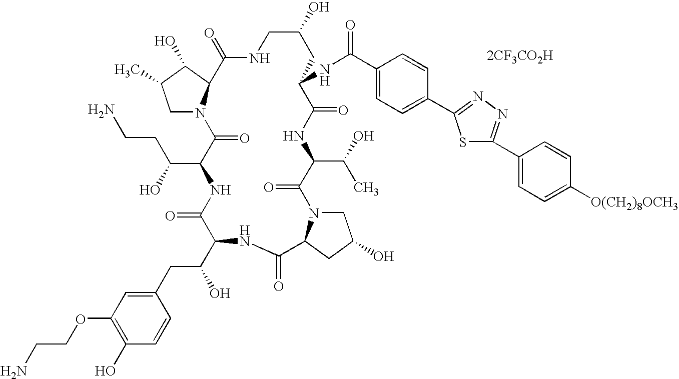 |
| 58 | 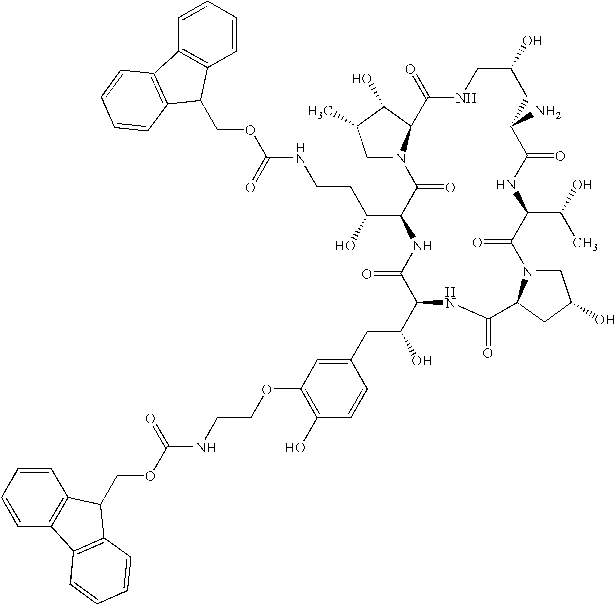 |
| | 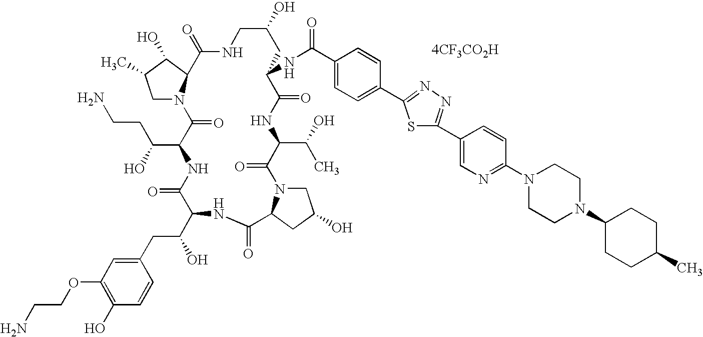 |
| 59 | 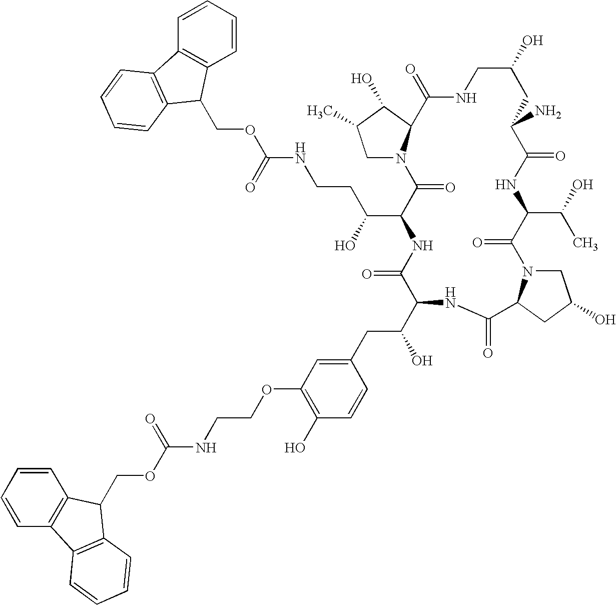 |
| | 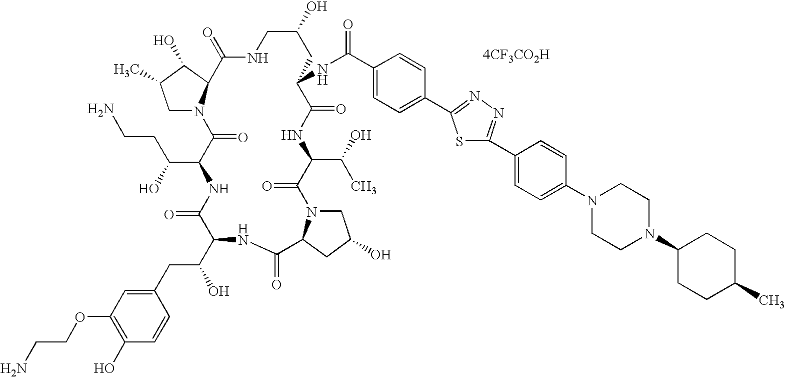 |
| 60 | 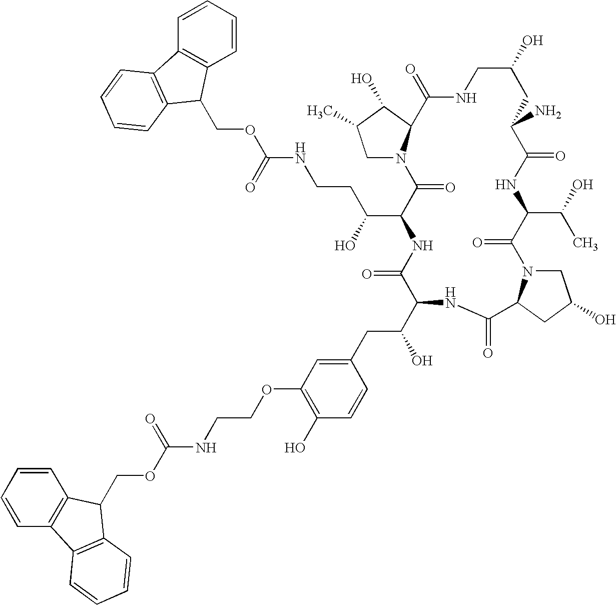 |
| | 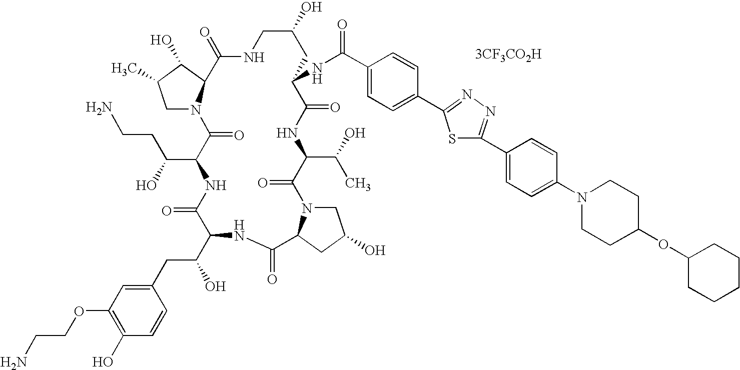 |
| 61 | 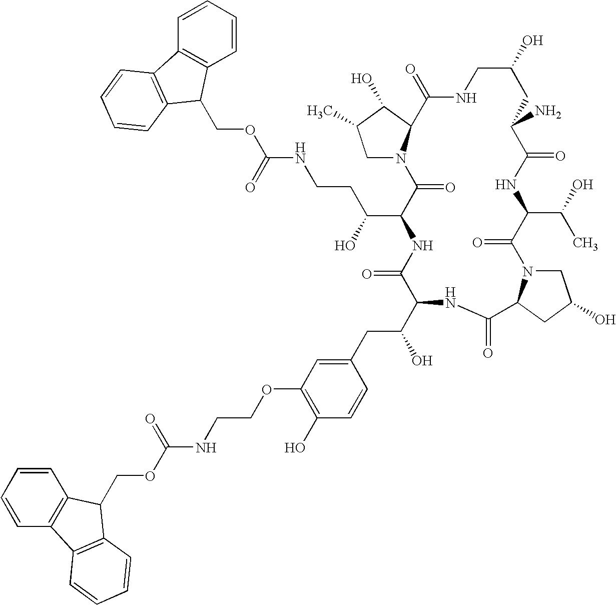 |
| | 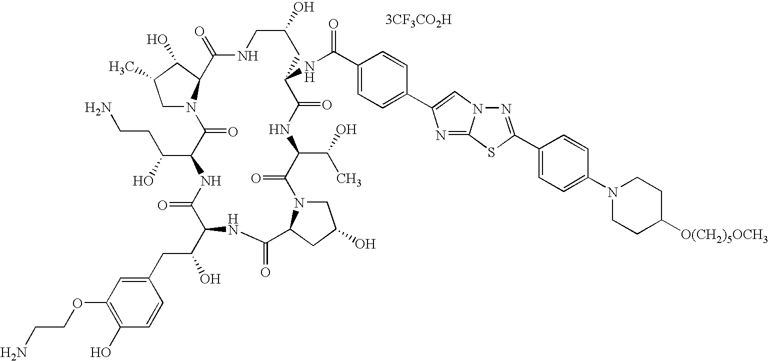 |
| 62 | 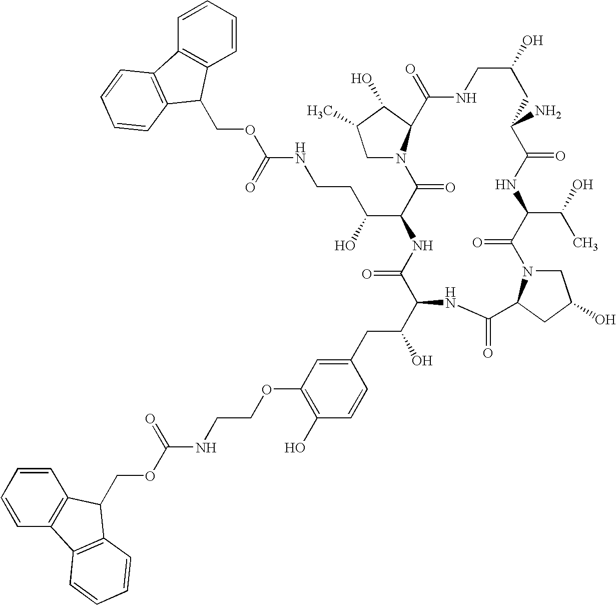 |
| | 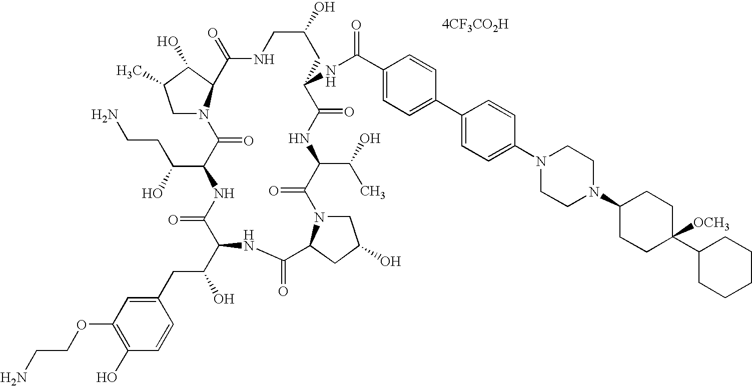 |
| 63 | 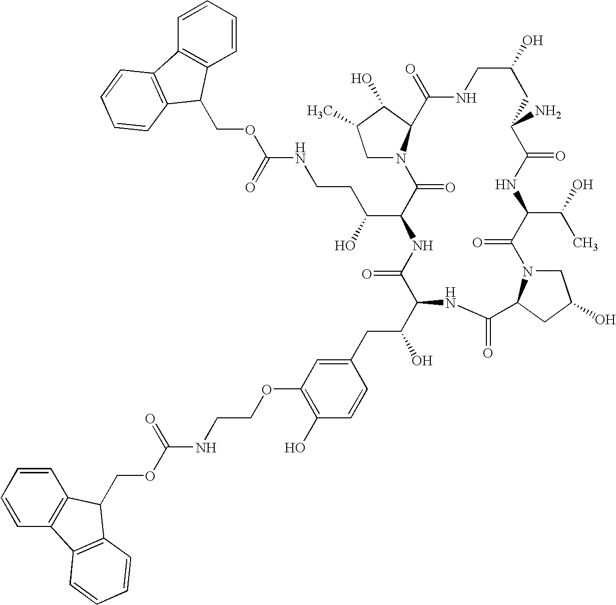 |
| | 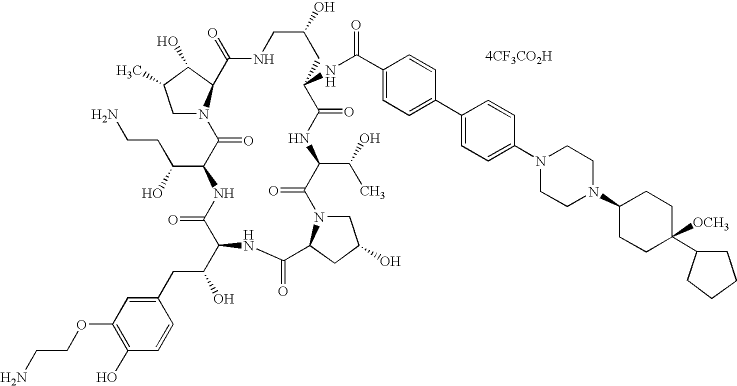 |
| 64 | 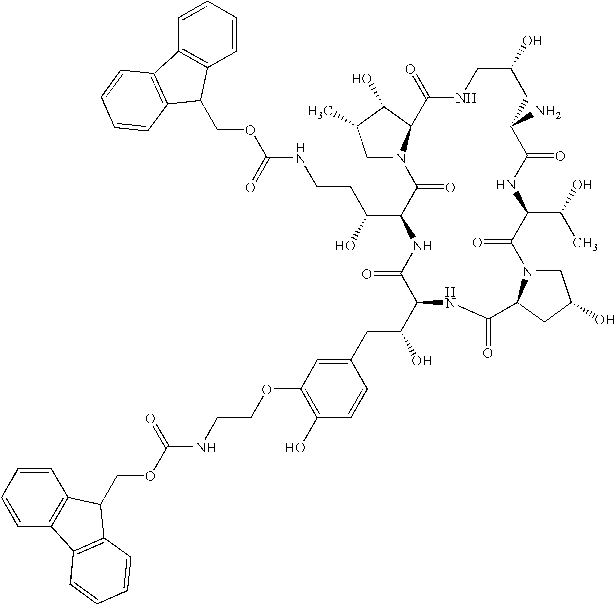 |
| | 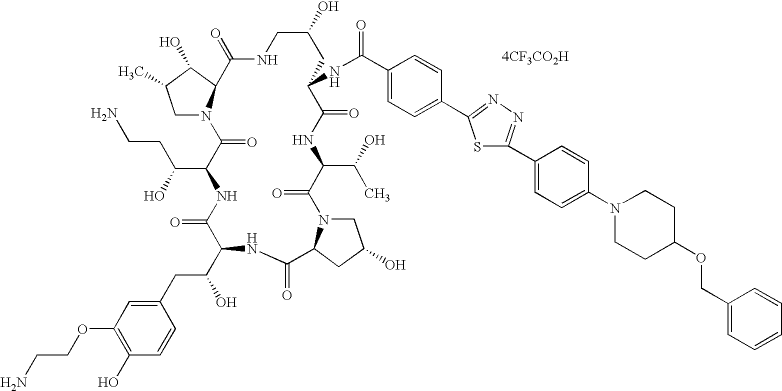 |
| 65 | 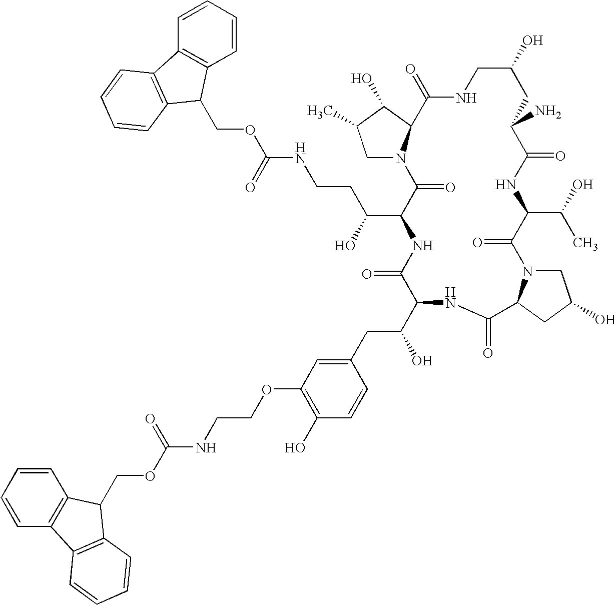 |
| | 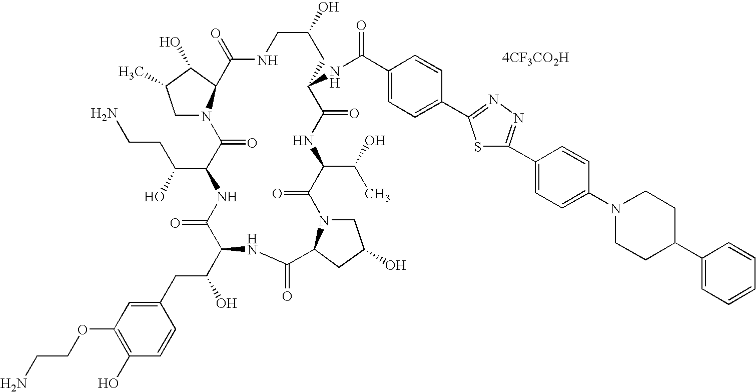 |
| 66 | 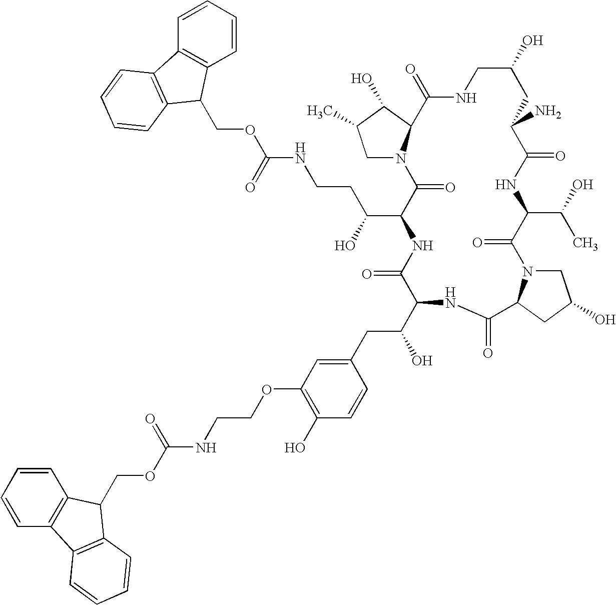 |
| | 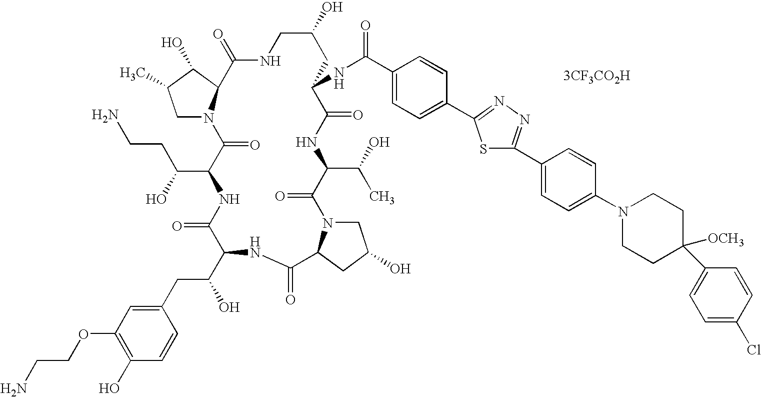 |
| 67 | 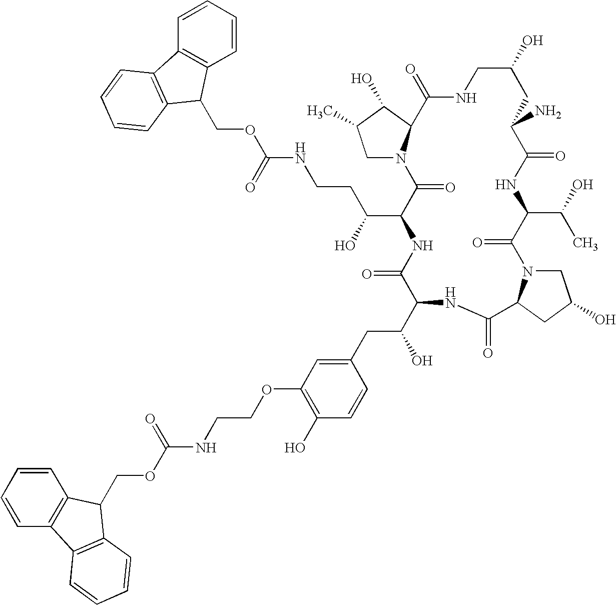 |
| | 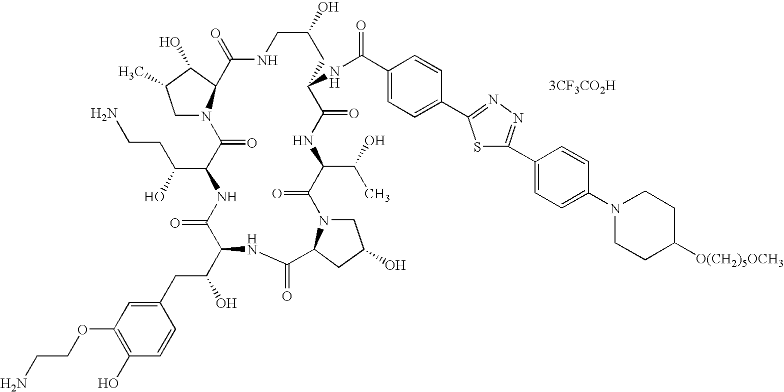 |
| 68 | 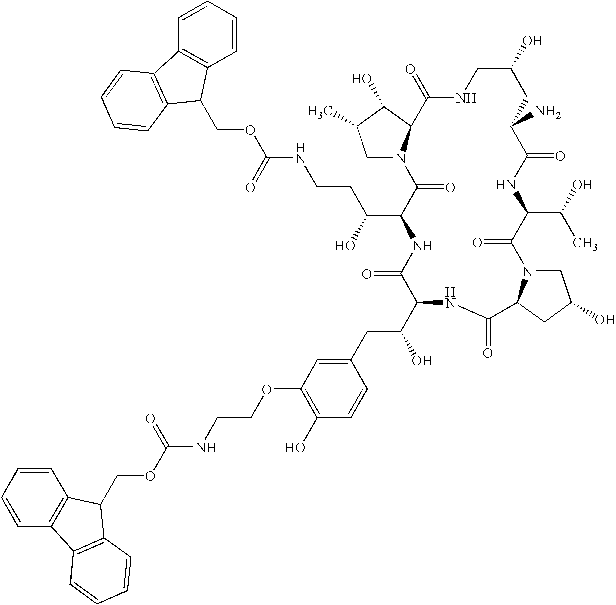 |
| | 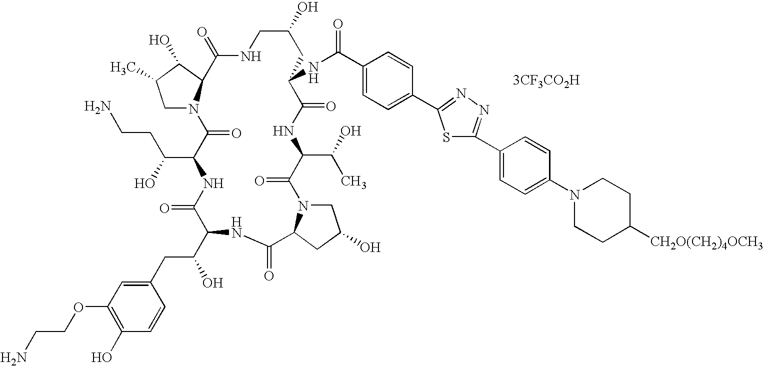 |
| 69 | 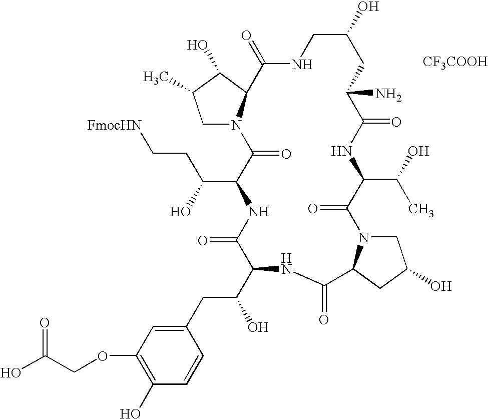 |
| | 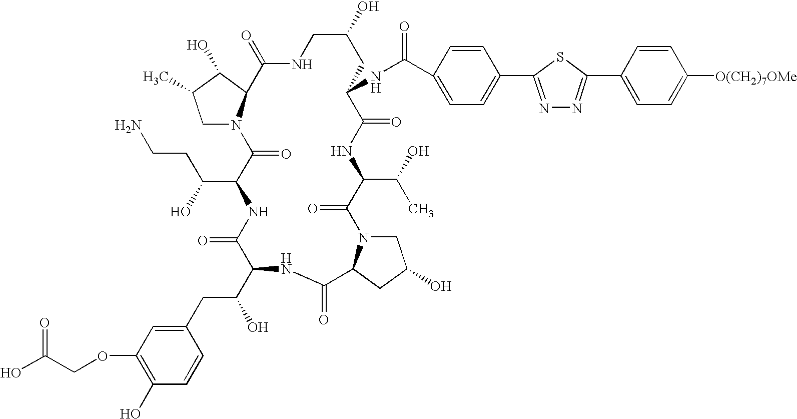 |
| 70 | 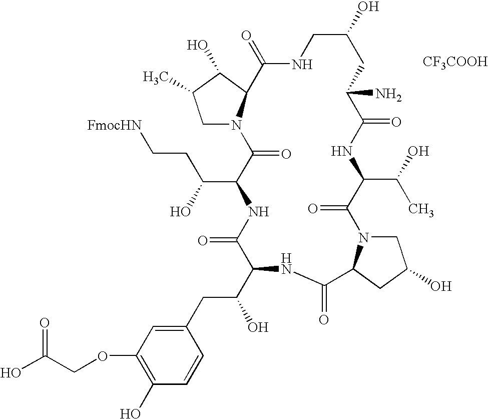 |
| | 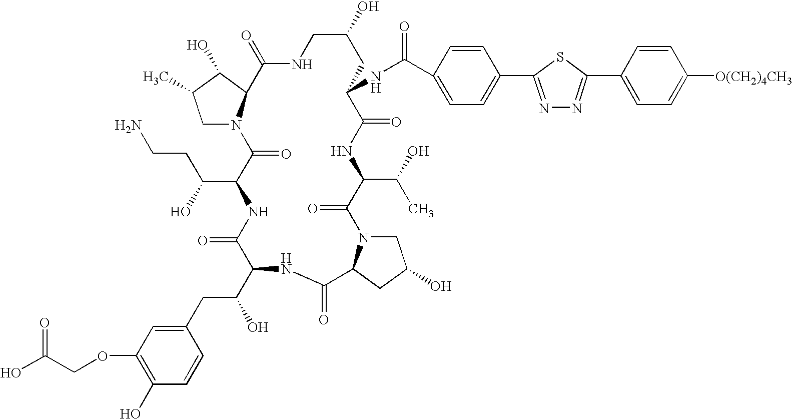 |
| 71 | 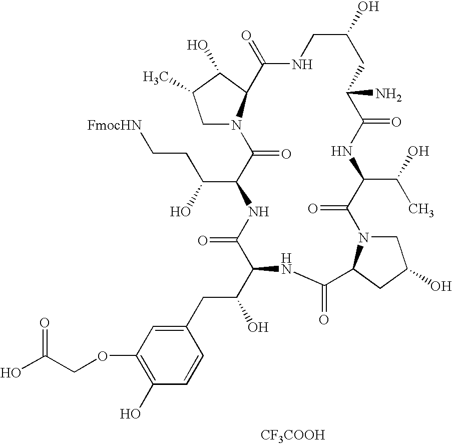 |
| | 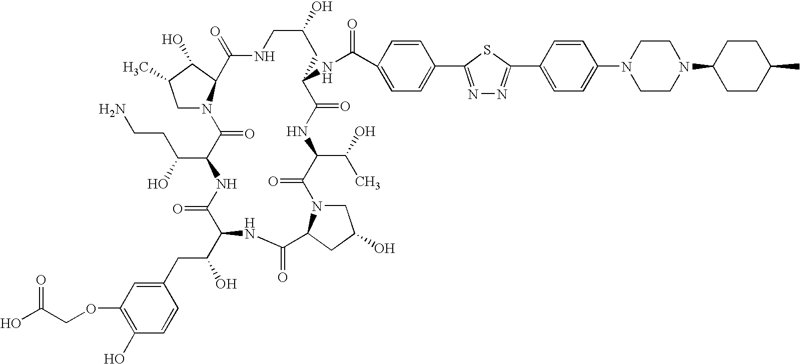 |
| 72 | 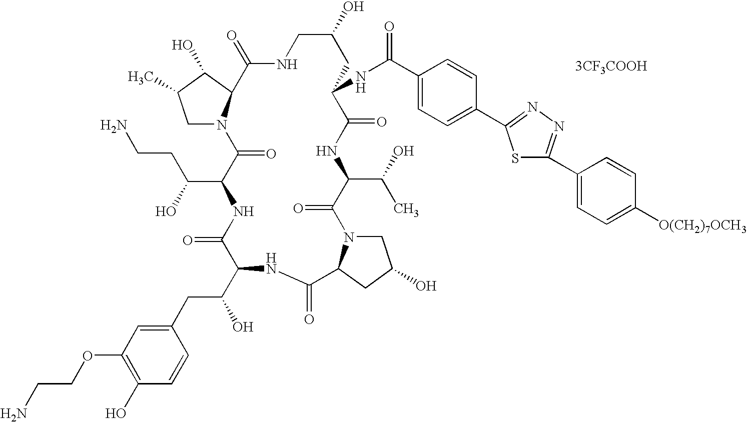 |
| | 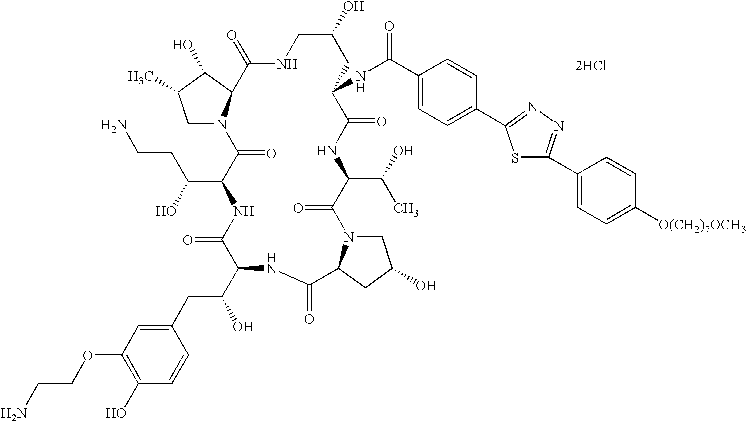 |
| 73 | 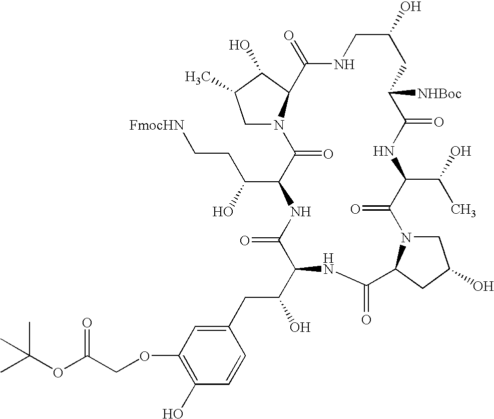 |
| | 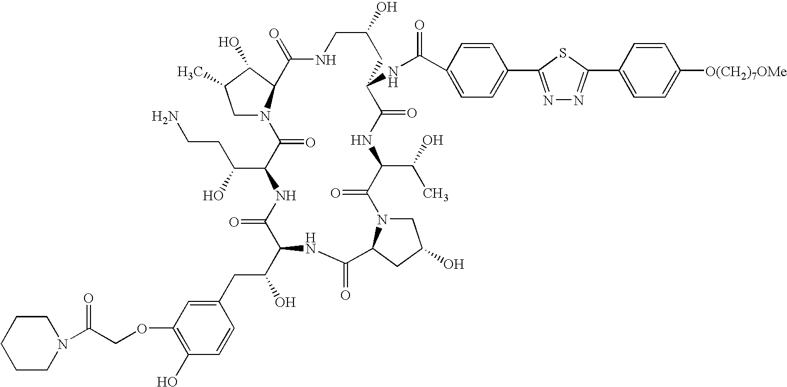 |
| 74 | 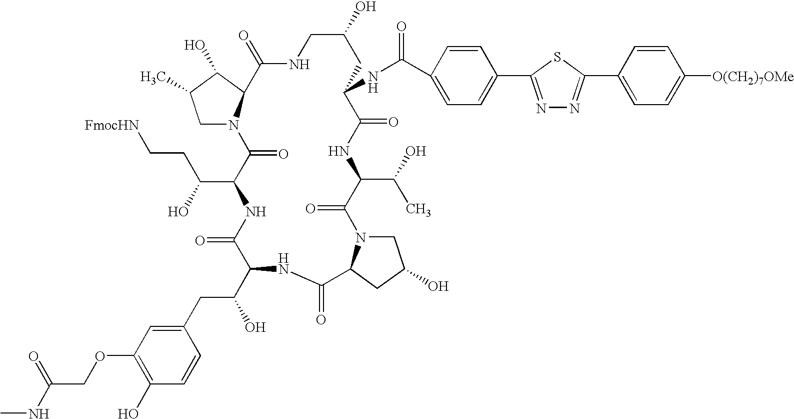 |
| | 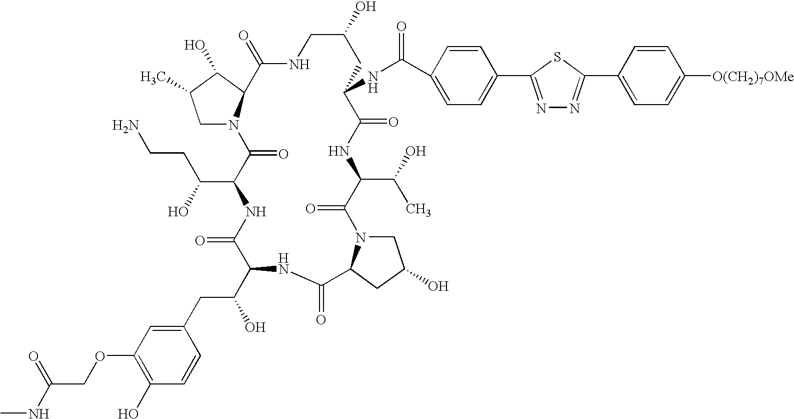 |
| 75 | 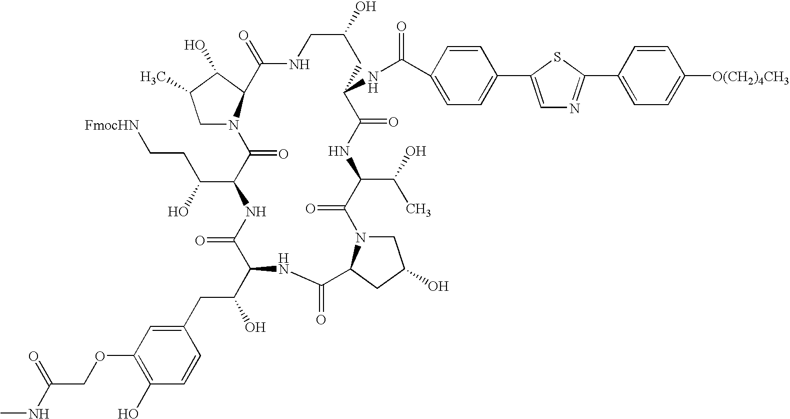 |
| | 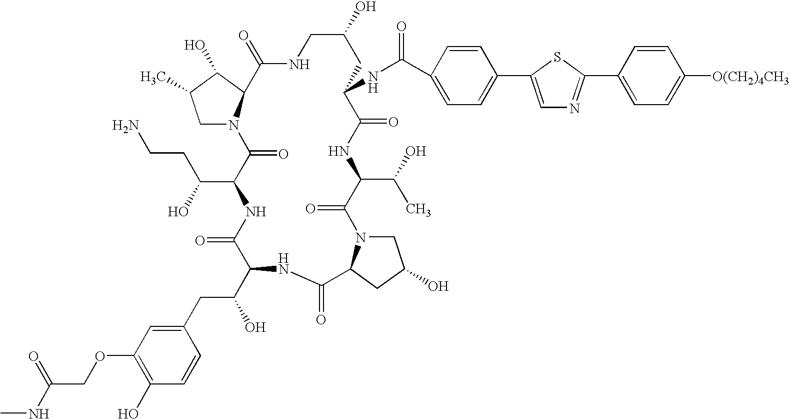 |
| 76 | 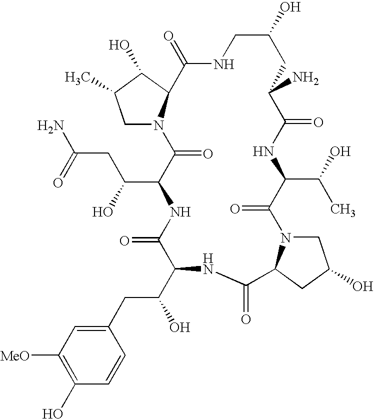 |
| | 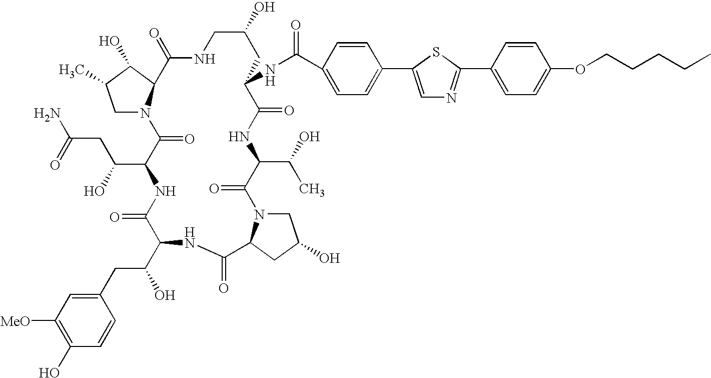 |
| 77 | 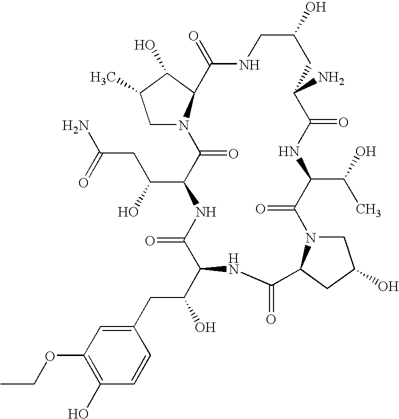 |
| | 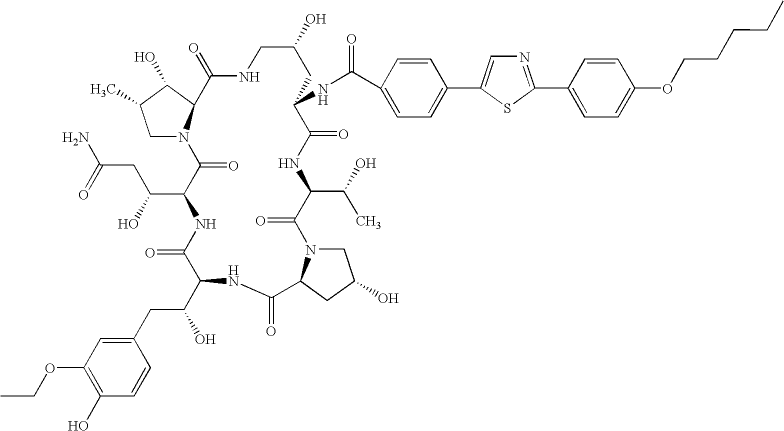 |
| 78 | 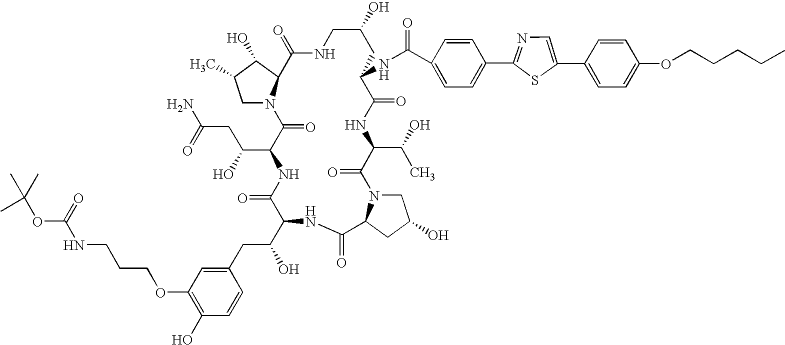 |
| | 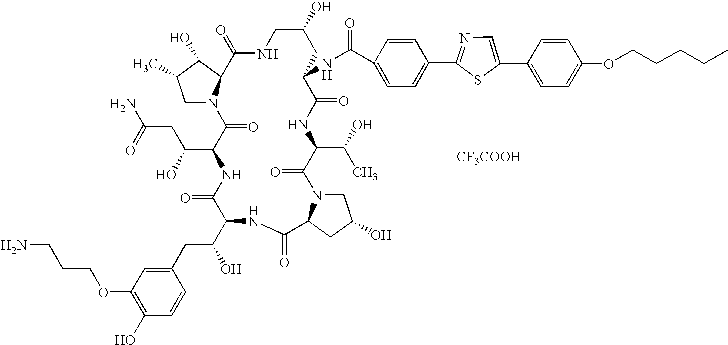 |
| 79 | 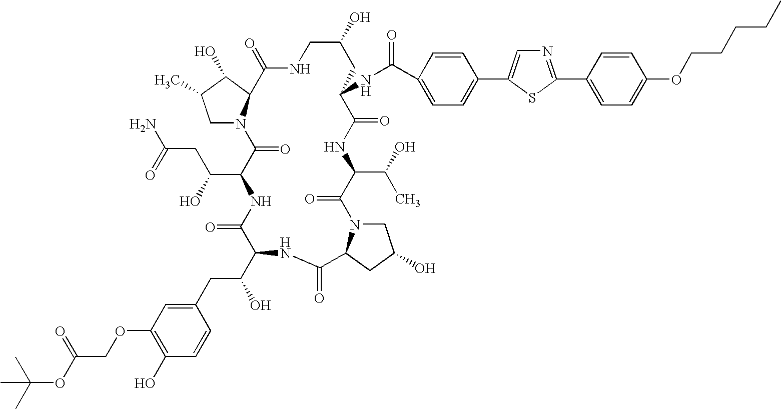 |
| | 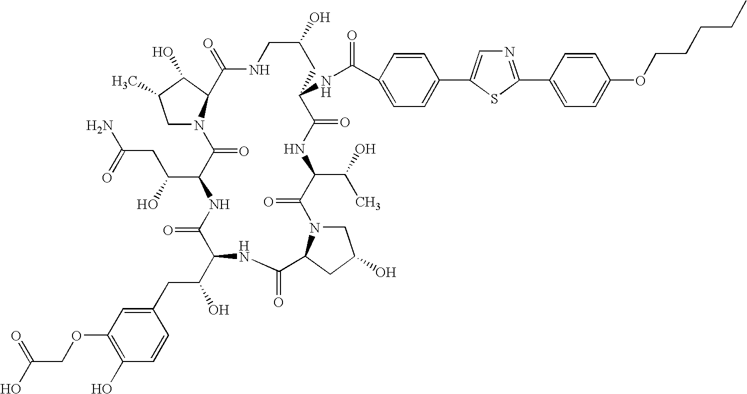 |
| 80 | 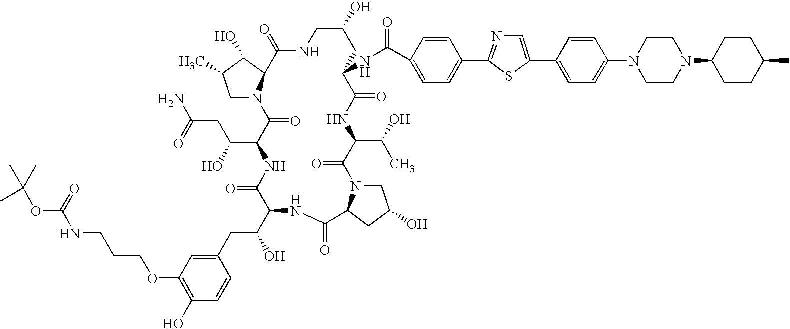 |
| | 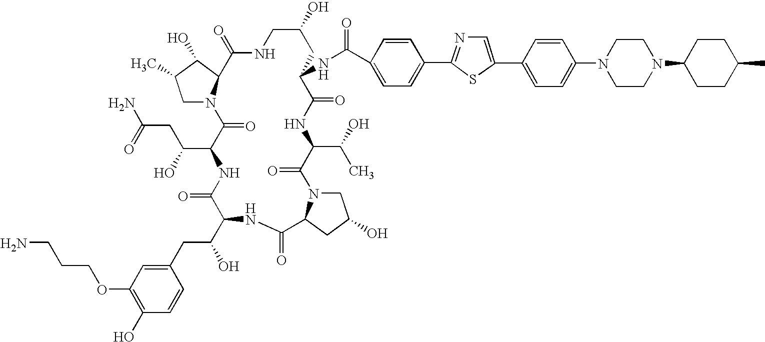 |
| 81 | 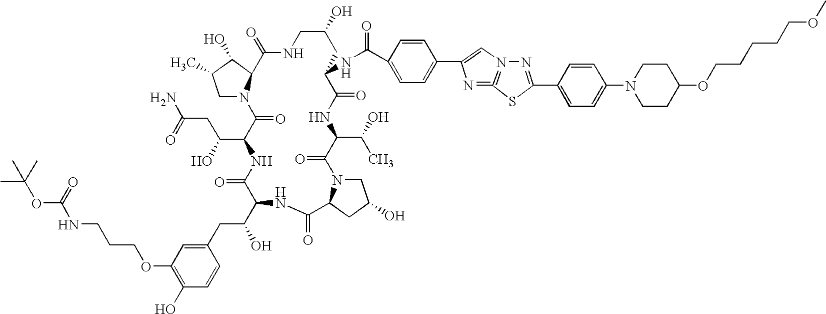 |
| | 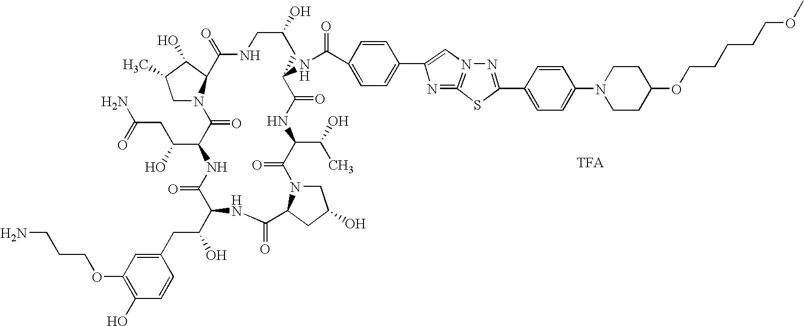 |
| 82 | 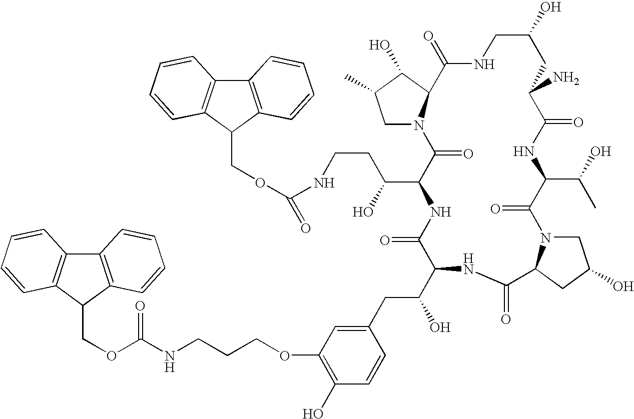 |
| | 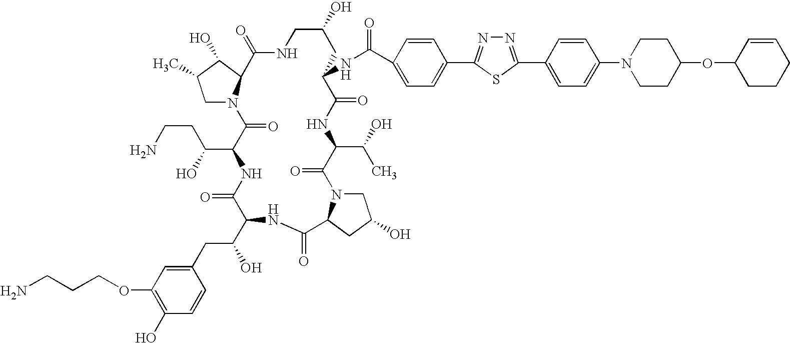 |
| 83 | 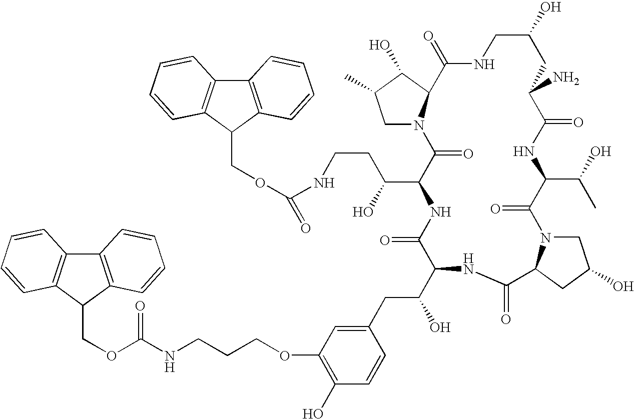 |
| | 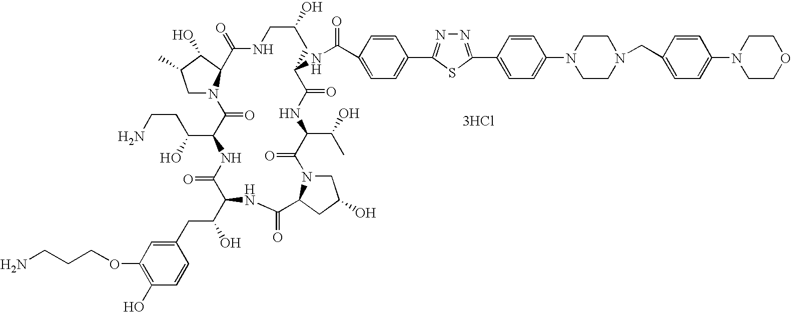 |
| 84 | 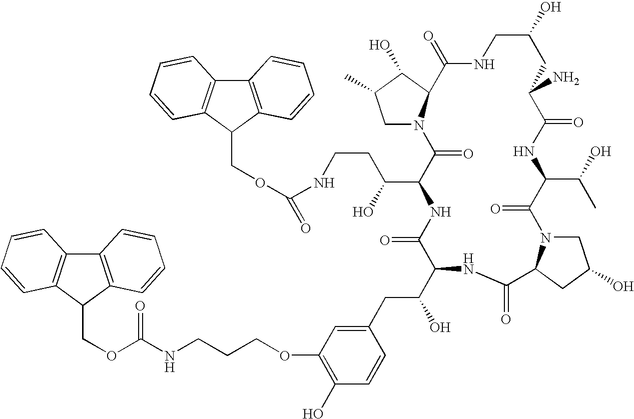 |
| | 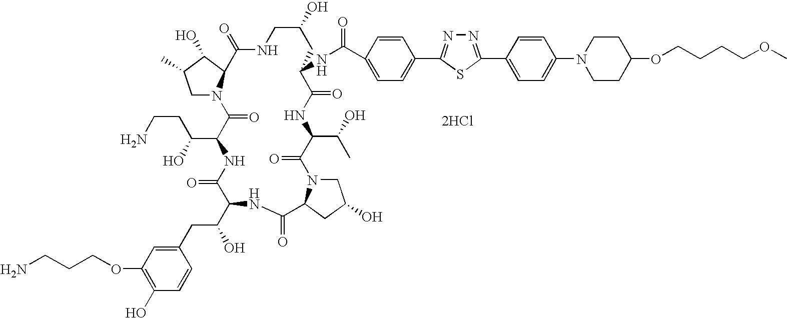 |
| 85 | 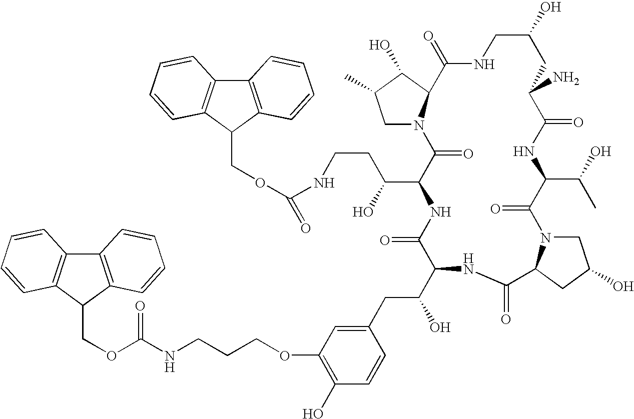 |
| | 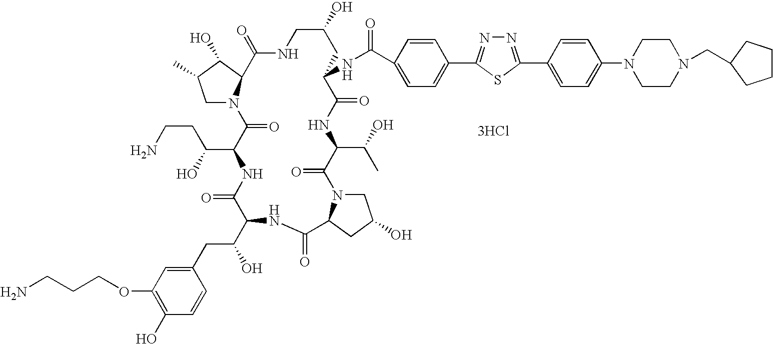 |
| 86 | 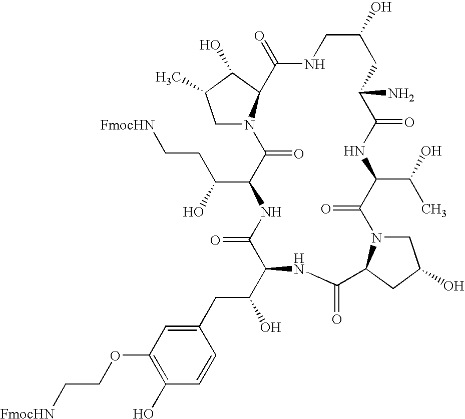 |
| | 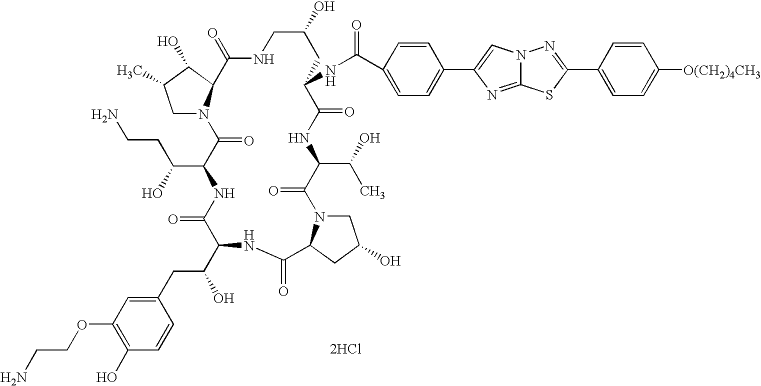 |
| 87 | 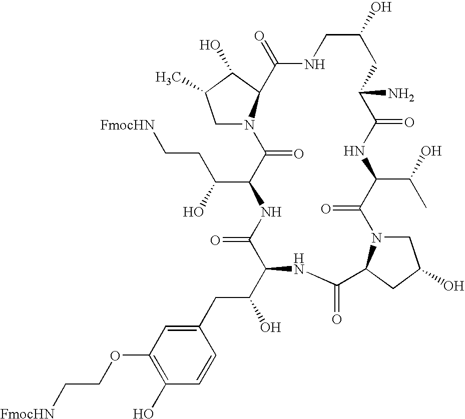 |
| | 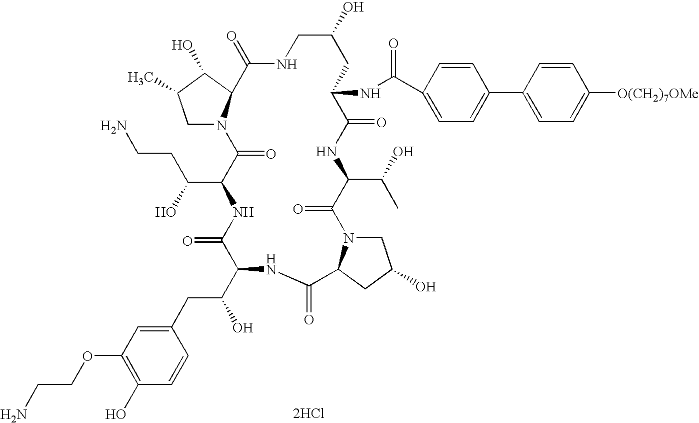 |
| 88 | 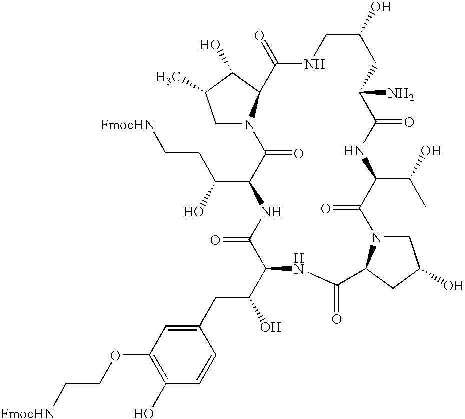 |
| | 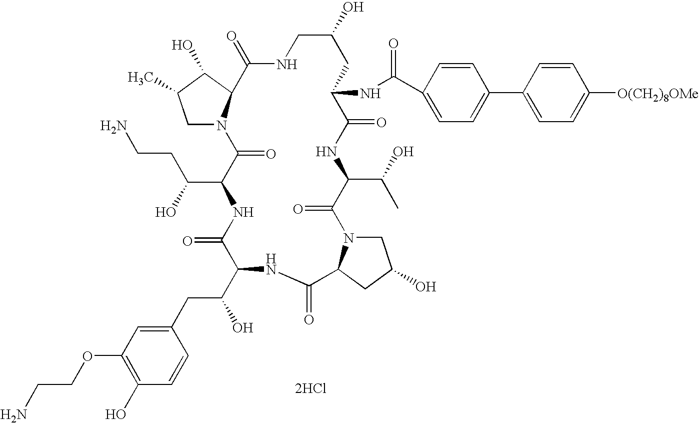 |
| 89 | 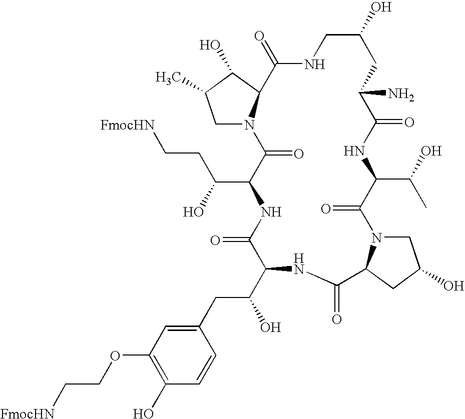 |
| | 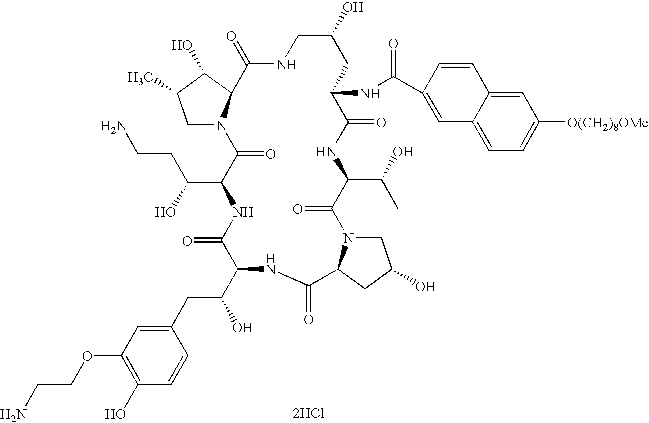 |
| 90 | 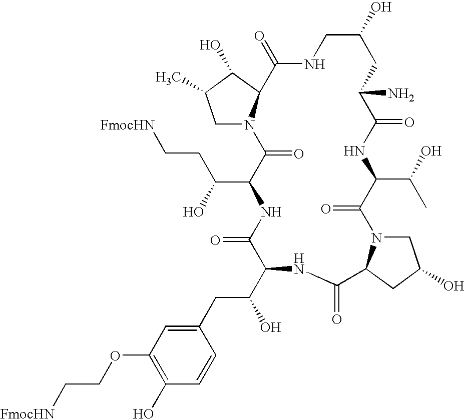 |
| | 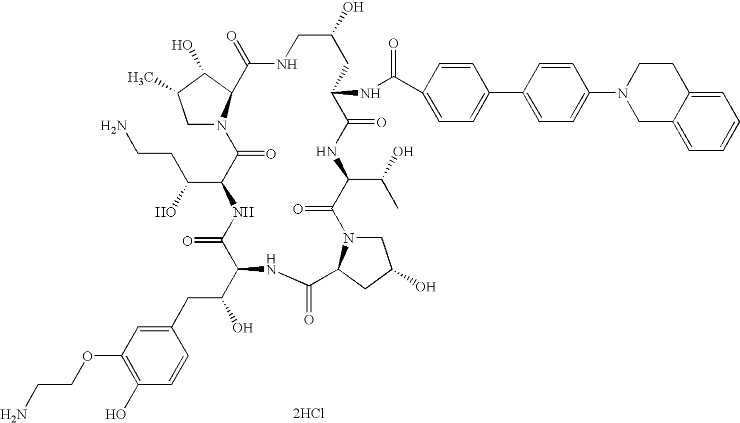 |
| 91 | 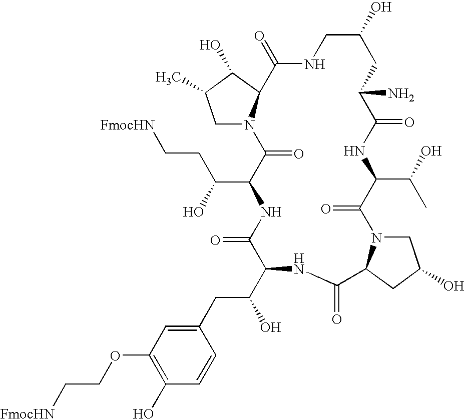 |
| | 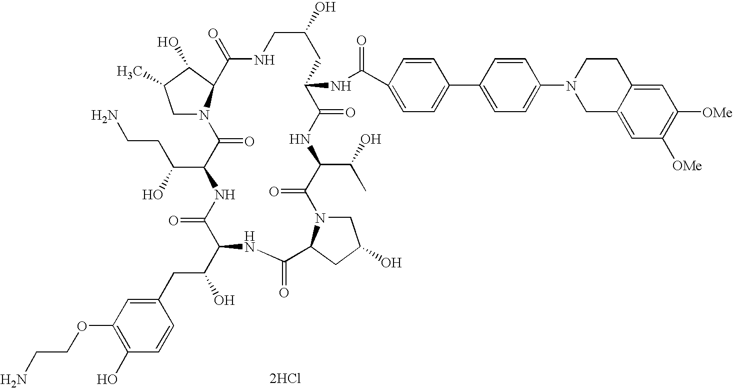 |
| 92 | 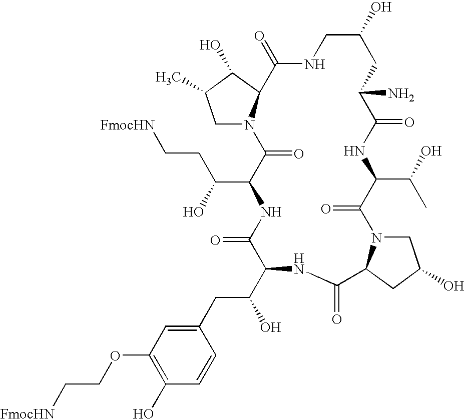 |
| | 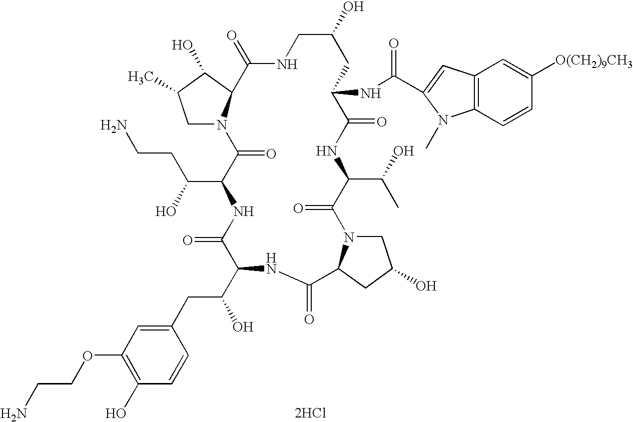 |
| 93 | 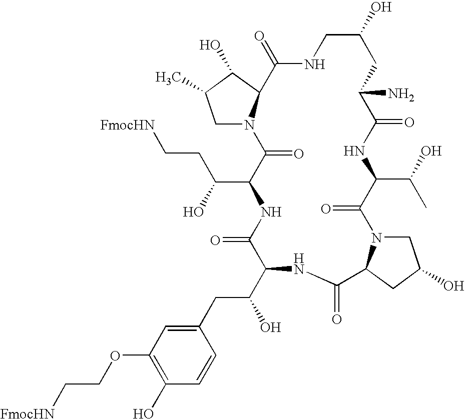 |
| | 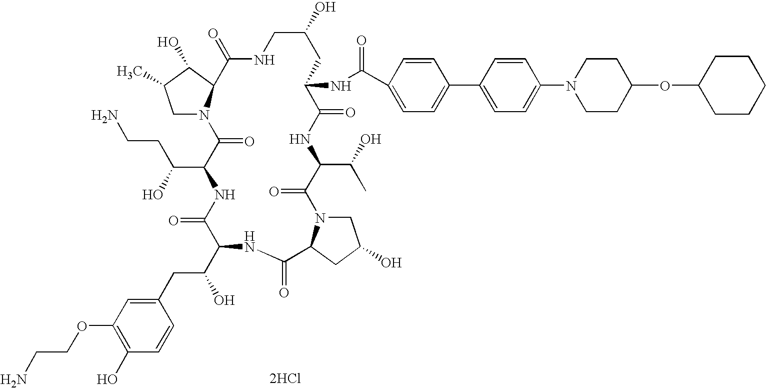 |
| 94 | 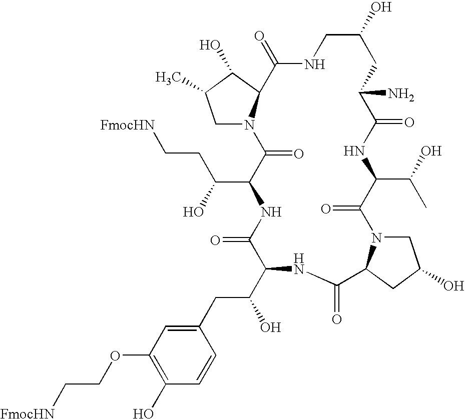 |
| | 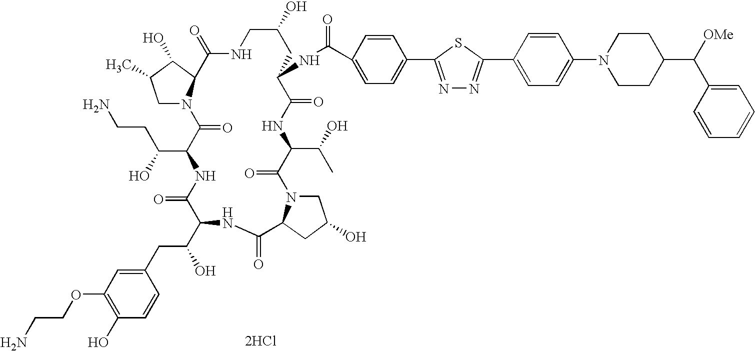 |
| 95 | 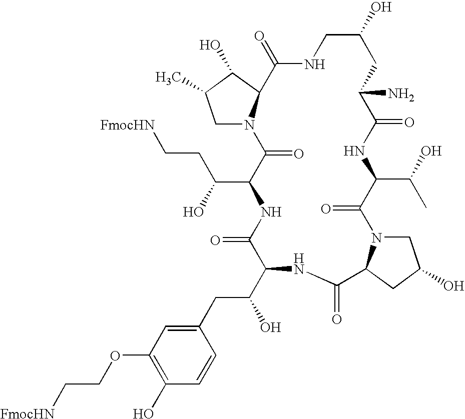 |
| | 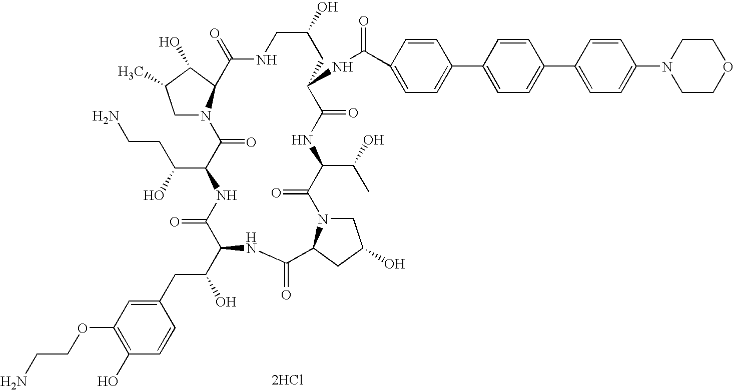 |
| 96 | 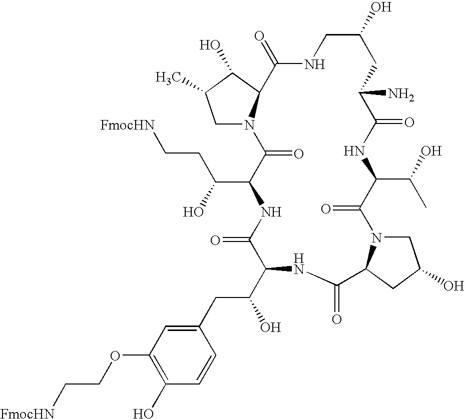 |
| | 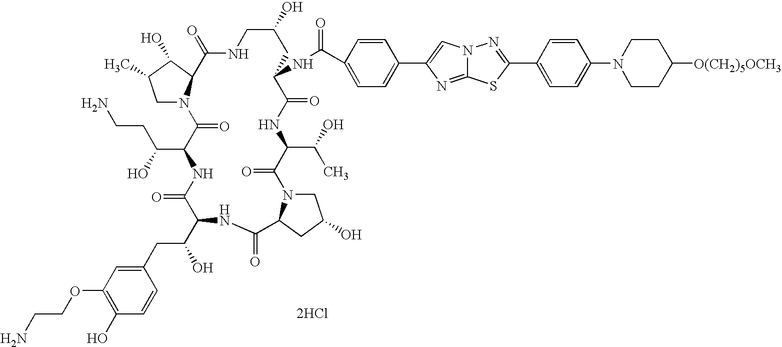 |
| 97 | 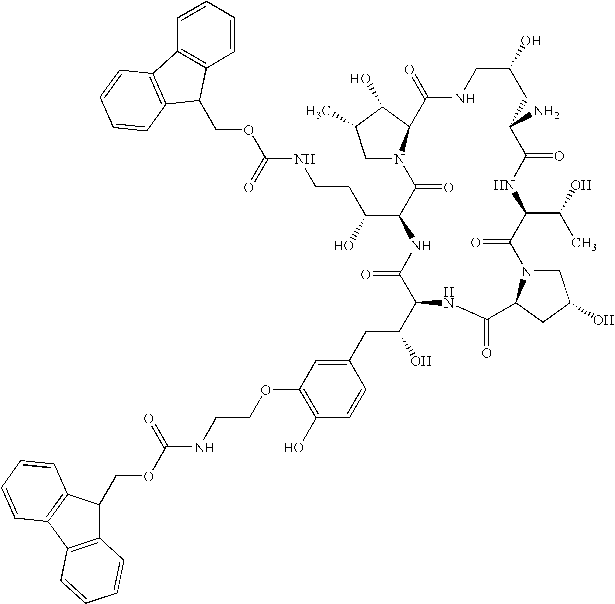 |
| | 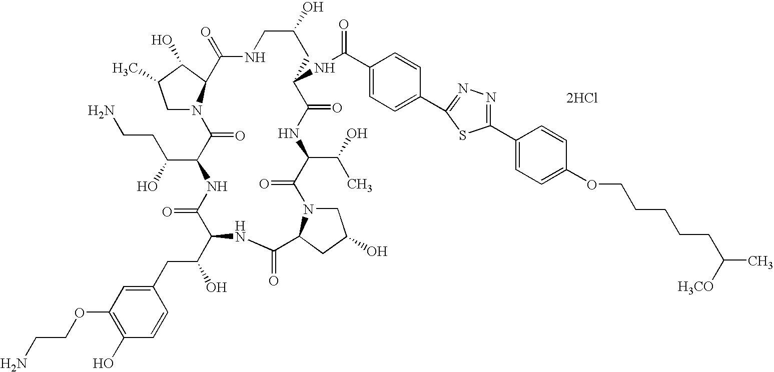 |
| 98 | 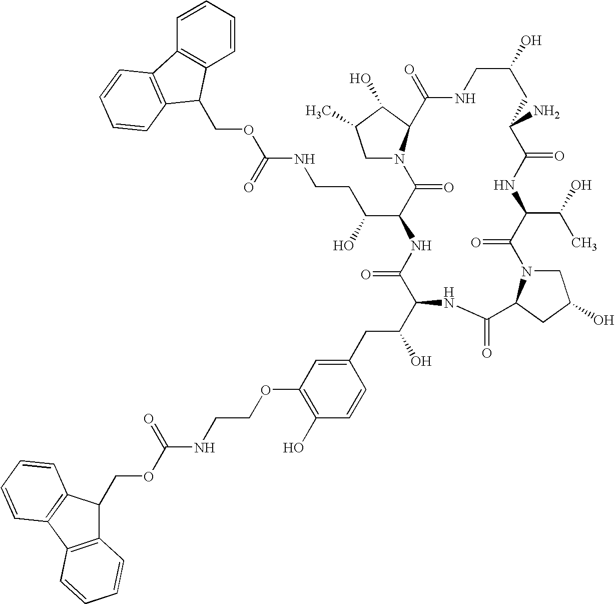 |
| | 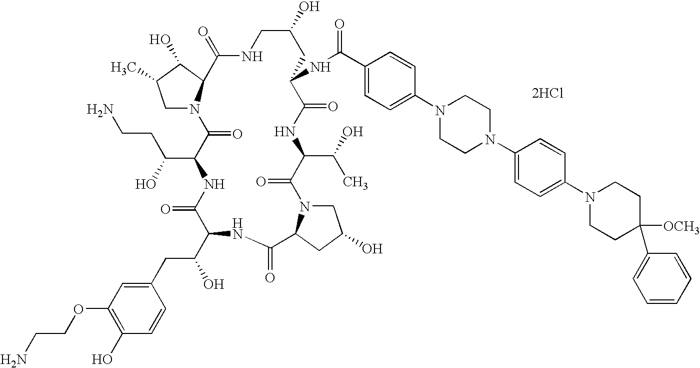 |
| 99 | 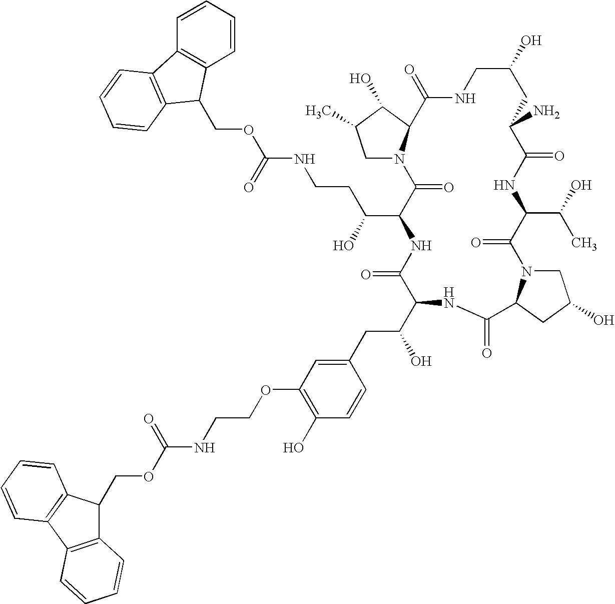 |
| | 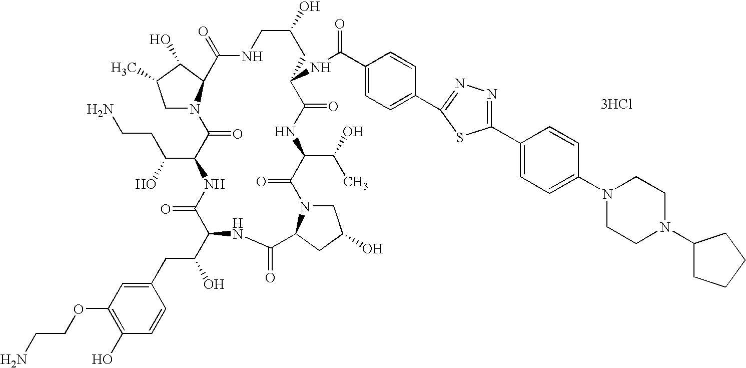 |
| 100 | 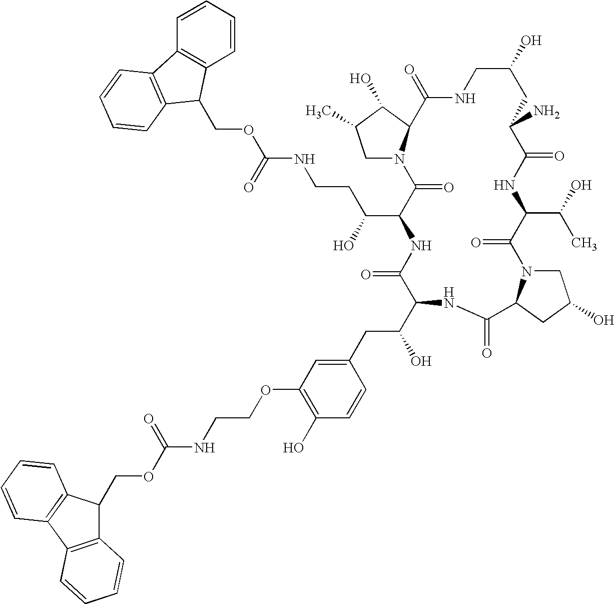 |
| | 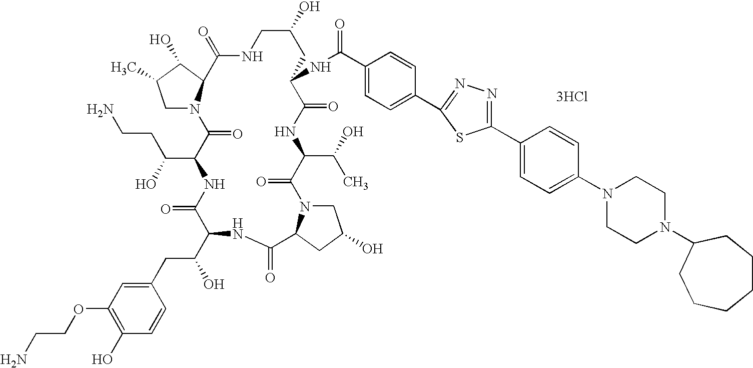 |
| 101 | 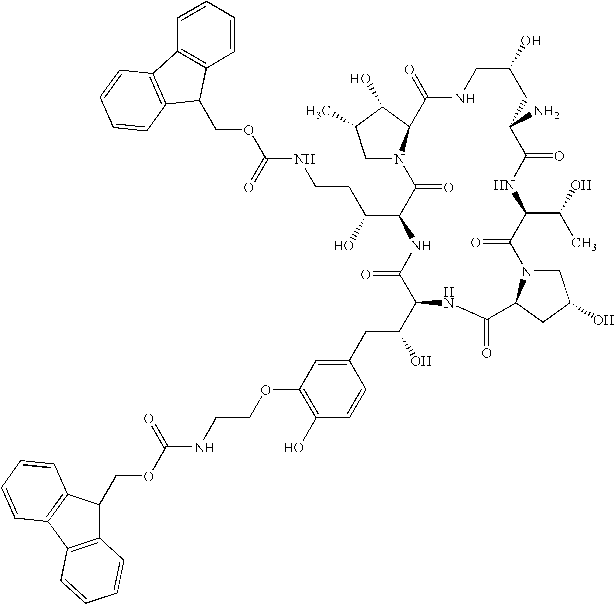 |
| | 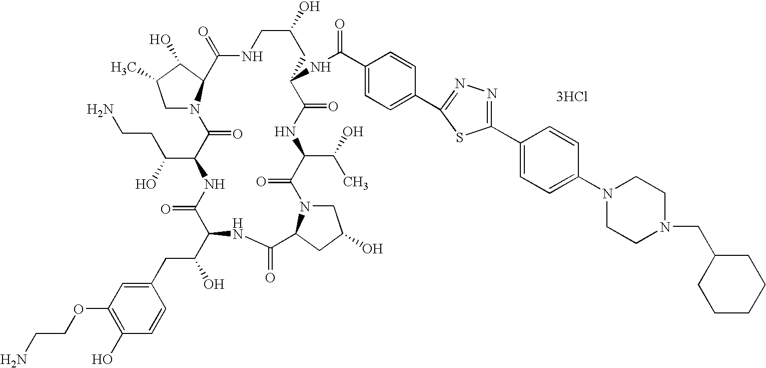 |
| 102 | 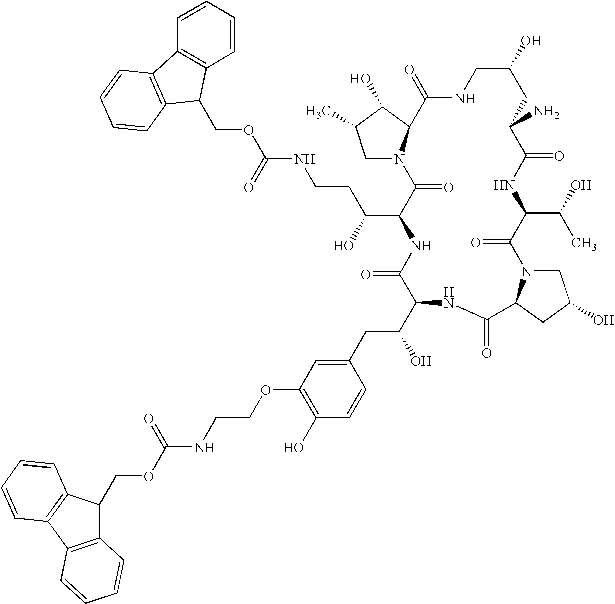 |
| | 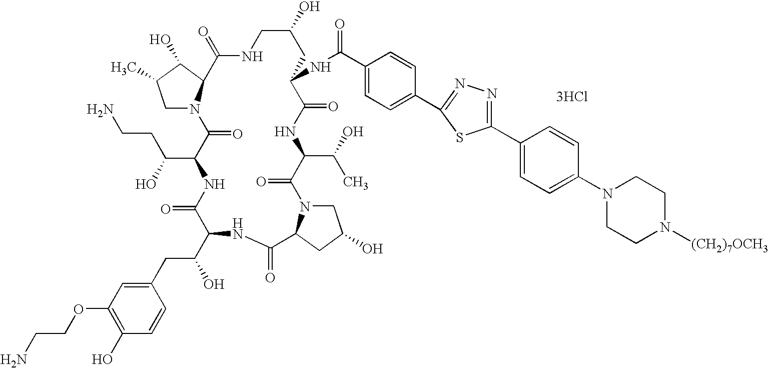 |
| 103 | 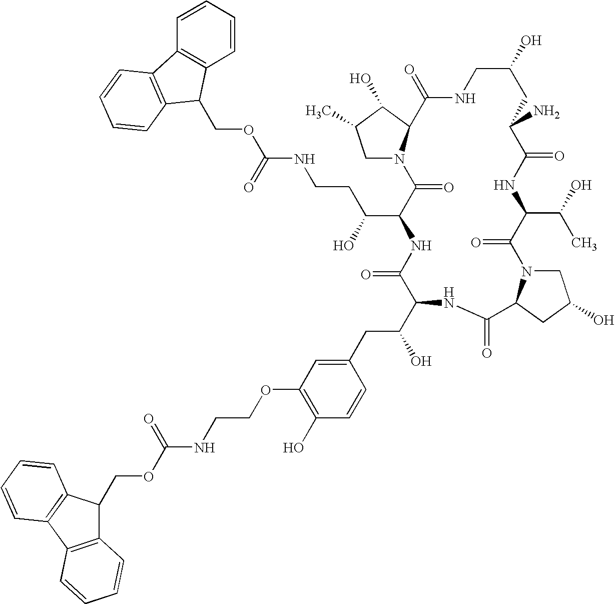 |
| | 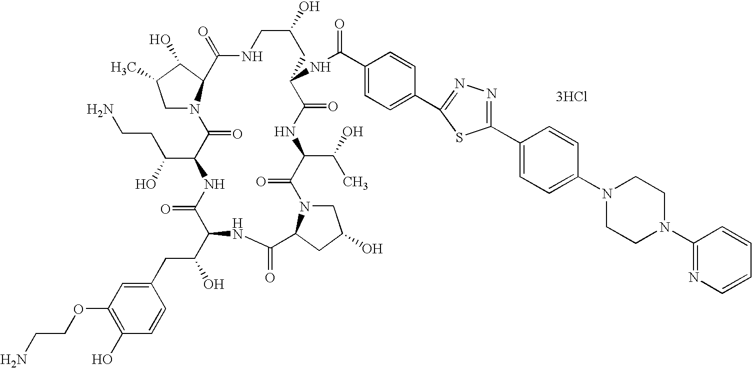 |
| 104 | 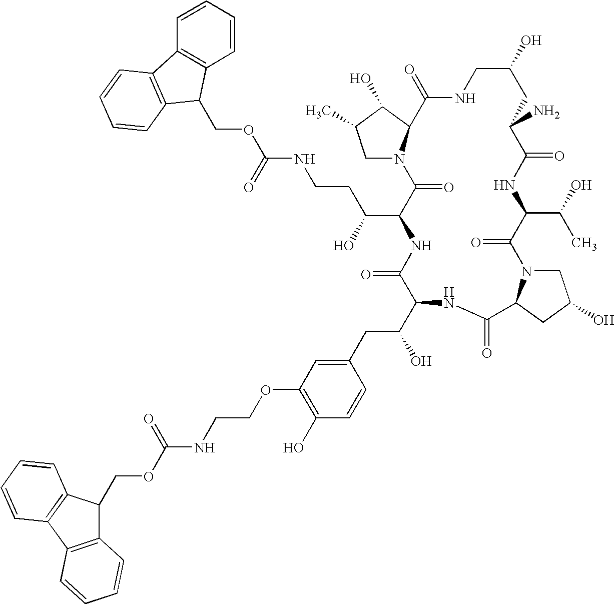 |
| | 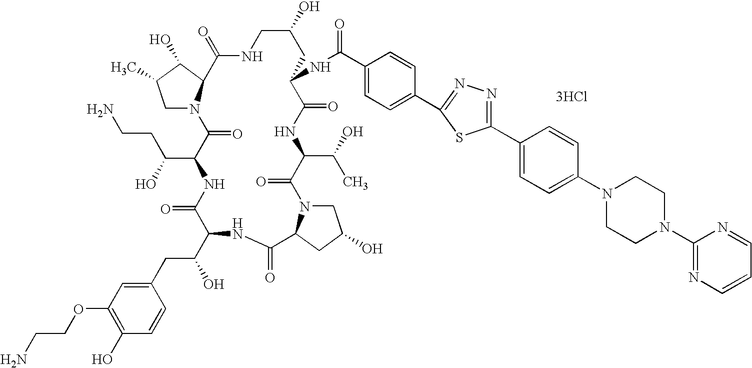 |
| 105 | 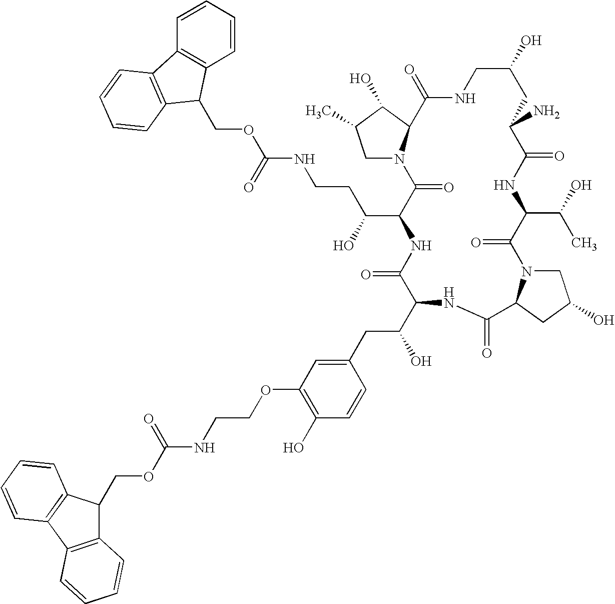 |
| | 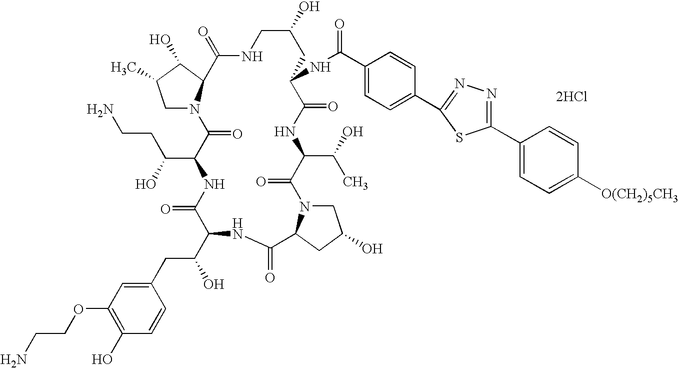 |
| 106 | 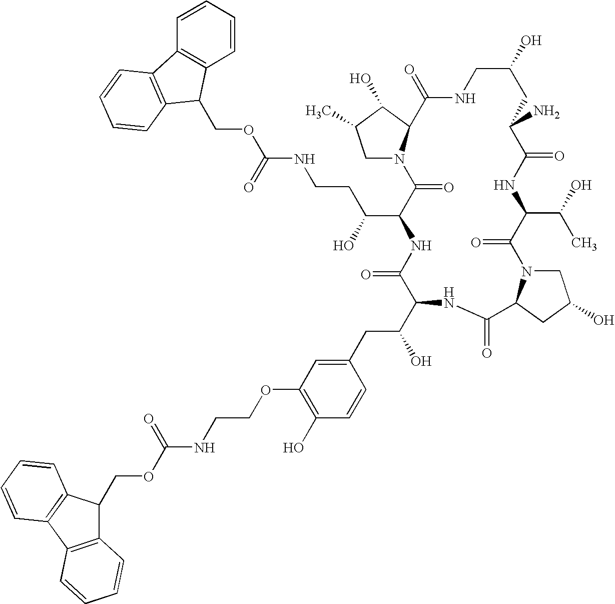 |
| | 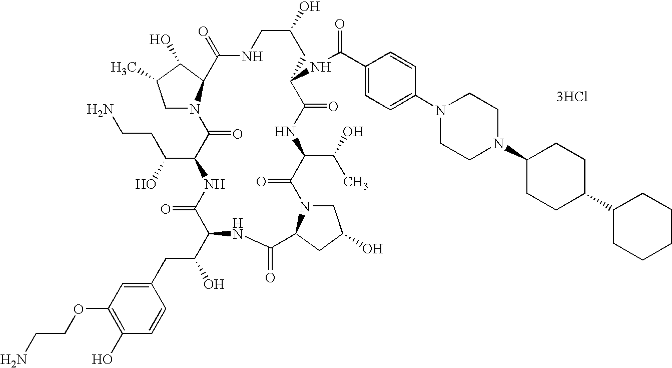 |
| 107 | 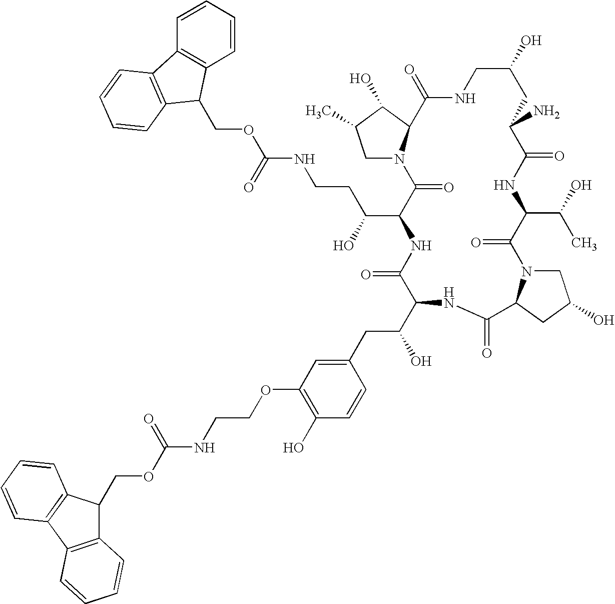 |
| | 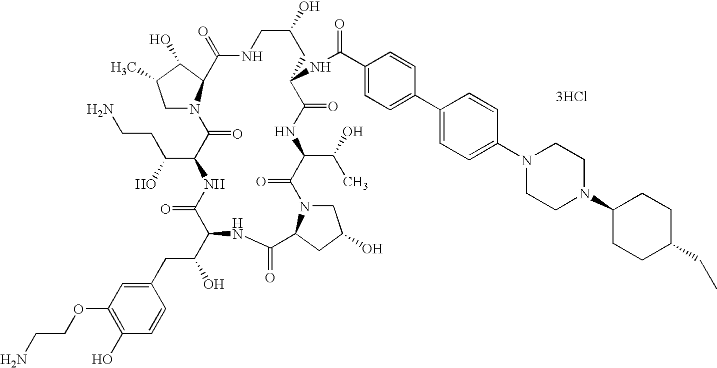 |
| 108 | 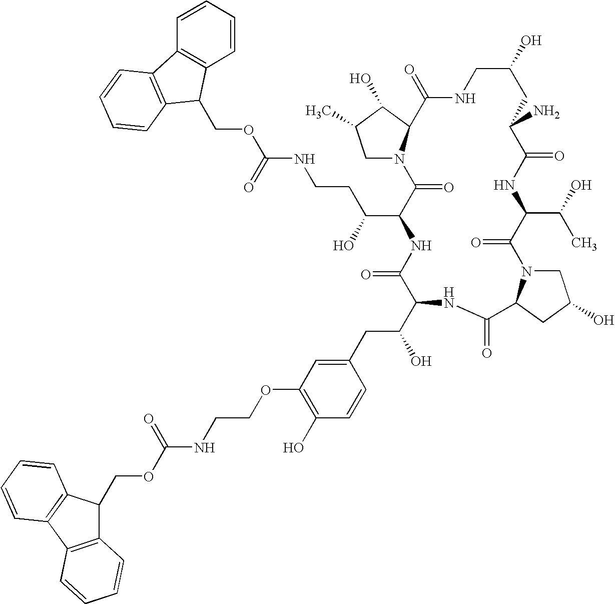 |
| | 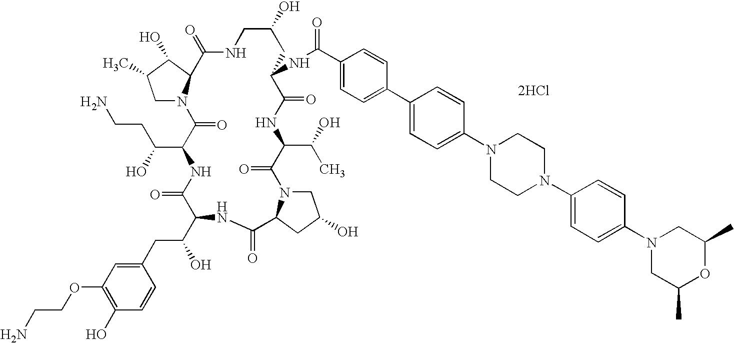 |
| 109 | 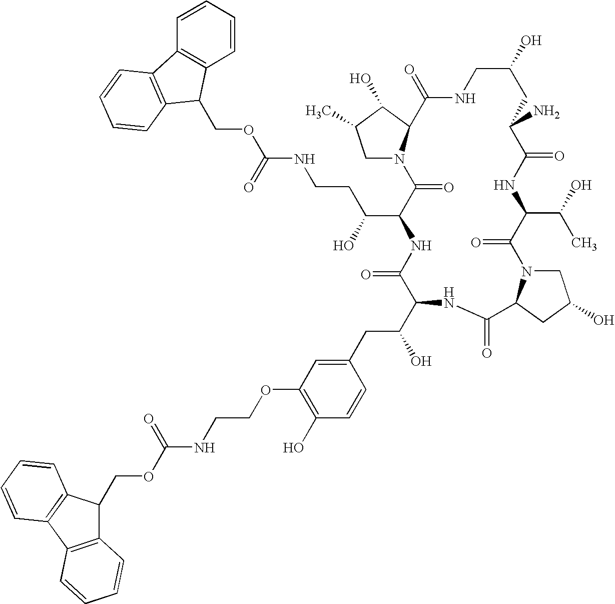 |
| | 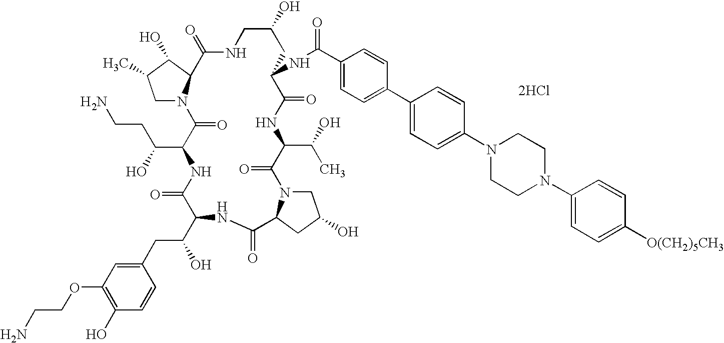 |
| 110 | 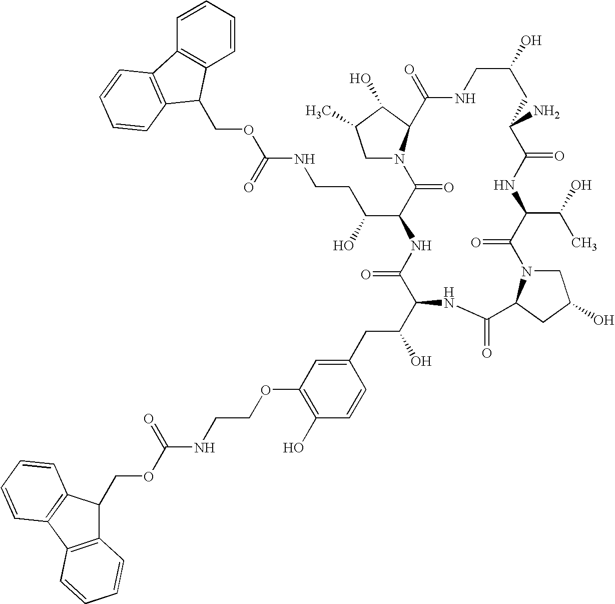 |
| | 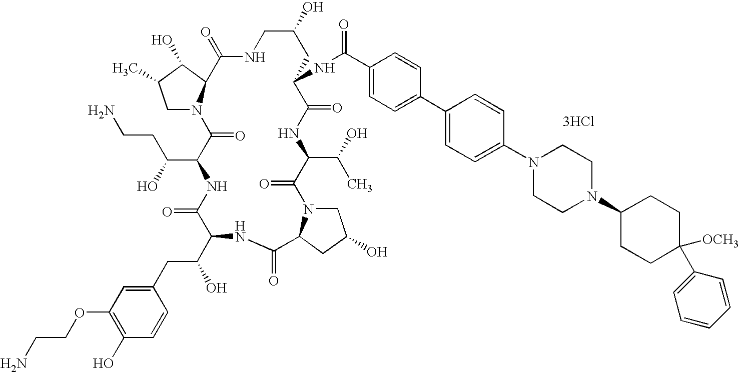 |
| 111 | 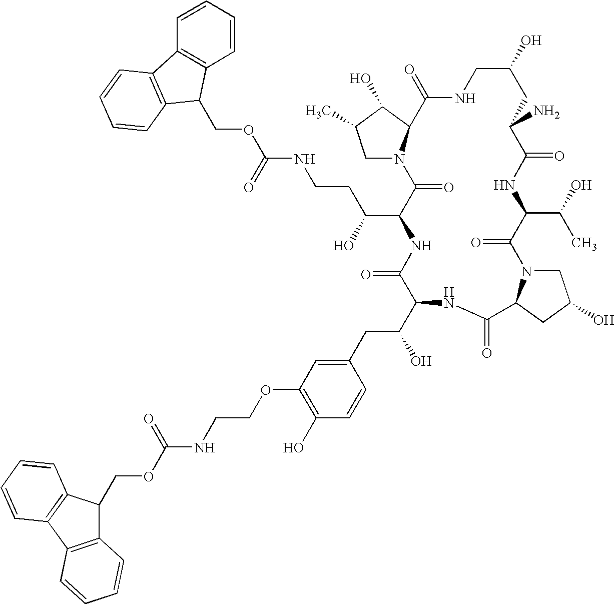 |
| | 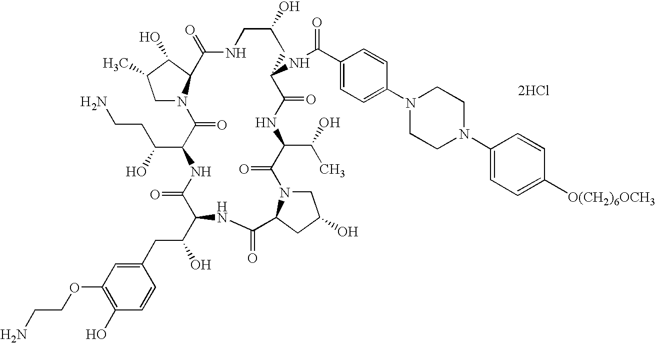 |
| 112 | 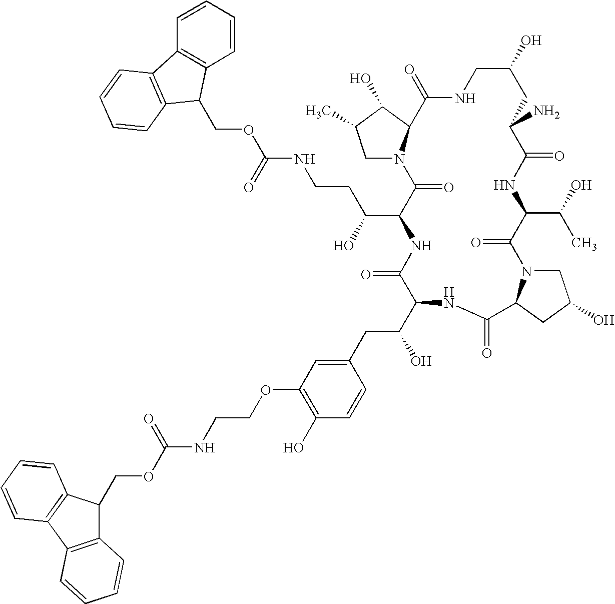 |
| | 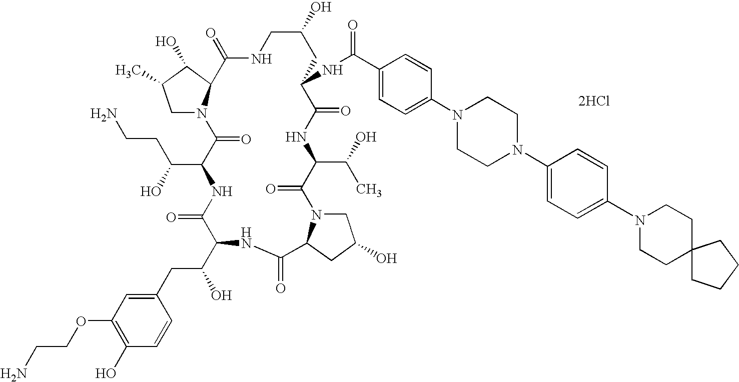 |
| 113 | 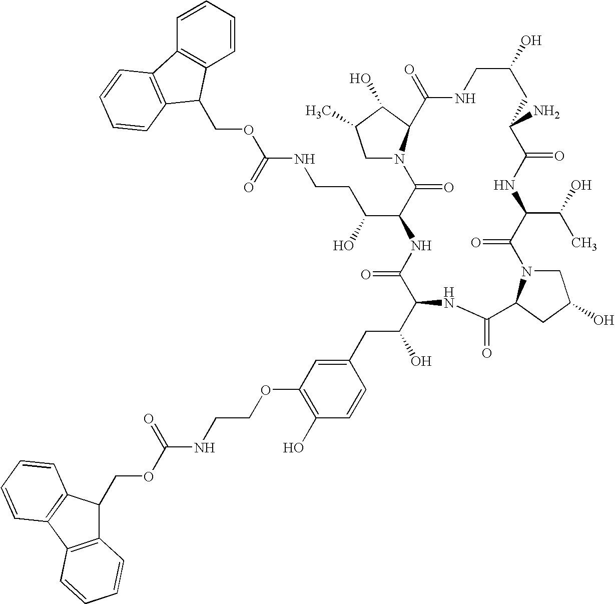 |
| | 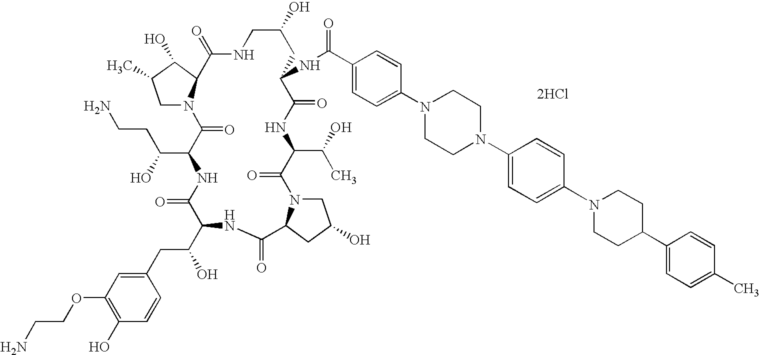 |
| 114 | 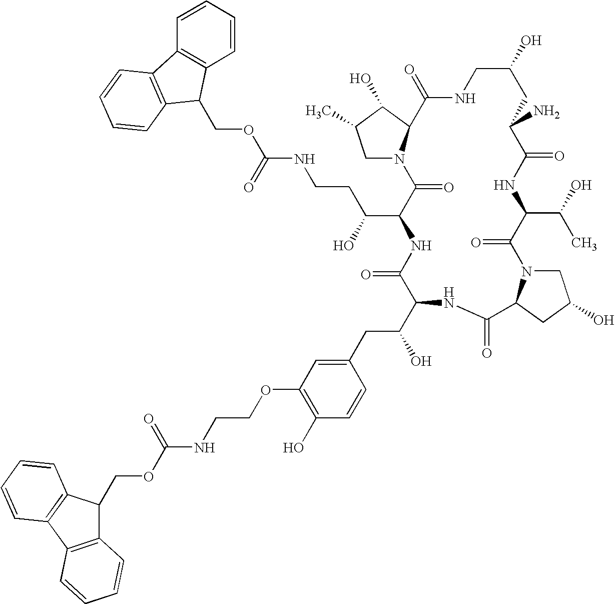 |
| | 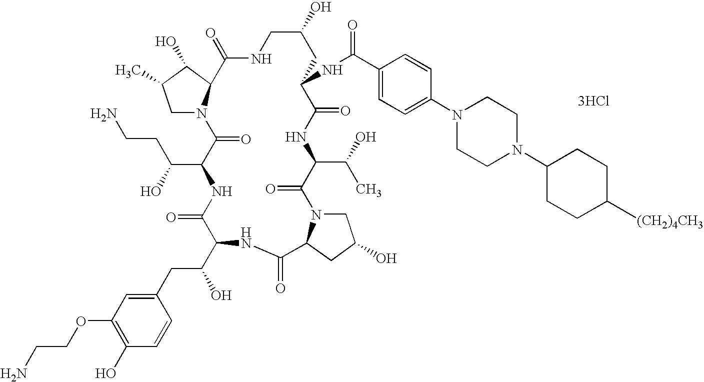 |
| 115 | 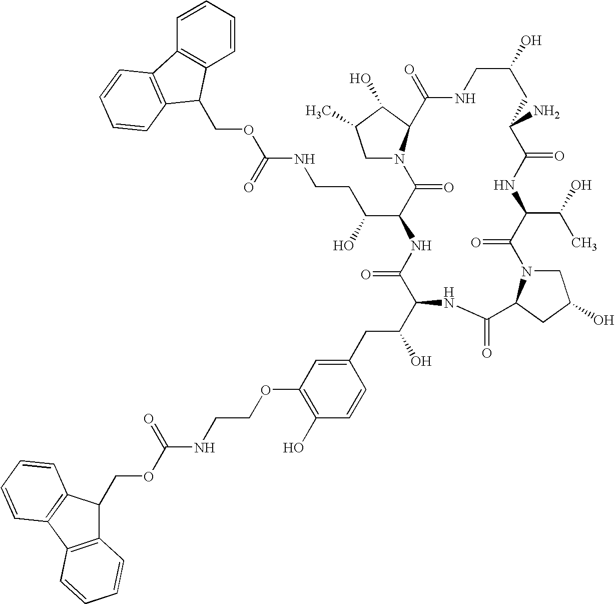 |
| | 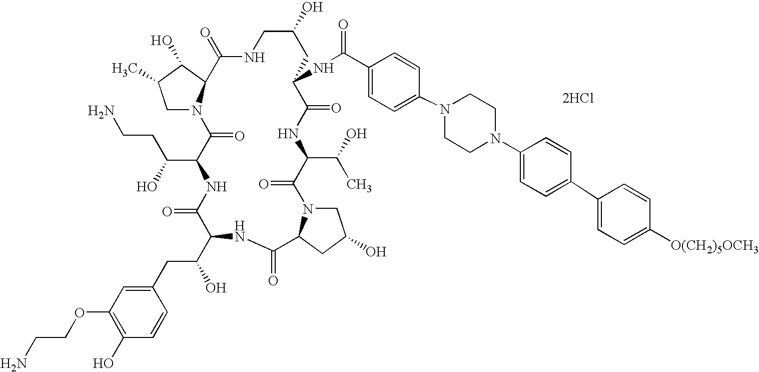 |
| 116 | 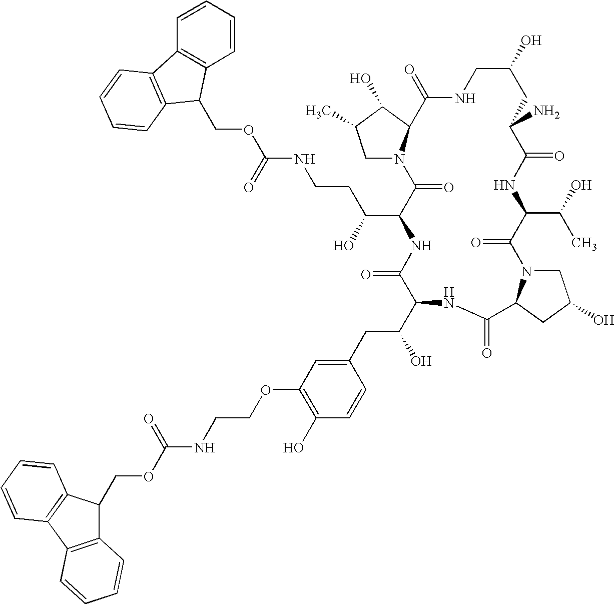 |
| | 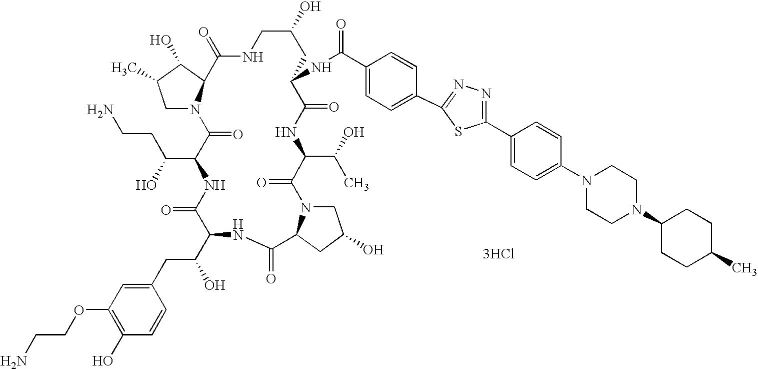 |
| 117 | 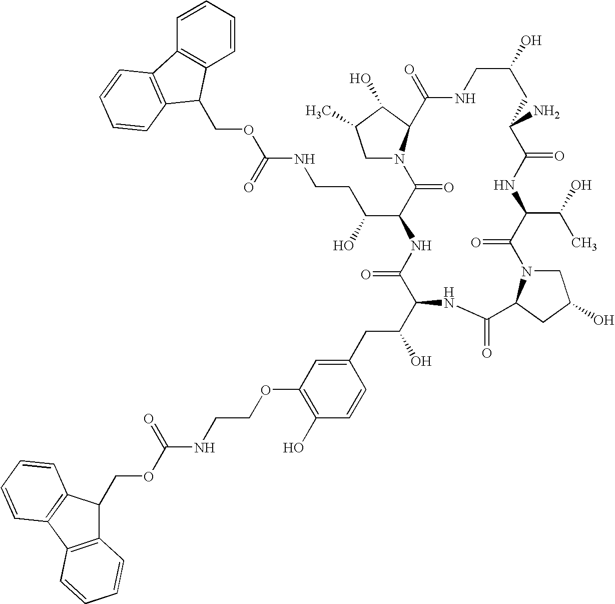 |
| | 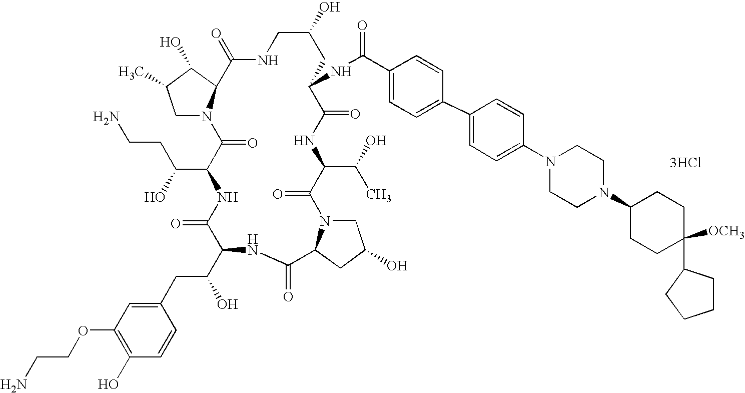 |
| 118 | 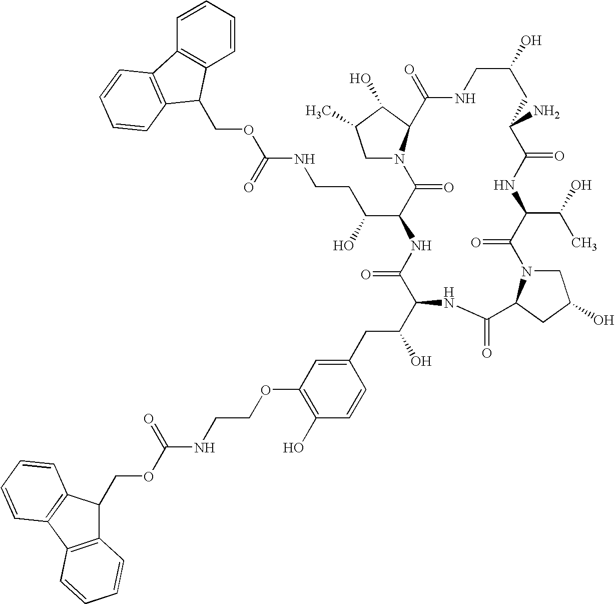 |
| | 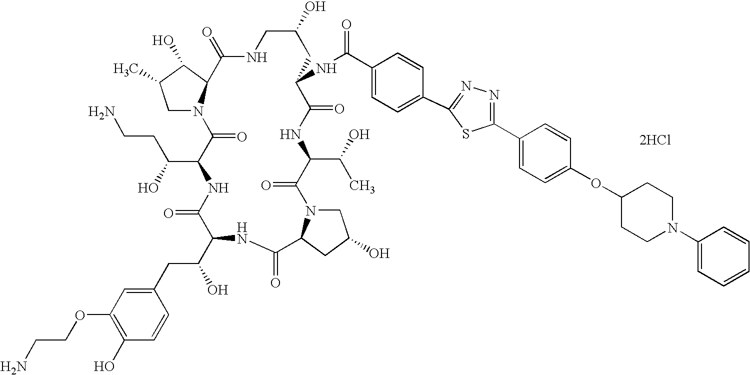 |
| 119 | 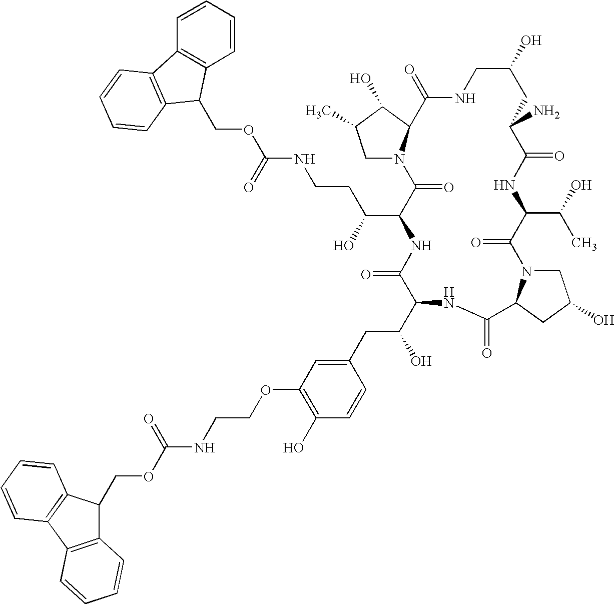 |
| | 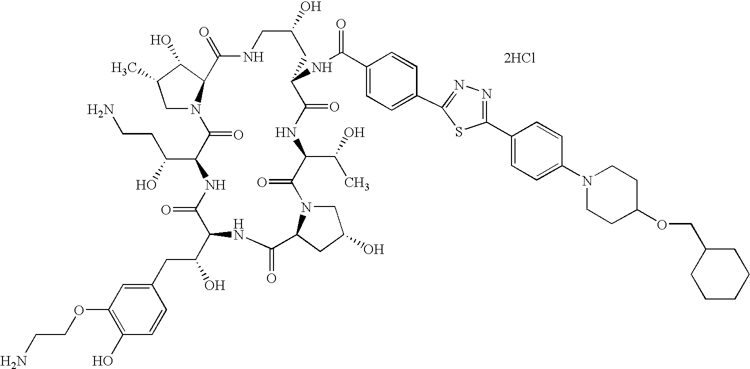 |
| 120 | 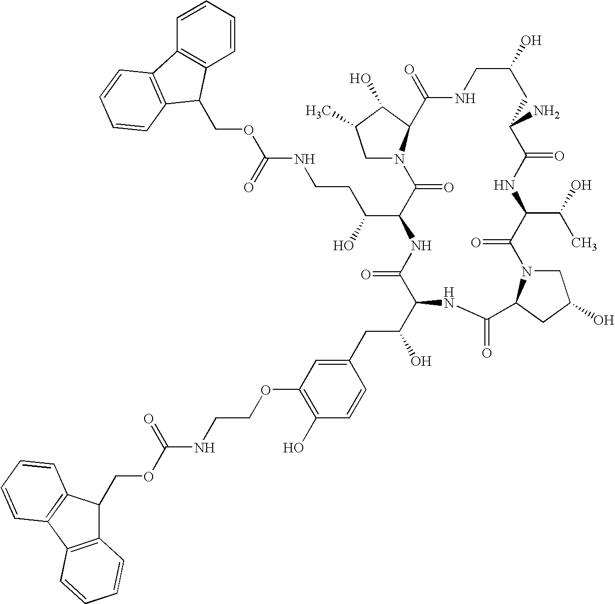 |
| | 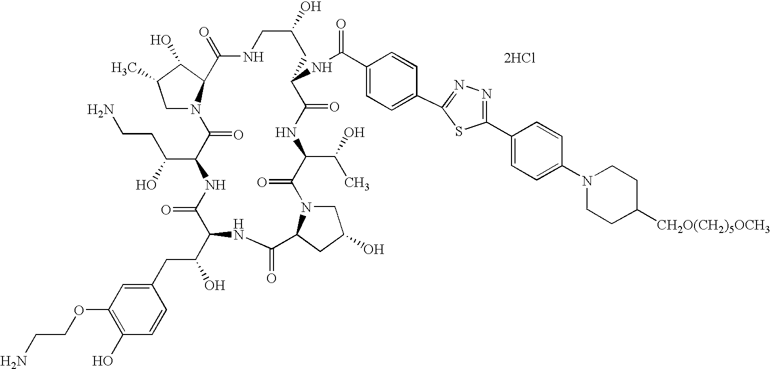 |
| 121 | 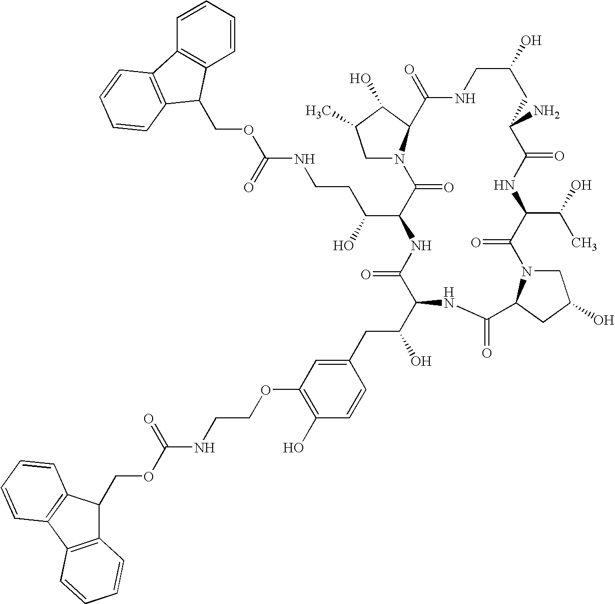 |
| | 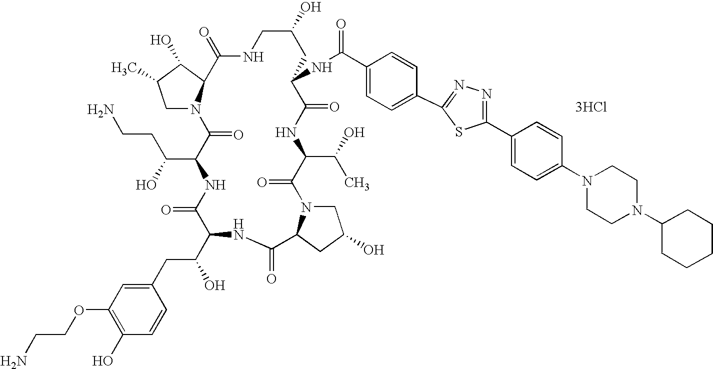 |
| 122 | 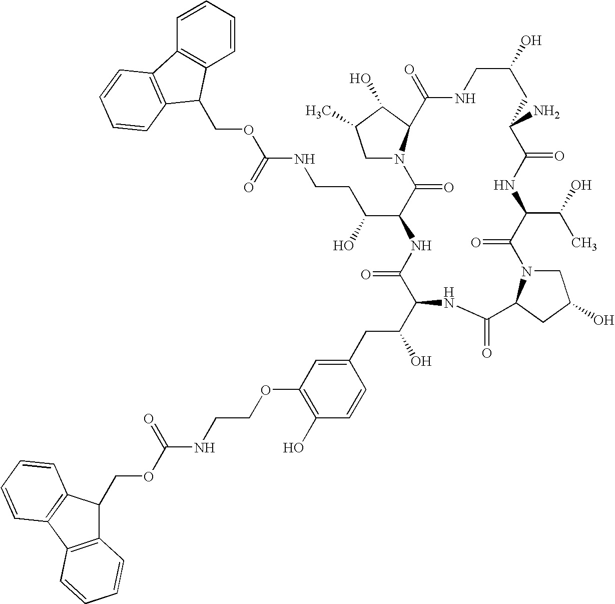 |
| | 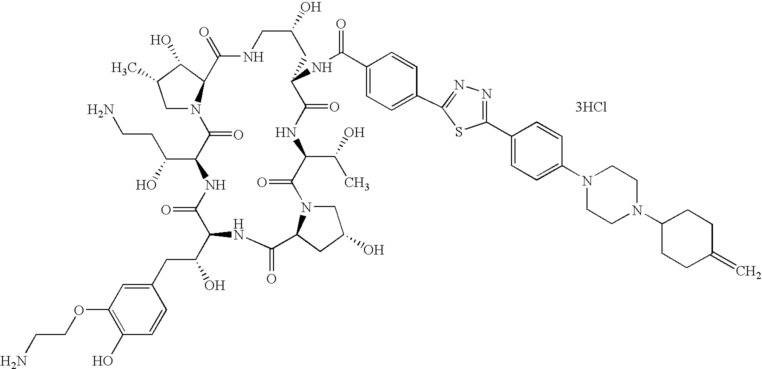 |
| 123 | 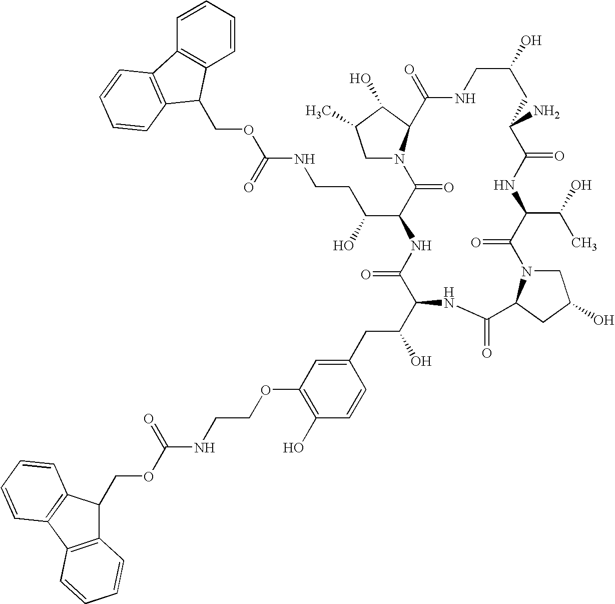 |
| | 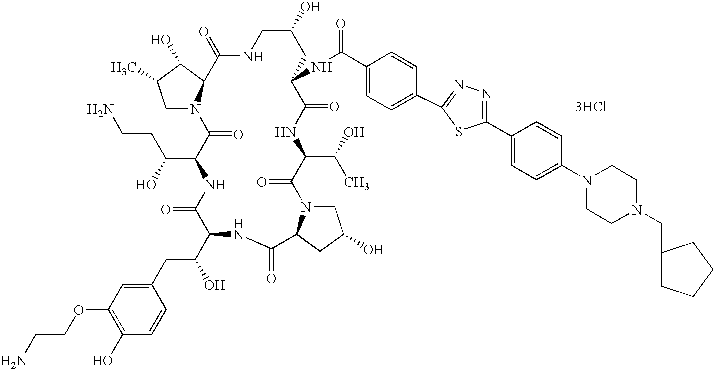 |
| 124 | 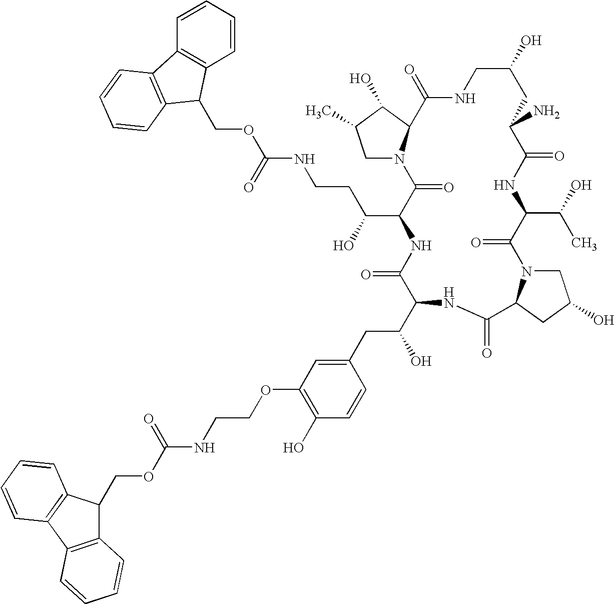 |
| | 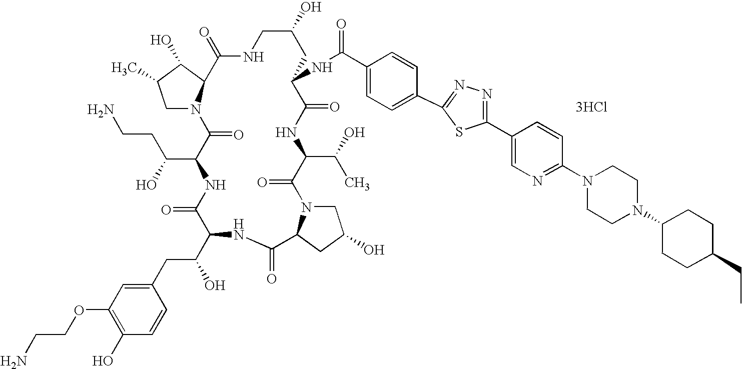 |
| 125 | 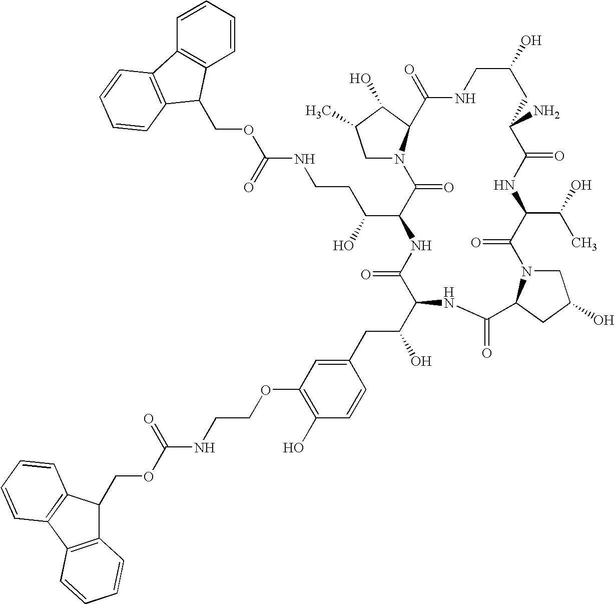 |
| | 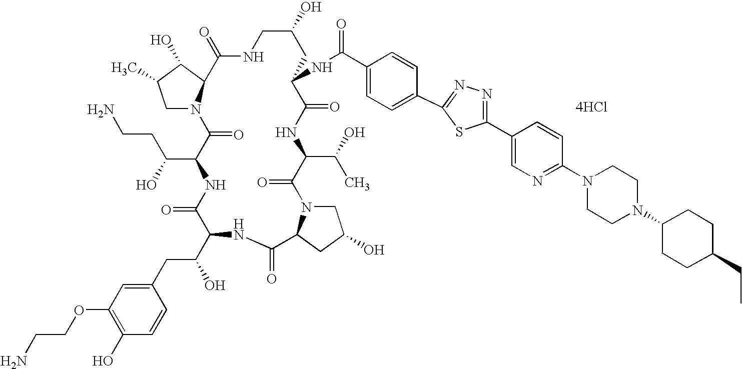 |
| 126 | 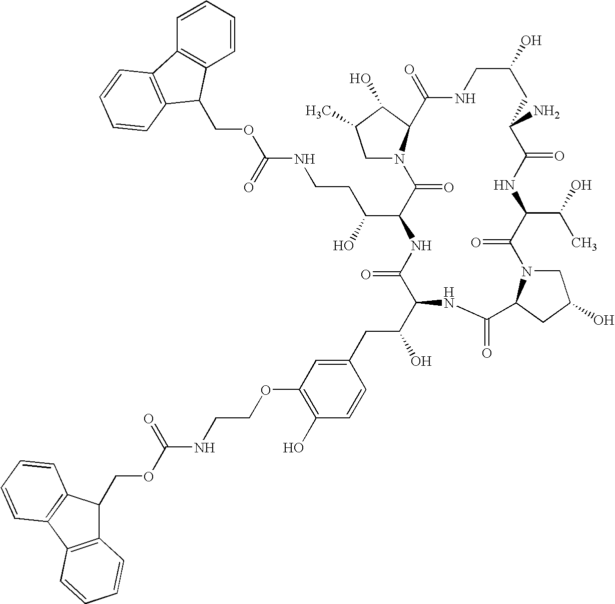 |
| | 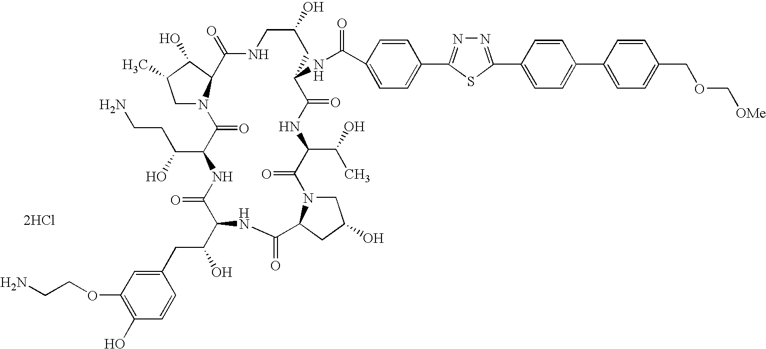 |
| 127 | 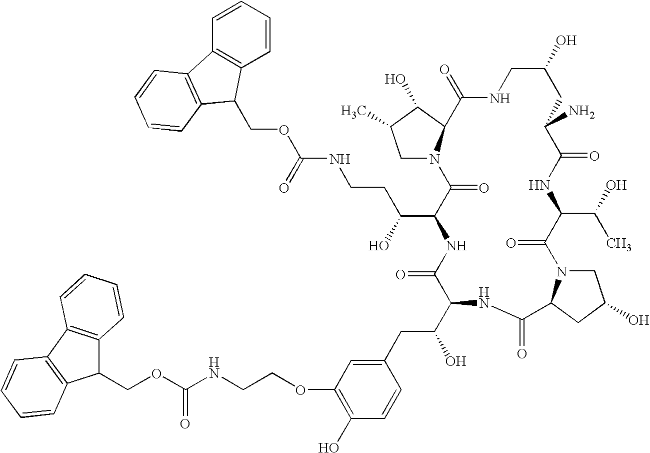 |
| | 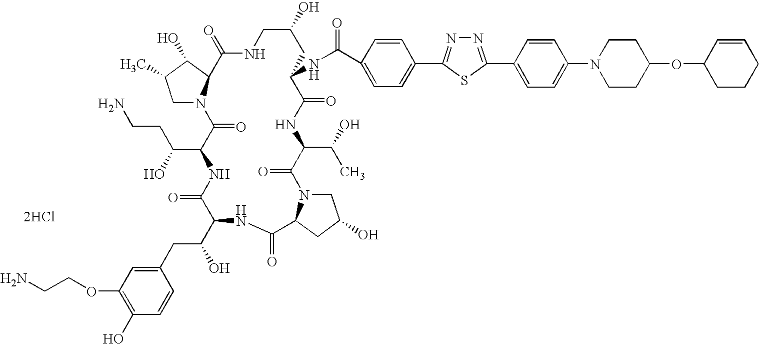 |
| 128 | 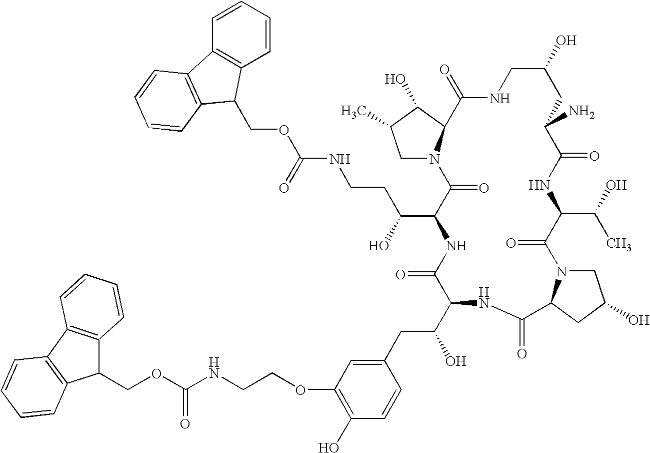 |
| | 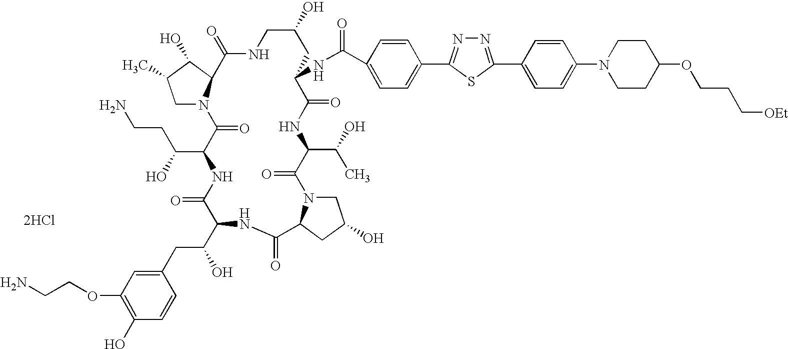 |
| 129 | 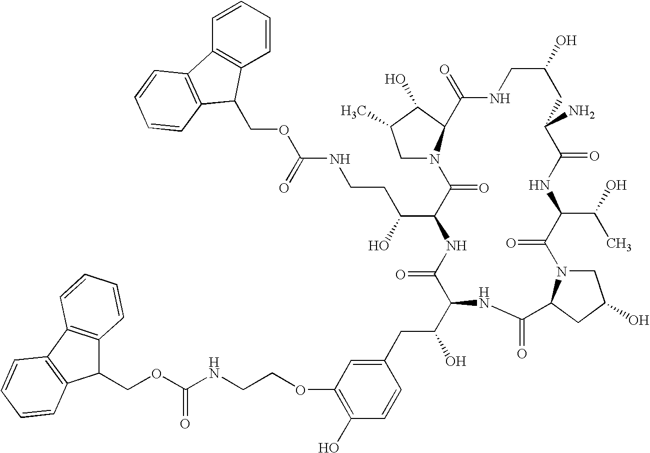 |
| | 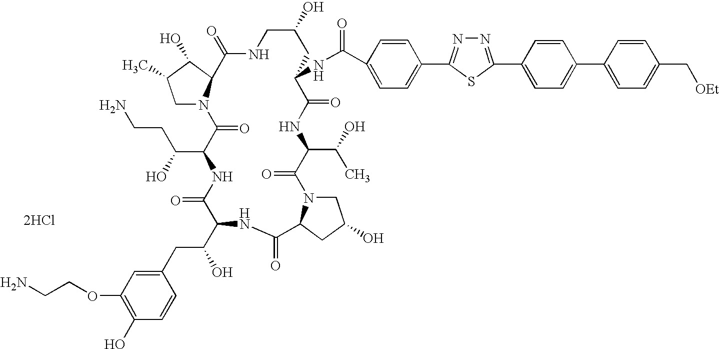 |
| 130 | 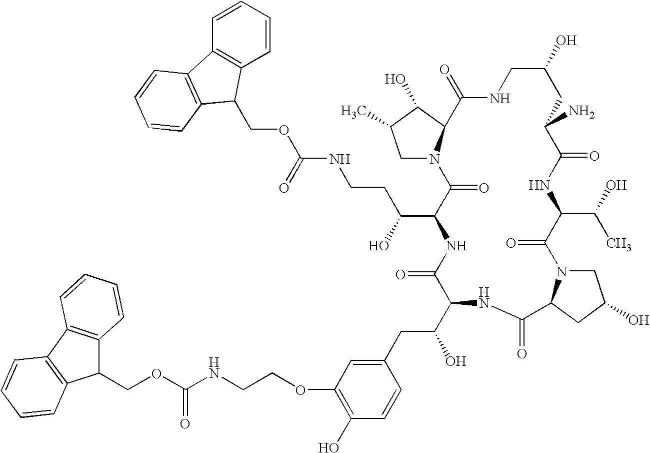 |
| | 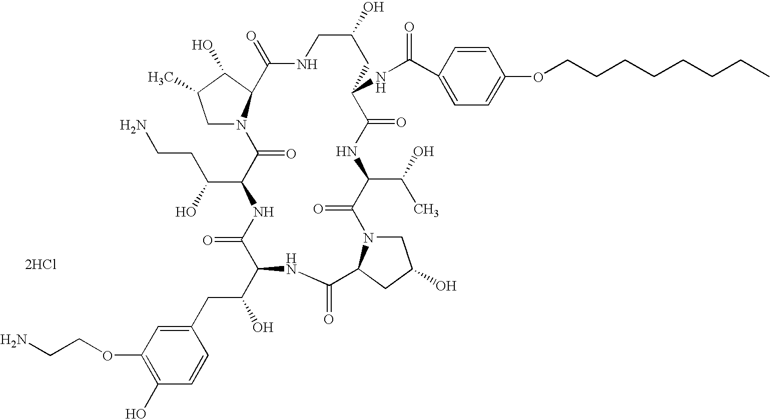 |
| 131 | 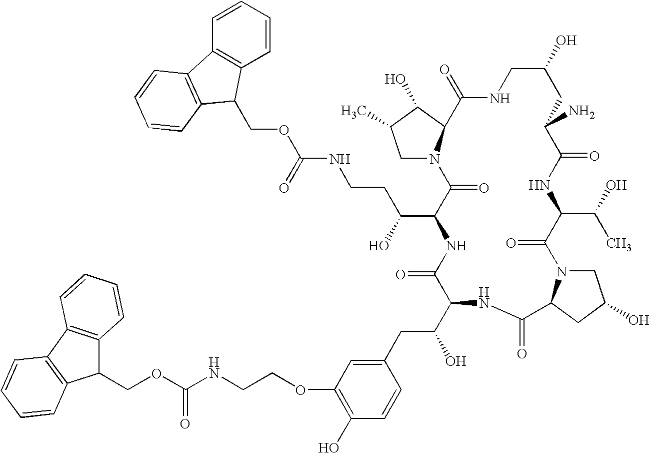 |
| | 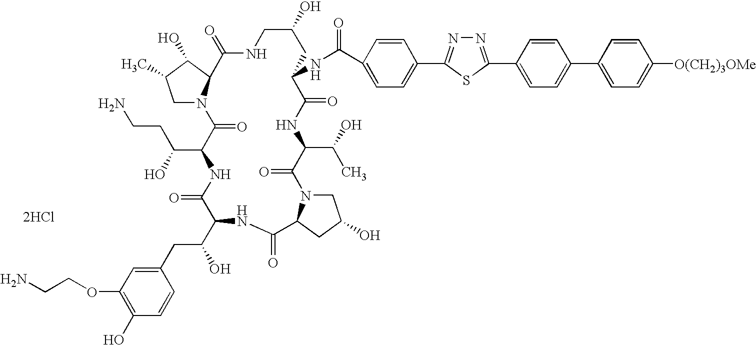 |
| 132 | 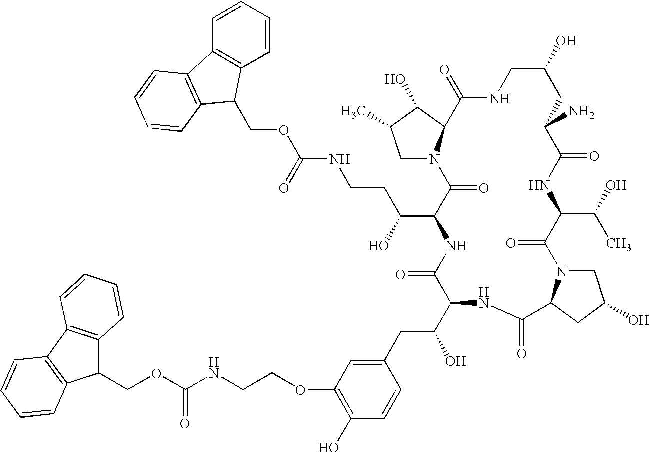 |
| | 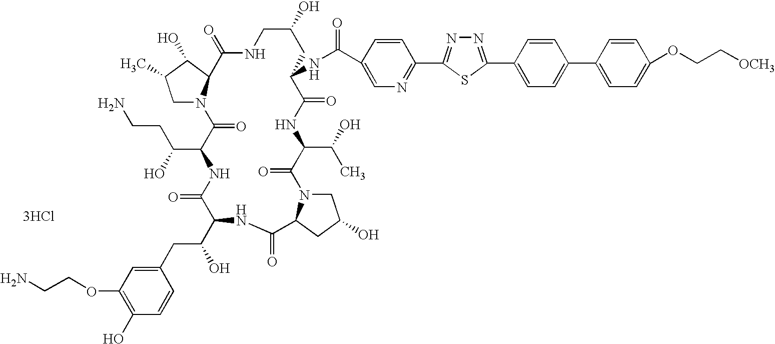 |
| 133 | 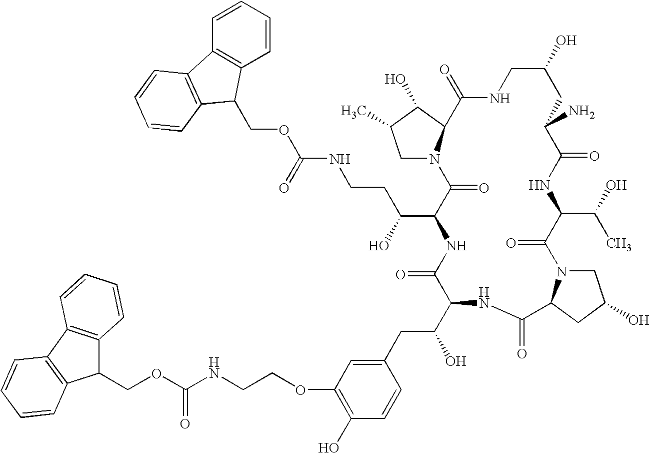 |
| | 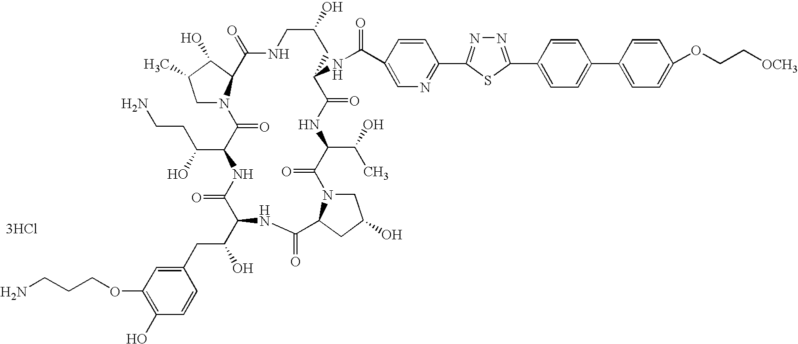 |
| 134 | 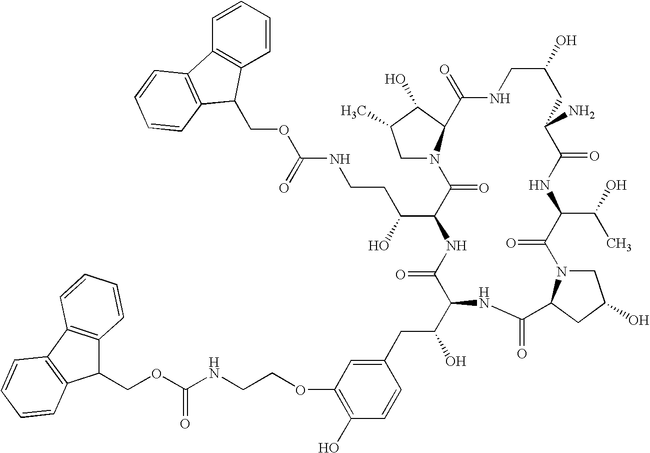 |
| | 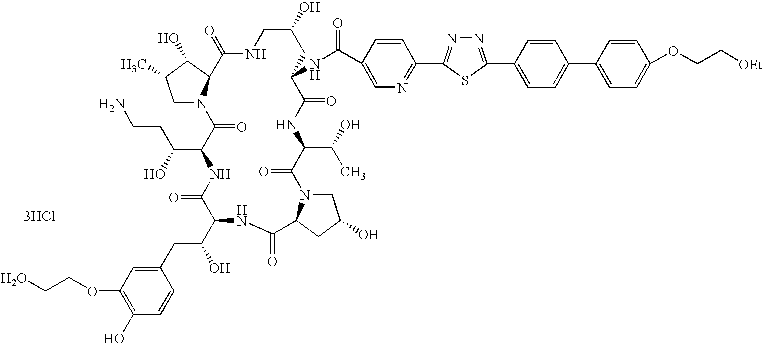 |
| 135 | 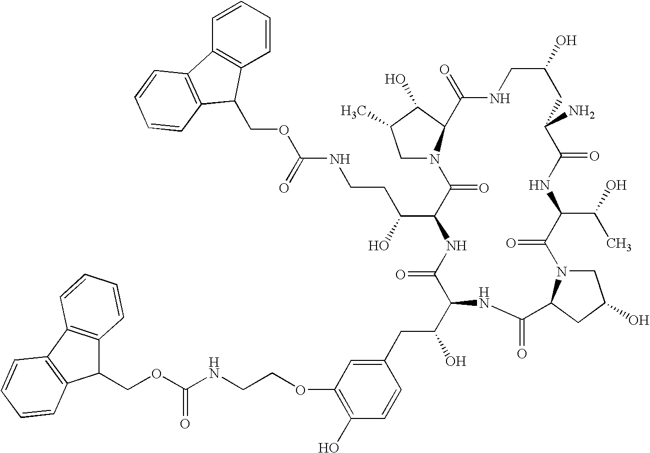 |
| | 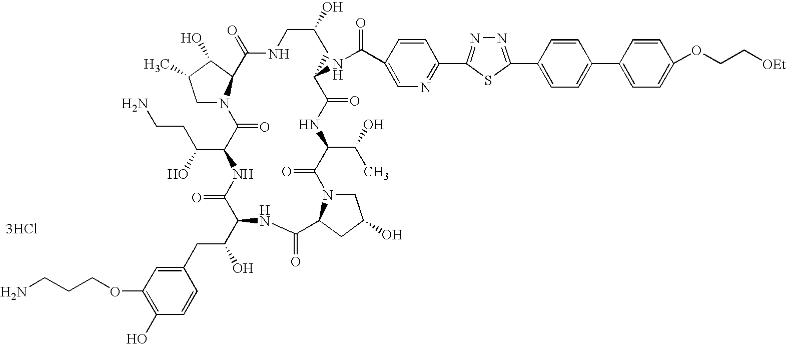 |
| 136 | 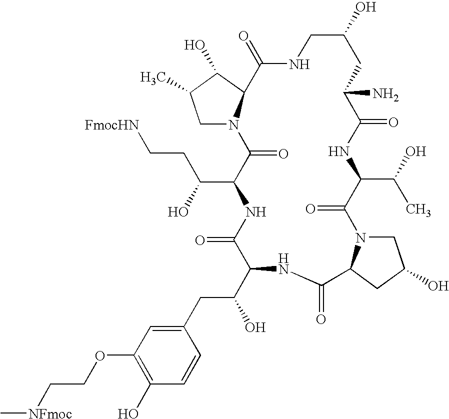 |
| | 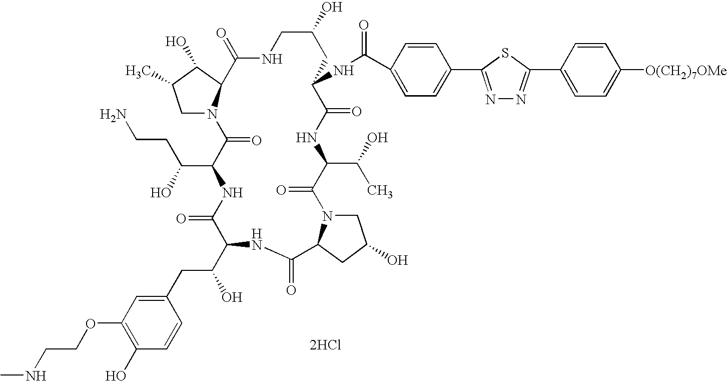 |
| 137 | 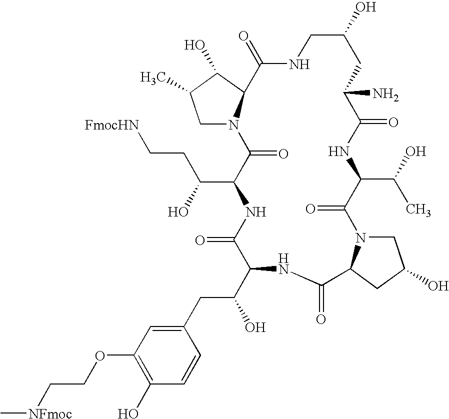 |
| | 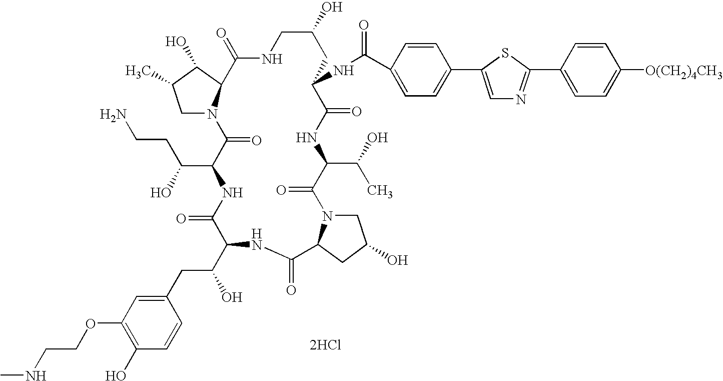 |
| 138 | 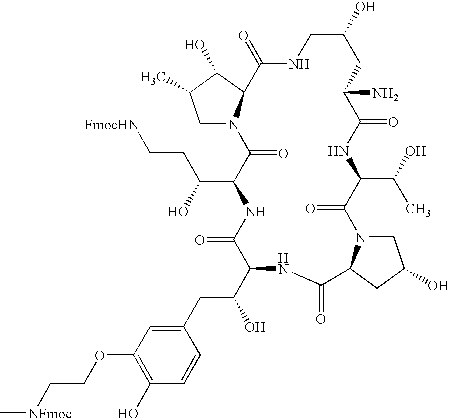 |
| | 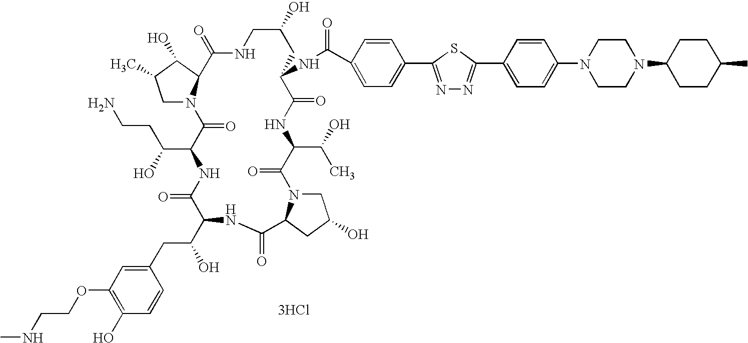 |
| 139 | 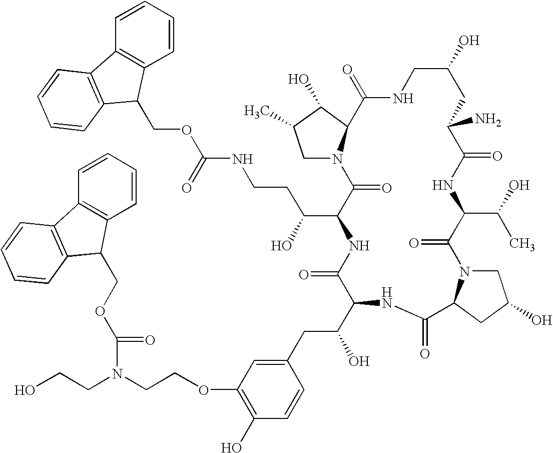 |
| | 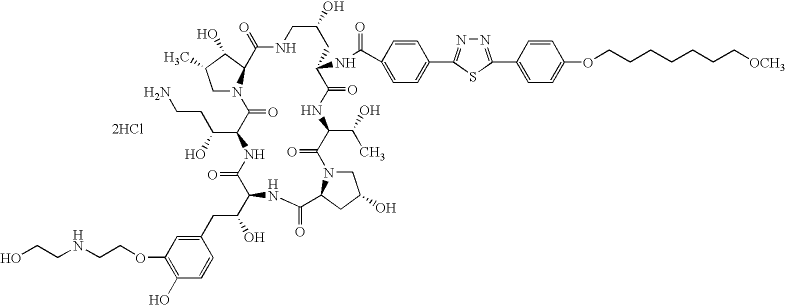 |
| 140 | 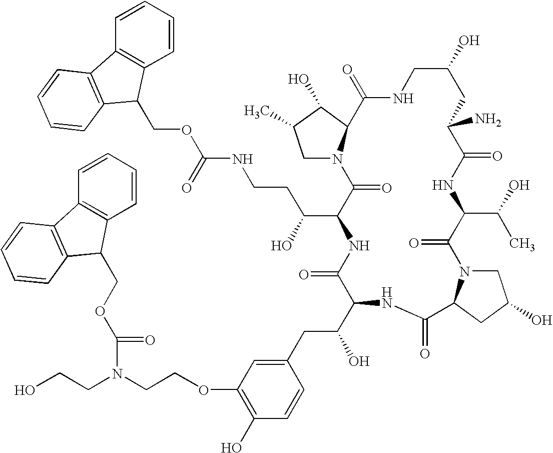 |
| | 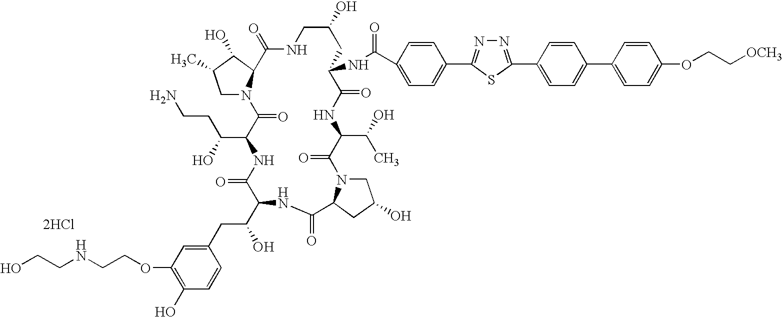 |
| 141 | 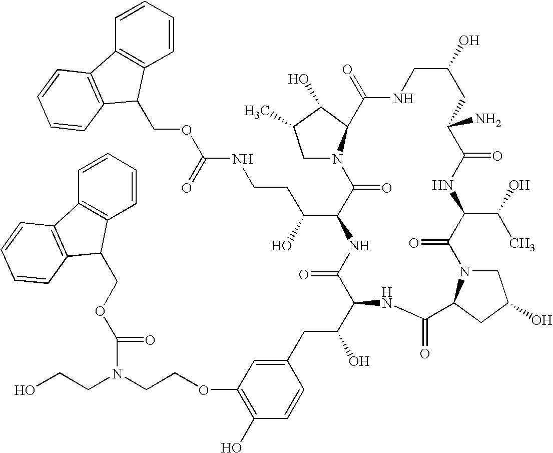 |
| | 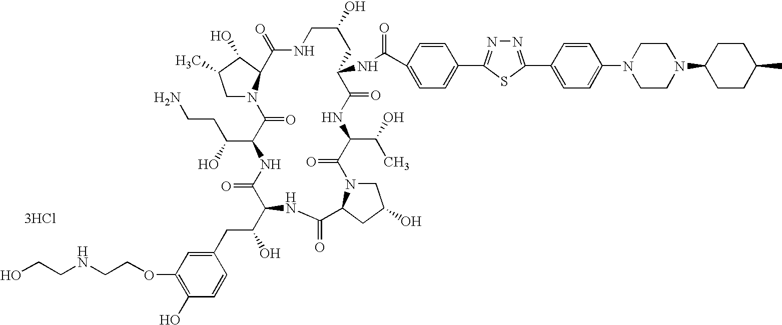 |
| 142 | 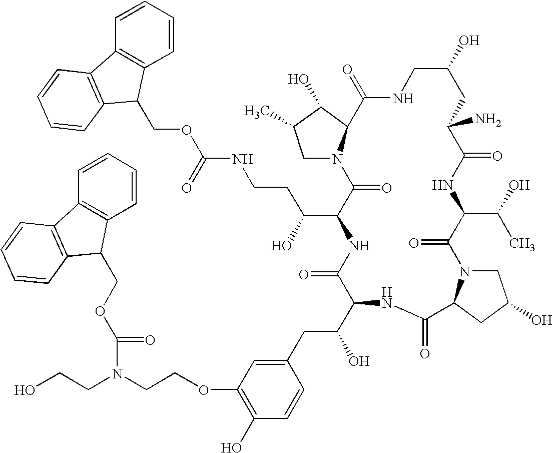 |
| | 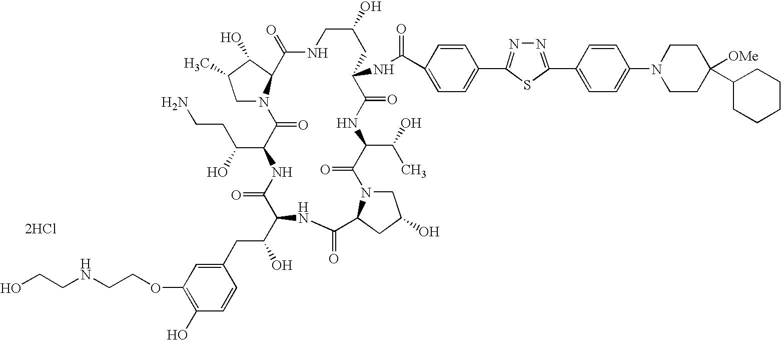 |
| 143 | 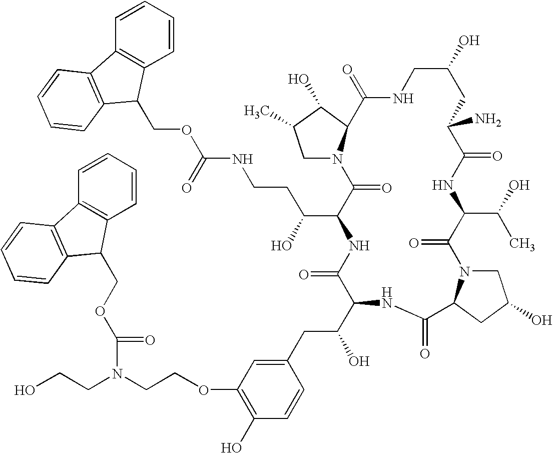 |
| | 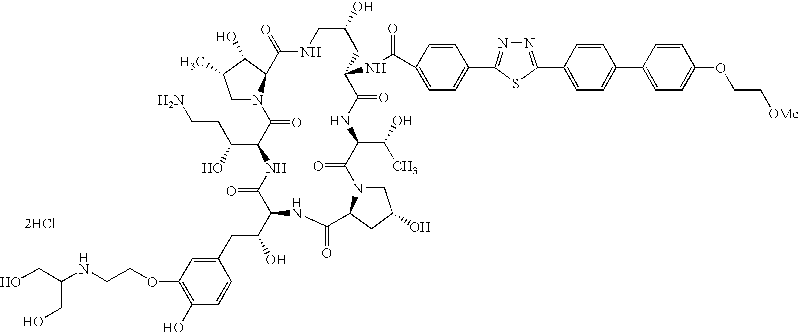 |
| 144 | 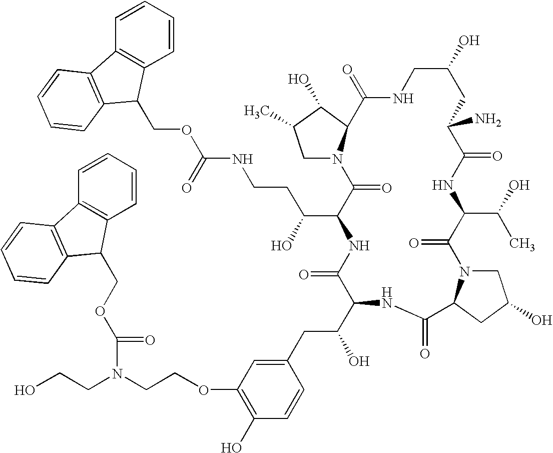 |
| | 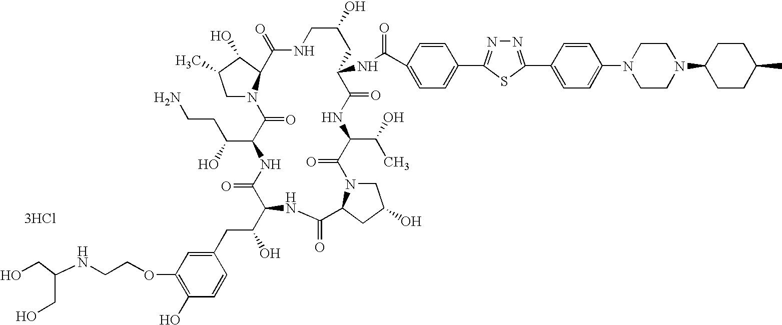 |
| 145 | 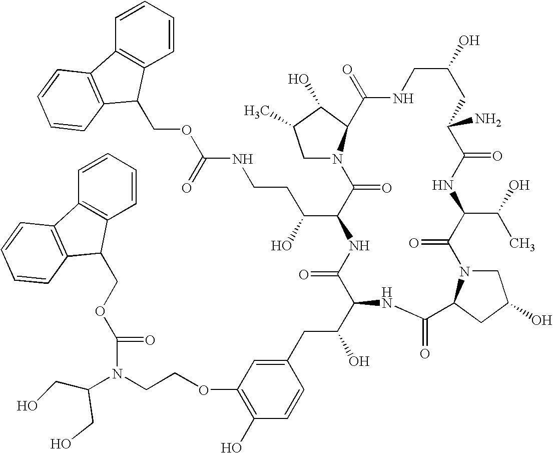 |
| | 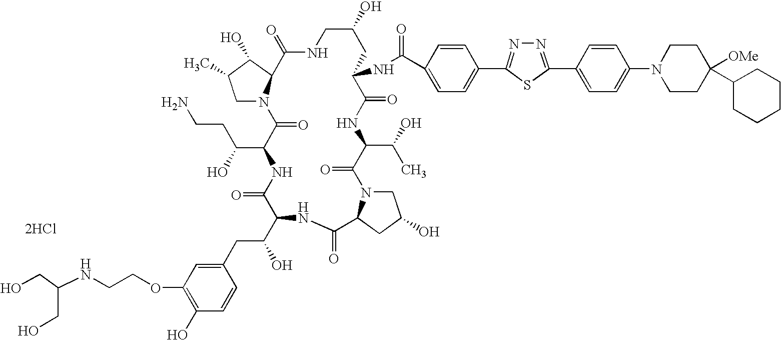 |
| 146 | 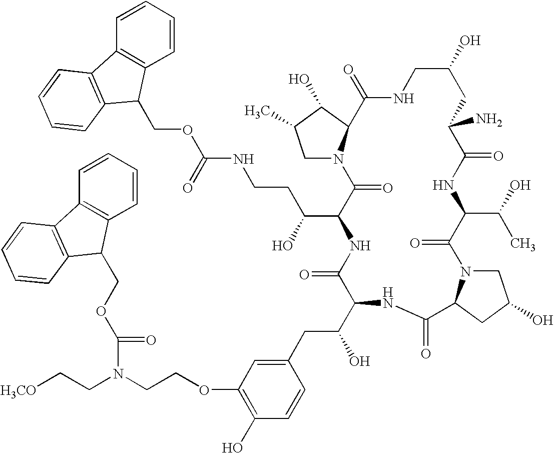 |
| | 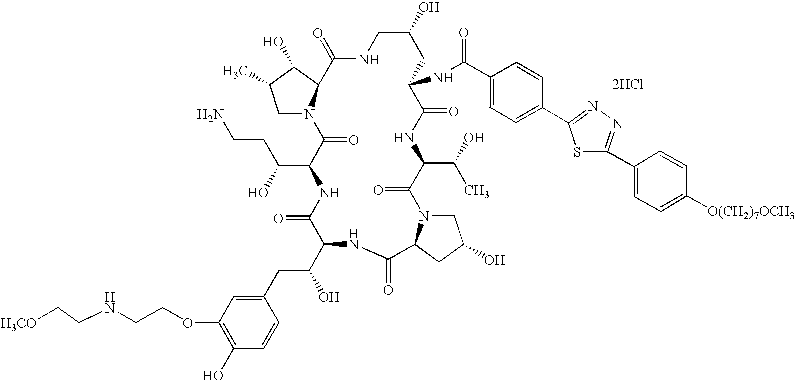 |
| 147 | 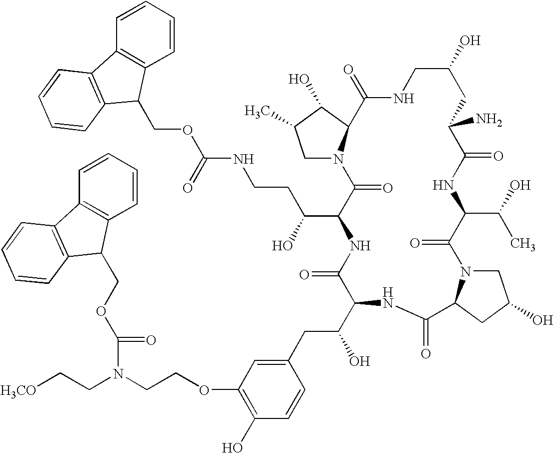 |
| | 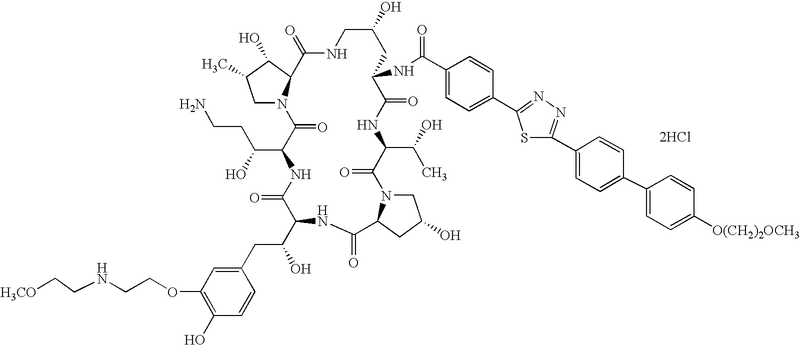 |
| 148 | 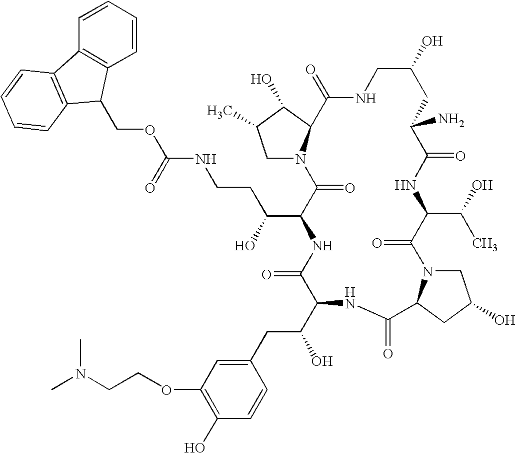 |
| | 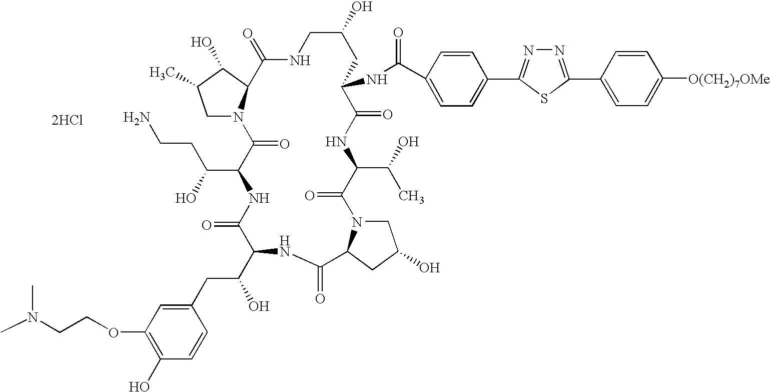 |
| 149 | 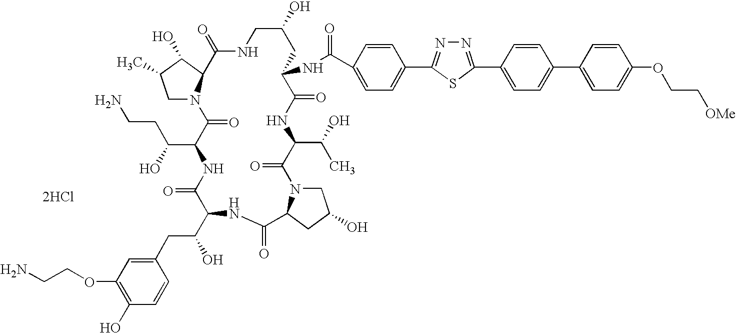 |
| | 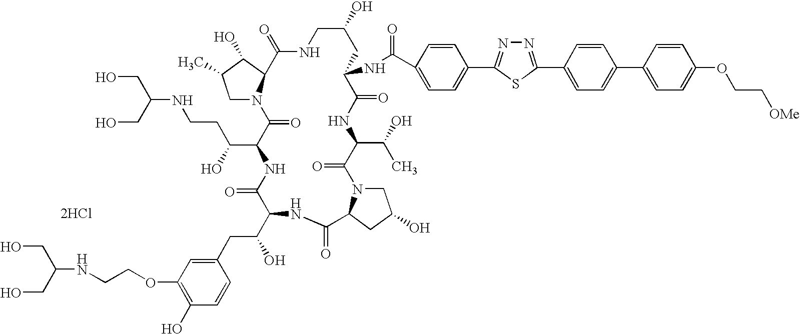 |
| 150 | 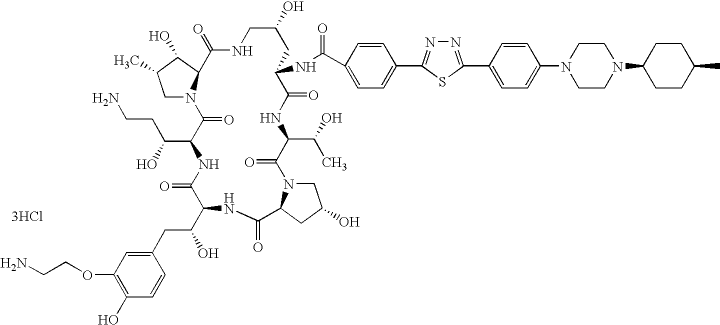 |
| | 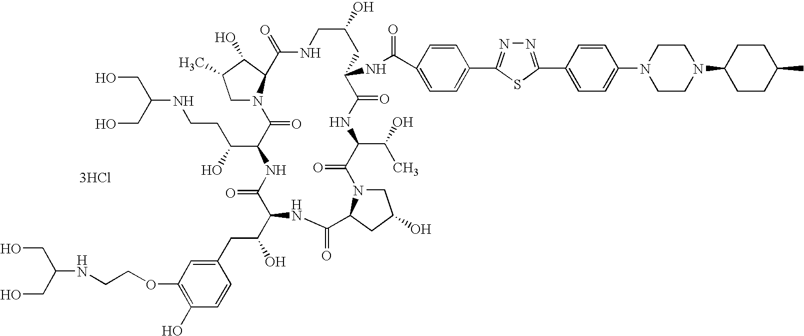 |
| 151 | 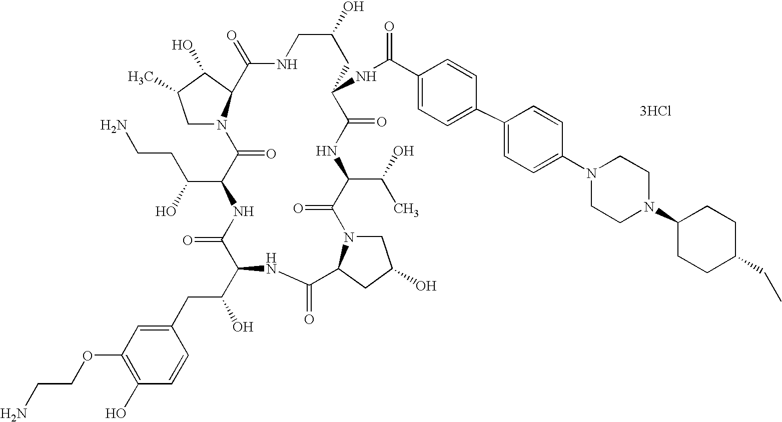 |
| | 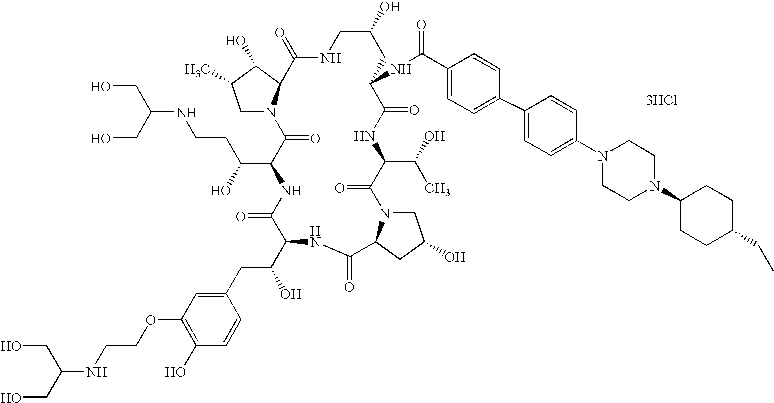 |
| 152 | 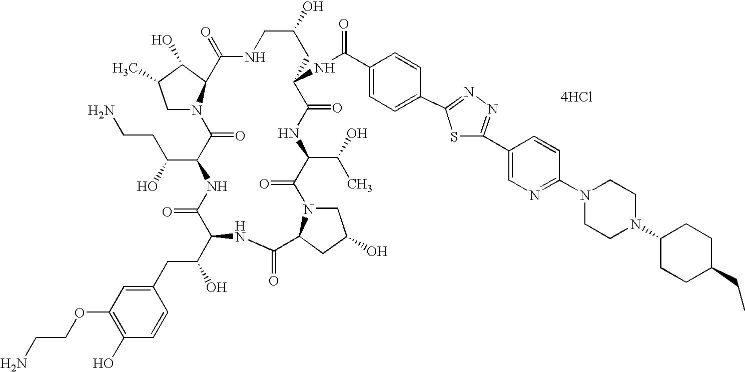 |
| | 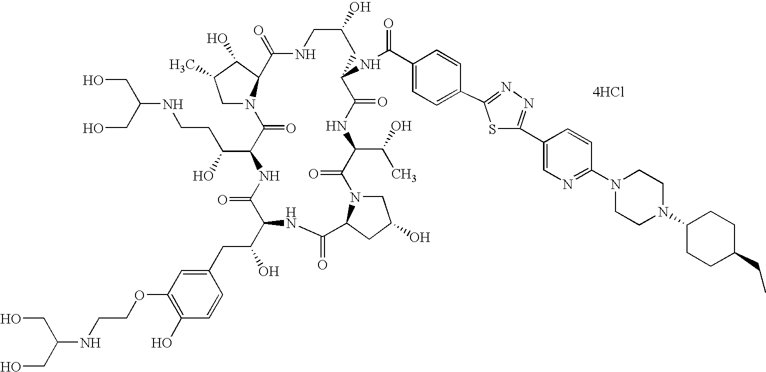 |
| 153 | 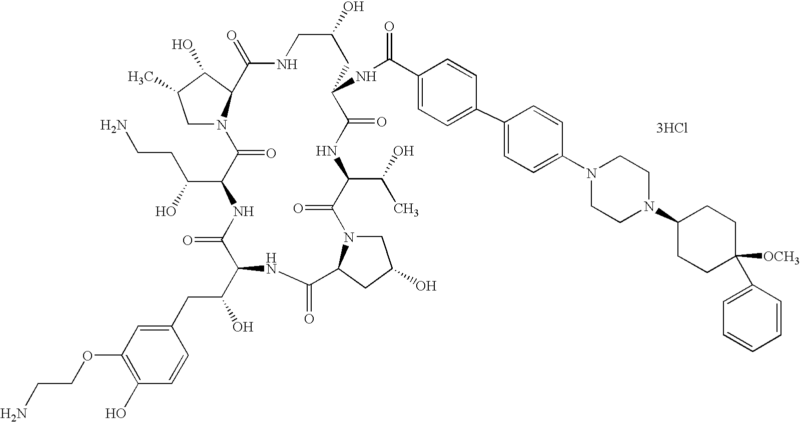 |
| | 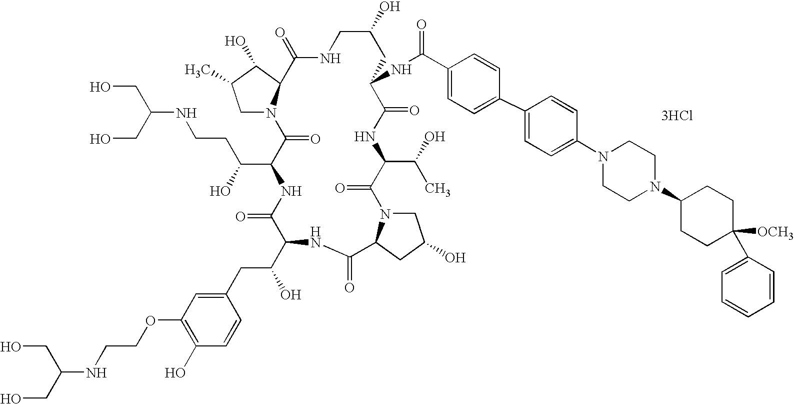 |
| 154 | 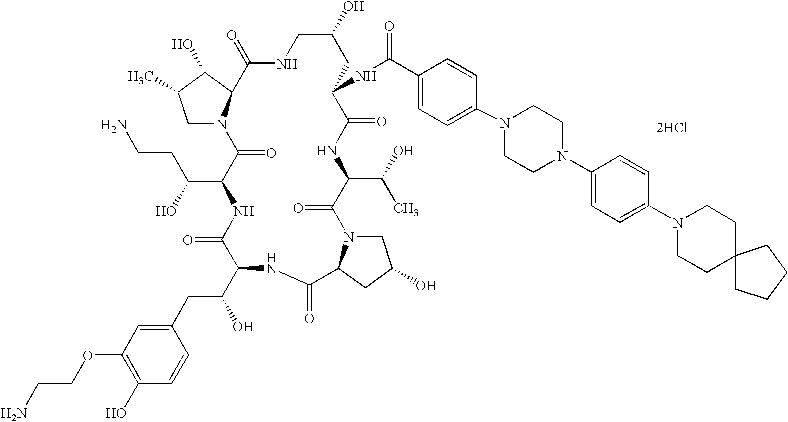 |
| | 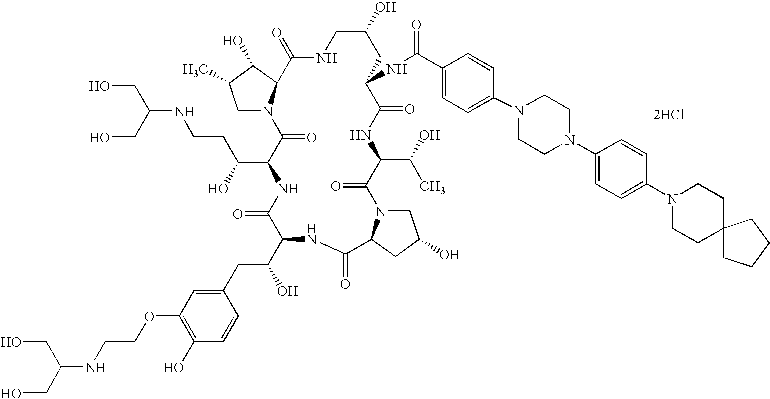 |
| 155 | 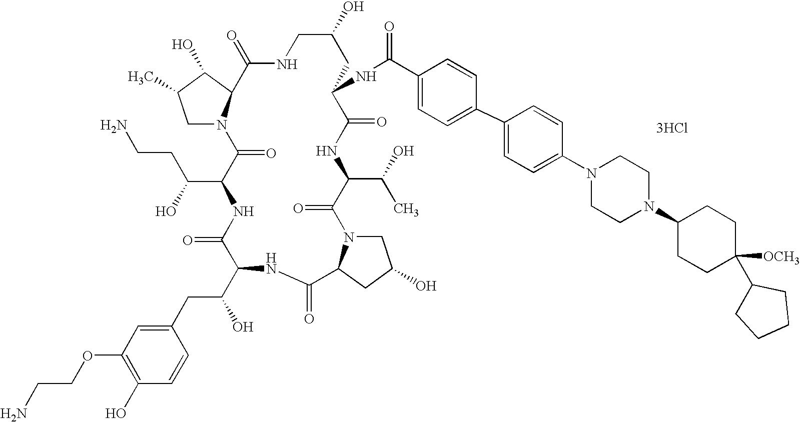 |
| | 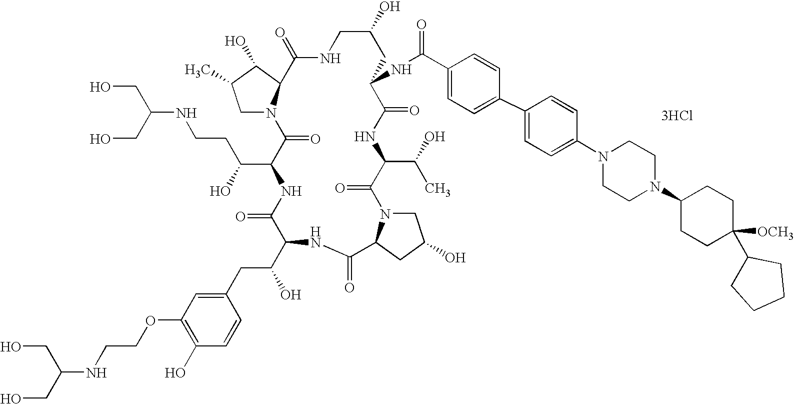 |
| 156 | 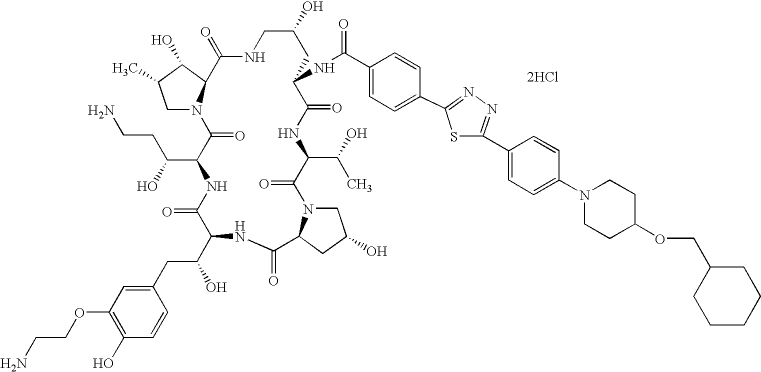 |
| | 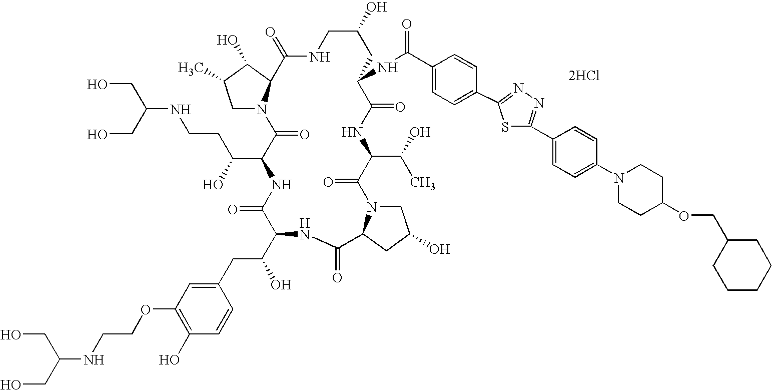 |
| 157 | 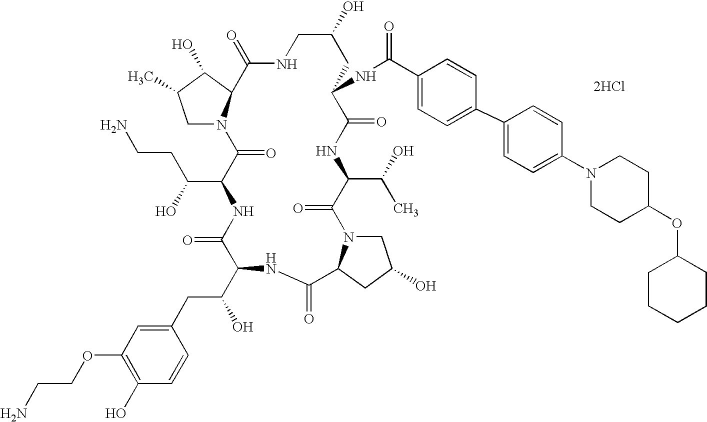 |
| | 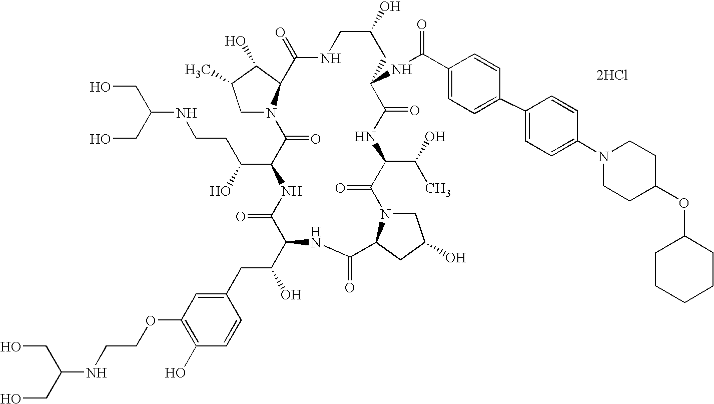 |
| 158 | 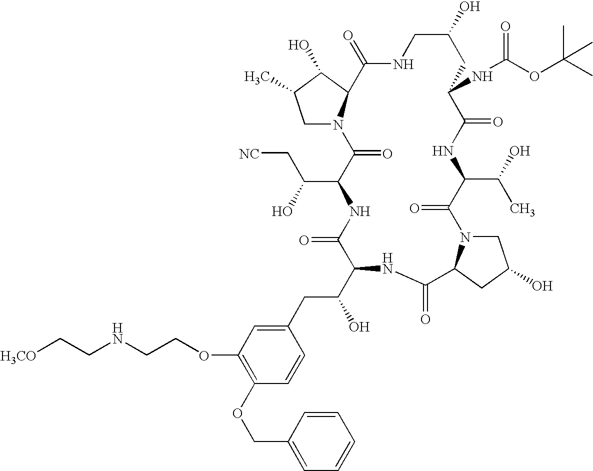 |
| | 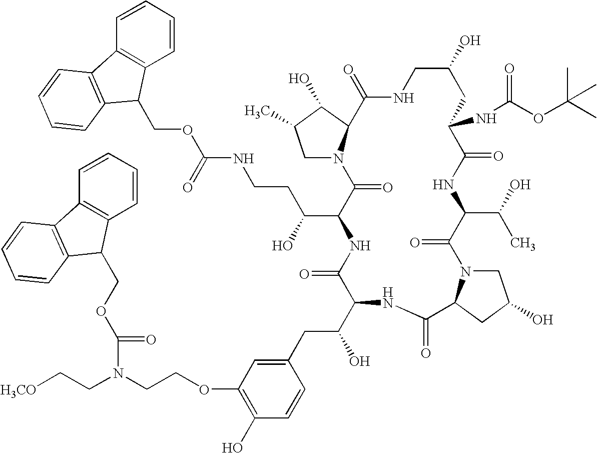 |
| 159 | 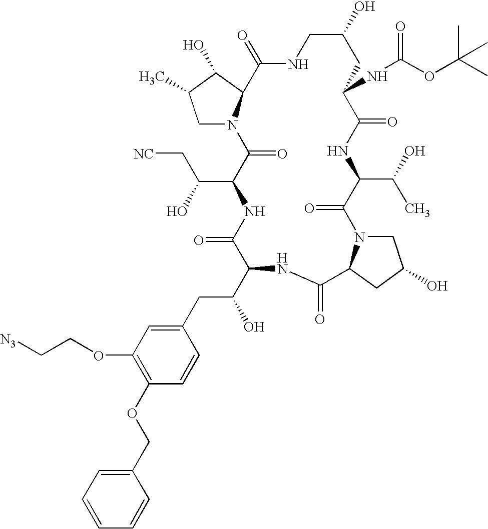 |
| | 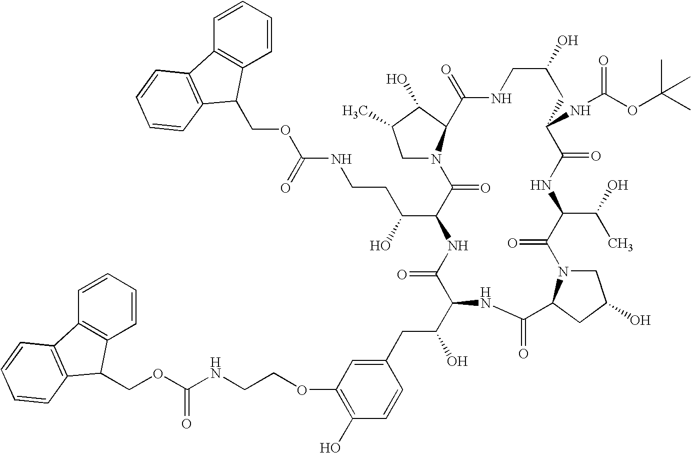 |
| 160 | 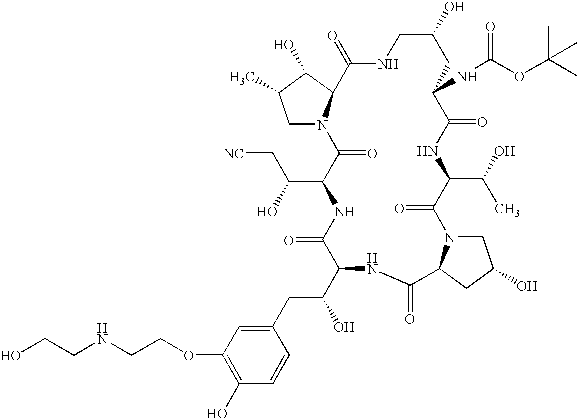 |
| | 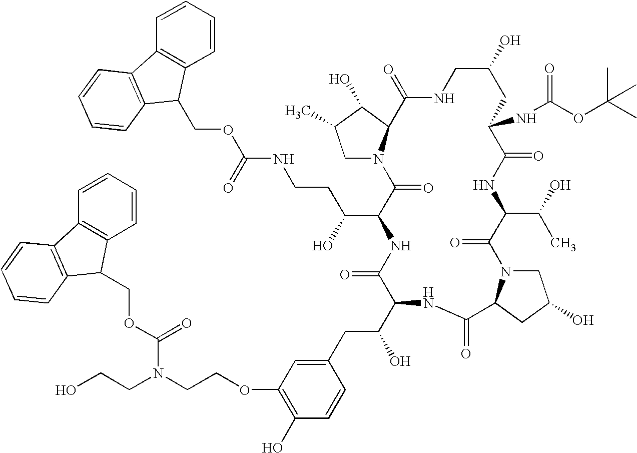 |
| 161 | 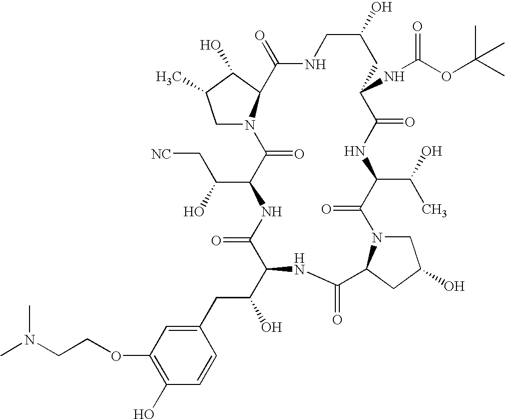 |
| | 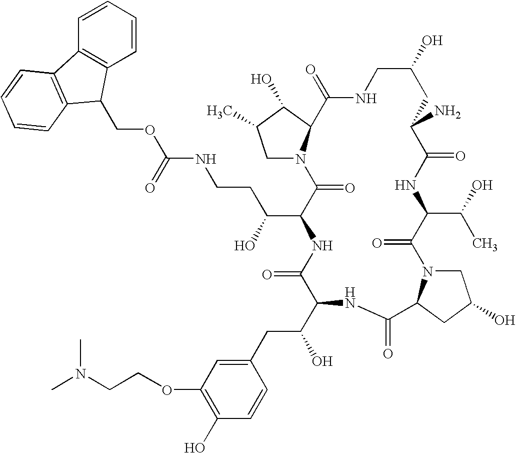 |
| 162 | 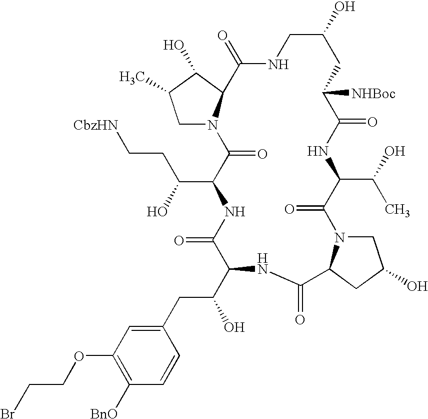 |
| | 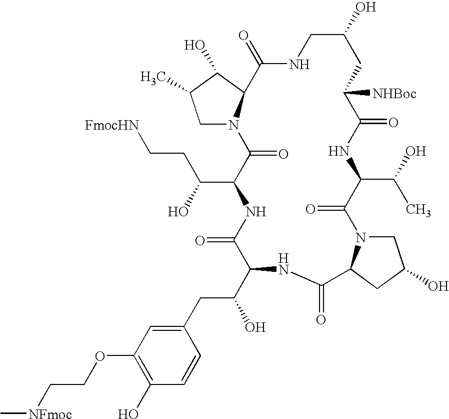 |
| 163 | 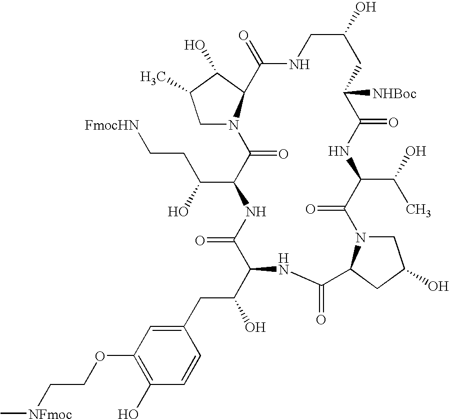 |
| | 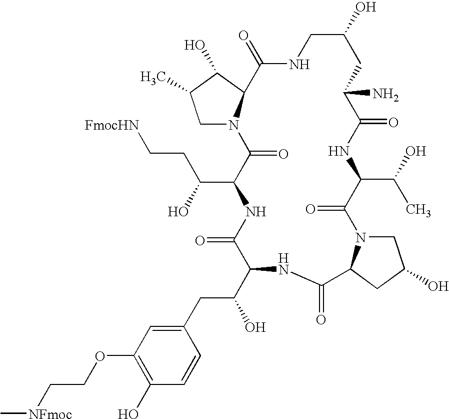 |
| 164 | 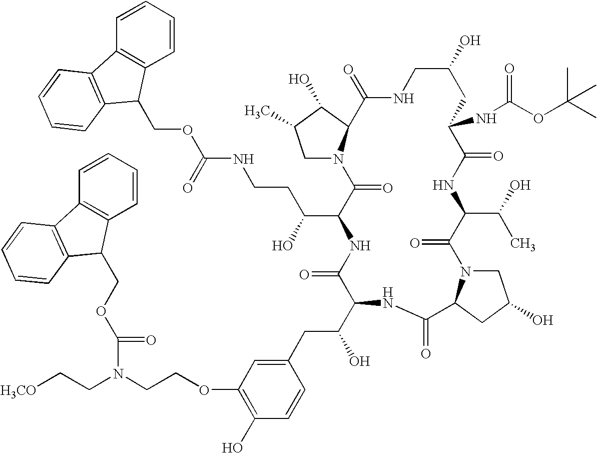 |
| | 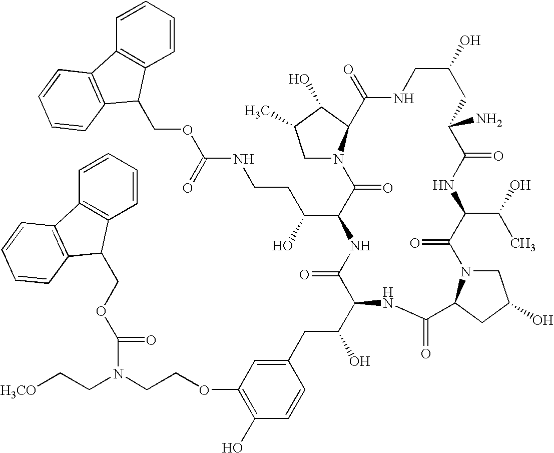 |
| 165 | 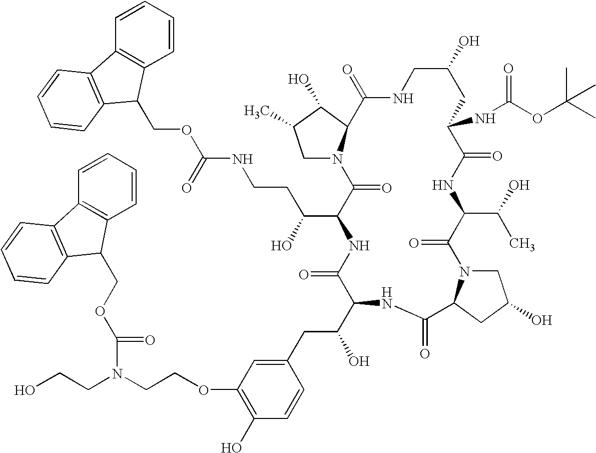 |
| | 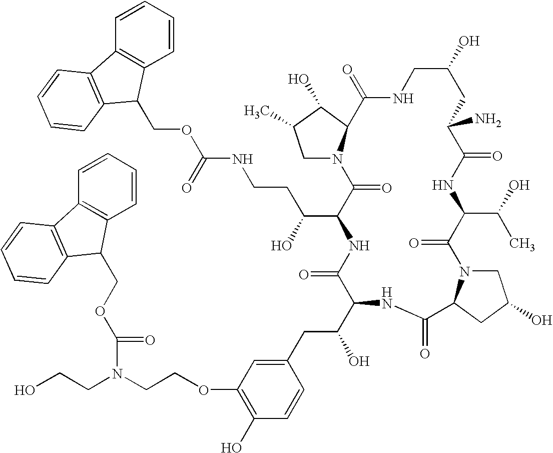 |
| 166 | 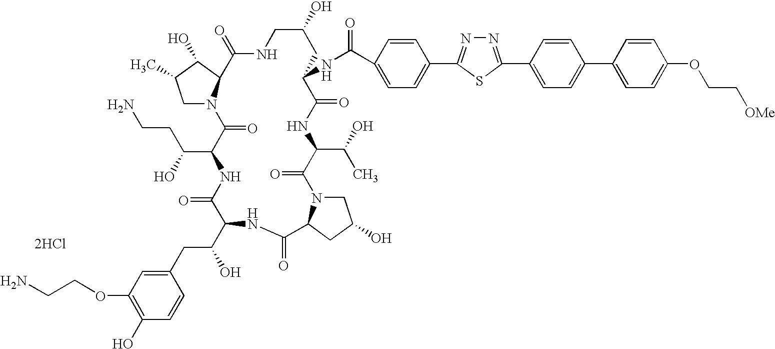 |
| | 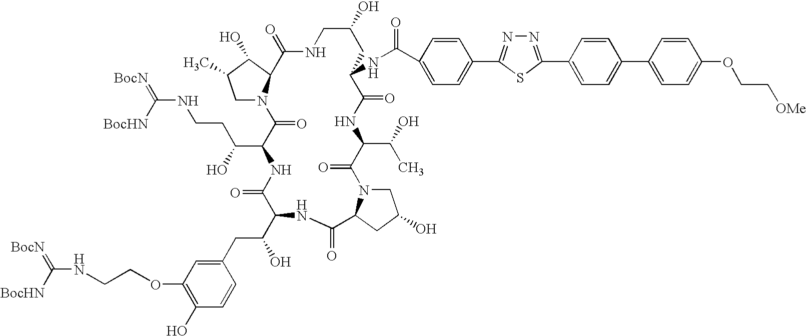 |
| 167 | 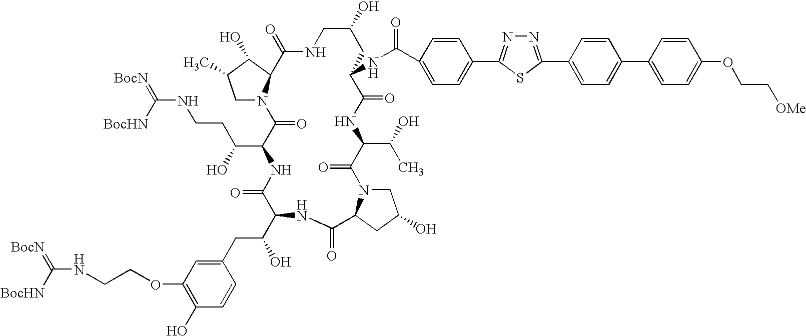 |
| | 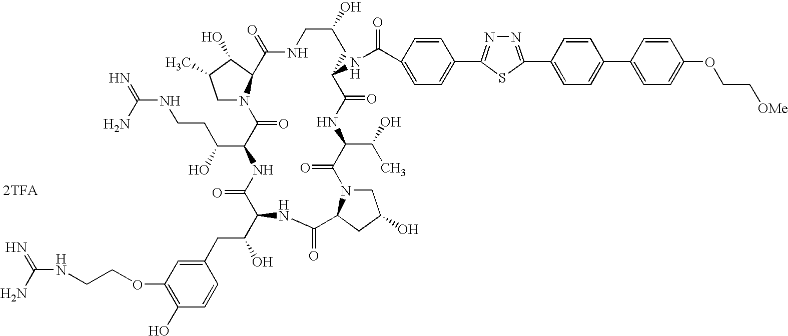 |
| 168 | 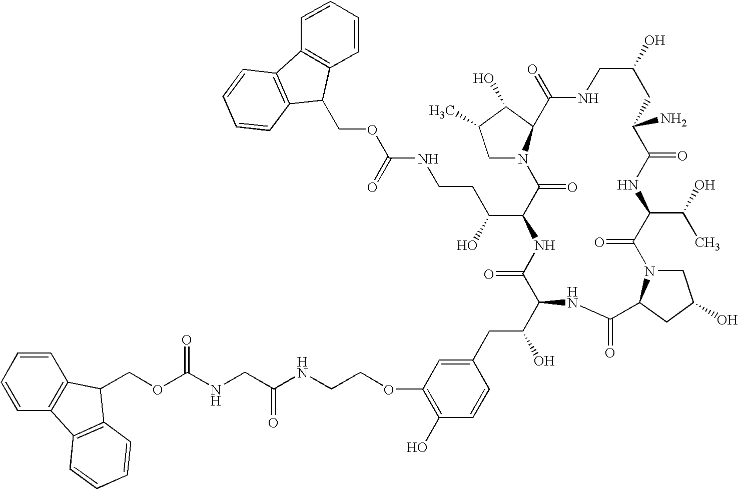 |
| | 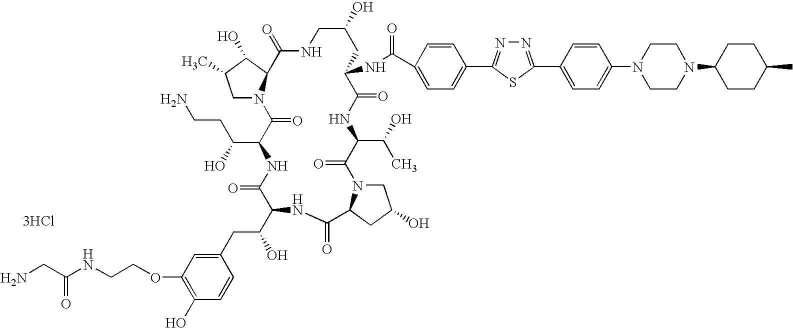 |
| 169 | 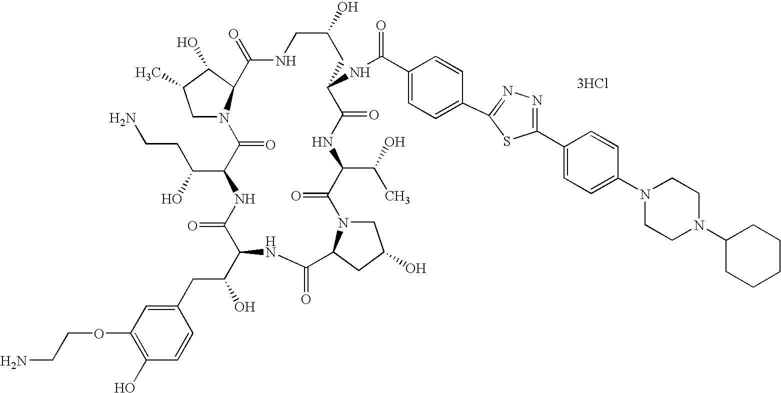 |
| | 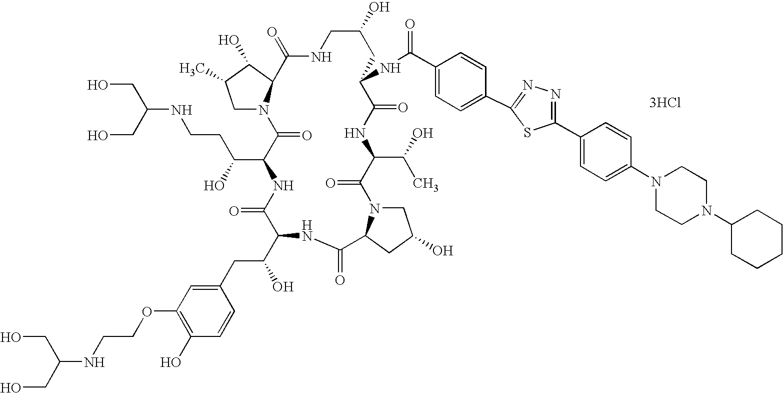 |
| 170 | 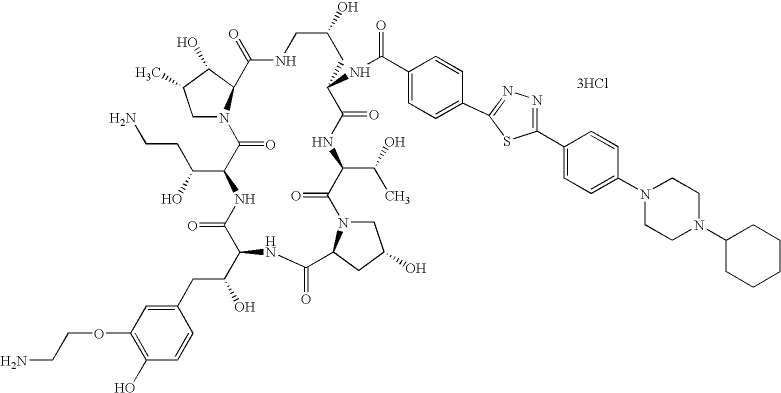 |
| | 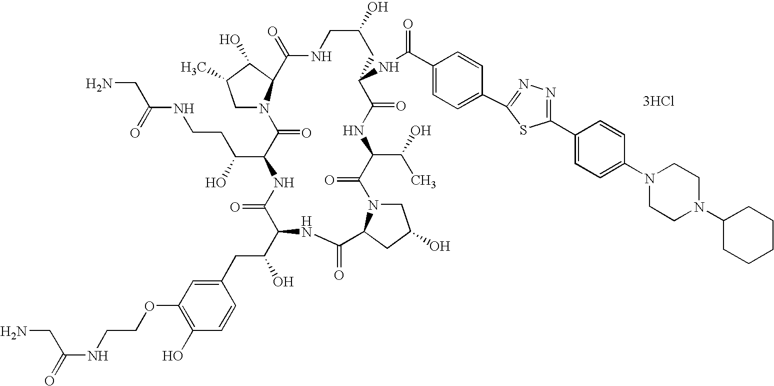 |

### EXAMPLE 1

To a solution of the starting compound (1) (900 mg) and cobalt(II)chloride hexahydrate (771 mg) in a mixture of methanol (27 ml) and water (5.4 ml) was added sodium borohydride (306 mg) with stirring at room temperature and the mixture was stirred at the same temperature for 2.5 hours. The reaction mixture was filtered through celite. To the filtrate was added 10% palladium on carbon (450 mg), and hydrogen gas at atmosphere pressure for 2 hours. The reaction mixture was filtered through celite. The filtrate was adjusted to pH 8.3 with 1N-hydrochloric acid, evaporated under reduced pressure to remove methanol and was lyophilized. To the resulting residue and N,N-diisopropylethylamine (0.423 ml) in N,N-dimethylformamide (9 ml) was added 9-fluorenylmethyl chloroformate (314 mg) at room temperature, and the mixture was stirred for 2.5 hours. Water was added and the resulting precipitate collected and dried to give the object compound (1) (794 mg) as a crude precipitate, that was used directly in the next step without purification.

### EXAMPLE 2

To a solution of the starting compound (2) (390 mg) and cobalt(II)chloride hexahydrate (347 mg) in a mixture of methanol (11.7 ml) and water (2.3 ml) was added sodium borohydride (276 mg) with stirring at room temperature and the mixture was stirred at the same temperature for 2.5 hours. The reaction mixture was filtered through celite. To the filtrate was added 10% palladium on carbon (195 mg), and hydrogen gas at atmosphere pressure for 2 hours. The reaction mixture was filtered through celite. The filtrate was adjusted to pH 8.3 with 1N-hydrochloric acid, evaporated under reduced pressure to remove methanol and was lyophilized. To the resulting residue and N,N-diisopropylethylamine (0.191 ml) in N,N-dimethylformamide (7.8 ml) was added 9-fluorenylmethyl chloroformate (142 mg) at room temperature, and the mixture was stirred for 6 hours. Water was added and the resulting precipitate collected and dried. The resulting precipitate was dissolved in 30% acetonitrile in water and the solution was subjected to column chromatography on ODS eluting with 40% acetonitrile in water. The fractions containing thd object compound were collected and evaporated under reduced pressure to remove acetonitrile. The residue was lyophilized to give the object compound (2) (162.4 mg).

NMR (DMSO-d₆+D₂O, δ): 0.8-2.5 (23H, m), 2.6-3.4 (9H, m), 3.5-4.9 (19H, m), 6.28 (1H, s), 6.55-6.8 (3H, m), 7.2-7.5 (4H, m), 7.67 (1H, d, J=7.1 Hz), 7.8-7.95 (3H, m)

MASS (m/z): 1226.4 (M⁺+Na)

### EXAMPLE 3

To a solution of the starting compound (3) and anisole (0.677 ml) in dichloromethane (1.5 ml) was added dropwise trifluoroacetic acid (0.75 ml) with stirring under ice-cooling, and the mixture was stirred at room temperature for 2 hours. The reaction mixture was evaporated under reduced pressure to give the object compound (3) (196 mg) as a crude precipitate, that was used directly in the next step without purification.

### EXAMPLE 4

A solution of the starting compound (4) (100 mg) in DMF was treated with 1-[4-[5-[4-(7-methoxyheptyloxy)phenyl]-1,3,4-thiadiazol-2-yl]benzoyloxy]-1H-1,2,3-benzotriazole (49.1 mg) and N,N-diisopropylethylamine (0.0429 ml) and stirred at room temperature overnight. To the mixture was added 30% acetonitrile in water. The solution was subjected to column chromatography on ODS eluting with 50% acetonitrile in water. The fractions containing the object compound were collected and evaporated under reduced pressure to remove acetonitrile. The residue was lyophilized to give the object compound (4) (20 mg).

MASS (m/z): 1534.5 (M⁺+Na)

The following compound was obtained according to a similar manner to that of Example 4.

### EXAMPLE 5

MASS (m/z): 1475.3 (M⁺+Na)

### EXAMPLE 6

To a solution of the starting compound (6) (2.48 g) and cobalt(II)chloride hexahydrate (2.12 g) in a mixture of methanol (74 ml) and water (15 ml) was added sodium borohydride (1.69 g) with stirring at room temperature and the mixture was stirred at the same temperature for 2.5 hours. The reaction mixture was filtered through celite. To the filtrate was added 10% palladium on carbon (1.24 g), and hydrogen gas at atmosphere pressure for 2 hours. The reaction mixture was filtered through celite. The filtrate was adjusted to pH 8.3 with 1N-hydrochloric acid, evaporated under reduced pressure to remove methanol and was lyophilized. To the resulting residue and N,N-diisopropylethylamine (1.17 ml) in N,N-dimethylformamide (50 ml) was added 9-fluorenylmethyl chloroformate (865 mg) at room temperature, and the mixture was stirred for 4 hours. Water was added and the resulting precipitate collected and dried. To a solution of the residue and anisole (0.892 ml) in dichloromethane (20 ml) was added dropwise trifluoroacetic acid (10 ml) with stirring under ice-cooling, and the mixture was stirred at room temperature for 2.5 hours. The reaction mixture was evaporated under reduced pressure. Diisopropyl ether was added and the resulting precipitate was collected and dissolved in water. The solution was subjected to column chromatography on ODS eluting with 30% acetonitrile in water. The fractions containing the object compound were collected and evaporated under reduced pressure to remove acetonitrile. The residue was lyophilized to give the object compound (6) (1.053 g).

NMR (DMSO-d₆+D₂O, δ): 0.8-4.55 (37H, m), 4.6-4.85 (2H, m), 6.28 (1H, s), 6.5-6.8 (3H, m), 7.2-7.55 (4H, m), 7.68 (1H, d, J=7.2 Hz), 7.8-8.0 (3H, m)

MASS (m/z): 1089.4 (M⁺−H)

The following compounds [Example 7 to 8] were obtained according to a similar manner to that of Preparation 17.

### EXAMPLE 7

IR (KBr): 3354, 1666, 1633, 1512, 1448, 1254, 1142, 1084, 1068 cm⁻¹

NMR (DMSO-d₆+D₂O, δ): 0.96 (3H, d, J=6.8 Hz), 1.05 (5.9 Hz), 1.42 (9H, s), 1.5-2.6 (8H, m), 2.8-3.0 (1H, m), 3.1-3.3 (1H, m), 3.4-4.5 (1-5H, m), 4.63 (2H, s), 4.8-5.1 (6H, m), 6.6-6.8 (2H, m), 6.96 (1H, d, J=8.3 Hz), 7.2-7.5 (10H, m)

ESI MASS (Positive): 1185.4 (M+Na)⁺

Analysis Calculated for C₅₆H₇₄N₈O₁₉.2.5H₂O: C, 55.67; H, 6.59; N, 9.27 Found: C, 55.78; H, 6.41; N, 9.23

### EXAMPLE 8

IR (KBr): 3354, 1668, 1633, 1529, 1512, 1448, 1435, 1261, 1167, 1086 cm⁻¹

NMR (DMSO-d₆+D₂O, δ): 0.96 (3H, d, J=6.7 Hz), 1.04 (3H, d, J=5.6 Hz), 1.37 (9H, s), 1.5-2.6 (10H, m), 2.8-3.3 (4H, m), 3.4-4.5 (17H, m), 4.7-5.1 (6H, m), 6.6-7.0 (3H, m), 7.2-7.5 (10H, m)

ESI MASS (Positive): 1228.3 (M+Na)⁺

Analysis Calculated for C₅₈H₇₉N₉O₁₉.2H₂O: C, 56.07; H, 6.73; N, 10.15 Found: C, 56.36; H, 6.90; N, 10.20

The following compounds [Example 9 to 10] were obtained according to a similar manner to that of Preparation 20.

### EXAMPLE 9

NMR (DMSO-d₆+D₂O, δ): 0.96 (3H, d, J=6.7 Hz), 1.09 (3H, d, J=6.1 Hz), 1.43 (9H, s), 1.8-2.8 (9H, m), 3.1-4.6 (18H, m), 4.7-4.9 (2H, m), 6.5-6.8 (3H, m)

ESI MASS (Positive): 961.3 (M+Na)⁺

### EXAMPLE 10

NMR (DMSO-d₆+D₂O, δ): 0.96 (3H, d, J=6.7 Hz), 1.09 (3H, d, J=6.0 Hz), 1.7-2.6 (10H, m), 2.6-2.8 (1H, m), 3.0-4.5 (20H, m), 4.7-4.9 (2H, m), 6.5-6.8 (3H, m)

ESI MASS (Positive): 1004.3 (M+Na)⁺

### EXAMPLE 11

A mixture of the starting compound (11) (0.6 g) and 1-[4-[2-(4-pentyloxyphenyl)-1,3-thiazol-5-yl]benzoyloxy]-1H-1,2,3-benzotriazole (0.34 g) in N,N-dimethylformamide (12 ml) was stirred for 68 hours at room temperature. To the reaction mixture was added water and the resulting precipitates were collected by filtration. The precipitates were purified by column chromatography on silica gel eluting with a mixture of dichloromethane and methanol (8:1→5:1). The eluted fractions containing the desired product were collected and evaporated in vacuo to give the object compound (11) (0.67 g).

IR (KBr): 3350, 2935, 1637, 1520, 1439, 1255, 1153 cm⁻¹

NMR (DMSO-d₆+D₂O, δ): 0.8-1.0 (6H, m), 1.07 (3H, d, J=5.9 Hz), 1.3-1.5 (4H, m), 1.44 (9H, s), 1.6-2.6 (10H, m), 2.8-3.0 (1H, m), 3.1-3.3 (1H, m), 3.4-4.7 (19H, m), 4.7-5.0 (2H, m), 6.5-6.8 (3H, m), 7.09 (2H, d, J=8.9 Hz), 7.7-8.0 (6H, m), 8.36 (1H, s)

ESI MASS (Positive): 1310.3 (M+Na)⁺

Analysis Calculated for C₆₂H₈₁N₉O₁₉S.2H₂O: C, 56.22; H, 6.47; N, 9.52 Found: C, 56.42; H, 6.54; N, 9.53

The following compounds [Example 12 to 14] were obtained according to a similar manner to that of Example 11.

### EXAMPLE 12

IR (KBr): 3346, 3325, 2933, 1672, 1664, 1641, 1630, 1606, 1522, 1443, 1417, 1275, 1240, 1198, 1165, 1090 cm⁻¹

NMR (DMSO-d₆+D₂O, δ): 0.8-1.0 (6H, m), 1.09 (3H, d, J=5.9 Hz), 1.3-2.7 (28H, m), 1.38 (9H, s), 2.8-4.5 (21H, m), 4.7-5.0 (2H, m), 6.5-6.7 (3H, m), 7.09 (2H, d, J=9.2 Hz), 7.87 (2H, d, J=8.6 Hz), 8.0-8.1 (4H, m)

ESI MASS (Positive): 1448.4 (M+Na)⁺

### EXAMPLE 13

IR (KBr): 3442, 3346, 2935, 1682, 1664, 1641, 1624, 1605, 1529, 1516, 1466, 1433, 1273, 1228, 1196, 1090 cm⁻¹

NMR (DMSO-d₆+D₂O, δ): 0.96 (3H, d, J=6.7 Hz), 1.09 (3H, d, J=5.9 Hz), 1.3-2.6 (20H, m), 1.38 (9H, s), 2.8-4.5 (30H, m), 3.21 (3H, s), 4.7-5.0 (2H, m), 6.5-6.7 (3H, m), 7.09 (2H, d, J=8.8 Hz), 7.76 (2H, d, J=8.6 Hz), 7.9-8.0 (4H, m), 8.78 (1H, s)

ESI MASS (Positive): 1506.4 (M+Na)⁺

### EXAMPLE 14

IR (KBr): 3354, 3344, 2935, 1664, 1637, 1518, 1439, 1254, 1174 cm⁻¹

NMR (DMSO-d₆+D₂O, δ): 0.8-1.0 (6H, m), 1.08 (3H, d, J=6.1 Hz), 1.3-1.5 (13H, m), 1.6-2.6 (12H, m), 2.8-4.5 (23H, m), 4.7-5.0 (2H, m), 6.5-6.8 (3H, m), 7.08 (2H, d, J=8.8 Hz), 7.7-8.0 (6H, m), 8.38 (1H, s)

ESI MASS (Positive): 1353.3 (M+Na)⁺

Analysis Calculated for C₆₄H₈₆N₁₀O₁₉S.4H₂O: C, 54.77; H, 6.75; N, 9.98 Found: C, 54.67; H, 6.71; N, 9.77

### EXAMPLE 15

To a solution of the starting compound (15) (7.8 g) and cobalt(II)chloride hexahydrate (9 g) in a mixture of methanol (230 ml) and water (46 ml) was added sodium borohydride (7.12 g) with stirring at ambient temperature and the mixture was stirred at the same temperature for 1 hour. The reaction mixture was filtered through celite. The filtrate was adjusted to pH 7.5 with 1N-hydrochloric acid (105 ml). To the mixture was added 10% palladium on carbon (0.78 g), and hydrogen gas at atmospheric pressure for 1.5 hour. The reaction mixture was filtered through celite and evaporated under reduced pressure to remove methanol. To the resulting residue and N,N-diisopropylethylamine (6.56 ml) in N,N-dimethylformamide (78 ml) was added 9-fluorenylmethyl chloroformate (3.9 g) at ambient temperature, and the mixture was stirred for 2 hours. Ethyl acetate (780 ml) was added and the resulting precipitate collected, and the powder was dissolved in a mixture of acetonitrile (300 ml) and water (450 ml). The solution was subjected to column chromatography on ODS (Daiso-gel, SP-120-40/60-ODS-B (Trademark: prepared by Daiso Co., Ltd.)) (500 ml) eluting with 60% acetonitrile in water. The fractions containing the object compound were collected and evaporated under reduced pressure to remove acetonitrile. The residue was lyophilized to give the object compound (15) (2.2 g).

IR (KBr): 3351.7, 1664.3, 1635.3, 1257.4 cm⁻¹

NMR (DMSO-d₆+D₂O, δ): 0.88-1.12 (6H, m), 1.34 (9H, s), 1.55-4.81 (39H, m), 6.28-7.90 (19H, m)

ESI MASS (Positive)(m/z): 1420.4 (M+Na)⁺

### EXAMPLE 16

To a solution of the starting compound (16) (3.37 g) and cobalt(II)chloride hexahydrate (2.7 g) in a mixture of methanol (100 ml) and water (20 ml) was added sodium borohydride (2.15 g) with stirring at ambient temperature and the mixture was stirred at the same temperature for 1.5 hour. The reaction mixture was filtered through celite. To the filtrate was added 10% palladium on carbon (0.337 g), and hydrogen gas at atmospheric pressure for 3 hours. The reaction mixture was filtered through celite and evaporated under reduced pressure to remove methanol. The residue was subjected to column chromatography on ODS (Daiso-gel, SP-120-40/60-ODS-B (Trademark: prepared by Daiso Co., Ltd.)) (60 ml) eluting with 50% acetonitrile in water. The fractions containing the object compound were collected and evaporated under reduced pressure to remove acetonitrile. The residue was lyophilized to give the object compound (16) (1.58 g).

IR (KBr): 3359.4, 1668.1, 1650.8, 1631.5, 1521.6 cm⁻¹

NMR (DMSO-d₆+D₂O, δ): 0.95 (3H, d, J=6.5 Hz), 1.06-1.09 (3H, m), 1.35 (9H, s), 1.46-4.80 (35H, m), 6.55-6.90 (3H, m)

ESI MASS (Positive)(m/z): 990.4 (M+Na)⁺, 968.5 (M+H)⁺

### EXAMPLE 17

A solution of the starting compound (17) (1.55 g) and N,N-diisopropylethylamine (1.39 ml) in DMF (15.5 ml) was added 9-fluorenylmethyl chloroformate (994 mg) and stirred at ambient temperature for 2.5 hours. The reaction mixture was concentrated in vacuo. The resulting residue was chromatographed on a column of silica gel (45 g) eluting with dichloromethane/methanol (5:1) to give the object compound (17) (1.25 g).

IR (KBr): 3367.1, 1699.0, 1668.1, 1633.4, 1515.8, 1255.4 cm⁻¹ NMR (DMSO-d₆+D₂O, δ): 0.89 (3H, d, J=6.5 Hz), 1.07-1.10 (3H, m), 1.20-1.34 (11H, m), 1.46-4.80 (39H, m), 6.55-6.90 (3H, m), 7.28-7.89 (16H, m)

ESI MASS (Negative)(m/z): 1410.6 (M+H)⁺

### EXAMPLE 18

A solution of the starting compound (18) (1.25 g) in dichloromethane (19 ml) was added dropwise trifluoroacetic acid (2.02 g) in dichloromethane (2 ml) with stirring under ice-cooling, and the mixture was stirred at ambient temperature overnight. The reaction mixture was concentrated in vacuo to give the object compound (18) (1.81 g) as a crude syrup, that was used directory in the next step without purification.

ESI MASS (Positive)(m/z): 1335.3 (M+Na)⁺, 1312.5 (M+H)⁺

### EXAMPLE 19

To a solution of the starting compound (19) (3.7 g) and triethylsilane (3.35 ml) in dichloromethane (56 ml) was added dropwise trifluoroacetic acid (6.05 ml) with stirring under ice-cooling, and the mixture was stirred at ambient temperature for 1.5 hours. The reaction mixture was added a solution of sodium hydrogen carbonate (8 g) in water (300 ml). The resulting precipitate was collected, the precipitate was dissolved in a mixture of 50% acetonitrile in water and the solution was subjected to column chromatography on ODS (Daiso-gel, SP-120-40/60-ODS-B (Trademark: prepared by Daiso Co., Ltd.)) (60 ml) eluting with 15% acetonitrile in water. The fractions containing the object compound were collected and evaporated under reduced pressure to remove acetonitrile. The residue was lyophilized to give the object compound (19) (0.99 g).

IR (KBr): 3365.2, 1668.1, 1650.8, 1633.4, 1265.1 cm⁻¹

NMR (DMSO-d₆+D₂O, δ): 0.89 (3H, d, J=6.6 Hz), 1.09 (3H, d, J=5.9 Hz), 1.35 (9H, s), 1.46-5.50 (47H, m), 6.54-6.70 (3H, m), 7.21-7.90 (16H, m)

ESI MASS (Positive)(m/z): 1334.4 (M+Na)⁺, 1312.4 (M+H)⁺

The following compound was obtained according to a similar manner to that of Example 19.

### EXAMPLE 20

IR (KBr): 3344.0, 1664.3, 1635.3, 1263.1 cm⁻¹

NMR (DMSO-d₆+D₂O, δ): 0.88-1.10 (6H, m), 1.40-4.80 (39H, m), 6.28-7.90 (19H, m)

ESI MASS (Positive)(m/z): 1320.4 (M+Na)⁺, 1298.4 (M+H)⁺

### EXAMPLE 21

A solution of Starting Compound (21) (0.4 g) in DMF (4 ml) was treated with 1-[4-[5-[4-[4-(4-methylcyclohexyl)-1-piperazinyl]phenyl]-1,3,4-thiadiazol-2-yl]benzoyloxy]-1H-1,2,3-benzotriazole (326 mg) and N,N-diisopropylethylamine (0.18 ml) and stirred at ambient temperature overnight. To the reaction mixture was added piperidine (0.28 ml) and stirred at ambient temperature for 2 hours. To the reaction mixture was added ethyl acetate (40 ml). The resulting precipitate was collected, the precipitate was dissolved in a mixture of 15% acetonitrile in water (30 ml) and 1N—HCl (1.4 ml) and the solution was subjected to column chromatography on ODS (Daiso-gel, SP-120-40/60-ODS-B (Trademark: prepared by Daiso Co., Ltd.)) (60 ml) eluting with 15% acetonitrile in water. The fractions containing the object compound were collected and evaporated under reduced pressure to remove acetonitrile. The residue was lyophilized to give the object compound (21) (59.3 mg).

IR (KBr): 3361.3, 1645.0, 1631.5, 1523.5, 1442.5 cm⁻¹ NMR (DMSO-d₆+D₂O, δ): 0.97 (3H, d, J=7.0 Hz), 1.08 (3H, d, J=5.9 Hz), 1.46-4.80 (58H, m), 6.59-6.74 (3H, m), 7.17-8.94 (8H, m)

ESI MASS (Positive)(m/z): 1334.3 (M+Na)⁺, 1312.5 (M+H)⁺

Elemental Analysis Calcd. for C₆₄H₉₂Cl₃N₁₃O₁₅S.9H₂O: C, 47.67; H, 6.44; N, 10.36 Found: C, 47.53; H, 6.97; N, 10.94

The following compounds [Example 22 to 54] were obtained according to a similar manner to that of Example 21.

### EXAMPLE 22

NMR (DMSO-d₆+D₂O, δ): 0.96 (3H, t, J=6.7 Hz), 1.08 (3H, t, J=6.7 Hz), 1.26-4.80 (44H, m), 6.64-8.13 (15H, m)

ESI MASS (Positive)(m/z): 1363.2 (M+Na)⁺

Elemental Analysis Calcd. for C₆₄H₈₄Cl₄N₁₂O₁₆S.13H₂O: C, 45.61; H, 6.58; N, 9.97 Found: C, 45.75; H, 6.24; N, 10.12

### EXAMPLE 23

NMR (DMSO-d₆+D₂O, δ): 0.96 (3H, t, J=6.8 Hz), 1.08 (3H, t, J=5.9 Hz), 1.25-4.80 (55H, m), 6.64-8.13 (11H, m)

ESI MASS (Positive)(m/z): 1340.4 (M+Na)⁺ (free)

Elemental Analysis Calcd. for C₆₃H₉₀Cl₂N₁₂O₁₇S.12H₂O: C, 47.10; H, 7.15; N, 10.46 Found: C, 46.90; H, 6.88; N, 10.20

### EXAMPLE 24

IR (KBr): 3356, 1635, 1529, 1520, 1443, 1275, 1250 cm⁻¹

NMR (DMSO-d₆+D₂O, δ): 0.97 (3H, d, J=6.8 Hz), 1.09 (3H, d, J=5.8 Hz), 1.5-2.7 (13H, m), 2.8-4.6 (22H, m), 3.33 (3H, s), 4.7-4.8 (2H, m), 6.6-6.8 (3H, m), 7.09 (2H, d, J=8.8 Hz), 7.74 (2H, d, J=8.8 Hz), 7.88 (2H, d, J=8.5 Hz), 8.0-8.2 (6H, m)

ESI MASS (Positive): 1291.4 (M+Na)⁺

Elemental Analysis Calcd. for C₆₁H₇₉Cl₂N₁₁O₁₇S.8H₂O: C, 49.32; H, 6.45; N, 10.37 Found: C, 49.19; H, 6.08; N, 10.28

### EXAMPLE 25

IR (KBr): 3361, 1635, 1529, 1518, 1443, 1275, 1252 cm⁻¹

NMR (DMSO-d₆+D₂O, δ): 0.97 (3H, d, J=6.7 Hz), 1.0-1.2 (6H, m), 1.5-2.7 (13H, m), 2.8-4.6 (24H, m), 4.7-4.9 (2H, m), 6.6-6.8 (3H, m), 7.10 (2H, d, J=8.8 Hz), 7.74 (2H, d, J=8.8 Hz), 7.88 (2H, d, J=8.5 Hz), 8.0-8.2 (6H, m)

ESI MASS (Positive): 1304.3 (M+Na)⁺

Elemental Analysis Calcd. for C₆₂H₈₁Cl₂N₁₁O₁₇S.9.5H₂O: C, 48.78; H, 6.60; N, 10.09 Found: C, 48.66; H, 6.12; N, 10.12

### EXAMPLE 26

IR (KBr): 2931, 1659, 1628, 1606, 1529, 1518, 1444, 1257 cm⁻¹

NMR (DMSO-d₆+D₂O, δ): 0.97 (3H, d, J=6.9 Hz), 1.06 (3H, d, J=6.1 Hz), 1.2-2.8 (20H, m), 2.8-4.9 (32H, m), 6.5-6.7 (1H, m), 6.8-6.9 (2H, m), 7.1-7.2 (2H, m), 7.9-8.2 (6H, m)

ESI MASS: 1299.5 (M+Na)+(Positive), (free (M)=1276.46)

### EXAMPLE 27

IR (KBr): 1659, 1628, 1606, 1529, 1518, 1444, 1257 cm⁻¹

NMR (DMSO-d₆+D₂O, δ): 0.97 (3H, d, J=6.7 Hz), 1.08 (3H, d, J=5.8 Hz), 1.3-2.7 (23H, m), 2.8-4.6 (22H, m), 3.22 (3H, s), 4.7-4.9 (2H, m), 6.5-6.8 (3H, m), 7.14 (2H, d, J=8.9 Hz), 7.98 (2H, d, J=8.7 Hz), 8.0-8.2 (4H, m)

ESI MASS (Positive): 1284.4 (M+Na)⁺

Elemental Analysis Calcd. for C₆₀H₈₅Cl₂N₁₁O₁₇S.7H₂O: C, 49.31; H, 6.83; N, 10.54 Found: C, 49.45; H, 6.68; N, 10.48

### EXAMPLE 28

NMR (DMSO-d₆+D₂O, δ): 0.96 (3H, d, J=6.8 Hz), 1.08 (3H, d, J=5.9 Hz), 1.33-4.80 (55H, m), 6.59-6.74 (3H, m), 7.14 (2H, d; J=8.9 Hz), 7.96-8.15 (6H, m)

ESI MASS (Positive)(m/z): 1298.4 (M+Na)⁺, 1276.5 (M+H)⁺ 1256.3 (M⁻−H) (Negative)

Elemental Analysis Calcd. for C₆₁H₈₇Cl₂N₁₁O₁₇S.7H₂O: C, 49.66; H, 6.90; N, 10.44 Found: C, 49.45; H, 6.59; N, 10.19

### EXAMPLE 29

IR (KBr): 3354, 1674, 1635, 1518, 1444, 1201, 1180, 1136 cm¹ t

NMR (DMSO-d₆+D₂O, δ): 0.97 (3H, ,d, J=6.7 Hz), 1.09 (3H, d, J=5.9 Hz), 1.2-2.6 (27H, m), 2.8-4.5 (22H, m), 3.21 (3H, s), 4.7-4.9 (2H, m), 6.5-6.8 (3H, m), 7.14 (2H, d, J=8.9 Hz), 7.9-8.2 (6H, m)

ESI MASS (Positive): 1312.4 (M+Na)⁺

Elemental Analysis Calcd. for C₆₆H₈₉F₆N₁₁O₂₁S.7H₂O: C, 48.20; H, 6.31; N, 9.37 Found: C, 47.91; H, 5.99; N, 9.20

### EXAMPLE 30

IR (KBr): 3373, 1674, 1635, 1518, 1439, 1201, 1136 cm⁻¹

NMR (DMSO-d₆+D₂O, δ): 0.8-1.2 (9H, m), 1.5-2.7 (28H, m), 2.8-4.9 (25H, m), 6.5-6.8 (3H, m), 7.1-8.9 (7H, m)

ESI MASS (Positive): 1335.4 (M+Na)⁺

Elemental Analysis Calcd. for C₇₁H₉₂F₁₂N₁₄O₂₃S.7H₂O: C 44.98, H 5.64, N 10.34 Found: C, 45.06; H, 5.47; N, 10.36

### EXAMPLE 31

IR (KBr): 3352, 1674, 1635, 1529, 1518, 1444, 1201, 1136 cm⁻¹

NMR (DMSO-d₆+D₂O, δ): 0.9-2.7 (29H, m), 0.97 (3H, d, J=6.8 Hz), 1.09 (3H, d, J=5.8 Hz), 2.8-4.6 (24H, m), 4.7-4.9 (2H, m), 6.5-6.8 (3H, m), 7.09 (2H, d, J=9.0 Hz), 7.85 (2H, d, J=8.8 Hz), 7.9-8.2 (4H, m)

ESI MASS (Positive): 1335.4 (M+Na)⁺

Elemental Analysis Calcd. for C₇₀H₉₁F₉N₁₂O₂₂S.7H₂O: C, 47.19; H, 5.94; N, 9.43 Found: C, 47.10; H, 5.64; N, 9.33

### EXAMPLE 32

IR (KBr): 3361.3, 1645.0, 1631.5, 1521.6 cm⁻¹

NMR (DMSO-d₆+D₂O, δ): 0.96 (3H, d, J=6.8 Hz), 1.08 (3H, d, J=5.9 Hz), 1.33-4.80 (63H, m), 6.59-6.74 (3H, m), 7.09-8.80 (9H, m)

ESI MASS (Positive)(m/z): 1392.5 (M+Na)⁺, 1370.6 (M+H)⁺

Elemental Analysis Calcd. for C₆₆H₉₃Cl₂N₁₃O₁₇S.11H₂O: C, 48.29; H, 7.06; N, 11.09 Found: C, 48.39; H, 6.61; N, 11.00

### EXAMPLE 33

NMR (DMSO-d₆+D₂O, δ): 0.96 (3H, d, J=6.8 Hz), 1.07 (3H, d, J=5.9 Hz), 1.35-4.98 (69H, m), 6.59-8.52 (11H, m)

ESI MASS (Positive)(m/z): 1348.5 (M+Na)⁺

### EXAMPLE 34

IR (KBr): 1674, 1635, 1529, 1444, 1201, 1136 cm⁻¹

NMR (DMSO-d₆+D₂O, δ): 0.97 (3H, d, J=6.7 Hz), 1.08 (3H, d, J=5.5 Hz), 1.2-2.7 (36H, m), 2.8-4.6 (23H, m), 3.10 (3H, s), 4.7-4.9 (2H, m), 6.6-6.8 (3H, m), 7.14 (2H, d, J=8.6 Hz), 7.6-7.8 (4H, m), 7.93 (2H d, J=8.4 Hz)

ESI MASS (Positive): 1312.6 (M+H)⁺

Elemental Analysis Calcd. for C₇₅H₁₀₁F₁₂N₁₁O₂₄.7H₂O: C, 47.54; H, 6.12; N, 8.13 Found: C, 47.75; H, 6.10; N, 8.20

### EXAMPLE 35

IR (KBr): 1659, 1635, 1605, 1529, 1518, 1444, 1417 cm⁻¹

NMR (DMSO-d₆+D₂O, δ): 0.8-2.7 (30H, m), 0.97 (3H, d, J=6.8 Hz), 1.08 (3H, d, J=5.9 Hz), 2.8-4.6 (22H, m), 3.10 (3H, s), 4.7-4.9 (2H, m), 6.5-6.8 (3H, m), 7.08 (2H, d, J=9.1 Hz), 7.84 (2H, d, J=8.8 Hz), 8.0-8.2 (4H, m)

ESI MASS (Positive): 1349.3 (M+Na)⁺

Elemental Analysis Calcd. for C₆₅H₉₃Cl₃N₁₂O₁₆S.7H₂O: C, 49.95; H, 6.90; N, 10.75 Found: C, 50.17; H, 6.75; N, 10.62

### EXAMPLE 36

IR (KBr): 3350, 2935, 1635, 1606, 1514, 1443, 1398, 1257, 1234 cm⁻¹

NMR (DMSO-d₆+D₂O, δ): 0.8-2.8 (19H, m), 2.8-4.9 (41H, m), 6.6-6.7 (1H, m), 6.72 (2H, d, J=8.1 Hz), 6.97 (2H, d, J=8.9 Hz), 7.20 (2H, d, J=8.9 Hz), 7.3-7.5 (2H, m), 7.9-8.0 (2H, m), 8.0-8.2 (4H, m)

ESI MASS: 1416.4 (M+Na)⁺ (Positive), (free (M)=1394.59)

Elemental Analysis Calcd. for C₆₈H₉₁N₁₃O₁₇S.3HCl.9H₂O: C, 49.02; H, 6.78; N, 10.93 Found: C, 49.02; H, 6.51; N, 10.92

### EXAMPLE 37

IR (KBr): 3352, 1674, 1635, 1529, 1518, 1444, 1201, 1134 cm⁻¹

NMR (DMSO-d₆+D₂O, δ): 0.97 (3H, d, J=6.8 Hz), 1.09 (3H, d, J=5.7 Hz), 1.5-2.7 (19H, m), 2.8-4.5 (23H, m), 4.57 (2H, s), 4.7-4.9 (2H, m), 6.5-6.8 (3H, m), 7.11 (2H, d, J=9.0 Hz), 7.2-7.4 (5H, m), 7.85 (2H, d, J=8.8 Hz), 8.0-8.2 (4H, m)

ESI MASS (Positive): 1343.3 (M+Na)⁺

Elemental Analysis Calcd. for C₇₁H₈₇F₉N₁₂O₂₂S.7H₂O: C, 47.65; H, 5.69; N, 9.39 Found: C, 47.77; H, 5.46; N, 9.36

### EXAMPLE 38

IR (KBr): 3356, 1674, 1635, 1529, 1518, 1444, 1201, 1136 cm⁻¹

NMR (DMSO-d₆+D₂O, δ): 0.97 (3H, d, J=6.7 Hz), 1.09 (3H, d, J=5.9 Hzl), 1.6-2.7 (19H, m), 2.7-4.5 (23H, m), 4.7-4.9 (2H, m), 6.5-6.8 (3H, m), 7.0-8.2 (13H, m)

ESI MASS (Positive): 1313.4 (M+Na)⁺

### EXAMPLE 39

IR (KBr): 1635, 1605, 1520, 1443, 1417, 1196, 1068 cm⁻¹

NMR (DMSO-d₆+D₂O, δ): 0.97 (3H, d, J=6.7 Hz), 1.09 (3H, d, J=5.9 Hz), 1.5-2.7 (19H, m), 2.8-4.5 (22H, m), 2.94 (3H, s), 4.7-4.9 (2H, m), 6.5-6.8 (3H, m), 7.15 (2H, d, J=8.9 Hz), 7.46 (4H, s), 7.87 (2H, d, J=8.8 Hz), 8.0-8.2 (4H, m)

ESI MASS (Positive): 1377.3 (M+Na)⁺

Elemental Analysis Calcd. for C₆₅H₈₆Cl₄N₁₂O₁₆S.4H₂O: C, 50.78; H, 6.16; N, 10.93 Found: C, 50.54; H, 6.24; N, 10.82

### EXAMPLE 40

IR (KBr): 1674, 1635, 1529, 1518, 1444, 1201, 1134 cm⁻¹

NMR (DMSO-d₆+D₂O, δ): 0.97 (3H, d, J=6.7 Hz), 1.09 (3H, d, J=5.8 Hz), 1.2-2.7 (24H, m), 2.7-4.5 (28H, m), 3.21 (3H, s), 4.7-4.9 (2H, m), 6.6-6.8 (3H, m), 7.08 (2H, d, J=9.0 Hz), 7.85 (2H, d, J=8.8 Hz), 8.0-8.2 (4H, m)

ESI MASS (Positive): 1353.4 (M+Na)⁺

Elemental Analysis Calcd. for C₇₀H₉₃F₉N₁₂O₂₃S.6H₂O: C, 47.19; H, 5.94; N, 9.43 Found: C, 47.19; H, 5.89; N, 9.45

### EXAMPLE 41

IR (KBr): 3349.7, 2950.6, 1658.5, 1633.4, 1440.6, 1070.3 cm⁻¹

NMR (DMSO-d₆+D₂O, δ): 0.96 (3H, t, J=6.7 Hz), 1.08 (3H, t, J=5.7 Hz), 1.51-4.80 (58H, m), 6.59-8.52 (11H, m)

ESI MASS (Positive)(m/z): 1335.6 (M+Na)⁺ (free)

Elemental Analysis Calcd. for C₆₄H₉₁Cl₃N₁₂O₁₆S.11H₂O: C, 47.42; H, 7.03; N, 10.37 Found: C, 47.16; H, 6.94; N, 10.17

### EXAMPLE 42

IR (KBr): 3354, 3327, 2935, 1635, 1527, 1443, 1273, 1246, 1176, 1084 cm⁻¹

NMR (DMSO-d₆+D₂O, δ): 0.97 (3H, d, J=6.7 Hz), 1.09 (3H, d, J=5.9 Hz), 1.2-2.7 (19H, m), 2.8-4.9 (27H, m), 6.62 (1H, d, J=7.8 Hz), 6.7-6.8 (2H, m), 7.08 (2H, d, J=8.7 Hz), 7.72 (2H, d, J=8.8 Hz), 7.87 (2H, d, J=8.4 Hz), 8.0-8.2 (6H, m)

ESI MASS: 1329.4 (M+Na)⁺ (Positive) (free (M)=1306.49)

Elemental Analysis Calcd. for C₆₅H₈₃N₁₁O₁₆S.2HCl.7H₂O: C, 51.86; H, 6.63; N, 10.23 Found: C, 52.07; H, 6.50; N, 10.24

### EXAMPLE 43

IR (KBr): 3334, 2935, 1635, 1529, 1518, 1441, 1277, 1082 cm⁻¹

NMR (DMSO-d₆+D₂O, δ): 0.97 (3H, d, J=6.7 Hz), 1.09 (3H, d, J=6.0 Hz), 1.5-2.7 (9H, m), 2.8-4.9 (35H, m), 6.5-6.7 (1H, m), 6.7-6.8 (2H, m), 7.49 (2H, d, J=8.5 Hz), 7.78 (2H, d, J=8.2 Hz), 7.93 (2H, d, J=8.4 Hz), 8.0-8.3 (6H, m)

ESI MASS: 1318.5 (M+Na)⁺ (Positive) (free (M)=1296.45)

Elemental Analysis Calcd. for C₆₃H₈₁N₁₁O₁₇S.2HCl.7H₂O: C, 50.60; H, 6.54; N, 10.30 Found: C, 50.66; H, 6.29; N, 10.35

### EXAMPLE 44

IR (KBr): 3367, 3348, 2974, 2935, 1635, 1529, 1518, 1441, 1277, 1086 cm⁻¹

NMR (DMSO-d₆+D₂O, δ): 0.97 (3H, d, J=6.8 Hz), 1.09 (3H, d, J=5.6 Hz), 1.19 (3H, t, J=7.0 Hz), 1.6-2.8 (9H, m), 2.8-4.9 (30H, m), 6.6-6.7 (1H, m), 6.7-6.8 (2H, m), 7.55 (2H, d, J=6.6 Hz), 7.78 (2H, d, J=8.1 Hz), 7.93 (2H, d, J=8.4 Hz), 8.0-8.3 (6H, m)

ESI MASS: 1288.4 (M+Na)⁺ (Positive)(free (M)=1266.42)

Elemental Analysis Calcd. for C₆₂H₇₉N₁₁O₁₆S.2HCl.7H₂O: C, 50.81; H, 6.53; N, 10.51 Found: C, 51.01; H, 6.38; N, 10.51

### EXAMPLE 45

IR (KBr): 3458, 3371, 3311, 3277, 3250, 2927, 1635, 1529, 1443 cm⁻¹

NMR (DMSO-d₆+D₂O, δ): 0.97 (3H, d, J=6.6 Hz), 1.0-1.2 (6H, m), 1.5-2.8 (11H, m), 2.8-4.9 (32H, m), 6.5-6.7 (1H, m), 6.7-6.8 (2H, m), 7.08 (2H, d, J=9.1 Hz), 7.74 (2H, d, J=8.7 Hz), 7.87 (2H, d, J=8.2 Hz), 8.0-8.2 (6H, m)

ESI MASS: 1333.4 (M+Na)⁺ (Positive)(free (M)=1310.47)

Elemental Analysis Calcd. for C₆₄H₈₃N₁₁O₁₇S.2HCl.6H₂O: C, 51.54; H, 6.56; N, 10.33 Found: C, 51.39; H, 6.39; N, 10.28

### EXAMPLE 46

IR (KBr): 3496, 3456, 3325, 2976, 2933, 1635, 1527, 1443, 1252 cm⁻¹

NMR (DMSO-d₆+D₂O, δ): 0.97 (3H, d, J=6.8 Hz), 1.0-1.2 (6H, m), 1.5-2.7 (9H, m), 2.8-4.9 (32H, m), 6.6-6.8 (3H, m), 7.10 (2H, d, J=8.9 Hz), 7.74 (2H, d, J=8.8 Hz), 7.88 (2H, d, J=80.5 Hz), 8.0-8.2 (6H, m)

ESI MASS: 1318.4 (M+Na)⁺ (Positive)(free (M)=1296.45)

Elemental Analysis Calcd. for C₆₃H₈₁N₁₁O₁₇S.2HCl.6H₂O: C, 51.21; H, 6.48; N, 10.43 Found: C, 51.18; H, 6.32; N, 10.44

### EXAMPLE 47

IR (KBr): 3456, 3340, 2931, 1635, 1529, 1443, 1250 cm⁻¹

NMR (DMSO-d₆+D₂O, δ): 0.9-1.1 (6H, m), 1.5-2.6 (9H, m), 2.8-4.9 (33H, m), 6.6-6.8 (3H, m), 7.0-7.2 (2H, m), 7.7-7.8 (2H, m), 7.8-7.9 (2H, m), 8.0-8.2 (6H,m)

ESI MASS: 1304.3 (M+Na)⁺ (Positive)(free (M)=1282.42)

Elemental Analysis Calcd. for C₆₂H₇₉N₁₁O₁₇S.2HC17H₂O: C, 50.27; H, 6.46; N, 10.40 Found: C, 50.51; H, 6.19; N, 10.46

### EXAMPLE 48

IR (KBr): 3475, 3456, 3277, 2935, 1635, 1529, 1441, 1273, 1250 cm⁻¹

NMR (CDCl₃, δ): 0.8-1.2 (9H, m), 1.3-2.8 (13H, m), 2.8-4.9 (28H, m), 6.6-6.7 (1H, m), 6.7-6.8 (2H, m), 7.08 (2H, d, J=8.8 Hz), 7.74 (2H, d, J=8.8 Hz), 7.87 (2H, d, J=8.7 Hz), 8.0-8.2 (6H, m)

ESI MASS: 1302.3 (M+Na)+(Positive)(free (M)=1280.45)

Elemental Analysis Calcd. for C₆₃H₈₁N₁₁O₁₆S.2HCl.7H₂O: C, 51.14; H, 6.61; N, 10.41 Found: C, 55.11; H, 6.38; N, 10.39

### EXAMPLE 49

IR (KBr): 3421, 3379, 3350, 3250, 1635, 1529, 1443, 1273, 1248 cm⁻¹

NMR (CDCl₃, δ): 0.97 (3H, d, J=6.8 Hz), 1.09 (3H, d, J=5.8 Hz), 1.37 (3H, t, J=6.8 Hz), 1.5-2.7 (9H, m), 2.7-4.9 (28H, m), 6.6-6.7 (1H, m), 6.7-6.8 (2H, m), 7.07 (2H, d, J=8.7 Hz), 7.74 (2H, d, J=8.7 Hz), 7.88 (2H, d, J=8.4 Hz), 8.0-8.2 (6H, m)

ESI MASS: 1275.4 (M+Na)⁺ (Positive)(free (M)=1252.39)

Elemental Analysis Calcd. for C₆₁H₇₇N₁₁O₁₆S.2HC₁₆H₂O: C, 51.11; H, 6.40; N, 10.75 Found: C, 51.07; H, 6.28; N, 10.72

### EXAMPLE 50

IR (KBr): 1664, 1635, 1606, 1444 cm⁻¹

NMR (DMSO-d₆+D₂O, δ): 0.8-2.7 (28H, m), 0.96 (3H, d, J=6.7 Hz), 1.08 (3H, d, J=5.7 Hz), 2.8-4.6 (22H, m), 3.10 (3H, s), 4.7-4.9 (2H, m), 6.6-6.8 (3H, m), 7.08 (2H, d, J=8.6 Hz), 7.8-8.3 (6H, m)

ESI MASS (Positive): 1335.5 (M+Na)⁺

Elemental Analysis Calcd. for C₆₄H₉₁Cl₃N₁₂O₁₆S.7H₂O: C, 49.62; H, 6.83; N, 10.85 Found: C, 49.44; H, 6.65; N, 10.78

### EXAMPLE 51

IR (KBr): 3373, 3331, 1635, 1529, 1520, 1443, 1275, 1250 cm⁻¹

NMR (DMSO-d₆+D₂O, δ): 0.97 (3H, d, J=6.8 Hz), 1.09 (3H, d, J=6.0 Hz), 1.5-2.7 (15H, m), 2.8-4.6 (22H, m), 3.27 (3H, s), 4.7-4.8 (2H, m), 6.6-6.8 (3H, m), 7.08 (2H, d, J=8.8 Hz), 7.73 (2H, d, J=8.8 Hz), 7.87 (2H, d, J=8.5 Hz), 8.0-8.2 (6H, m)

ESI MASS (Positive): 1304.4 (M+Na)⁺

Elemental Analysis Calcd. for C₆₂H₈₁Cl₂N₁₁O₁₇S.8H₂O: C, 49.66; H, 6.52; N, 10.28 Found: C, 49.39; H, 6.48; N, 10.13

### EXAMPLE 52

IR (KBr): 3344, 1632, 1518, 1441, 1275 cm⁻¹

NMR (DMSO-d₆+D₂O, δ): 0.9-2.7 (27H, m), 0.96 (3H, d, J=6.7 Hz), 1.07 (3H, d, J=5.7 Hz), 2.8-4.5 (23H, m), 4.7-4.9 (2H, m), 6.6-6.8 (3H, m), 7.12 (2H, d, J=8.8 Hz), 7.6-7.8 (4H, m), 7.96 (2H, d, J=8.3 Hz)

ESI MASS (Positive): 1222.5 (M+Na)⁺

Elemental Analysis Calcd. for C₆₀H₈₉Cl₄N₁₁O₁₅S.17H₂O: C, 43.61; H, 7.50; N, 9.32 Found: C, 43.72; H, 7.21; N, 9.32

### EXAMPLE 53

IR (KBr): 3352, 1635, 1529, 1518, 1444 cm⁻¹

NMR (DMSO-d₆+D₂O, δ): 0.97 (3H, d, J=6.7 Hz), 1.08 (3H, d, J=5.5 Hz), 1.2-2.7 (27H, m), 2.8-4.6 (27H, m), 3.20 (3H, s), 4.7-4.9 (2H, m), 6.4-6.8 (3H, m), 7.10 (2H, d, J=8.9 Hz), 7.86 (2H, d, J=8.6 Hz), 7.9-8.2 (4H, m)

ESI MASS (Positive): 1367.3 (M+Na)⁺

Elemental Analysis Calcd. for C₆₅H₉₅Cl₃N₁₂O₁₇S.9H₂O: C, 48.28; H, 7.04; N, 10.39 Found: C, 48.12; H, 6.83; N, 10.68

### EXAMPLE 54

IR (KBr): 3347.8, 2946.7, 1685.5, 1627.6, 1444.4, 1070.3 cm⁻¹

NMR (DMSO-d₆+D₂O, δ): 0.96 (3H, t, J=6.8 Hz), 1.08 (3H, t, J=5.9 Hz), 1.32-4.80 (56H, m), 6.64-8.55 (11H, m)

ESI MASS (Positive)(m/z): 1321.4 (M+Na)⁺ (free)

Elemental Analysis Calcd. for C₆₃H₈₉Cl₃N₁₂O₁₆S.14H₂O: C, 45.55; H, 7.10; N, 10.12 Found: C, 45.77; H, 6.71; N, 10.27

### EXAMPLE 55

A solution of the starting compound (55) (0.1 g) in DMF (1 ml) was treated with 1-[4-[5-[4-(7-methoxyheptyloxy)-phenyl]-1,3,4-thiadiazol-2-yl]benzoyloxy]-1H-1,2,3-benzotriazole (54.4 mg) and N,N-diisopropylethylamine (19.9 mg) and stirred at ambient temperature overnight. To the reaction mixture was added piperizine (65.6 mg) and stirred at ambient temperature for 2 hours. To the reaction mixture was added ethyl acetate (10 ml). The resulting precipitate was collected, the precipitate was dissolved in a mixture of 15% acetonitrile in water (30 ml) and trifluoroacetic acid (18 mg) and the solution was subjected to column chromatography on ODS (Daiso-gel, SP-120-40/60-ODS-B (Trademark: prepared by Daiso Co., Ltd.)) (60 ml) eluting with 40% acetonitrile in (0.1% trifluoroacetic acid) water. The fractions containing the object compound were collected and evaporated under reduced pressure to remove acetonitrile. The residue was lyophilized to give the object compound (55) (70.0 mg).

IR (KBr): 3409.5, 1673.9, 1635.3, 1201.4 cm⁻¹

NMR (DMSO-d₆+D₂O, δ): 0.96 (3H, d, J=6.9 Hz), 1.09 (3H, d, J=5.7 Hz), 1.35-4.83 (50H, m), 6.64-8.99 (11H, m)

ESI MASS (Positive)(m/z): 1284.4 (M+Na)⁺, 1262.3 (M+H)⁺ (free)

Elemental Analysis Calcd. for C₆₄H₈₅F₆N₁₁O₂₁S.8H₂O: C, 47.03; H, 6.23; N, 9.43 Found: C, 46.93; H, 5.96; N, 9.26 The following compounds [Example 56 to 71] were obtained according to a similar manner to that of Example 55.

### EXAMPLE 56

IR (KBr): 3400, 1672.0, 1635.3, 1199.5, 1182.2 cm⁻¹

NMR (DMSO-d₆+D₂O, δ): 0.96 (3H, d, J=6.7 Hz), 1.09 (3H, d, J=5.7 Hz), 1.39-4.81 (48H, m), 6.68-8.51 (11H, m)

ESI MASS (Positive)(m/z): 1270.2 (M⁺Na)+(free)

### EXAMPLE 57

IR (KBr): 3400, 1672.0, 1664.3, 1201.4 cm⁻¹

NMR (DMSO-d₆+D₂O, δ): 0.96 (3H, d, J=6.7 Hz), 1.09 (3H, d, J=5.8 Hz), 1.30-4.81 (52H, m), 6.64-8.49 (11H, m)

Elemental Analysis Calcd. for C₆₅H₈₇F₆N₁₁O₂₁S.5.5H₂O: C, 48.69; H, 6.16; N, 9.61 Found: C, 48.68; H, 5.96; N, 9.34

### EXAMPLE 58

IR (KBr): 3455.8, 1672.0, 1664.3, 1201.4 cm⁻¹

NMR (DMSO-d₆+D₂O, δ): 0.84 (3H, d, J=7.7 Hz), 0.96 (3H, d, J=6.9 Hz), 1.09 (3H, d, J=5.8 Hz), 1.39-4.80 (51H, m), 6.64-8.81 (10H, m)

Elemental Analysis Calcd. for C₇₀H₉₀F₁₂N₁₄O₂₃S-5H₂O: C, 45.55; H, 5.46; N, 10.62 Found: C, 45.42; H, 5.56; N, 10.42

### EXAMPLE 59

IR (KBr): 3409.5, 1673.9, 1635.3, 1201.4 cm⁻¹

NMR (DMSO-d₆+D₂O, δ): 0.97 (3H, d, J=7.0 Hz), 1.09 (3H, d, J=5.8 Hz), 1.58-4.79 (54H, m), 6.64-8.96 (11H, m)

ESI MASS (Positive)(m/z): 1321.3 (M+Na)⁺ (free) Elemental Analysis Calcd. for C₇₁H₉₁F₁₂N₁₃O₂₃S.4CF₃CO₂H.6H₂O: C, 45.78; H, 5.57; N, 9.78 Found: C, 45.76; H, 5.62; N, 9.78

### EXAMPLE 60

IR (KBr): 3378.7, 1673.9, 1635.3, 1201.4 cm⁻¹

NMR (DMSO-d₆+D₂O, δ): 0.96 (3H, d, J=6.8 Hz), 1.10 (3H, d, J=5.5 Hz), 1.10-4.80 (53H, m), 6.64-8.52 (11H, m)

ESI MASS (Positive)(m/z): 1321.4 (M+Na)⁺, 1298.3 (M)+(free)

Elemental Analysis Calcd. for C₆₉H₈₉F₉N₁₂O₂₂S.3CF₃CO₂H.10H₂O: C, 45.49; H, 6.03; N, 9.23 Found: C, 45.39; H, 5.45; N, 8.93

### EXAMPLE 61

IR (KBr): 3400, 1673.9, 1635.3, 1201.4 cm⁻¹

NMR (DMSO-d₆+D₂O, δ): 0.96 (3H, d, J=6.7 Hz), 1.08 (3H, d, J=5.6 Hz), 1.33-4.81 (55H, m), 6.64-8.79 (12H, m)

ESI MASS (Positive)(m/z): 1378.3 (M+Na)⁺, 1356.4 (M+H)⁺ (free)

Elemental Analysis Calcd. for C₇₁H₉₂F₉N₁₃O₂₃S.8.5H₂O: C, 46.05; H, 5.93; N, 9.83 Found: C, 46.01; H, 5.70; N, 9.73

### EXAMPLE 62

IR (KBr): 3357.5, 1673.9, 1635.3, 1201.4 cm⁻¹

NMR (DMSO-d₆+D₂O, δ): 0.96 (3H, d, J=6.7 Hz), 1.08 (3H, d, J=5.8 Hz), 1.10-4.81 (64H, m), 6.63-8.44 (11H, m) ESI MASS (Positive)(m/z): 1334.6 (M+Na)+(free) Elemental Analysis Calcd. for C₇₅H₁₀₁F₁₂N₁₁O₂₄.9H₂O: C, 46.66; H, 6.21; N, 7.98 Found: C, 46.62; H, 5.82; N, 7.63

### EXAMPLE 63

IR (KBr): 3380, 1627.0, 1664.3, 1201.4 cm⁻¹

NMR (DMSO-d₆+D₂O, δ): 0.96 (3H, d, J=6.8 Hz), 1.09 (3H, d, J=5.7 Hz), 1.30-5.34 (62H, m), 6.62-8.45 (11H, m)

Elemental Analysis Calcd. for C₇₄H₉₉F₁₂N₁₁O₂₄S.5H₂O: C, 48.18; H, 5.96; N, 8.35 Found: C, 48.37; H, 6.01; N, 8.29

### EXAMPLE 64

IR (KBr): 3457.7, 1672.0, 1664.3, 1201.4 cm⁻¹

NMR (DMSO-d₆+D₂O, δ): 0.96 (3H, d, J=6.7 Hz), 1.10 (3H, d, J=5.6 Hz), 1.57-5.04 (48H, m), 6.62-8.46 (16H, m)

ESI MASS (Positive)(m/z): 1330.6 (M+Na)+(free)

Elemental Analysis Calcd. for C₇₀H₈₅F₉N₁₂O₂₂S-8.5H₂O: C, 46.64; H, 5.70; N, 9.32 Found: C, 46.67; H, 5.24; N, 8.91

### EXAMPLE 65

IR (KBr): 3351.7, 1673.9, 1664.3, 1201.4 cm⁻¹

NMR (DMSO-d₆+D₂O, δ): 0.96 (3H, d, J=6.7 Hz), 1.09 (3H, d, J=5.3 Hz), 1.24-4.81 (42H, m), 6.63-8.47 (16H, m)

### EXAMPLE 66

IR (KBr): 3400, 1672.0, 1664.3, 1635.3, 1201.4 cm⁻¹

NMR (DMSO-d₆+D₂O, 6): 0.96 (3H, d, J=7.0 Hz), 1.09 (3H, d, J=5.1 Hz), 1.24-4.80 (44H, m), 6.64-8.54 (15H, m)

Elemental Analysis Calcd. for C₇₀H₈₄ClF₉N₁₂O₂₂S.8H₂O: C, 45.99; H, 5.51; N, 9.19 Found: C, 46.02; H, 5.14; N, 8.99

### EXAMPLE 67

NMR (DMSO-d₆+D₂O, δ): 0.96 (3H, d, J=6.7 Hz), 1.09 (3H, d, J=5.8 Hz), 1.24-4.81 (55H, m), 6.64-8.12 (11H, m)

### EXAMPLE 68

IR (KBr): 3400, 1664.3, 1635.3, 1201.4 cm⁻¹

NMR (DMSO-d₆+D₂O, δ): 0.96 (3H, d, J=6.7 Hz), 1.09 (3H, d, J=5.9 Hz), 1.22-4.80 (55H, m), 6.64-8.52 (11H, m)

Elemental Analysis Calcd. for C₆₉H₉₁F₉N₁₂O₂₃S.6H₂O: C, 46.88; H, 5.87; N, 9.51 Found: C, 46.97; H, 5.76; N, 9.26

### EXAMPLE 69

NMR (DMSO-d₆+D₂O, δ): 0.98 (3H, d, J=6.7 Hz), 1.10 (3H, d, J=5.9 Hz), 1.2-2.5 (19H, m), 2.85-3.4 (10H, m), 3.5-4.55 (17H, m), 4.7-4.85 (2H, m), 6.6-6.75 (3H, m), 7.14 (2H, d, J=8.8 Hz), 7.98 (2H, d, J=8.8 Hz), 8.05 (2H, d, J=8.7 Hz), 8.12 (2H, d, J=8.7 Hz)

MASS (m/z): 1275.4 (M⁺−H)

### EXAMPLE 70

NMR (DMSO-d₆+D₂O, δ): 0.8-4.55 (46H, m), 4.7-4.9 (2H, m), 6.5-6.75 (3H, m), 7.08 (2H, d, J=8.7 Hz), 7.80 (2H, d, J=8.2 Hz), 7.85-8.05 (4H, m), 8.38 (1H, s)

MASS (m/z)): 1216.3 (M⁺−H)

Elemental Analysis Calcd. for C₅₈H₇₅N₉O₁₈S 6H₂O: C, 52.52; H, 6.61; N, 9.50 Found: C, 52.44; H, 6.35; N, 9.28

### EXAMPLE 71

NMR (DMSO-d₆+D₂O, δ): 0.90 (3H, d, J=6.7 Hz), 0.98 (3H, d, J=6.8 Hz), 1.12 (3H, d, J=5.7 Hz), 1.3-4.6 (47H, m), 4.7-4.9 (2H, m), 6.55-6.85 (3H, m), 7.08 (2H, d, J=8.8 Hz), 7.86 (2H, d, J=8.9 Hz), 8.0-8.2 (4H, m)

MASS (m/z): 1311.3 (M⁺−H)

Elemental Analysis Calcd. for C₆₃H₈₄N₁₂O₁₇S.6H₂O: C, 53.23; H, 6.81; N, 11.82 Found: C, 53.04; H, 6.56; N, 11.40

### EXAMPLE 72

The starting compound (72) was dissolved in water, and subjected to column chromatography on ion exchange resin (AMBERLYST (Trademark: prepared by Organo)) eluting with 1N hydrochloric acid. The fractions containing the object compound were combined, and evaporated under reduced pressure to remove acetonitrile. The residue was lyophilized to give the object compound (72) (6.0 mg).

NMR (DMSO-d₆+D₂O, δ): 0.96 (3H, d, J=6.5 Hz), 1.09 (3H, d, J=5.8 Hz), 1.35-4.80 (50H, m), 6.63-8.14 (11H, m)

### EXAMPLE 73

To a solution of the starting compound (73) (100 mg) and anisole (0.436 ml) in dichloromethane (1 ml) was added dropwise trifluoroacetic acid (0.419 ml) with stirring under ice-cooling, and the mixture was stirred at room temperature for 3 hours. The reaction mixture was evaporated under reduced pressure. A solution of the residue in DMF was treated with 1-[4-[5-[4-(7-methoxyheptyloxy)phenyl]-1,3,4-thiadiazol-2-yl]benzoyloxy]-1H-1,2,3-benzotriazole (48 mg) and N,N-diisopropylethylamine (0.0419 ml) and stirred at room temperature for 1 hour. To the reaction mixture was added piperidine (0.0079 ml) and stirred at room temperature for 2 hours. To the mixture was added 30% acetonitrile in water. The solution was subjected to column chromatography on ODS eluting with 50% acetonitrile in water. The fractions containing the object compound were collected and evaporated under reduced pressure to remove acetonitrile. The residue was lyophilized to give the object compound (73) (16 mg).

NMR (DMSO-d₆+D₂O, δ): 0.97 (3H, d, J=6.7 Hz), 1.10 (3H, d, J=5.6 Hz), 1.2-4.9 (49H, m), 6.6-6.8 (3H, m), 7.14 (2H, d, J=8.6 Hz), 7.85-8.2 (6H, m)

MASS (m/z): 1344.5 (M⁺+H)

### EXAMPLE 74

To the solution of the starting compound (74) (20 mg) in DMF (1 ml) was added piperidine (0.0131 ml) and stirred at room temperature for 2.5 hours. To the mixture was added water. The solution was subjected to column chromatography on ODS eluting with 50% acetonitrile in water. The fractions containing the object compound were collected and evaporated under reduced pressure to remove acetonitrile. The residue was lyophilized to give the object compound (74) (6 mg).

NMR (DMSO-d₆+D₂O, δ): 0.96 (3H, d, J=6.8 Hz), 1.12 (3H, d, J=6.2 Hz), 1.2-2.5 (22H, m), 2.6-3.4 (20H, m), 3.5-3.9 (18H, m), 6.55-6.8 (3H, m), 7.14 (2H, d, J=8.9 Hz), 7.85-8.15 (6H, m)

MASS (m/z): 1312.4 (M+Na)⁺

The following compound was obtained according to a similar manner to that of Example 74.

### EXAMPLE 75

MASS (m/z): 1253.3 (M⁺+Na)

### EXAMPLE 76

A mixture of the starting compound (76) (0.2 g) and 1-[4-[2-(4-pentyloxyphenyl)-1,3-thiazol-5-yl]benzoyloxy]-1H-1,2,3-benzotriazole (0.13 g) in N,N-dimethylformamide (4 ml) was stirred for 24 hours at room temperature. To the reaction mixture was added ethyl acetate and the resulting precipitates were collected by filtration. The precipitates were purified by column chromatography on silica gel eluting with a mixture of chloroform and methanol (5:1). The eluted fractions containing the desired product were collected and evaporated in vacuo to give the object compound (76) (0.16 g).

IR (KBr): 3350, 1659, 1635, 1520, 1439, 1255 cm⁻¹

NMR (DMSO-d₆+D₂O, δ): 0.8-1.0 (6H, m), 1.08 (3H, d, J=6.0 Hz), 1.3-1.5 (4H, m), 1.6-2.7 (10H, m), 2.8-3.0 (1H, m), 3.1-3.3 (1H, m), 3.4-4.5 (17H, m), 3.75 (3H, s), 4.7-4.9 (2H, m), 6.5-6.8 (3H, m), 7.08 (2H, d, J=8.9 Hz), 7.81 (2H, d, J=8.4 Hz), 7.9-8.0 (4H, m), 8.36 (1H, s)

ESI MASS (Positive): 1210.3 (M+Na)⁺

Elemental Analysis Calcd. for C₅₇H₇₃N₉O₁₇S.4.5H₂O: C, 53.93; H, 6.51; N, 9.93 Found: C, 54.00; H, 6.37; N, 9.91

The following compound was obtained according to a similar manner to that of Example 76.

### EXAMPLE 77

IR (KBr): 3350, 2935, 1637, 1518, 1439, 1255 cm⁻¹

NMR (DMSO-d₆+D₂O, δ): 0.8-1.0 (6H, m), 1.09 (3H, d, J=6.0 Hz), 1.2-1.5 (7H, m), 1.8-2.6 (10H, m), 2.8-4.5 (21H, m), 4.7-5.0 (2H, m), 6.5-6.8 (3H, m), 7.08 (2H, d, J=8.9 Hz), 7.7-8.0 (6H, m), 8.38 (1H, s)

ESI MASS (Positive): 1224.3 (M+Na)⁺

Elemental Analysis Calcd. for C₅₈H₇₅N₉O₁₇S.3H₂O: C, 55.45; H, 6.50; N, 10.03 Found: C 55.75, H 6.38, N 10.04

### EXAMPLE 78

To a solution of a mixture of the starting compound (78) (0.25 g) and anisole (0.2 ml) in dichloromethane (3 ml) was added trifluoroacetic acid (3 ml) under ice-cooling and the mixture was stirred for 2 hours at the same temperature. To the reaction mixture was added diisopropyl ether. The resulting precipitates were collected by filtration and dried in vacuo to give the object compound (78) (0.24 g).

IR (KBr): 3356, 3342, 2937, 1670, 1637, 1520, 1439, 1255, 1201, 1180, 1134 cm⁻¹

NMR (DMSO-d₆+D₂O, δ): 0.8-1.2 (9H, m), 1.2-1.6 (4H, m), 1.6-2.6 (12H, m), 2.8-4.5 (23H, m), 4.7-4.9 (2H, m), 6.5-6.8 (3H, m), 7.08 (2H, d, J=8.7 Hz), 7.7-8.0 (6H, m), 8.37 (1H, s)

ESI MASS (Positive): 1253.2 (M+Na)⁺

Elemental Analysis Calcd. for C₆₁H₇₉F₃N₁₀O₁₉S.4.5H₂O: C, 51.36; H, 6.22; N, 9.82 Found: C, 51.34; H, 6.19; N, 9.57 The following compounds [Example 79 to 81] were obtained according to a similar manner to that of Example 78.

### EXAMPLE 79

IR (KBr): 3350, 2935, 1635, 1520, 1439, 1254, 1068 cm⁻¹

NMR (DMSO-d₆+D₂O, δ): 0.8-1.0 (6H, m), 1.07 (3H, d, J=5.4 Hz), 1.2-1.5 (4H, m), 1.6-2.6 (10H, m), 2.8-4.6 (21H, m), 4.7-5.0 (2H, m), 6.6-6.8 (3H, m), 7.08 (2H, d, J=8.8 Hz), 7.7-8.0 (6H, m), 8.36 (1H, s)

ESI MASS (Negative): 1230.3 (M−H)⁻

Elemental Analysis Calcd. for C₅₈H₇₃N₉O₁₉S.6H₂O: C, 51.97; H, 6.39; N, 9.40 Found: C, 51.84; H, 6.29; N, 9.32

### EXAMPLE 80

IR (KBr): 3346, 1649, 1632, 1603, 1539, 1514, 1441, 1254, 1211 cm⁻¹

NMR (DMSO-d₆+D₂O, δ): 0.8-1.2 (9H, m), 1.4-2.7 (28H, m), 2.8-4.6 (21H, m), 4.7-5.0 (2H, m), 6.5-6.8 (3H, m), 7.1-7.3 (2H, m), 7.8-8.2 (6H, m)

ESI MASS (Positive): 1348.2 (M+Na)⁺

Elemental Analysis Calcd. for C₆₈H₈₉F₆N₁₃O₂₀S.14H₂O: C, 45.20; H, 6.53; N, 10.08 Found: C, 45.02; H, 6.40; N, 10.23

### EXAMPLE 81

IR (KBr): 3350, 2937, 1649, 1633, 1520, 1439, 1273, 1086 cm⁻¹

NMR (DMSO-d₆+D₂O, δ): 0.96 (3H, d, J=6.7 Hz), 1.08 (3H, d, J=5.9 Hz), 1.2-2.7 (20H, m), 2.8-4.5 (30H, m), 3.21 (3H, s), 4.7-5.0 (2H, m), 6.5-6.8 (3H, m), 7.12 (2H, d, J=9.0 Hz), 7.77 (2H, d, J=8.7 Hz), 7.9-8.1 (4H, m), 8.79 (1H, s)

ESI MASS (Positive): 1406.3 (M+Na)⁺

Elemental Analysis Calcd. for C₆₈H₉₀F₃N₁₃O₂₀S.12H₂O: C, 47.63; H, 6.70; N, 10.62 Found: C, 47.54; H, 6.51; N, 10.70

The following compounds [Example 82 to 148] were obtained according to a similar manner to that of Example 21.

### EXAMPLE 82

IR (KBr): 2935, 1635, 1529, 1518, 1444, 1277, 1080 cm⁻¹

NMR (DMSO-d₆+D₂O, δ): 0.9-1.2 (6H, m), 1.2-2.7 (19H, m), 2.7-4.9 (32H, m), 5.6-5.9 (2H, m), 6.5-6.8 (3H, m), 7.0-7.2 (2H, m), 7.85 (2H, d, J=8.8 Hz), 8.0-8.2 (4H, m)

ESI MASS (Positive): 1333.5 (M+Na)⁺ (free [M]=1311.50)

Elemental Analysis Calcd. for C₆₄H₈₆N₁₂O₁₆S.2HCl.11H₂O: C, 48.57; H, 7.01; N, 10.62 Found: C, 48.20; H, 6.60; N, 10.62

### EXAMPLE 83

IR (KBr): 3350, 2974, 2935, 1633, 1606, 1518, 1443, 1419, 1261 cm⁻¹

NMR (DMSO-d₆+D₂O, δ): 0.97 (3H, d, J=6.6 Hz), 1.08 (3H, d, J=5.4 Hz), 1.6-2.8 (9H, m), 2.8-4.9 (44H, m), 6.5-6.7 (1H, m), 6.72 (2H, d, J=7.9 Hz), 7.03 (2H, d, J=8.7 Hz), 7.16 (2H, d, J=8.9 Hz), 7.45 (2H, d, J=8.6 Hz), 7.92 (2H, d, J=8.4 Hz), 8.0-8.2 (4H, m)

ESI MASS (Positive): 1413.6 (M+Na)⁺ (free [M]=1391.59)

Elemental Analysis Calcd. for C₆₈H₉₁N₁₄O₁₆S.3HCl.14H₂O: C, 46.59; H, 6.96; N, 11.19 Found: C, 46.20; H, 6.44; N, 11.05

### EXAMPLE 84

IR (KBr): 3358, 2937, 1635, 1518, 1443, 1321, 1277, 1084 cm⁻¹

NMR (DMSO-d₆+D₂O, δ): 0.97 (3H, d, J=6.8 Hz), 1.08 (3H, d, J=5.7 Hz), 1.4-2.8 (17H, m), 2.8-4.9 (38H, m), 6.6-6.8 (3H, m), 7.0-7.2 (2H, m), 7.8-7.9 (2H, m), 8.0-8.2 (4H, m)

ESI MASS (Positive): 1339.5 (M+Na)⁺ (free [M]=1317.51)

### EXAMPLE 85

IR (KBr): 2944, 1635, 1608, 1527, 1520, 1443, 1419, 1273 cm⁻¹

NMR (DMSO-d₆+D₂O, δ): 0.96 (3H, d, J=6.7 Hz), 1.08 (3H, d, J=5.6 Hz), 1.1-2.7 (18H, m), 2.7-4.9 (36H, m), 6.5-6.8 (3H, m), 7.19 (2H, d, J=9.1 Hz), 7.93 (2H, d, J=8.8 Hz), 8.0-8.2 (4H, m)

ESI MASS (Positive): 1321.4 (M+Na)+(free [M]=1298.51)

Elemental Analysis Calcd. for C₆₃H₈₇N₁₃O₁₅S.3HCl.9H₂O: C, 48.19; H, 6.93; N, 11.60 Found: C, 48.13; H, 6.64; N, 11.47

### EXAMPLE 86

NMR (DMSO-d₆+D₂O, δ): 0.8-1.05 (6H, m), 1.08 (3H, d, J=5.7 Hz), 1.25-2.7 (14H, m), 2.75-4.5 (25H, m), 4.65-4.85 (2H, m), 6.6-6.85 (3H, m), 7.15 (2H, d, J=8.8 Hz), 7.8-8.1 (6H, m), 8.86 (1H, s)

MASS (m/z): 1265.4 (M+Na)

Elemental Analysis Calcd. for C₅₉H₈₀Cl₂N₁₂O₁₆S.6H₂O: C, 49.75; H, 6.51; N, 11.80 Found: C, 49.65; H, 6.58; N, 11.72

### EXAMPLE 87

NMR (DMSO-d₆+D₂O, δ): 0.96 (3H, d, J=6.7 Hz), 1.07 (3H, d, J=6.0 Hz), 1.2-1.7 (18H, m), 1.75-4.55 (30H, m), 4.7-4.9 (2H, m), 6.6-6.8 (3H, m), 7.05 (2H, d, J=8.8 Hz), 7.6-7.8 (4H, m), 7.96 (2H, d, J=8.4 Hz)

MASS (m/z): 1200.4 (M+Na)

Elemental Analysis Calcd. for C₅₈H₈₅Cl₂N₉O₁₇.7H₂O: C, 50.58; H, 7.24; N, 9.15 Found: C, 50.63; H, 7.30; N, 9.13

### EXAMPLE 88

NMR (DMSO-d₆+D₂O, δ): 0.96 (3H, d, J=6.7 Hz), 1.07 (3H, d, J=6.0 Hz), 1.2-2.7 (20H, m), 2.75-4.55 (30H, m), 4.7-4.9 (2H, m), 6.6-6.8 (3H, m), 7.05 (2H, d, J=8.8 Hz), 7.6-7.8 (4H, m), 7.96 (2H, d, J=8.4 Hz)

MASS (m/z): 1214.5 (M+Na)

Elemental Analysis Calcd. for C₅₉H₈₇Cl₂N₉O₁₇.7H₂O: C, 50.93; H, 7.32; N, 9.06 Found: C, 51.20; H, 7.40; N, 9.11

### EXAMPLE 89

NMR (DMSO-d₆+D₂O, δ): 0.96 (3H, d, J=6.8 Hz), 1.08 (3H, d, J=6.0 Hz), 1.2-3.05 (24H, m), 3.15-4.55 (26H, m), 4.7-4.85 (2H, m), 6.6-6.8 (3H, m), 7.15-7.45 (2H, m), 7.8-8.0 (3H, m), 8.44 (1H, s)

MASS (m/z): 1188.4 (M+Na)

Elemental Analysis Calcd. for C₅₇H₈₅Cl₂N₉O₁₇.7H₂O: C, 50.14; H, 7.31; N, 9.23 Found: C, 50.33; H, 7.43; N, 9.27

### EXAMPLE 90

NMR (DMSO-d₆+D₂O, δ): 0.96 (3H, d, J=6.8 Hz), 1.07 (3H, d, J=5.7 Hz), 1.6-2.7 (8H, m), 2.8-3.35 (9H, m), 3.45-4.6 (20H, m), 4.7-4.9 (2H, m), 6.6-6.85 (3H, m), 7.05-7.35 (6H, m), 7.66 (2H, d, J, 8.7 Hz), 7.72 (2H, d, J=8.4 Hz), 7.94 (2H, d, J=8.4 Hz)

MASS (m/z): 1187.3 (M+Na)

### EXAMPLE 91

NMR (DMSO-d₆+D₂O, δ): 0.96 (3H, d, J=6.7 Hz), 1.07 (3H, d, J=6.0 Hz), 1.6-2.7 (8H, m), 2.75-3.35 (9H, m), 3.4-4.6 (26H, m), 4.7-4.9 (2H, m), 6.6-6.8 (4H, m), 6.86 (1H, s), 7.17 (2H, d, J=8.9 Hz), 7.6-7.8 (4H, m), 7.94 (2H, d, J=8.5 Hz)

MASS (m/z): 1247.4 (M+Na)

### EXAMPLE 92

NMR (DMSO-d₆+D₂O, δ): 0.86 (3H, t, J=6.3 Hz), 0.96 (3H, d, J=6.8 Hz), 1.10 (3H, d, J=5.8 Hz), 1.2-1.55 (14H, m), 1.6-2.7 (10H, m), 2.75-3.35 (7H, m), 3.4-4.55 (21H, m), 4.7-4.9 (2H, m), 6.6-6.8 (3H, m), 6.9-7.15 (3H, m), 7.42 (1H, d, J=9.2 Hz)

MASS (m/z): 1189.4 (M+Na)

### EXAMPLE 93

NMR (DMSO-d₆+D₂O, δ): 0.97 (3H, d, J=6.7 Hz), 1.07 (3H, d, J=5.3 Hz), 1.1-1.35 (6H, m), 1.4-2.7 (16H, m), 2.75-4.55 (29H, m), 4.7-4.9 (2H, m), 6.7-6.8 (3H, m), 7.24 (2H, d, J=8.8 Hz), 7.6-7.8 (4H, m), 7.95 (2H, d, J=8.4 Hz)

MASS (m/z): 1237.4 (M+Na)

### EXAMPLE 94

NMR (DMSO-d₆+D₂O, δ): 0.97 (3H, d, J=6.8 Hz), 1.08 (3H, d, J=5.5 Hz), 1.15-1.4 (4H, m), 1.45-3.4 (21H, m), 3.5-4.55 (19H, m), 4.65-4.9 (2H, m), 6.6-6.8 (3H, m), 7.06 (2H, d, J=8.9 Hz), 7.2-7.4 (5H, m), 7.84 (2H, d, J=8.6 Hz), 7.95-8.2 (4H, m)

MASS (m/z): 1343.5 (M+Na)

### EXAMPLE 95

NMR (DMSO-d₆+D₂O, δ): 0.97 (3H, d, J=6.7 Hz), 1.08 (3H, d, J=5.8 Hz), 1.6-2.75 (8H, m), 2.8-3.4 (1H, m), 3.5-4.6 (20H, m), 4.7-4.95 (2H, m), 6.6-6.85 (3H, m), 7.08 (2H, d, J=8.9 Hz), 7.6-7.9 (8H, m), 8.00 (2H, d, J=8.3 Hz)

MASS (m/z): 1217.4 (M+Na)

### EXAMPLE 96

NMR (DMSO-d₆+D₂O, δ): 0.96 (3H, d, J=6.7 Hz), 1.08 (3H, d, J=5.8 Hz), 1.2-4.5 (53H, m), 4.65-4.85 (2H, m), 6.55-6.8 (3H, m), 7.09 (2H, d, J=8.8 Hz), 7.76 (2H, d, J=8.8 Hz), 7.85-8.0 (4H, m), 8.80 (1H, s)

MASS (m/z)): 1378.4 (M+Na)

Elemental Analysis Calcd. for C₆₅H₉₁Cl₂N₁₃O₁₇S.6H₂O: C, 50.78; H, 6.75; N, 11.84 Found: C, 50.99; H, 6.79; N, 11.62

### EXAMPLE 97

IR (KBr): 3350, 1666.2, 1648.8, 1631.5, 1538.9, 1515.8, 1450.2 cm⁻¹

NMR (DMSO-d₆, δ): 0.96 (3H, d, J=6.7 Hz), 1.04-1.07 (6H, m), 1.38-5.61 (56H, m), 6.63-8.94 (18H, m)

ESI MASS (Positive)(m/z): 1262.2 (M+H)⁺, 1284.4 (M+Na)⁺

### EXAMPLE 98

IR (KBr): 3350, 1648.8, 1631.5, 1538.9, 1513.8, 1454.1 cm⁻¹

NMR (DMSO-d₆, 6): 0.96 (3H, d, J=6.8 Hz), 1.05 (3H, d, J=5.8 Hz), 1.72-4.79 (65H, m), 6.64-8.60 (23H, m)

ESI MASS (Positive)(m/z): 1329.4 (M+Na)⁺

### EXAMPLE 99

IR (KBr): 3350, 1648.8, 1631.5, 1538.9, 1521.6, 1452.1 cm⁻¹

NMR (DMSO-d₆, 6): 0.96 (3H, d, J=6.8 Hz), 1.08 (3H, d, J=5.4 Hz), 1.59-4.78 (59H, m), 6.64-8.89 (18H, m)

ESI MASS (Positive)(m/z): 1292.5 (M+Na)⁺

### EXAMPLE 100

IR (KBr): 3350, 1648.8, 1631.5, 1538.9, 1521.6, 1452.1 cm⁻¹

NMR (DMSO-d₆, δ): 0.96 (3H, d, J=6.8 Hz), 1.08 (3H, d, J=5.3 Hz), 1.27-4.78 (63H, m), 6.67-8.93 (18H, m)

ESI MASS (Positive)(m/z): 1320.5 (M+Na)⁺

### EXAMPLE 101

IR (KBr): 3350, 1648.8, 1631.5, 1538.9, 1521.6, 1442.5 cm⁻¹

NMR (DMSO-d₆, δ): 0.96 (3H, d, J=6.8 Hz), 1.08 (3H, d, J=5.9 Hz), 1.20-5.59 (63H, m), 6.67-8.92 (18H, m)

ESI MASS (Positive)(m/z): 1320.6 (M+Na)⁺

### EXAMPLE 102

NMR (DMSO-d₆, δ): 0.96 (3H, d, J=6.7 Hz), 1.08 (3H, d, J=5.5 Hz), 1.24-5.59 (67H, m), 6.63-8.92 (18H, m)

ESI MASS (Positive)(m/z): 1352.5 (M+Na)⁺

### EXAMPLE 103

IR (KBr): 3305.4, 1637.3, 1604.5, 1519.6, 1442.5 cm⁻¹

NMR (DMSO-d₆, δ): 0.96 (3H, d, J=6.7 Hz), 1.08 (3H, d, J=5.6 Hz), 1.24-4.76 (47H, m), 6.67-8.93 (25H, m)

ESI MASS (Positive)(m/z): 1302.2 (M+Na)⁺

### EXAMPLE 104

IR (KBr): 3396.0, 1631.5, 1604.5, 1519.6, 1444.4 cm¹

NMR (DMSO-d₆, δ): 0.96 (3H, d, J=6.7 Hz), 1.08 (3H, d, J=5.5 Hz), 1.23-4.76 (47H, m), 6.63-8.93 (24H, m)

ESI MASS (Positive)(m/z): 1302.3 (M+Na)⁺

### EXAMPLE 105

IR (KBr): 3350, 1650.8, 1540.8, 1508.1, 1456.0 cm⁻¹

NMR (DMSO-d₆, δ): 0.89 (3H, t, J=6.6 Hz), 0.96 (3H, d, J=6.7 Hz), 1.08 (3H, d, J=5.8 Hz), 1.33-5.58 (52H, m), 6.63-9.53 (20H, m)

ESI MASS (Positive)(m/z): 1222.3 (M+Na)⁺

### EXAMPLE 106

IR (KBr): 3350, 1648.8, 1631.5, 1540.8, 1513.8, 1454.1 cm⁻¹

NMR (DMSO-d₆, δ): 0.96 (3H, d, J=6.7 Hz), 1.07 (3H, d, J=5.7 Hz), 1.26-4.79 (71H, m), 6.63-8.77 (14H, m)

ESI MASS (Positive)(m/z): 1228.4 (M+Na)⁺

### EXAMPLE 107

IR (KBr): 3350, 1648.8, 1631.5, 1538.9, 1515.8, 1456.0 cm⁻¹

NMR (DMSO-d₆, δ): 0.87 (3H, t, J=7.0 Hz), 0.96 (3H, d, J=6.7 Hz), 1.07 (3H, d, J=5.6 Hz), 1.15-4.78 (62H, m), 6.67-8.76 (18H, m)

ESI MASS (Positive)(m/z): 1250.5 (M+Na)⁺

### EXAMPLE 108

IR (KBr): 3350, 1648.8, 1631.5, 1538.9, 1515.8, 1454.1 cm⁻¹

NMR (DMSO-d₆, 8): 0.96 (3H, d, J=6.7 Hz), 1.07 (3H, d, J=5.6 Hz), 1.15 (3H, s), 1.18 (3H, s), 1.24-4.78 (56H, m), 6.63-8.76 (22H, m)

ESI. MASS (Positive)(m/z): 1329.6 (M+Na)⁺

### EXAMPLE 109

IR (KBr): 3350.0, 1648.8, 1538.9, 1513.8, 1456.0 cm⁻¹

NMR (DMSO-d₆, δ): 0.93-1.30 (9H, m), 1.40-4.77 (60H, m), 6.67-9.36 (22H, m)

ESI MASS (Positive)(m/z): 1316.5 (M+Na)⁺

### EXAMPLE 110

IR (KBr): 3350.0, 1648.8, 1631.5, 1538.9, 1515.8, 1456.0 cm⁻¹

NMR (DMSO-d₆, δ): 0.96 (3H, d, J=6.7 Hz), 1.07 (3H, d, J=5.7 Hz), 1.24-4.79 (62H, m), 6.67-8.76 (23H, m)

ESI MASS (Positive)(m/z): 1328.5 (M+Na)⁺

### EXAMPLE 111

IR (KBr): 3350, 1648.8, 1631.5, 1540.8, 1513.8, 1456.0 cm⁻¹

NMR (DMSO-d₆, 8): 0.96 (3H, d, J=6.8 Hz), 1.05 (3H, d, J=5.6 Hz), 1.33-4.77 (65H, m), 6.63-8.53 (18H, m)

ESI MASS (Positive)(m/z): 1270.4 (M+Na)⁺

### EXAMPLE 112

IR (KBr): 3350, 1648.8, 1631.5, 1540.8, 1513.8, 1454.1 cm⁻¹

NMR (DMSO-d₆, δ): 0.96 (3H, d, J=6.8 Hz), 1.05 (3H, d, J=5.8 Hz), 1.49-4.91 (66H, m), 6.67-8.51 (18H, m)

ESI MASS (Positive)(m/z): 1277.4 (M+Na)⁺

### EXAMPLE 113

IR (KBr): 3350, 1648.8, 1631.5, 1540.8, 1513.8, 1456.0 cm⁻¹

NMR (DMSO-d₆, δ): 0.96 (3H, d, J=6.8 Hz), 1.05 (3H, d, J=5.5 Hz), 1.25-4.90 (62H, m), 6.67-8.52 (22H, m)

ESI MASS (Positive)(m/z): 1313.5 (M+Na)⁺

### EXAMPLE 114

IR (KBr): 3350, 1648.8, 1631.5, 1540.8, 1513.8, 1454.1 cm⁻¹

NMR (DMSO-d₆, δ): 0.86 (3H, t, J=7.0 Hz), 0.95 (3H, d, J=6.8 Hz), 1.04 (3H, d, J=5.9 Hz), 1.16-4.75 (68H, m), 6.66-8.53 (14H, m)

ESI MASS (Positive)(m/z): 1216.4 (M+Na)⁺

### EXAMPLE 115

IR (KBr): 3359.4, 1631.5, 1610.3, 1506.1, 1454.1 cm⁻¹

NMR (DMSO-d₆, δ): 0.96 (3H, d, J=6.8 Hz), 1.05 (3H, d, J=5.7 Hz), 1.45-4.79 (63H, m), 6.64-8.52 (22H, m)

ESI MASS (Positive)(m/z): 1332.4 (M+Na)⁺

### EXAMPLE 116

IR (KBr): 3345.9, 1633.4, 1606.4, 1517.7, 1442.5 cm⁻¹

NMR (DMSO-d₆, δ): 0.92-0.98 (6H, m), 1.08 (3H, d, J=5.6 Hz), 1.23-5.60 (60H, m), 6.63-8.93 (18H, m)

ESI MASS (Positive)(m/z): 1320.4 (M+Na)⁺

Elemental Analysis Calcd. for C₆₃H₉₀Cl₃N₁₃O₁₅S.5H₂O: C, 50.51; H, 6.73; N, 12.16 Found: C, 50.24; H, 7.09; N, 11.95

### EXAMPLE 117

IR (KBr): 3340.1, 1633.4, 1517.7, 1496.5, 1442.5 cm⁻¹

NMR (DMSO-d₆, 6): 0.96 (3H, d, J=6.7 Hz), 1.07 (3H, d, J=5.4 Hz), 1.30-4.78 (71H, m), 6.67-8.75 (18H, m)

ESI MASS (Positive)(m/z): 1320.6 (M+Na)⁺

### EXAMPLE 118

IR (KBr): 3350, 1648.8, 1538.9, 1515.8, 1452.1 cm⁻¹

NMR (DMSO-d₆, δ): 0.96 (3H, d, J=6.7 Hz), 1.08 (3H, d, J=5.4 Hz), 1.24-4.88 (51H, m), 6.63-8.93 (23H, m)

ESI MASS (Positive)(m/z): 1293.4 (M+H)⁺, 1315.3 (M+Na)⁺

### EXAMPLE 119

IR (KBr): 3350, 1648.8, 1631.5, 1538.9, 1521.6, 1452.1 cm⁻¹

NMR (DMSO-d₆, δ): 0.96 (3H, d, J=6.7 Hz), 1.08 (3H, d, J=5.5 Hz), 1.14-4.76 (6H, m), 6.63-8.92 (18H, m)

ESI MASS (Positive)(m/z): 1313.3 (M+H)⁺, 1335.4 (M+Na)⁺

### EXAMPLE 120

IR (KBr): 3350, 1668.1, 1650.8, 1540.8, 1515.8, 1452.1 cm⁻¹

NMR (DMSO-d₆, 6): 0.96 (3H, d, J=6.7 Hz), 1.08 (3H, d, J=5.4 Hz), 1.31-4.77 (66H, m), 6.63-8.92 (18H, m)

ESI MASS (Positive)(m/z): 1331.3 (M+H)⁺, 1353.4 (M+Na)⁺

### EXAMPLE 121

IR (KBr): 3350, 1648.8, 1540.8, 1521.6, 1454.1 cm⁻¹

NMR (DMSO-d₆, δ): 0.96 (3H, d, J=6.7 Hz), 1.08 (3H, d, J=5.5 Hz), 1.18-4.78 (61H, m), 6.67-8.92 (18H, m)

ESI MASS (Positive)(m/z): 1306.4 (M+Na)⁺

### EXAMPLE 122

IR (KBr): 3350, 1648.8, 1538.9, 1521.6, 1452.1 cm¹

NMR (DMSO-d₆, 8): 0.96 (3H, d, J=6.7 Hz), 1.08 (3H, d, J=5.4 Hz), 1.52-4.72 (61H, m), 6.67-8.93 (18H, m)

ESI MASS (Positive)(m/z): 1296.3 (M+H)⁺

### EXAMPLE 123

IR (KBr): 3350, 1648.8, 1540.8, 1521.6, 1454.1 cm⁻¹

NMR (DMSO-d₆, δ): 0.96 (3H, d, J=6.7 Hz), 1.08 (3H, d, J=5.6 Hz), 1.28-4.78 (61H, m), 6.67-8.92 (18H, m)

ESI MASS (Positive)(m/z): 1284.3 (M+H)⁺, 1306.3 (M+Na)⁺

### EXAMPLE 124

IR (KBr): 3350, 1648.8, 1631.5, 1538.9, 1513.8, 1452.1 cm⁻¹

NMR (DMSO-d₆, δ): 0.86 (3H, t, J=7.2 Hz), 0.96 (3H, d, J=6.7 Hz), 1.08 (3H, d, J=5.7 Hz), 1.32-4.78 (62H, m), 6.67-8.89 (17H, m)

ESI MASS (Positive)(m/z): 1313.4 (M+H)⁺, 1335.4 (M+Na)⁺

### EXAMPLE 125

IR (KBr): 3350, 1650.8, 1540.8, 1456.0 cm⁻¹

NMR (DMSO-d₆, δ): 0.86 (3H, t, J=6.9 Hz), 0.96 (3H, d, J=6.6 Hz), 1.08 (3H, d, J=5.7 Hz), 1.21-4.61 (62H, m), 6.67-8.89 (17H, m)

ESI MASS (Positive)(m/z): 1313.5 (M+H)⁺, 1336.4 (M+Na)⁺

### EXAMPLE 126

IR (KBr): 3348, 3331, 1633, 1529, 1518, 1443, 1277, 1082 cm⁻¹

NMR (DMSO-d₆+D₂O, δ): 0.97 (3H, d, J=6.7 Hz), 1.09 (3H, d, J=5.7 Hz), 1.5-2.7 (13H, m), 2.8-4.6 (24H, m), 3.34 (3H, s), 4.7-4.9 (2H, m), 6.6-6.8 (3H, m), 7.48 (2H, d, J=8.3 Hz), 7.78 (2H, d, J=8.2 Hz), 7.93 (2H, d, J=8.4 Hz), 8.0-8.2 (6H, m)

ESI MASS (Positive): 1305.4 (M+Na)⁺

Elemental Analysis Calcd. for C₆₂H₈₁Cl₂N₁₁O₁₇S.10H₂O: C, 48.50; H, 6.63; N, 10.03 Found: C, 48.28; H, 6.21; N, 10.13

### EXAMPLE 127

IR (KBr): 3446, 3413, 3377, 3352, 1635, 1443, 1277, 1082 cm⁻¹

NMR (DMSO-d₆+D₂O, δ): 0.97 (3H, d, J=6.7 Hz), 1.09 (3H, d, J=5.7 Hz), 1.4-2.7 (23H, m), 2.8-4.5 (24H, m), 4.7-4.9 (2H, m), 5.6-5.8 (2H, m), 6.6-6.8 (3H, m), 7.09 (2H, d, J=8.9 Hz), 7.85 (2H, d, J=8.7 Hz), 8.0-8.1 (4H, m)

ESI MASS (Positive): 1319.5 (M+Na)⁺

Elemental Analysis Calcd. for C₆₃H₈₆Cl₂N₁₂O₁₆S.12H₂O: C, 47.69; H, 6.99; N, 10.59 Found: C, 47.75; H, 6.45; N, 10.66

### EXAMPLE 128

IR (KBr): 3412, 3379, 3354, 3332, 1635, 1527, 1522, 1443, 1275, 1250, 1086 cm⁻¹ NMR (DMSO-d₆+D₂O, δ): 0.97 (3H, d, J=6.8 Hz), 1.0-1.2 (6H, m), 1.5-2.7 (15H, m), 2.8-4.6 (24H, m), 4.7-4.9 (2H, m), 6.6-6.8 (3H, m), 7.08 (2H, d, J=8.8 Hz), 7.74 (2H, d, J=8.7 Hz), 7.87 (2H, d, J=8.4 Hz), 8.0-8.2 (6H, m)

ESI MASS (Positive): 1318.2 (M+Na)⁺

Elemental Analysis Calcd. for C₆₃H₈₃Cl₂N₁₁O₁₇S.8H₂O: C, 50.00; H, 6.59; N, 10.18 Found: C, 50.06; H, 6.32; N, 10.13

### EXAMPLE 129

IR (KBr): 3356, 1635, 1529, 1518, 1443, 1277, 1086 cm⁻¹

NMR (DMSO-d₆+D₂O, δ): 0.97 (3H, d, J=6.7 Hz), 1.09 (3H, d, J=5.7 Hz), 1.19 (3H, d, J=7.0 Hz), 1.5-2.7 (13H, m), 2.8-4.6 (22H, m), 4.7-4.9 (2H, m), 6.6-6.8 (3H, m), 7.48 (2H, d, J=8.3 Hz), 7.78 (2H, d, J=8.1 Hz), 7.93 (2H, d, J=8.4 Hz), 8.0-8.2 (6H, m)

ESI MASS (Positive): 1274.3 (M+Na)⁺

Elemental Analysis Calcd. for C₆₁H₇₉Cl₂N₁₁O₁₆S.8H₂O: C, 49.86; H, 6.52; N, 10.49 Found: C, 49.70; H, 6.20; N, 10.41

### EXAMPLE 130

IR (KBr): 3361, 1632, 1537, 1512, 1452, 1441, 1255 cm⁻¹

NMR (DMSO-d₆+D₂O, δ): 0.8-0.9 (3H, m), 0.96 (3H, d, J=6.8 Hz), 1.05 (3H, d, J=6.1 Hz), 1.2-1.5 (10H, m), 1.6-1.8 (3H, m), 1.8-2.1 (4H, m), 2.2-2.4 (2H, m), 2.4-2.7 (3H, m), 2.8-3.0 (3H, m), 3.2-3.3 (3H, m), 3.6-3.8 (1H, m), 3.9-4.3 (12H, m), 4.3-4.5 (4H, m), 4.7-4.8 (2H, m), 6.6-6.8 (3H, m), 6.98 (2H, d, J=9.0 Hz), 7.86 (2H, d, J=8.8 Hz)

ESI MASS (Positive): 1108.4 (M+Na)⁺

Elemental Analysis Calcd. for C₅₂H₈₁Cl₂N₉O₁₆.5.5H₂O: C, 49.64; H, 7.37; N, 10.02 Found: C, 49.62; H, 7.47; N, 10.03

### EXAMPLE 131

ESI MASS (Positive): 1296.4 (M+H)⁺, 1318.5 (M+Na)⁺

### EXAMPLE 132

NMR (DMSO-d₆+D₂O, δ): 0.97 (3H, d, J=6.8 Hz), 1.10 (3H, d, J=6.0 Hz), 1.68-4.84 (40H, m), 6.65-9.16 (14H, m)

### EXAMPLE 133

NMR (DMSO-d₆+D₂O, δ): 0.97 (3H, d, J=6.8 Hz), 1.09 (3H, d, J=5.9 Hz), 1.68-4.82 (42H, m), 6.60-9.16 (14H, m)

### EXAMPLE 134

NMR (DMSO-d₆+D₂, δ): 0.97 (3H, d, J=6.8 Hz), 1.10 (3H, d, J=5.9 Hz), 1.15 (3H, t, J=7.0 Hz), 1.60-4.83 (39H, m), 6.65-9.16 (14H, m)

### EXAMPLE 135

NMR (DMSO-d₆+D₂O, δ): 0.97 (3H, d, J=6.8 Hz), 1.10 (3H, d, J=5.9 Hz), 1.15 (3H, t, J=7.0 Hz), 1.60-4.82 (41H, m), 6.60-9.16 (14H, m)

### EXAMPLE 136

NMR (DMSO-d₆+D₂O, δ): 0.96 (3H, d, J=6.8 Hz), 1.09 (3H, d, J=6.1 Hz), 1.2-4.55 (51H, m), 4.7-4.9 (2H, m), 6.6-6.8 (3H, m), 7.14 (2H, d, J=8.8 Hz), 7.98 (2H, d, J=8.8 Hz), 8.06 (2H, d, J=9.0 Hz), 8.12 (2H, d, J=9.0 Hz)

MASS (m/z): 1298.4 (M+Na)⁺

Elemental Analysis Calcd. for C₆₁H₈₇Cl₂N₁₁O₁₇S-7H₂O: C, 49.66; H, 6.90; N, 10.44 Found: C, 49.92; H, 6.89; N, 10.36

### EXAMPLE 137

NMR (DMSO-d₆+D₂O, δ): 0.85-1.05 (6H, m), 1.08 (3H, d, J=5.8 Hz), 1.2-1.5 (4H, m), 1.6-4.5 (38H, m), 4.7-4.85 (2H, m), 6.6-6.8 (3H, m), 7.09 (2H, d, J=8.8 Hz), 7.81 (2H, d, J=8.5 Hz), 7.92 (2H, d, J=8.8 Hz), 7.98 (2H, d, J=8.5 Hz), 8.37 (1H, s)

MASS (m/z): 1239.4 (M+Na)

Elemental Analysis Calcd. for C₆₁H₈₇Cl₂N₁₁O₁₇S.7H₂O: C, 50.03; H, 6.83; N, 9.89 Found: C, 50.11; H, 6.72; N, 9.75

### EXAMPLE 138

NMR (DMSO-d₆+D₂O, δ): 0.85-1.05 (6H, m), 1.08 (3H, d, J=5.9 Hz), 1.30-4.55 (52H, m), 4.7-4.9 (2H, m), 6.6-6.8 (3H, m), 7.12 (2H, d, J=8.5 Hz), 7.89 (2H, d, J=8.5 Hz), 8.0-8.15 (4H, m) MASS (m/z): 1334.4 (M+Na) Elemental Analysis Calcd. for C₆₄H₉₂Cl₃N₁₃O₁₅S.7H₂O: C, 49.66; H, 6.90; N, 11.76 Found: C, 49.76; H, 6.93; N, 11.78

### EXAMPLE 139

IR (KBr): 3347.8, 1633.4, 1517.7, 1442.5, 1257.4 cm⁻¹

NMR (DMSO-d₆+D₂O, δ): 0.96 (3H, d, J=6.7 Hz), 1.08 (3H, d, J=5.8 Hz), 1.33-4.79 (54H, m), 6.64-8.14 (11H, m)

ESI MASS (Positive)(m/z): 1328.4 (free)(M+Na)⁺

Elemental Analysis Calcd. for C₆₂H₈₉Cl₂N₁₁O₁₈S.5H₂O: C, 50.68; H, 6.79; N, 10.48 Found: C, 50.83; H, 6.72; N, 10.27

### EXAMPLE 140

IR (KBr): 3376.7, 1633.4, 1442.5, 1249.6 cm⁻¹

NMR (DMSO-d₆+D₂O, δ): 0.97 (3H, d, J=6.3 Hz), 1.09 (3H, d, J=5.7 Hz), 1.73-4.80 (41H, m), 6.63-8.18 (15H, m)

ESI MASS (Positive)(m/z): 1334.4 (free)(M+Na)⁺

Elemental Analysis Calcd. for C₆₃H₈₃Cl₂N₁₁O₁₈S.4H₂O: C, 51.92; H, 6.29; N, 10.57 Found: C, 51.83; H, 6.32; N, 10.13

### EXAMPLE 141

IR (KBr): 1633, 1608, 1522, 1443, 1421, 1277, 1254, 1198, 1082 cm⁻¹

NMR (DMSO-d₆+D₂O, δ): 0.8-1.2 (9H, m), 1.4-2.7 (25H, m), 2.8-4.6 (28H, m), 2.7-2.9 (2H, m), 6.6-6.8 (3H, m), 7.19 (2H, m), 7.94 (2H, m), 8.0-8.2 (4H, m)

ESI MASS (Positive): 1364.3 (M+Na)⁺

Elemental Analysis Calcd. for C₆₅H₉₄Cl₃N₁₃O₁₆S.10H₂O: C, 47.83; H, 7.04; N, 11.16 Found: C, 48.00; H, 7.12; N, 11.03

### EXAMPLE 142

IR (KBr): 3363, 1632, 1537, 1520, 1443, 1277, 1240, 1072 cm⁻¹

NMR (DMSO-d₆+D₂O, δ): 0.8-2.7 (30H, m), 0.96 (3H, d, J=6.7 Hz), 1.08 (3H, d, J=5.9 Hz), 2.8-4.6 (24H, m), 3.14 (3H, s), 4.7-4.9 (2H, m), 6.6-6.8 (3H, m), 7.12 (2H, d, J=9.0 Hz), 7.86 (2H, d, J=8.7 Hz), 8.0-8.2 (4H, m)

ESI MASS (Positive): 1379.4 (M+Na)⁺

Elemental Analysis Calcd. for C₆₆H₉₄Cl₂N₁₂O₁₇S.10H₂O: C, 49.22; H, 7.13; N, 10.44 Found: C, 49.26; H, 7.28; N, 10.41

### EXAMPLE 143

IR (KBr): 1633, 1537, 1524, 1443, 1275, 1250, 1084, 1063 cm⁻¹

NMR (DMSO-d₆+D₂O, δ): 0.97 (3H, d, J=6.8 Hz), 1.09 (3H, d, J=5.9 Hz), 1.6-2.7 (14H, m), 2.8-4.5 (26H, m), 3.33 (3H, s), 4.7-4.9 (2H, m), 6.6-6.8 (3H, m), 7.10 (2H, d, J=8.8 Hz), 7.74 (2H, d, J=8.8 Hz), 7.88 (2H, d, J=8.5 Hz), 8.0-8.2 (6H, m)

ESI MASS (Positive): 1364.3 (M+Na)⁺

Elemental Analysis Calcd. for C₆₄H₈₅Cl₂N₁₁O₁₉₉S6.5H₂O: C, 50.16; H, 6.45; N, 10.05 Found: C, 50.07; H, 6.59; N, 9.97

### EXAMPLE 144

IR (KBr): 3363, 1645, 1632, 1539, 1522, 1443, 1421, 1277, 1082 cm⁻¹

NMR (DMSO-d₆+D₂O, δ): 0.9-1.2 (9H, m), 1.5-2.7 (24H, m), 2.8-4.5 (30H, m), 4.7-4.9 (2H, m), 6.6-6.8 (3H, m), 7.19 (2H, d, J=8.9 Hz), 7.94 (2H, d, J=8.6 Hz), 8.0-8.2 (4H, m)

ESI MASS (Positive): 1395.4 (M+Na)⁺

Elemental Analysis Calcd. for C₆₆H₉₆Cl₃N₁₃O₁₇S.8H₂O: C, 48.75; H, 6.94; N, 11.20 Found: C, 48.66; H, 7.06; N, 11.14

### EXAMPLE 145

IR (KBr): 3350, 1632, 1537, 1522, 1443, 1072 cm⁻¹

NMR (DMSO-d₆+D₂O, δ): 0.9-2.7 (29H, m), 0.96 (3H, d, J=6.8 Hz), 1.08 (3H, d, J=5.9 Hz), 2.8-4.5 (26H, m), 3.10 (3H, s), 4.7-4.9 (2H, m), 6.6-6.8 (3H, m), 7.11 (2H, d, J=9.0 Hz), 7.85 (2H, d, J=8.8 Hz), 8.0-8.2 (4H, m)

ESI MASS (Positive): 1409.5 (M+Na)⁺

Elemental Analysis Calcd. for C₆₇H₉₆Cl₂N₁₂O₁₈S.9H₂O: C, 49.59; H, 7.08; N, 10.36 Found: C, 49.76; H, 7.04; N, 10.30

### EXAMPLE 146

IR (KBr): 3367.1, 1633.4, 1537.0, 1517.7, 1442.5 cm⁻¹

NMR (DMSO-d₆, δ): 0.96 (3H, d, J=6.7 Hz), 1.08 (3H, d, J=5.7 Hz), 1.33-5.24 (66H, m), 6.62-8.95 (17H, m)

ESI MASS (Positive)(m/z): 1320.4 (M+H)⁺

Elemental Analysis Calcd. for C₆₃H₉₁Cl₂N₁₁O₁₈S.4H₂O: C, 51.63; H, 6.81; N, 10.51 Found: C, 51.37; H, 7.08; N, 10.28

### EXAMPLE 147

IR (KBr): 3344.0, 1633.4, 1523.5, 1442.5 cm⁻¹

NMR (DMSO-d₆, δ): 0.96 (3H, d, J=6.7 Hz), 1.09 (3H, d, J=5.5 Hz), 1.50-5.27 (56H, m), 6.63-8.96 (21H, m)

ESI MASS (Positive)(m/z): 1326.4 (M+H)⁺, 1348.3 (M+Na)⁺

Elemental Analysis Calcd. for C₆₄H₈₅Cl₂N₁₁O₁₈S-4H₂O: C, 52.24; H, 6.37; N, 10.47 Found: C, 52.22; H, 6.53; N, 10.03

### EXAMPLE 148 ESI MASS (Positive): 1312.5 (M+Na)⁺

### EXAMPLE 149

To a mixture of starting compound (149) (0.1 g), dihydroxyacetone (27 mg) and acetic acid (26 μl) in N,N-dimethylformamide (0.7 ml) and methanol (1.5 ml) was added sodium cyanoborohydride (19 mg). After stirring for 144 hours at room temperature, the reaction mixture was poured into ethyl acetate. The resulting precipitate was collected and washed with ethyl acetate. The precipitate was dissolved in water (100 ml) that was adjusted to pH 1 and the solution was subjected to column chromatography on ODS (Daiso-gel, SP-120-40/60-ODS-B) (50 ml) eluting with 30% acetonitrile in water. The eluted fractions containing the desired product were collected and evaporated in vacuo. The residue was lyophilized to give object compound (149) (96.5 mg).

NMR (DMSO-d₆+D₂O, δ): 0.97 (3H, d, J=6.6 Hz), 1.10 (3H, d, J=5.7 Hz), 1.6-2.7 (13H, m), 2.8-4.5 (32H, m), 3.33 (3H, s), 4.7-4.9 (2H, m), 6.6-6.8 (3H, m), 7.10 (2H, d, J=8.8 Hz), 7.74 (2H, d, J=8.6 Hz), 7.88 (2H, d, J=8.3 Hz), 8.0-8.2 (6H, m)

ESI MASS (Positive): 1438.4 (M+Na)⁺

The following compounds [Example 150 to 157] were obtained according to a similar manner to that of Example 149.

### EXAMPLE 150

IR (KBr): 3350, 1649, 1632, 1539, 1524, 1450, 1441, 1421 cm⁻¹

NMR (DMSO-d₆+D₂O, δ): 0.9-1.2 (9H, m), 1.5-2.7 (25H, m), 2.8-4.6 (34H, m), 4.7-4.9 (2H, m), 6.6-6.8 (3H, m), 7.19 (2H, d, J=8.8 Hz), 7.94 (2H, d, J=8.7 Hz), 8.0-8.2 (4H, m)

ESI MASS (Positive): 1469.3 (M+Na)⁺

Elemental Analysis Calcd. for C₆₉H₁₀₂Cl₃N₁₃O₁₉S.10H₂O: C, 47.73; H, 7.08; N, 10.49 Found: C, 47.55; H, 7.09; N, 10.50

### EXAMPLE 151

IR (KBr): 3344.0, 1631.5, 1517.7, 1442.5 cm⁻¹ NMR (DMSO-d₆, δ): 0.87 (3H, t, J=7.0 Hz), 0.96 (3H, d, J=6.5 Hz), 1.06 (3H, d, J=5.0 Hz), 1.21-5.08 (76H, m), 6.63-8.85 (16H, m)

ESI MASS (Positive)(m/z): 1376.5 (M+H)⁺

### EXAMPLE 152

IR (KBr): 3342.0, 1627.6, 1546.6, 1517.7, 1444.4 cm⁻¹

NMR (DMSO-d₆, δ): 0.86 (3H, t, J=7.0 Hz), 0.96 (3H, d, J=6.6 Hz), 1.07 (3H, d, J=5.1 Hz), 1.14-4.63 (76H, m), 6.63-8.90 (15H, m) ESI MASS (Positive)(m/z): 1403.4 (M+H)⁺, 1483.5 (M+Na)⁺

### EXAMPLE 153

IR (KBr): 3342.0, 1633.4, 1519.6, 1496.5, 1442.5 cm⁻¹

NMR (DMSO-d₆, δ): 0.96 (3H, d, J=6.7 Hz), 1.07 (3H, d, J=5.5 Hz), 1.76-5.00 (76H, m), 6.63-8.82 (21H, m)

ESI MASS (Positive)(m/z): 1476.4 (M+Na)⁺

### EXAMPLE 154

IR (KBr): 3355.5, 1631.5, 1515.8, 1444.4 cm⁻¹

NMR (DMSO-d₆, δ): 0.96 (3H, d, J=6.5 Hz), 1.05 (3H, d, J=5.4 Hz), 1.40-4.76 (80H, m), 6.63-8.85 (16H, m) ESI MASS (Positive)(m/z): 1403.4 (M+H)⁺, 1425.4 (M+Na)⁺

### EXAMPLE 155

IR (KBr): 3367.1, 1633.4, 1519.6, 1440.6 cm⁻¹

NMR (DMSO-d₆, δ): 0.96 (3H, d, J=6.7 Hz), 1.06 (3H, d, J=5.4 Hz), 1.30-4.78 (85H, m), 6.63-9.00 (16H, m)

ESI MASS (Positive)(m/z): 1446.8 (M+H)⁺

### EXAMPLE 156

IR (KBr): 3328.5, 1633.4, 1517.7, 1444.4 cm⁻¹

NMR (DMSO-d₆, δ): 0.96 (3H, d, J=6.8 Hz), 1.08 (3H, d, J=5.2 Hz), 1.14-4.80 (78H, m), 6.64-8.90 (16H, m)

ESI MASS (Positive)(m/z): 1483.6 (M+Na)⁺

### EXAMPLE 157

IR (KBr): 3336.2, 1631.5, 1533.1, 1517.7, 1440.6 cm⁻¹

NMR (DMSO-d₆, δ): 0.96 (3H, d, J=6.7 Hz), 1.07 (3H, d, J=5.2 Hz), 1.35 (9H, s), 1.22-4.78 (76H, m), 6.63-8.82 (16H, m)

ESI MASS (Positive)(m/z): 1385.5 (M+Na)⁺

### EXAMPLE 158

To a solution of starting compound (158) (0.84 g) and cobalt(II) chloride hexahydrate (0.73 g) in a mixture of methanol (25 ml) and water (5 ml) was added sodium borohydride (0.58 g) with stirring at ambient temperature, and the mixture was stirred at the same temperature for 2 hours. The reaction mixture was filtered through celite. The filtrate was adjusted to pH 8 with 1N-hydrochloric acid, and evaporated under reduced pressure to remove solvent. To the resulting residue in a mixture of methanol (52 ml) and water (15 ml) was added 10% palladium on carbon (0.17 g), and hydrogen gas at atmosphere pressure for 2 hours. The reaction mixture was filtered through celite and evaporated under reduced pressure to remove methanol. To the resulting residue and N,N-diisopropylethylamine (0.67 ml) in N,N-dimethylformamide (17 ml) was added 9-fluorenylmethyl chloroformate (0.43 g) at ambient temperature, and the mixture was stirred for 16 hours. Water (160 ml) was added, and the mixture was adjusted to pH 7 with 1N-hydrochloric acid. The redulting precipitate was collected by filtration, and dried under reduced pressure to give object compound (158) (0.95 g).

IR (KBr): 3371.0, 1670.1, 1633.4, 1515.8, 1448.3 cm⁻¹

NMR (DMSO-d₆, δ): 0.89 (3H, d, J=6.3 Hz), 1.12 (3H, d, J=5.6 Hz), 1.34-5.21 (64H, m), 6.28-8.69 (23H, m)

ESI MASS (Positive)(m/z): 1478.3 (M+Na)⁺

The following compounds [Example 159 to 161] were obtained according to a similar manner to that of Example 158.

### EXAMPLE 159

NMR (DMSO-d₆+D₂O, δ): 0.98 (3H, d, J=6.3 Hz), 1.09 (3H, d, J=5.3 Hz), 1.35 (9H, s), 1.4-4.8 (35H, m), 6.2-6.8 (7H, m), 7.3-7.5 (8H, m), 7.8-7.9 (8H, m)

ESI MASS (Positive): 1420.4 (M+Na)⁺

### EXAMPLE 160

ESI MASS (Positive): 1464.4 (M+Na)⁺

### EXAMPLE 161

ESI MASS (Positive): 1126.3 (M+Na)⁺

### EXAMPLE 162

A mixture of starting compound (162) (790 mg), 2M methylamine in tetrahydrofuran (4.8 ml) and tetrahydrofuran (7.9 ml) was stirred for 25 hours at room temperature. To the solution was added 10% palladium on carbon (450 mg), methanol (24 ml), water (5 ml) and hydrogen gas at atmosphere pressure for 5.5 hours. The reaction mixture was filtered through celite. The filtrate was evaporated under reduced pressure to remove methanol and tetrahydrofuran and was lyophilized. To the resulting residue and N,N-diisopropylethylamine (0.332 ml) in N,N-dimethylformamide (16 ml) was added 9-fluorenylmethyl chloroformate (395 mg) at room temperature, and the mixture was stirred for 24 hours. 30% acetonitrile in water was added to the mixture. The solution was subjected to column chromatography on ODS eluting with 50% acetonitrile in water. The fractions containing the object compound were collected and evaporated under reduced pressure to remove acetonitrile. The residue was lyophilized to give object compound (162) (616 mg).

NMR (DMSO-d₆+D₂O, δ): 0.90 (3H, d, J=6.7 Hz), 1.10 (3H, d, J=5.1 Hz), 1.34 (9H, s), 1.4-4.5 (40H, m), 4.6-4.9 (2H, m), 6.25-7.95 (19H, m)

MASS (m/z): 1434.5 (M+Na)

### EXAMPLE 163

To a solution of starting compound (163) (570 mg) and triethylsirane (0.516 ml) in dichloromethane (8.5 ml) was added dropwise trifluoroacetic acid (0.933 ml) with stirring under ice-cooling, and the mixture was stirred at room temperature for 6.5 hours. Water was added to the mixture. The solution was subjected to column chromatography on ODS eluting with 30-50% acetonitrile in water. The fractions containing the object compound were collected and evaporated under reduced pressure to remove acetonitrile. The residue was lyophilized to give object compound (163) (480 mg).

NMR (DMSO-d₆+D₂O, δ): 0.90 (3H, d, J=6.6 Hz), 1.09 (3H, d, J=5.7 Hz), 1.15-4.5 (40H, m), 4.65-4.85 (2H, m), 6.25-7.95 (19H, m)

ESI MASS (m/z): 1334.4 (M+Na)

### EXAMPLE 164

To a solution of starting compound (164) (0.93 g) and triethylsilane (0.82 ml) in dichloromethane (14 ml) was added dropwise trifluoroacetic acid (1.48 ml) with stirring under ice-cooling, and the mixture was stirred at ambient temperature for 6 hours. The reaction mixture was poured into water. The resulting precipitate was collected by filtration, and the precipitate was dissolved in a mixture of 35% acetonitrile in water and 1N-sodiumhydroxide solution. The solution was subjected to column chromatography on ODS (Daiso-gel, SP-120-40/60-ODS-B (Trademark: prepared by Daiso Co., Ltd.)) (25 ml) eluting with 50% acetonitrile in water. The fractions containing the object compound were collected and evaporated under reduced pressure to remove acetonitrile. The residue was lyophilized to give object compound (164) (0.63 g).

IR (KBr): 3361.3, 1673.9, 1639.2, 1517.7, 1444.4 cm⁻¹

NMR (DMSO-d₆, 6): 0.90 (3H, d, J=6.6 Hz), 1.11 (3H, d, J=5.8 Hz), 1.23-5.21 (54H, m), 6.28-8.68 (25H, m) ESI MASS (Positive)(m/z): 1356.5 (M+H)⁺, 1378.4 (M+Na)⁺

The following compound was obtained according to a similar manner to that of Example 164.

### EXAMPLE 165

IR (KBr): 3394.1, 3332.4, 1675.8, 1633.4, 1515.8, 1446.4, 1270.9 cm⁻¹

NMR (DMSO-d₆+D₂O, δ): 0.90 (3H, d, J=6.5 Hz), 1.11 (3H, d, J=5.9 Hz), 1.50-4.82 (43H, m), 6.51-7.90 (19H, m)

ESI MASS (Positive)(m/z): 1364.4 (M+Na)⁺, 1342.5 (M+H)⁺

### EXAMPLE 166

A mixture of starting compound (166) (0.1 g), N,N′-bis(tert-butoxycarbonyl)-N″-triflylguanidine (0.15 g) and triethylamine (52 μl) in N,N-dimethylformamide (2 ml) was stirred for 79 hours at room temperature. The reaction mixture was poured into water (40 ml). The resulting precipitate was collected and washed with water. The precipitate was purified by column chromatography on silica gel eluting with a mixture of dichloromethane and methanol (5:1). The eluted fraction containing the desired product were collected and evaporated in vacuo to give object compound (166) (42.2 mg).

NMR (DMSO-d₆+D₂O, δ): 0.96 (3H, d, J=6.8 Hz), 1.14 (3H, d, J=6.0 Hz), 1.40 (9H, s), 1.41 (9H, s), 1.42 (9H, s), 1.48 (9H, s), 1.5-2.6 (9H, m), 2.9-5.8 (28H, m), 3.33 (3H, s), 6.5-6.8 (3H, m), 7.09 (2H, d, J=8.8 Hz), 7.74 (2H, d, J=8.8 Hz), 7.87 (2H, d, J=8.5 Hz), 8.0-8.2 (6H, m)

ESI MASS (Positive): 1774.6 (M+Na)⁺

The following compound was obtained according to a similar manner to that of Example 19.

### EXAMPLE 167

NMR (DMSO-d₆+D₂O, δ): 0.9-1.2 (6H, m), 1.5-2.8 (9H, m), 3.1-4.9 (28H, m), 3.33 (3H, s), 6.6-6.8 (3H, m), 7.10 (2H, d, J=8.6 Hz), 7.74 (2H, d, J=8.5 Hz), 7.88 (2H, d, J=8.3 Hz), 8.0-8.2 (6H, m)

ESI MASS (Positive): 1374.4 (M+Na)⁺

The following compound was obtained according to a similar manner to that of Example 21.

### EXAMPLE 168

IR (KBr): 3359, 3352, 1633, 1518, 1443, 1275 cm⁻¹

NMR (DMSO-d₆+D₂O, δ): 0.9-1.2 (9H, m), 1.5-2.7 (21H, m), 2.8-4.5 (30H, m), 4.7-4.9 (2H, m), 6.5-6.8 (3H, m), 7.19 (2H, d, J=9.0 Hz), 7.94 (2H, d, J=8.7 Hz), 8.0-8.2 (4H, m)

ESI MASS (Positive): 1377.6 (M+Na)⁺

The following compound was obtained according to a similar manner to that of Example 149.

### EXAMPLE 169

IR (KBr): 3344.0, 1633.4, 1604.5, 1517.7, 1442.5 cm⁻¹

NMR (DMSO-d₆, δ): 0.96 (3H, d, J=6.8 Hz), 1.08 (3H, d, J=5.5 Hz), 1.19-5.68 (77H, m), 6.64-8.85 (14H, m)

ESI MASS (Positive)(m/z): 1455.4 (M+Na)⁺

### EXAMPLE 170

A solution of starting compound (170) (100 mg), N-(tert-butoxycarbonyl)glycine (27.7 mg), 1-ethyl-3-(3′-dimethylaminopropyl)carbodiimide (27.8 mg) and 1-hydroxybenzotriazole (24.2 mg) in DMF (2 ml) was stirred at room temperature for 2.5 hours. To the reaction mixture was added ethyl acetate (20 ml). The resulting precipitate was collected by filtration. The precipitate was dissolved methanol (3 ml). To the solution was added 10% hydrogenchloride in methanol (1 ml), and stirred at room temperature overnight. To the reaction mixture was added ethyl acetate (50 ml); The resulting precipitate was collected by filtration. The precipitate was dissolved water (20 ml), and the solution was subjected to column chromatography on ODS (Daiso-gel, SP-120-40/60-ODS-B (Trademark: prepared by Daiso Co., Ltd.)) (20 ml) eluting with 10% acetonitrile in diluted HCl (pH 3). The fractions containing the object compound were collected and evaporated under reduced pressure to remove acetonitrile, residue was lyophilized to give object compound (170) (52 mg).

IR (KBr): 3311.2, 1633.4, 1602.6, 1513.8, 1440.6 cm⁻¹

NMR (DMSO-d₆, δ): 0.96 (3H, d, J=6.6 Hz), 1.10 (3H, d, J=5.3 Hz), 1.19-4.75 (69H, m), 6.58-8.79 (18H, m), 11.08 (1H, bs)

ESI MASS (Positive)(m/z): 1420.4 (M+Na)⁺

1. A lipopeptide compound of the following general formula (I): 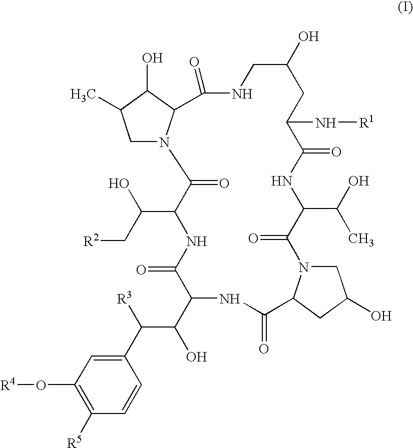
wherein
R¹ is hydrogen or acyl group,
R² is carbamoyl, protected amino(lower)alkyl, amino(lower)alkyl, protected guanidino(lower)alkyl, guanidino(lower)alkyl or lower alkylamino(lower)alkyl substituted with one or more hydroxy,
R³ is hydrogen or hydroxy,
R⁴ is protected amino (lower)alkyl, amino (lower)alkyl, heterocycliccarbonyl(lower)alkyl, lower alkylcarbamoyl(lower)alkyl, protected carboxy(lower)alkyl, carboxy(lower)alkyl, protected guanidino(lower)alkyl, guanidino(lower)alkyl, di-lower alkylamino(lower)alkyl, lower alkylamino(lower)alkyl optionally substituted with one or more suitable substituent(S) selected from the group consisting of hydroxy and lower alkoxy, amino(lower)alkanoylamino(lower)alkyl or hydroxy(lower)alkyl, and
R⁵ is protected hydroxy or hydroxy, or a salt thereof.

2. A compound of claim 1, wherein
R¹ is acyl group,
R² is amino(lower)alkyl or lower alkylamino(lower)alkyl substituted with two hydroxy,
R³ is hydrogen,
R⁴ is amino(lower)alkyl or lower alkylamino(lower)alkyl substituted with one or two hydroxy, and
R⁵ is hydroxy.

3. A compound of claim 2, wherein
R¹ is benzoyl substituted with a substituent selected from the group consisting of
1) thiadiazolyl substituted with phenyl substituted with piperidyl substituted with one or two substituent(s) selected from the group consisting of lower alkoxy, cyclo(lower)alkyl, lower alkoxy(lower)alkoxy(lower)alkyl, lower alkoxy(lower)alkoxy, lower alkoxy(lower)alkyl substituted with phenyl and cyclo(lower)alkyl(lower)alkyloxy,
2) thiadiazolyl substituted with phenyl substituted with phenyl substituted with lower alkoxy(lower)alkoxy,
3) thiadiazolyl substituted with phenyl substituted with piperazinyl substituted with a substituent selected from the group consisting of cyclo(lower)alkyl substituted with lower alkyl, cyclo(lower)alkyl(lower)alkyl, cyclo(lower)alkyl, cyclo(higher)alkyl, lower alkoxy(higher)alkyl and cyclo(lower)alkyl substituted with lower alkylidene,
4) thiadiazolyl substituted with pyridyl substituted with piperazinyl substituted with cyclo(lower)alkyl substituted with lower alkyl,
5) imidazothiadiazolyl substituted with phenyl substituted with piperidyl substituted with lower alkoxy(lower)alkoxy,
6) phenyl substituted with piperazinyl substituted with cyclo(lower)alkyl optionally substituted with one or two substituent(s) selected from the group consisting of cyclo(lower)alkyl, lower alkoxy, lower alkyl and phenyl,
7) imidazothiadiazolyl substituted with phenyl substituted with lower alkoxy,
8) phenyl substituted with a substituent selected from the group consisting of piperidyl substituted with cyclo(lower)alkyloxy and phenyl substituted with morpholino,
8) piperazinyl substituted with phenyl substituted with phenyl substituted with lower alkoxy(lower)alkoxy
9) piperazinyl substituted with cyclo(lower)alkyl substituted with lower alkyl, and
10) phenyl substituted with piperazinyl substituted with phenyl substituted with heterocyclic group substituted with lower alkyl and lower alkoxy, or heterocycliccarbonyl substituted with heterocyclic group substituted with lower alkoxy(lower)alkoxy.

4. A compound of claim 3, wherein
R¹ is benzoyl substituted with a substituent selected from the group consisting of
1) thiadiazolyl substituted with phenyl substituted with piperidyl substituted with a substituent selected from the group consisting of cyclo(lower)alkyl substituted with lower alkyl and cyclo(lower)alkyl, and
2) thiadiazolyl substituted with pyridyl substituted with piperazinyl substituted with cyclo(lower)alkyl substituted with lower alkyl.

5. A process for preparing a lipopeptide compound (I) of claim 1, or a salt thereof,
which comprises,
i) reacting a compound (II) of the formula: 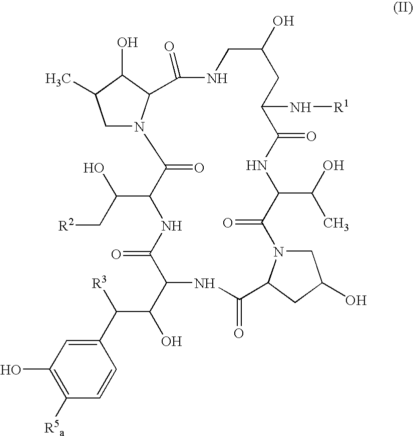
wherein R¹, R² and R³ are defined in claim 1,
Rₐ⁵ is protected hydroxy,
or its reactive derivative at the hydroxy group or a salt thereof, with a compound (VIII) of the formula:
R⁴—X (VII)
wherein R⁴ is defined in claim 1,
X is halogen,
or its reactive derivative or a salt thereof, to give a compound (Ia) of the formula: 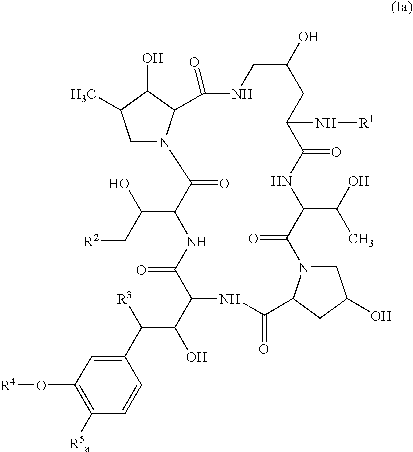
wherein R¹, R², R³ and R⁴ are defined in claim 1,
Ra⁵ is defined above,
or a salt thereof, or
ii) subjecting a compound (Ia) of the formula: 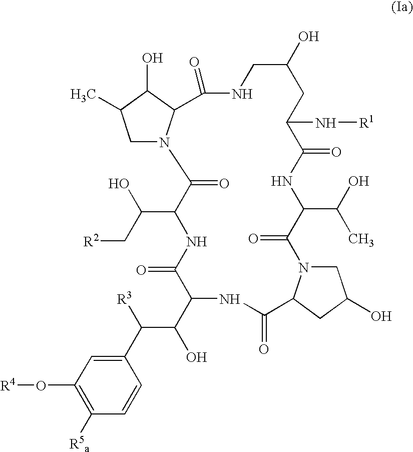
wherein R¹, R², R³ and R⁴ are defined in claim 1,
Rₐ⁵ is protected hydroxy,
or a salt thereof, to elimination reaction of the hydroxy protective group, to give a compound (Ib) of the formula: 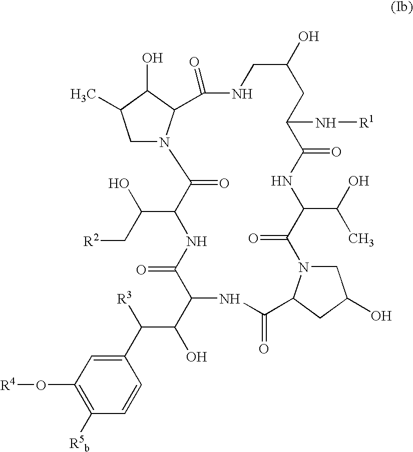
wherein R¹, R², R³ and R⁴ are defined in claim 1,
R_{b}⁵ is hydroxy,
or a salt thereof.
iii) subjecting a compound (Ic) of the formula: 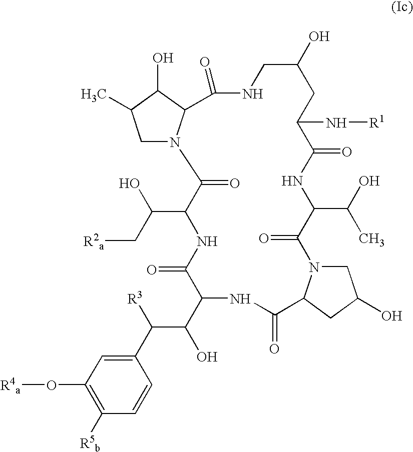
wherein R¹ and R³ are defined in claim 1,
Rₐ² is protected amino(lower)alkyl or protected guanidino(lower)alkyl,
Rₐ⁴ is protected amino(lower)alkyl or protected guanidino(lower)alkyl, and
R_{b}⁵ is hydroxy,
or a salt thereof, to elimination reaction of the amino protective group,
to give a compound (Id) of the formula: 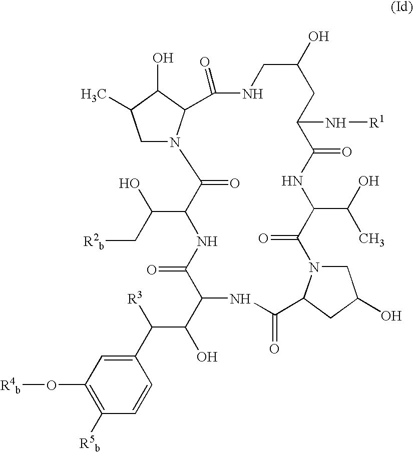
wherein R¹ and R³ are defined in claim 1,
R_{b}² is amino(lower)alkyl or guanidino(lower)alkyl,
R_{b}⁴ is amino(lower)alkyl or guanidino(lower)alkyl and,
R_{b}⁵ is hydroxy,
or a salt thereof, or
iv) reacting a compound (Ie) of the formula: 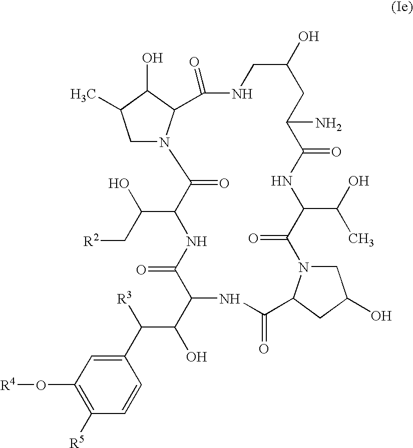
wherein R², R³, R⁴ and R⁵ are defined in claim 1, or its reactive derivative at the amino group or a salt thereof, with a compound (VIII) of the formula:
Rₐ¹—OH (VIII)
wherein Rₐ¹ is acyl group,
or its reactive derivative at the carboxy group or a salt thereof, to give a compound (If) of the formula: 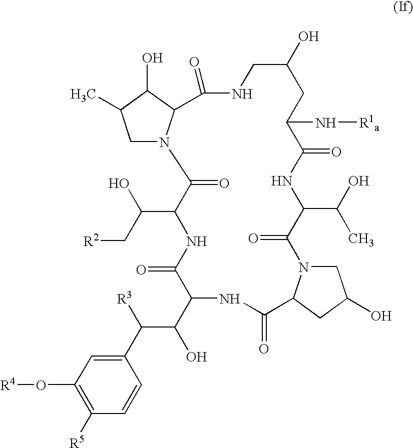
wherein R², R³, R⁴ and R⁵ are defined in claim 1,
Rₐ¹ is defined above,
or a salt thereof, or
v) reacting a compound (Ig) of the formula: 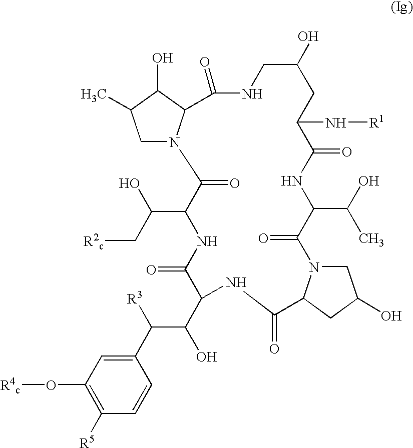
wherein R¹, R³ and R⁵ are defined in claim 1,
R_{c}² is amino(lower)alkyl, and
R_{c}⁴ is amino(lower)alkyl
or its reactive derivative at the amino group or a salt thereof, with a compound (IX) of the formula:
R⁶═O (IX)
wherein R⁶ is lower alkyl substituted with one or more hydroxy,
or its reactive derivative or a salt thereof, to give a compound (Ih) of the formula: 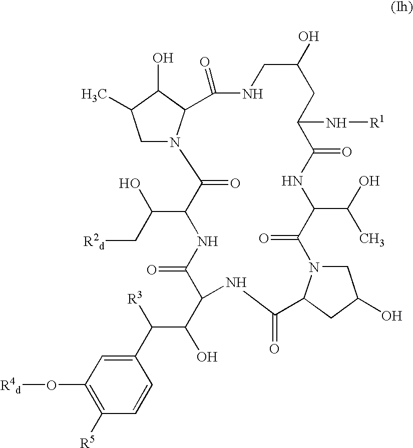
wherein R¹, R³ and R⁵ are defined above,
R_{d}² is lower alkylamino(lower)alkyl substituted with one or more hydroxy, and
R_{d}⁴ is lower alkylamino(lower)alkyl substituted with one or more hydroxy
or a salt thereof.

6. A pharmaceutical composition which comprises, as an active ingredient, a compound of claim 1 or a pharmaceutically acceptable salt thereof in admixture with pharmaceutically acceptable carriers or excipients.

7. Use of a compound of claim 1 or a pharmaceutically acceptable salt thereof for the manufacture of a medicament.

8. A compound of claim 1 or a pharmaceutically acceptable salt thereof for use as a medicament.

9. A method for the prophylactic and/or therapeutic treatment of infectious diseases caused by pathogenic microorganisms, which comprises administering a compound of claim 1 or a pharmaceutically acceptable salt thereof to a human being or an animal.

10. A commercial package comprising the pharmaceutical composition of claim 6 and a written matter associated therewith, wherein the written matter states that the pharmaceutical composition can or should be used for preventing or treating infectious disease.

11. An article of manufacture, comprising packaging material and the compound (I) identified in claim 1 contained within said packaging material, wherein said compound (I) is therapeutically effective for preventing or treating infectious diseases, and wherein said packaging material comprises a label or a written material which indicates that said compound (I) can or should be used for preventing or treating infectious diseases.
